SEQUENCE LISTING (450 sequences)

SEQ ID NO: 1 - DNA, 279 bp, Triticum aestivum
atgtcgagccgtaggtcaaggtcaaggcagtccggctcgtcgaggatcactgacgagcaaatcagcgaccttgtctccaagttgcaggacctccttcccgaggcgcgtctccggggcaatgatagagtgccatcttcaagggtgctgcaggagacgtgcacctacatcaggagcctgcaccgggaggtggacgacctgagcgagaggctgtcggagctgctggcgacctcggacatgagcagcgcgcaagcggccatcatccgcagcttgctgatgtag

SEQ ID NO: 2 - protein, 92 aa, Triticum aestivum
MSSRRSRSRQSGSSRITDEQISDLVSKLQDLLPEARLRGNDRVPSSRVLQETCTYIRSLHREVDDLSERLSELLATSDMSSAQAAIIRSLLM

SEQ ID NO: 3 - DNA, 54 bp, Artificial sequence
  primer: prm09663
ggggacaagtttgtacaaaaaagcaggcttaaacaatgtcgagccgtaggtcaa

SEQ ID NO: 4 - DNA, 48 bp, Artificial sequence
  primer: prm09664
ggggaccactttgtacaagaaagctgggtccggctctacatcagcaag

SEQ ID NO: 5 - DNA, 2194 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctccttctcccatctataaattcctccccccttttcccctctctatataggaggcatccaagccaagaagagggagagcaccaaggacacgcgactagcagaagccgagcgaccgccttctcgatccatatcttccggtcgagttcttggtcgatctcttccctcctccacctcctcctcacagggtatgtgcctcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggatcggaatcttgcgattttgtgagtaccttttgtttgaggtaaaatcagagcaccggtgattttgcttggtgtaataaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 6 - DNA, 2527 bp, Artificial sequence
  expression cassette
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctcctcctcccatctataaattcctccccccttttcccctctctatataggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttcatttaaatcaactagggatatcacaagtttgtacaaaaaagcaggcttaaacaatgtcgagccgtaggtcaaggtcaaggcagtccggctcgtcgaggatcactgacgagcaaatcagcgaccttgtctccaagttgcaggacctccttcccgaggcgcgtctccggggcaatgatagagtgccatcttcaagggtgctgcaggagacgtgcacctacatcaggagcctgcaccgggaggtggacgacctgagcgagaggctgtcggagctgctggcgacctcggacatgagcagcgcgcaagcggccatcatccgcagcttgctgatgtagag

SEQ ID NO: 7 - protein, 16 aa, Artificial sequence
  motif 1
  VARIANT (1)..(1): / replace = “Asp” / replace = “Asn”
  UNSURE (2)..(2): Xaa can be any naturally occurring amino acid
  VARIANT (3)..(3): / replace = “Gln”
  VARIANT (4)..(4): / replace = “Val” / replace = “Met”
  UNSURE (5)..(5): Xaa can be any naturally occurring amino acid
  VARIANT (6)..(6): / replace = “Asp” / replace = “Gln” / replace = “Ala” / replace =“Asn”
  VARIANT (7)..(7): / replace = “Phe” / replace = “Ile”
  VARIANT (8)..(8): / replace = “Val” / replace = “Leu” / replace = “Met”
  VARIANT (9)..(9): / replace = “Ile” / replace = “Thr” / replace = “Leu” /  replace =“Tyr”
  UNSURE (10)..(10): Xaa can be any naturally occurring amino acid
  VARIANT (12)..(12): / replace = “Arg” / replace = “His”
  UNSURE (13)..(13): Xaa can be any naturally occurring amino acid
  VARIANT (14)..(14): / replace = “Phe” / replace = “Ile” / replace = “Ser”
  VARIANT (15)..(15): / replace = “Val” / replace = “Ile”
  VARIANT (16)..(16): / replace = “Ala”
EXEIXELISXLQXLLP

SEQ ID NO: 8 - protein, 16 aa, Artificial sequence
  motif 2
  VARIANT (1)..(1): / replace = “Thr” / replace = “Ser”
  UNSURE (2)..(2): Xaa can be any naturally occurring amino acid
  VARIANT (3)..(3): / replace = “Arg” / replace = “Asn” / replace = “Ser”
  VARIANT (4)..(4): / replace = “Leu” / replace = “Ile” / replace = “Met” / replace = “Ala”
  VARIANT (6)..(6): / replace = “Lys” / replace = “Arg” / replace = “Glu” / replace = “His”
  VARIANT (7)..(7): / replace = “Asp” / replace = “Tyr” / replace = “Gln”
  VARIANT (10)..(10): / replace = “Ser” / replace = “Thr” / replace = “Ile” / replace = “Ala”
  VARIANT (11)..(11): / replace = “Ser” / replace = “Cys”
  VARIANT (12)..(12): / replace = “Phe” / replace = “Val”
  VARIANT (13)..(13): / replace = “Lys” / replace = “Gly”
  VARIANT (14)..(14): / replace = “Asn” / replace = “Asp” / replace = “Thr” / replace = “Arg”
  VARIANT (15)..(15): / replace = “Ser”
  VARIANT (16)..(16): / replace = “Gln” / replace = “Asn” / replace = “Ser”
AXKVLQETCNYIRSLH

SEQ ID NO: 9 - protein, 8 aa, Artificial sequence
  motif 3
  VARIANT (1)..(1): / replace = “Gln”
  VARIANT (3)..(3): / replace = “Glu”
EAAIIRSL

SEQ ID NO: 10 - protein, 11 aa, Artificial sequence
  motif 4
  VARIANT (3)..(3): / replace = “Gly”
  VARIANT (5)..(5): / replace = “Lys”
  VARIANT (11)..(11): / replace = “Thr”
MSSRRSRSRQS

SEQ ID NO: 11 - protein, 8 aa, Artificial sequence
  motif 5
  VARIANT (1)..(1): / replace = “Gln”
  VARIANT (3)..(3): / replace = “His”
  VARIANT (4)..(4): / replace = “Gln” / replace = “Arg”
KLQDLLPE

SEQ ID NO: 12 - protein, 8 aa, Artificial sequence
  motif 6
  VARIANT (3)..(3): / replace = “Asp”
  VARIANT (6)..(6): / replace = “Asn” / replace = “Ser”
LQETCTYI

SEQ ID NO: 13 - protein, 13 aa, Artificial sequence
  motif 7
  VARIANT (3)..(3): / replace = “Gly”
  VARIANT (11)..(11): / replace = “Gln”
EVDDLSERLSELL

SEQ ID NO: 14 - protein, 9 aa, Artificial sequence
  motif 8
  VARIANT (4)..(4): / replace = “Val” / replace = “Leu”
  VARIANT (7)..(7): / replace = “Asn” / replace = “Arg”
QAAIIRSLL

SEQ ID NO: 15 - protein, 90 aa, Populus trichocarpa
MSSRRPRQSSVPRITDDQIIDLVSKLRQLLPEISQRRSDKVSASKVLQETCNYIRNLHREVDDLSERLSQLLATIDADSPEAAIIRSLIM

SEQ ID NO: 16 - DNA, 383 bp, Populus trichocarpa
cagacgcgtaacaaaaatccgtgtgtaggcatgtctagcagaaggccaaggcaatctagcgttccaaggatcactgatgatcagatcatcgaccttgtctccaaattacgccagcttctccctgagattagtcaaaggcgctccgataaggtatcagcttccaaggtcctacaagagacttgcaattatatcaggaacttgcacagggaggttgatgacttaagtgagcgattgtctcagcttttggcaacaattgatgctgatagtcctgaagcagcgataataaggagtttaattatgtaatatcaattaattagatgatcaggcaccggcccttaaaccgatttatatctattttcagtttaataatttgttagtaggct

SEQ ID NO: 17 - protein, 86 aa, Allium cepa
MSSRRSRISEEEIGELISKLQSLLPDSRRRGSNRASASKLLKETCNYIKSLHREVDDLSERLSELISTMDNGSEQAEIIRSLLRSN

SEQ ID NO: 18 - DNA, 522 bp, Allium cepa
aattcttcctctctctcatttcacactttgcattttccacaatgtcgagtcgaaggtccagaattagcgaggaagagatcggagagctcatttcaaagctgcagtctctccttcccgattcacgtaggcgcggttcaaaccgggcatcggcgtccaagttgctaaaggagacgtgcaactacattaagagcttgcacagagaagtcgacgacttgagtgagaggctttctgaactgatctctaccatggacaatggaagcgagcaagctgagatcattcgaagcttgcttcgttctaactaaagtatggtcatgactgatttgaattgaattactcttaaatatgatatattttagcttttgaaagtttagctactagtggcagttgtagtagaaatgttggtgttttttttttccctttctttttcatctttatattaattgtgtacatttattttaatggtttggatcgagtttgttgcttctataaatgacaaaccgaccaacatctctccaaaaaaaa

SEQ ID NO: 19 - protein, 91 aa, Antirrhinum majus
MSGRRSRQSTGSSRISNDQIIDLVSKLHQLLPEIGNRRRSNKTSANKVLQETCNYIKNLHKEVDDLSERLSQLLSTIDADSPEAAIIRSLI

SEQ ID NO: 20 - DNA, 676 bp, Antirrhinum majus
ccctctgtacaactaaacttttatctcaagtcttcttttcacttttctgcgccctgtttcttatattaatctactacctatttaattattaactagtttaattagactttttataaaaaagaaaagaagagaatatttttaaggatgtctggaagaagatcaaggcagtcaacggggagttcaaggatttcaaatgatcaaatcattgaccttgtgtccaaactccaccagctccttcctgaaattggcaacagaaggcgttcaaacaagacatcagccaataaagttcttcaggagacttgcaactacatcaagaacttgcacaaagaagtggatgatttgagcgagaggctttcccagctactgtctactatagatgcggatagcccagaggccgcaataatcaggagtttaatttagttaattagtgtaataatgaagttattattggaagaagccaattttatgtattaattagctagatttttatctaggctgtgacttctgcatgggtttaatcaggcattaagacctaattactagtaggtttccctagccattaattgttgggtgcaactatatatgcagcaattaagtttgtagtttaattcgtactgtgtaataagggagctgtactttgcgatagttcctatattgattgtgttgtatttaaatt

SEQ ID NO: 21 - protein, 94 aa, Arabidopsis thaliana
MSSRRSSRSRQSGSSRISDDQISDLVSKLQHLIPELRRRRSDKVSASKVLQETCNYIRNLHREVDDLSDRLSELLASTDDNSAEAAIIRSLLNY

SEQ ID NO: 22 - DNA, 689 bp, Arabidopsis thaliana
aacaccttcttctccactctcattctctctttctgacacattaactacttatccttcttgcattcttctctctctctacacccaaacaaacacacttataatatatcaagaaagaagatgtctagcagaagatcatcacgttcaagacagtcaggaagctcaagaatctctgacgatcagatttccgatcttgtttctaagctccaacacctcatccctgaacttcgccgccgccgttctgacaaggtgtcagcatctaaggtactacaagagacttgcaactacatcaggaacttacacagagaggttgatgacctcagtgaccgtttgtcggaactcttggcttcgacggacgacaacagcgccgaagcagccatcattaggagcttgcttaattattaaatccgcattacttaatctgagagctattaatcatccgtttccggccaccaaatttatcttattatgggtatcgtctgtttacttctacatcatatattatgagatatagctagggtttcgggtcattgttaggccaactcatatatttatatttaatatatggttatgtatgtatgtatgcatgttaattgtatctgagggtccagacctggcgtatagtagcctgtgtatcatgagatcctctaatatttatgattaatgacacggtccgtttccttttttactat

SEQ ID NO: 23 - protein, 93 aa, Arabidopsis thaliana
MSGRRSRSRQSSGTSRISEDQINDLIIKLQQLLPELRDSRRSDKVSAARVLQDTCNYIRNLHREVDDLSERLSELLANSDTAQAALIRSLLTQ

SEQ ID NO: 24 - DNA, 558 bp, Arabidopsis thaliana
atactatcaacttttctctatctatctctctctcttctttttccggcataacttctgtgttaccctaaactccataacctgtttcaccgataaagtgcctttgcttctatctctgtcactcttactacttgttgaacaatattctacaaaaaaatgtcgggaagaagatcacgttcgaggcaatcatcaggaacttcaaggatctcagaagatcaaatcaatgatctgattatcaagttgcaacagcttcttcctgagctcagggacagtcgtcgttccgacaaggtttcagcagcgagggtgttacaagatacgtgcaactacatacggaatctgcatagagaggttgatgatctaagtgagaggctatctgagttactagcaaactcagacactgcacaagctgctttaatcagaagcttacttacccaataattcctatctatctttttcttcttcttcttttttttgtttactataataataataatagtttgcgggtttttttttctatagatgttgatgaccttataaacgtttaatgatacgagttcgtca

SEQ ID NO: 25 - protein, 92 aa, Arabidopsis thaliana
MSSRKSRSRQTGASMITDEQINDLVLQLHRLLPELANNRRSGKVSASRVLQETCSYIRNLSKEVDDLSERLSQLLESTDSAQAALIRSLLMQ

SEQ ID NO: 26 - DNA, 279 bp, Arabidopsis thaliana
atgtctagcagaaaatcacgttcaagacaaactggagcttccatgatcacggatgaacaaatcaacgatcttgtcctccagcttcatcggcttctccccgaacttgctaacaacagacgctctggaaaggtttcagcatcaagggtattacaagagacatgcagttacataaggaacttgagcaaagaagtggatgatcttagtgaaagattgtctcaacttttggaatcaactgattcagctcaagctgcactaatccgaagtttgcttatgcagtag

SEQ ID NO: 27 - protein, 92 aa, Arabidopsis thaliana
MSNRRSRQTSNASRISDDQMIDLVSKLRQFLPEIHERRRSDKVSASKVLQETCNYIRKLHREVDNLSDRLSQLLDSVDEDSPEAAVIRSLLM

SEQ ID NO: 28 - DNA, 739 bp, Arabidopsis thaliana
tatatctcgaagtgtctctattacccgaaacactttcttacaattttctcttctcttctcttttcgttgctcttctttttctttctttcacacctcttcaacacaaatataaaacctgtagaataaacacaaaccttctacataacttctctcacttttttttttttaaaactctcttcttaataacaaacccttctctctcaatctcttctctattatctaatctagaaaagagagaaagcatacaacataaaggttattttcttgcggcattgtagtgttacacctaatcacaaagtaaaaacaagaaaatgtctaacagaagatcaagacaaacttcgaatgcttcgaggatctccgatgaccagatgatcgacctcgttagtaagctccgtcagtttttgccggagattcacgaacggcgtcgttctgataaggtgtcagcatcaaaggtactacaagagacatgcaactacataagaaaattgcatagagaagttgacaatctcagtgatcgtttgtcgcagcttcttgactctgttgatgaagatagccctgaagctgccgtgattagaagcttactcatgtaaccttccaatattttttattataacttcttaatatagtatttattaatttatctatatatgtaatctttatcgtcctttatatatcaagcgacgtgcttttatcttttatgaactttggaattttggtacagaaatttacattaatttct

SEQ ID NO: 29 - protein, 92 aa, Arabidopsis thaliana
MSNRRSRQSSSAPRISDNQMIDLVSKLRQILPEIGQRRRSDKASASKVLQETCNYIRNLNREVDNLSERLSQLLESVDEDSPEAAVIRSLLM

SEQ ID NO: 30 - DNA, 703 bp, Arabidopsis thaliana
gtgtattcaaaaccccaaaacacttttctcattctcttctctattttcttcttgctctctagtttttctttcttcttggtcgtttcctttcagcataaaaaccttataaaatcataaaagcttacacctacttgccacatagacatagccgatctcattatatctctatttctatttctcaatagaacttgtttgagctagtgtgagagaagtaaagaaagagagaagaatccacaacttagttagggtcttttcttgccacattgttgaacatgtcgaacagaagatcaaggcaatcttcaagtgctccaaggatctccgataatcaaatgattgacctcgtatctaagctccgtcaaattttgccggagattggtcaacgacgtcgttctgataaggcatcagcctcgaaagtattgcaagagacatgcaattacatacgaaatttgaacagagaagttgacaatctgagcgagcgtttgtctcagcttctcgaatctgtcgatgaagatagccctgaagccgccgttattagaagcctactcatgtaatcttttttgttcttttgtttgtttttgacaagcctatccatgtaatcttaaatgatcgctctataataattatatttttaacataatcgtcttattatgtaaaattcaaagagatgggcttgatctttaatgacatacgaatttcatagggt

SEQ ID NO: 31 - protein, 94 aa, Arabidopsis thaliana
MSSSRRSRQASSSSRISDDQITDLISKLRQSIPEIRQNRRSNTVSASKVLQETCNYIRNLNKEADDLSDRLTQLLESIDPNSPQAAVIRSLING

SEQ ID NO: 32 - DNA, 601 bp, Arabidopsis thaliana
ctccctttctttcgacaagcacaaacaaagccatcaagagaagaaagccttttcttggattcacatatatataagaatattttttcaaatcaaacatgtcttctagcagaaggtcgagacaagcaagctcatcatcaagaattagcgatgaccagatcactgatctcatctcaaagctccgacagtccattccggagattcgccagaaccgtcgttccaacacggtatcagcgtcgaaagtgttacaagagacttgcaactacataagaaacttgaacaaggaagccgatgacctcagtgatcgattgactcagcttctggaatccattgatcctaatagcccacaagccgcagttattaggagcttgattaatggataattaagatataaattgattagttgtgctttatatatataagcttaaaatctcgttgggaggttgatccatcagggtgttgcataattatatatctattttatgtttcttatatattatttacaatcctatctagttagggttcatattttgacccttttttggtttaacgtcatgcatgcaattccattaagcttaaaaattataataaataagatttcgag

SEQ ID NO: 33 - protein, 90 aa, Brachypodium distachyon
MSGRRSSSRGNSVSEEEINELISKLQSLLPASARRRGSSQASTTKLLKETCSYIKSLHREVDDLSDRLSDLMATMDHNSPGAEIIRSLLR

SEQ ID NO: 34 - DNA, 603 bp, Brachypodium distachyon
ccacgcgtccgcaaaaacaaacttcagctaaccggccactcgatctacttttgggatcacacgcgcctagcttctcgtcgatcgtcttcaagctcagttcagtcctctttctgccgggctaggcgcgggctgcattattcagagacgtagtacgacgatgtcgggcaggaggtcgtcgtcccgcggtaactccgtgtcggaggaggagatcaacgagctcatctccaagctccagtctttgctcccggccagcgcgcgccgccgcggcagcagccaggcgtcgacgacgaagctgctcaaggagacgtgcagctacatcaagagcctgcaccgggaagtggacgacctgagcgaccggctctccgacctcatggccaccatggaccacaacagccccggcgccgagatcatccgcagccttctccgctagcttaattctctcatgcatgcatggtcgaccacgcccggcctcctgatagatcgatgtgatgtcctaattaattaagctagctcctcacctatataaatatatatgtatacatatacacatgatatatctgtgtccatcgatcgatctctgcatatacatgccgatcgatcgat

SEQ ID NO: 35 - protein, 92 aa, Cathamus tinctorius
MSSRRSRQSSSGGPRITDDQIIQLVSKLQQLLPGTRIQRSNKASASKVLQETCNYVRSLHREVDDLSDRLSQLLSTIDADSPEASIIRSLIM

SEQ ID NO: 36 - DNA, 585 bp, Cathamus tinctorius
  misc_feature (487)..(487): n is a, c, g, or t
tcgggcaccaccactttgcgctccttaatgtcgagtagaagatcaagacaatcgtcatcagggggtccgaggatcacagatgaccaaatcatacaactcgtctccaagttacaacaacttcttcctggaactcgcatccaacgatctaacaaggcatcggcttcaaaggtgttacaagagacttgcaactacgtcagaagcttgcatagggaggttgatgatctcagtgaccgactatcgcagttattatccaccattgacgctgatagccccgaagcttcgattattcgaagcttaattatgtaatatgcaaatctctacatataaattattcgttagcttattgattaagcataattatggtttcttaatcttatagttaattatctccatagggtttaatttaattaatagcccatgttacatgtagacttgtcccagtacttgtcgaaatgataataacaataataattacgttgagaaactnagaaaaaaaaaaaaaaagaggggtagaaggcaactagaaaaaaacattatgaatgttaaaaaagggcggctaagaatttattttttccaattaagaaa

SEQ ID NO: 37 - protein, 91 aa, Camellia sinensis
MSSRRSRSRQSGSSRITDDQINDLVSKLQQLLPELRNNRSDKVSAGKVLQETCNYIRSLHREVDDLSERLSELLATTDTAQAAIIRSLLMQ

SEQ ID NO: 38 - DNA, 527 bp, Camellia sinensis
caaattaaaaatattatataagatgtccagcagaagatcaagatcaaggcaatcaggaagctcaaggatcactgatgatcagatcaatgaccttgtctccaaattgcaacagcttcttcctgagcttcgcaataaccgctctgacaaggtttcggcggggaaggtcttacaagagacctgcaactacattagaagcttgcacagagaggtagatgatcttagcgagagactgtctgagctactggcaactactgacactgcacaagctgcaataatccggagcttactcatgcaatagacctgaatccatactagttcattttgtttatgcaattaatagacagccagtcctctatcttcttcatttctgtgcgtctccaggtcttcgtcaagagagtgatattttgaacttatgtagttgcagttgatcgcttagagagaatctttttctttgcaaagttgtgttttgagtaacgaatataataaaaagtacttctggcctcagacacaatgtttctcaaaaaaaa

SEQ ID NO: 39 - protein, 91 aa, Camellia sinensis
MSSRRSRQAAGVSRISDDQIIELVSKLRQLLPEIRDRRPQKVSASKVLQETCNYIRSLHREVDDLSERLSQLLSTIDADSPEAAIIRSLIM

SEQ ID NO: 40 - DNA, 746 bp, Camellia sinensis
  misc_feature (55)..(57): n is a, c, g, or t
gaggaatgcacttgtcttcttcatccaacatcactgtctttgttgtggtccaatnnntctttataactgatctcttatcacattctccatatagctctttaagtccatcttgcttctcttgccctctcttgaacttcatttcagagttcattctgcgcaaccccttcggccttcagtatctttcttttttatttttcccaagtgaatattgcaagtgccttttaattagctcatttacattaatatatacaaagcaagtcagcagctagctccaaggatcatcatgtctagcagaaggtcgaggcaagctgccggtgtatcgaggatcagtgatgatcagatcattgaacttgtctcaaagctacgccaactcctccctgagattcgcgatagacgcccacaaaaggtttcagcttctaaggttctacaggaaacttgcaattatattagaagcttgcacagggaagttgatgacctaagtgagcgactatcccagcttttatctactatagatgctgatagtcccgaagctgcgataattaggagtttaattatgtaattctgtagccttaattacttaattatctttctagttcttctctactttaatcttactaattaagttctggtcactacatagatcaaacaagaactagaatgtattgtaactataattaagtttgtataataaaaggacttgcactagcaaagcccaagttataatcaatattataatatatttttac

SEQ ID NO: 41 - protein, 93 aa, Coffea canephora
MSSRRSRQSSGSSRITDDQIIELVSKLQQLLPEIRTRRSNKASASKVLQDTCNYIRSLHKEVDDLSDRLSQLLSTIDADSPEAAIIRSLLAES

SEQ ID NO: 42 - DNA, 764 bp, Coffea canephora
catttgcgctgcttaattaataaccattagtgatcgacagcacttgaagttcccgtaggagtcaaaacaaccagcttaaagaatcaagtttggagccctcttatcttatactaacataagcatgtctagcagaaggtcaaggcaatcatcgggttcttcaaggatcacggatgatcagataattgagcttgtctccaagttacaacaacttcttcccgagattcgtactaggcgctcgaacaaggcatcggcgtctaaggttctccaggatacttgcaactacattcgaagcctgcacaaagaggtggatgacctcagtgaccgtctctctcagctactgtcgacaattgatgctgatagcccagaggctgccatcattaggagcttattagctgaatcttgaccatctcctacgatcgatctcgatctctctataatcatcatcgtcgtcatcatcatcattccagtacgtagcctgctcttgctatgccgcctgcttccttatcaagagctagagctgaaaagaatacatatagatatatatatatatgcattacttatgtatggtacttgcgttatgaaacttagacgttggattacgactccaagtcctagtcctggctctctggttagctagttcttgcaatctaagctctgtaaaaataaaagatgttgctgtacttgtacatggcaacaccttgcactcgcatgtatgtatgtacctagtaattaagctatattatatatgaagttttttttttttt

SEQ ID NO: 43 - protein, 94 aa, Fragaria vesca
MSSRRSSRQSSGSTPSIKDDQIIELVSKLRQLVPEIRDRRSDKVSASKVLQETCSYIRNLHREVDDLSERLSQLLATIDADSAEAAIIRSLIMQ

SEQ ID NO: 44 - DNA, 827 bp, Fragaria vesca
catgctcatatatatcttactcccatcttacatttttctaagacaaactaccactgctactactcctacttcctctgcttcttcttctgctctttcttcctcggccccttctcttctatctcagaacttgcttctctaggtttttctcctcctccggtaccggtactactctactacgtactatataatctactctaggttgctcataagctttggcaaacgctaggttgatatataaactaagtagctatatctagctgctgagctgatacatatagaaggaatcagtttgtctgggaaacacagtccgatcgatcatgtctagcagaaggtcatcaaggcagtcatcgggaagtactccatcaatcaaagatgaccagatcatcgagctcgtctccaagttgcgccagctggttcctgagattcgcgacaggcgctccgataaggtatcagcatccaaggtcctacaagagacctgcagctacatcagaaacttacacagagaagttgacgacttgagcgagaggctgtcccaactgctcgctacaattgacgctgatagcgctgaggccgccattattaggagcttgattatgcagtagatcgacgtgtactctataactctataaatatcgttattttagttgatttataaatatctatatagttgcactacatctttatattacttaatttctaggtttcgatcaccatgatcatcaagcacggttaattgagcactactacgtactcatgtacgtacccatctagacaagagctcatgtatggatcagtttgttgatataaaagactgcactagctagcaa

SEQ ID NO: 45 - protein, 93 aa, Gerbera hybrid
MSSRRSRSRQSGVSRISDDQIANLVSKLQQLIPHNLHTSPSDKVSASKVLQETCNYIRSLHKEVDDLSERLSELLQLTDTNSAEAAIIRSLFM

SEQ ID NO: 46 - DNA, 541 bp, Gerbera hybrid
atttgtaaagcttctctgacaaaaatagaaagaaaaataaaagaatccgtcttactatctcaattgtctctgataatgtctagcagaagatcgcgttcacgtcaatctggagtgtcaaggatcagcgacgaccagatcgccaatctcgtgtccaagttacaacaactcattccacacaaccttcacaccagcccttctgacaaggtttcagcttcaaaagttctgcaagagacttgcaattatatcagaagcttacacaaagaagtggatgatttaagtgagagattatcagagcttttacaactcacagacaccaacagtgctgaagcagccattattaggagcttatttatgtaaccatttcttatattatatactaattaattaagcaatcatgagtttttgtgctttttaatcaattatgtcctaaccatgttttagctaaatatttatatgcatatattaattattaaaataaaatgtaatttaagtttcataattattttttgtgcactgttatttattatttctatattgcgtt

SEQ ID NO: 47 - protein, 92 aa, Gerbera hybrid
MSSRRSRQSSSGASRITDDQIIQLLSKLQQLLPEIRNRRSNKASASKVLQETCNYVRSLHKEVDDLSDRLSGLLSTIDADSPEASIIRSLFM

SEQ ID NO: 48 - DNA, 476 bp, Gerbera hybrid
gctcctctctctttcatttcattcttccaaaacaccaaacttgatcatcggtctatataaaccctctcacttaacaaacacataattattgacaacatacagatttgatcataatgtcgagtagaaggtcgagacaatcgtcaagtggagcttcgaggatcactgatgatcaaatcatacaactactatcgaagttgcaacaacttcttcctgaaattcgtaatcgtcgttccaacaaggcatcggcttcgaaggtgttacaagaaacctgcaattatgtgagaagcttacacaaagaggttgatgatcttagcgaccgattgtcggggttattatccaccattgatgctgatagccccgaagcttcaattattcgaagtctatttatgtaaattttattaactaattagcttttttattatatataaattattaatacagtgtttaagttaactgttttcctgaatgtttctcta

SEQ ID NO: 49 - protein, 93 aa, Glycine max
MSSRRSRSRQSGVSTEITDAQITDLISKLQQLIPELRARRSDKVSASKVLQETCNYIKSLHREVDDLSDRLSQLLATTDSNSAQAAIIRSLLM

SEQ ID NO: 50 - DNA, 997 bp, Glycine max
cgggcacgaggccaacacccactagactggcacattctctcaactctctcaataagctttctctcatgctcatggcctctaccactacctttatctctctctcttcctagttcattcattctcctctctcaaaaacataaacatcagcacttctctcttttgaatattcctcaattttatagctacctagctacctagctacctagctaagctatacttggttttctttaatttctctgacaaatattccaacttcttttctatataggctctagtagcttagtagttatctttcagttaccttgaacaaatcaacagcaaaatatatttctgacacactcatcagagaccattttaaatttaattaacaacaaacaatgtctagcagaagatctcgttcgagacaatcgggtgtttccactgagatcactgatgctcagatcactgatctcatctcaaagttacaacaactgatccctgagctacgcgcaagacgttctgacaaggtttcagcttccaaggtgttgcaagagacttgcaactacatcaaaagcttgcacagagaggttgatgatctaagtgaccggttgtcacaacttttggccaccaccgactccaacagtgcccaagcagccattattaggagcttacttatgtaatatataataatattctaataattactattaattatagagctttaattttatgtgtcgtttgcatgtccatgttaattttttttattgtcatgatatcctctattctgggtagggtttggtttttaagaccaaagcaaaaaggccaccgtgggctccatgttctcccttacttagttttatcagacactttatattattgactatcatcaaattgtattactaaaataaaatgaccttgcatcttgatgatcgagtctttagtttgaatgtaaagtcatatatattaatgtgtataaatatatatacatgaatctgtacccgagtagaacatatatgac

SEQ ID NO: 51 - protein, 93 aa, Glycine max
MSSRRSRSRQSGASAEITDAQITDLVSKLQQLIPELRARRSDKVSAAKVLQETCNYIKNLHREVDDLSDRLSELLANTDSNSAQAAIIRSLLM

SEQ ID NO: 52 - DNA, 827 bp, Glycine max
ctctctttttgagataagtttgtttagtttcattttctgtgattctcgtctgacaagcccccccccccccccccccaatttcctgcttcttcttggccctccaatttcccctcagaccttgttctctcatcactcactcactcacaacaacaacaacaacaacactctctttcctctctcatttttaattattctcttcaattccttaagtcattaagaggtagctagaagtagtagctcgcaacagcaaatatttctgacacaaacatcatcacaaaagggtagtagtggacgttgttgttaataaattgttcctctcattaattaattgacaatgtctagcagaagatctcgttcaagacaatccggtgcttccgctgagatcactgatgctcaaatcaccgatctcgtttccaagttacaacaacttatccctgagcttcgtgctaggcgctccgacaaggtttcagctgctaaggtattgcaggagacatgcaactacataaagaacttgcacagagaggttgatgatctaagtgaccgattatcggagcttttggctaacacagactccaacagtgctcaagcagccattattaggagcttacttatgtaatagtctagtctagtgcattaatttgtgtcgtgtgcatgtccatgtctctctcacttttttttttttttttatcactctgggtagggtttggtttgtactttgtttatcaacgcaaaggactaccatcggatccatgtgaattagttcttaattagttaattttaattatcatgtggcactttgtagtaattaatatcaacagcttctacgg

SEQ ID NO: 53 - protein, 92 aa, Glycine max
MSSRRSRSRQTSSSRNITDDQINDLVSKLQQLLPEIRDRRSDKVSASKVLQETCNYIRSLHREVGDLSERLSELLDTTDTAQAAIIRNLLMQ

SEQ ID NO: 54 - DNA, 821 bp, Glycine max
gatttctttctcgatccccaacatcactagctagctccttgtacacactctacaaccccacctagctacatcacttaattagttttaccaatttcaaattctcacctgtcactagctatatttcataactgatcattaccaactcatcactacatattattggctaggattcaccattagacttaagattagttgatttattacatatataagatgtctagcaggaggtcacggtcaaggcaaacaagtagttcaaggaatatcaccgatgatcagatcaatgatcttgtctctaagttgcaacagcttcttccagagattcgcgataggcgctctgacaaggtttcagcttccaaggtgttgcaagagacatgcaactatattagaagcttacacagggaagtgggtgacctaagcgagcgtttatctgagctcctggatacaactgacacggctcaagctgcaataattagaaatttactgatgcaatagatcggtgcagttgttaatttatcgtataattcatagttaacacttcagtacttgtgaaccgatccagtcactggtcgtgtatttcttattctcttttcgtttcactttttttttttttttttgtgctggttcttgtccactaatatgaatgattactgcttttgcaaagcccaatttccttatatattaaataaaagtttcagagttcgtgctttgctaaattaaatatacattttctctttctaagcacacttaatattagatggacatttttttaaaaaatatttgtctgaaatttgaccctatcgtttttaattatttaccagggg

SEQ ID NO: 55 - protein, 93 aa, Glycine max
MSSRRSRSSQSDVSTEITDAQITDIISKLQQLIPELDARRSDKVSASKVLQETCNYIKSLHREVDDLSDRLSQLLATTDSNSAQAAIIRSLLL

SEQ ID NO: 56 - DNA, 642 bp, Glycine max
gctacctctataggctctagctagcttagtagtcagaagtagttagtacttatctttcagttaccttgaacaaatcaagtgtacacaacagcaaaatatttctgtcacacaaacacactcgtcacaaacccttttaaatttaaaattaacaacaatgtctagcagaagatctcgttcgagtcaatcggatgtttccactgagatcactgatgcccagatcactgatatcatctcaaagttacaacaactaatccctgaactagatgcaaggcgttcagacaaggtttcagcttccaaggtgttgcaggagacttgcaactacatcaaaagcttgcacagagaggttgatgatctaagtgaccggttgtcacaacttttggccaccacagattccaacagtgcccaagcagccataattagaagcttacttttgtaataataatattattctaatacttattacttatagagctttaatttatgtgacgtgtgcatgttatttttggttataagaccaaattgtattactataaataaaatgagctggcatcttgatgatcgagttccagttagttcgaatagttatattaatgtgtataaatatattatacatgaatctgtacccgtgtagaaatatatgac

SEQ ID NO: 57 - protein, 91 aa, Glycine max
MSSRRSRQQSASTRISDDQIIDLVSKLRQLVPEIRDRRSDKVSASKVLQETCNYIRSLHREVDDLSERLSQLLATIDADSPEAAIIRSLIN

SEQ ID NO: 58 - DNA, 835 bp, Glycine max
gcaacactactatatcttagccttttctctccctcccttctcccatattatatagcttgtcttttatttcttagactccatccattcttctccccaattgaatcttctttattttgtttcttcactgtctcgttatggctatagttttgcatagtaaataaactgaactgaagctatctatatagcagcaagtgttgatttaattacttactttagacaataattatattaattacaccaatttataagctctttatctatctatctagctagggaaaattaaaatgtctagcagaaggtccaggcagcaatctgcatccacaaggatctccgatgaccaaatcatcgacctcgtttcaaagttgcgtcaacttgttcctgagattcgcgataggcgctctgacaaggtatcagcatctaaggtcctacaagagacctgcaactacatcagaagcttacacagagaagtggatgacttaagcgaacgactgtctcagttgttggccacaatcgatgctgatagccctgaagctgccatcattaggagcctaattaactaataatatatattaagcgcaagtaatcatctaattttcctatattcaaggagatatattataagagtgtattaatttcttcttctaaattaggtggcatagagtgcagtttgaggtgcgtacgtacgtccttccaatatattatagtacatggcaggaatggtgcacttgtgtaagttaaaggtttttgcaataagaactaaggactctctgtattatggctatagtgctatataataatatatgcatgccacatttatagatggcctccaaaaaaa

SEQ ID NO: 59 - protein, 92 aa, Glycine soja
MSSRRSRSRQTSSSRNITDDQINDLVSKLQQLLPEIRDRRSDKVSASKVLQETCNYIRSLHREVDDLSERLSELLATTDTAQAAIIRNLLMQ

SEQ ID NO: 60 - DNA, 704 bp, Glycine soja
ctcgatccccaacatcactagctagctccttttgtacacactctacaaccccacctagctacatcacttaattagttttcccatatctataaccaatttcaaattctcacccttaactagctagctatatttcataactgattattaccaactcactacatattattggctaggattcaccattagacttaaaagtagttgatttattatatatataagatgtctagcaggaggtcacggtcaaggcaaacaagtagttcaaggaatatcaccgatgatcagatcaatgatcttgtctccaagttgcaacagcttcttccagagattcgcgataggcgctctgacaaggtttcagcttccaaggtgttgcaagagacatgcaactatattagaagcttacacagggaagtggatgacctaagcgagcgtttatctgagctcttggctacaactgacacagcacaagctgcaataattagaaatctactaatgcaatagatcggtgcagtagttaatttatcgcataattcatagttagcacttcagtacttgtgaaccgatccagtcagtagtcgcgtatttcttattctctttttgtttcactttttttttctggtttttgtccactaatatgcatgattactgcttttgcaaagcccattttcctaagatattaaataaaagtctgagtttgcgctttgcta

SEQ ID NO: 61 - protein, 91 aa, Gossypium arboreum
MSSRRWRESSRTNIWEEQITQLLSTLRQLLPEIPHSHSHKASSAAKVLEQTCNYIKTLHREVDDLSDRLSQLLATIDADSAEAAIIRSLFN

SEQ ID NO: 62 - DNA, 487 bp, Gossypium arboreum
  misc_feature (486)..(486): n is a, c, g, or t
gttataggcagtgataagatgtcaagcagaaggtggagggagtcgtccaggaccaatatttgggaggagcagatcactcaacttctctctaccttacgccaacttcttcccgagattcctcattcccattctcacaaggcatcatcagcggccaaggttttagaacagacatgcaattacataaaaaccttgcatcgtgaagttgatgatctgagtgaccggctgtcccagctcctagccaccatcgatgccgacagtgccgaagccgccatcatccgaagcttatttaattaatacaaaaaaaaaaaaacctccttctgtattccttcaattctctctgctttgtctatacttttagtttttactagctaggctctaatgaatcattacatcgtacacacatgactatatattttgagacactatgcttccctttcgtgagactgtaaataaaagatatttgcactagcaaacgcctttttgctnt

SEQ ID NO: 63 - protein, 92 aa, Gossypium hirsutum
MSSRRSRSRQSGASRITDDQIIDLVSKLQQLIPELRGRRPDKVSASKVLQETCNYIRSLHREVDGLSDRLSQLLASTDTDSDQAAIIRSLLM

SEQ ID NO: 64 - DNA, 663 bp, Gossypium hirsutum
atgcatttgtcccttataattctactacccaagcatatctcttctctacctttcttgctcttgttttttacttcgtttctaatttcctcctctcatcccattttccccttaaacttatatttataatatccttaaactttcactttgttgattactttcctgagccaaatatcagtacaatgtcaagcagaagatcacgttctaggcaatcaggtgcttcaaggatcactgatgatcagatcatcgatcttgtttccaagttgcaacagcttatccctgagcttcgtggaagacgccccgacaaggtatcagcttctaaggtcttacaggaaacctgcaactatatcagaagcttacacagagaagttgacggcttaagcgatcggttatctcagctattagcttccacagacaccgatagcgaccaagcagccattatcaggagtttacttatgtaatgatcagacctagattaagtattactcactttttccaggtctagggtttgtttatcatcgcttgtattgtagtaatgcagaacaagggtggaatagtggctacagtgaaccatgtttcgtaatccttgtcatcaagagacttgtataccgttcgagtttatcctcgtatatttataaaataaaaataaattatgagttgcggggg

SEQ ID NO: 65 - protein, 92 aa, Gossypium hirsutum
MSSRRSRQSTAGVSRISDDQIIELVSKLRQLLPEIRDRRSDKVSASKVLQETCNYIRSLHREVDDLSERLSQLLATIDADSAEAAIIRSLIM

SEQ ID NO: 66 - DNA, 602 bp, Gossypium hirsutum
ctactctctactctttctttctttcttcttcttcttcttcttcttcttgccttttccaaaccataaccatttttctacaccatatatacttatttttccttctgcatttccatatcttgggggtattattttggaacttactgaatattttagaacaattcttggtttttggtcattagggtgttattattactttaaatcccccacaagatgtctagcagaaggtcgagacagtcaacagcaggtgtttcgaggatttcagatgatcaaatcattgaacttgtatcaaagttacgacagcttctgcctgagattcgtgataggcgatctgataaggtatcagcatccaaggtcttacaagagacttgcaattacattagaagcttgcatagggaggtggacgacctaagtgaacggctctcacagttgttggccaccattgatgctgatagtgccgaggctgctattattaggagtttaattatgtaataatatttattataaaccaatgttttttattattactagggttatatatatatatatgatattatatatttggatttatatatagtttaagatgcttcctccatgtggaaggg

SEQ ID NO: 67 - protein, 93 aa, Gossypium hirsutum
MSGRRSRSKQSSVSSITDNQITDLVSKLQHLIPELRRRRFDKVSTSKVLQETCNYIRSLHREVEDLSDRLSQLLASTDGGSDQAAIIRSLLMQ

SEQ ID NO: 68 - DNA, 717 bp, Gossypium hirsutum
aaagcaaagcatcataacctctcttctctttgttcttggttaactcatctctttgctcttttcccagcttaagtttccaggtcctttatgcatatattatcgtattcccttttttcttaggttttcttcggtgacaaatccacatcccatttgtcaatttaatcaagagtttcagatatcctcactttccttaagccttcattcatttagtttcttgacacaaaaaatatagtagaagttattttaaaacacaatgtcaggcagaagatcacgttccaagcagtcaagtgtttccagtatcactgacaatcagatcaccgatcttgtttccaagctgcaacaccttatccctgaacttcgtcgaaggcgattcgacaaggtatcaacttccaaggtgttacaggagacttgcaactatatcagaagcttacatagagaagtggaggacctaagcgaccggttatcccagctattagcttccacagacggcggtagcgaccaagcagccattataaggagtttacttatgcaataaacacccattttcattcacctttggttttactatattaagtactacacaccttttccatatcttaggatttccggtagtaatgcagaagagataggaaaagggtgaaacatggagtaccatgcatgttttatgattcttgtcatcaacagactctgtaaatcattcaagtctatcattatata

SEQ ID NO: 69 - protein, 94 aa, Gossypium hirsutum
MSSRRPSSRQSSGGVSRISDDQIIALVSKLRHLLPEIRDNRSDKVSASKVLQETCNYIRSLHKEVEDLSDRLSQLLATIDAESAEAAIIRSLLM

SEQ ID NO: 70 - DNA, 638 bp, Gossypium hirsutum
atctcttacatctaacactgctcattcacgttgttgtcaaaaccactacatgttccatttatacacttgctagcttggcttttgttggttttaattgaatatataaacccctcgccaaccctcctaccaacagatacaagaaacttggtcttaatttcccaaagatgtcgagccgaaggccgtcgtcgaggcagtccagcggcggcgtttcccggatttcagatgatcagataattgcacttgtctccaagttacgccaccttcttcctgagattcgtgacaaccgatctgacaaggtatcagcatcgaaggttttacaagagacgtgcaattacattagaagcttgcataaagaggtggaagatctaagtgaccgactctctcagcttttggctaccattgatgccgaaagcgccgaggctgctattattaggagtttacttatgtaataataatccaactttactattattatttattaggttcggtcgctaggtccaacataaactagattattgtttaagatgcttacccccatgtgcattgtaattacttagtataataaaaagacttgcaatagcaaagtcttttaattgtaatgacaatatggatgacttatataattatgtttagctttt

SEQ ID NO: 71 - protein, 92 aa, Hedyotis terminalis
MSSRRSRQSSSGSTRITDDQIIELVSKLQQLLPEIRTRRSNKASASKVLQETCNYIRNLHREVDDLSDRLSQLLSTIDAESPEAAIIRSLLS

SEQ ID NO: 72 - DNA, 605 bp, Hedyotis terminalis
gcagaaacgaacagagtatcccagtacttccttcttaattggtttcattccgactggattttgtaattccactgtatcatcattcattagaaaacattttaatatgcttatgatttatgaaacatacatgttttaccgttctgtaatttatctgagtatttctttacgctagatacaaagaaaaacttataataaaaaaatttcaaactgcatccagcagctctgtagtattctatcaggtgaaaagaaaaaaaaacggtagcaataaaataatgtctagcagaaggtccaggcaatcatcatcagggtctactaggattacagatgatcagattatcgagctggtctcaaagttgcagcaacttcttcctgagattcgtactaggcgttccaacaaggcatcggcatctaaggtgctgcaagagacttgcaattacatccgaaacttgcacagagaagttgatgaccttagcgaccgtctttcacaactcctgtccaccattgatgctgaaagcccagaggctgccattattaggagcttactatcttaatccttccatgtagtaataactaagtattagcaagctgcgttgtaatcagccagc

SEQ ID NO: 73 - protein, 91 aa, Helianthus paradoxus
MSSRRSRQSSSGARISDDQIIQLISKLQQLLPGTRIQRSNKASASKVLQETCTYVRSLHREVDDLSDRLSQLLSTIDADSPEASIIRSLIM

SEQ ID NO: 74 - DNA, 541 bp, Helianthus paradoxus
tatctacataatctaattataaagatacgatcgataatactacgatgtcaagcagaagatcgaggcaatcatcatcgggggctaggatctcagatgatcaaatcatacagctcatctccaagctacaacaacttcttcccgggactcgtatacaaagatctaacaaggcgtcggcttcgaaggtgctacaagagacttgcacatatgttagaagcttgcatagggaggttgatgaccttagtgatcgactatcgcagttattatccaccattgatgccgatagccctgaagcttctattattcgaagtttaattatgtagaatactcatttctacttataattatgttatttgttagcttcttgattaaggataaatgtggtacgttaatctctaaattaatcatatgtgtttctagggtttaatctaattaataaccctagtttcatgtagtgatattattttagatatatgtcaatagttttgatgatgatgataataataagagctacaccggtagtgtattaaaagttttctcataa

SEQ ID NO: 75 - protein, 97 aa, Helianthus paradoxus
MSTSRSRYRQTRPSRITDDQIADLIYELQQLIPNDHRRKGSSAKVLEETCSYVGSSQREVEDLSQRLSKLLQPMDTNSPQAAIIRTLLMSPKLIYDY

SEQ ID NO: 76 - DNA, 715 bp, Helianthus paradoxus
gtttgtttgatcacctccaagtgtctttatttttctcatcttctccattatttttctccccataacttgtcggcatcattcaattaacaccttcatatataccactttcacactaataccactatatcaaaccatctactatcttagttatcacctcatctctttgctaactcatccatgtctacctcaagatcacgctaccgacaaacccggccatcgaggatcaccgacgaccaaatcgcggatctcatatacgagctacaacaactcattcctaacgatcatcgtcgcaaggggtcaagtgcaaaggtattggaggagacatgtagttacgttggaagttcacagagagaagttgaagacttgagtcaaaggctatcaaagcttttgcagcccatggacaccaacagtccacaagcagccatcattagaaccttacttatgtcacccaagctaatatatgattattaatgatcttgatatatgtgatttgatcaatgtcattttgatttcttctgcatgtacgtgcattttccttgagtatgttcgcttgctgagaatttctctggcagcgatgagcacaagacttcagcccctgccaccataggcggtggtgaatatggatataaggaacgagaggaaggacatgagaagaaaggactgatggataagattaaggaaaggctacctggcggcgatcatggcagggatgagc

SEQ ID NO: 77 - protein, 91 aa, Helianthus tuberosus
MSTSRPRYRQTRPSRITDDQIADLIYKLQQLIPNDHHRKGSSAKVLEETCSYVRSLQREVEDLSQRLSELLQSMDTNSPQAAIIRSLLMSP

SEQ ID NO: 78 - DNA, 494 bp, Helianthus tuberosus
cgggatgcccttgagttaatcaactccaatgtctttatttttctcatcttctccattatttttctccccataacttgtcggaatcattcaacaccttcatatatatcactttctcacgaataccattatatcaaaccatcttatcacctcatctctttgctaactcatctatgtctacctcaagaccacgctaccgacaaaccagaccatcgaggatcaccgacgaccaaatcgcggatctcatatacaagttacaacaactcattcctaacgatcatcatcgcaagggttcaagtgcaaaggtattggaggagacatgtagttacgttagaagtttacagagagaagttgaagacttgagtcaaaggctatcagagcttttgcagtccatggacaccaacagtccacaagcagccatcattagaagcttacttatgtcaccctagcttgctaatatatttatttatctttatatatgttatttgatcaatgtc

SEQ ID NO: 79 - protein, 88 aa, Hordeum vulgare
MSSRRSSRGAISDEEINELISKLQSLLPNSRRRGSSQASTTKLLKETCSYIKSLHREVDDLSDRLSDLMSTMDHNSAGAEIIRSILRS

SEQ ID NO: 80 - DNA, 1139 bp, Hordeum vulgare
  misc_feature (3)..(3): n is a, c, g, or t
  misc_feature (11)..(11): n is a, c, g, or t
  misc_feature (1105)..(1105): n is a, c, g, or t
  misc_feature (1123)..(1123): n is a, c, g, or t
ggnctgcaggnaattcggcacgaggagcgattgcacaactacaagttatatccagcagcaaagtatccttgagttgctcgacgtcagcgagggcctcccttcgctcactggccaactgcccgccgctccttgagcatacacactgtgtggctttacttgccggcggcagtctgcagtctctgttagctccggccggcggtagctagcttgtcatcccggccggcgacgcagcggcggctaggaaggaagacgatgtcgagcagaaggtcgtcgcgcggcgccatctccgacgaggagatcaacgagctcatctccaagctccagtctctgctccccaactctcgccgccgcggctccagccaggcgtcgacgacgaagctgctcaaggagacgtgcagctacatcaagagcctccaccgggaggtggacgacctcagcgaccggctgtcagacctcatgtcgaccatggaccacaatagcgccggagcagagatcatccgcagcatcctccgctcgtgatcgtacgtactgaagtgccggcaggtcggcgaggactaaaccgccgggacgattaagcggcggcggcgccatgggtttctccggccagccggacacgtacgcacgagagctttgcttagctagggtatatatatttgtcctccacatatttaaatatgtatctctttcctgctccctttctgcctagatcgatctgatcgtgtagatcgaaaaatgtactccgtgtccctaagcttcactccttctgctgtactgcgtagggcattagcttagctagcgtccctaccttgggccaaagcttatcctcgcgcgctggctgccgcttgagttaatctctcgatcgtctcctccgtgtgcgtgtttccctcgctagctcggagctggatagatggctccgttcctcctcctgtctgcctcttcccctcttttgttctccctttctcgatctactactcgatatgtaaatttagttggtggcattggatcgagttgtgtcctctatagacaaccgaccgaccactactacggtactactcctcctactattacctagagcaaactaatatcaacgccatgttgtaccatnccaggtttaacttttgtngaatacgtactacgag

SEQ ID NO: 81 - protein, 90 aa, Hordeum vulgare
MSGRRSRGSVSEEEINELISRLQTLLPTTRRRGSSSSSSQASTTKMLKETCSYIKSLHREVDDLSDRLSDLMSTMDNNSPAAEIIRSLLR

SEQ ID NO: 82 - DNA, 901 bp, Hordeum vulgare
  misc_feature (862)..(862): n is a, c, g, or t
cagctagccgcccactctactccttccgatcacacaggacacagaagcctctccgtcgacctcggccttaacttgctcgcgcctcattattatcatcggacgacggcgatgtcgggcaggaggtcgcgcggctccgtgtcggaggaggagatcaacgagctcatctccaggctccagaccctgctccccaccacgcgccgccgcggcagcagcagcagcagcagccaggcgtcgacgacgaaaatgctcaaggagacgtgcagctacatcaagagcctgcacagggaggtggacgacctgagcgaccgcctctccgacctcatgtccaccatggacaacaacagccccgccgccgagatcatccgcagcctcctccgctagctagctagagctacctgcagctcaactgctcatcatcatcatcatcatcgatcacgtccagctgattgtcctaagctagctcatcatctacttacagctcgtgcatgcctagctaagcccgcgcatatatctacatatacatacaagtatggtatatatgtcgtccgtcgatctctgcaaggcatatatatgcagatcgatcgacacctaaggactgcatgcatatgcatccatgctaaaggctggtctaatttactttggtagggcatcattagctagggccgcctaattaagttcactatagctggttaattagctcatcccgctagcttcttgcatgcatgtggtttcctcccagcttccctctctcttgttttcatttgtttttccctgtgtaaacttacttatttgcagtctggatcgagttgtttccctagagacaccgaccgaccgctagctacccatccgtggtactgcatgnctatatatatatagcactagtaccatcacacatgcatga

SEQ ID NO: 83 - protein, 96 aa, Lactuca sativa
MVMSSRMSRQSTTGASKITDDEIMQLLSQLQQLLPEITNRRSDTASASKVLQETCSYVRSLHREVDDLSDRLSQLLSTIDARQPPSFNYRKFNDLI

SEQ ID NO: 84 - DNA, 681 bp, Lactuca sativa
ttcttatattcacatcgttgggcatgcttccaagaatcatatcgtactctttctctttcttagagtaaattgttgcatcactgaccttctgtctaaaaatactgcgatatattgacgtgaaactgaaactattttgactaaacattgtatttatacatcattgccacctctctcaaaacacctctatttctttctcacctacttgtcatacacatacaaattttatggtaatgtcaagcagaatgtcaaggcaatcgacaaccggagcttccaagatcaccgatgatgagatcatgcaacttctctcacagttgcagcaacttcttcccgagataacaaatcggcgttccgacacggcgtcggcttcaaaggtgctacaagagacttgcagctatgtgagaagcttgcatagggaagttgacgatcttagcgaccgattgtcgcaactattgtccaccattgacgctcggcagcccccaagcttcaattatcgaaagtttaatgatttgatttgaatggacttttttctaattacatgaaataatattgtatttcagttaattaacaatctgagtgatttctgggggtacgaaatacccttgtggggatcatttatgactaccgcttgtaattatatatgaccaccgattgtactttctatatctaacattcaccttcgtc

SEQ ID NO: 85 - protein, 93 aa, Lactuca virosa
MSGRRSRSRQTGVSRISDDQIADLVSKLQQIIPHNIHATRSDKVSASRVLQETCNYIRSLHREVDDLSERLSELLQSTDANSAEAAIIRSLFM

SEQ ID NO: 86 - DNA, 459 bp, Lactuca virosa
gcggggatcttgtgatcattaatttgtaagacttctctgacaaattaataaggaaagaaaccccttcttataataccatctcagttgtccctctagatctcaatgtctggcagaagatctcgatcacggcaaactggagtttccaggatcagcgacgatcagattgccgacctcgtctccaagttacaacaaattatacctcataatatccacgcaacccgttctgacaaggtttcagcttcaagagtgttgcaggagacatgcaattatatcagaagcttacacagagaagtggatgatttaagcgaaagactttcagagcttttgcaatctacggacgccaacagtgctgaagcagccattattaggagcttatttatgtaactatattgttaacaaattaagcagttaatgtaaggcttttctgtgttaataaatcagcataaatatgttttcttt

SEQ ID NO: 87 - protein, 96 aa, Malus x domestica
MSSRRSSRSRQSSSRNNNSISDDQITDLVSKLQQLLPEIRPRRSNKASASKVLQETCNYIRNLHREVDDLSERLSELLATTDMDNDQAAITRSLLL

SEQ ID NO: 88 - DNA, 455 bp, Malus x domestica
tcccgctcatttgtctctcatagttttttctctagaagaacttcaaactctaatattattatttcttctgagttttctgtcagaacttcaaactctaatattattatttcttctgagttttctgtcagaacttcaaactctccaaatatttgacaatgtcgagcagaagatcctctcggtcgaggcagtcgtcgagtaggaacaacaacagtatcagtgatgaccagatcactgatctcgtatccaagttacagcagcttcttcctgagattcgccctaggcgttccaacaaggcgtcggcgtcgaaggttttgcaagagacttgcaattatattagaaacttacacagagaggtggatgacctaagtgagcgcttatcagagcttttggccacgacggacatggataacgaccaagcagccattactaggagtttacttttgtgatggtgtctg

SEQ ID NO: 89 - protein, 91 aa, Malus x domestica
MSSRRSRQSGTPAIKDDQIIELVSKLRQLVPEIRDRRSNKAPASKVLQETCSYIRNLHREVDDLSERLSQLLSTIDADSPEAAIIRSLIMQ

SEQ ID NO: 90 - DNA, 586 bp, Malus x domestica
aggcaggctgatcatacatacgaacatatatatatgttctgagaacgatatataaaactagagctacttgtctagctagctagctaggtgaaggggatcatgtctagcagaaggtcgaggcagtcaggtactccagcgatcaaagatgaccaaatcatcgaactggtgtccaaattacgtcaactggttcctgagattcgagataggcgctccaacaaggcaccagcatctaaggtcctacaagagacttgcagctacatcagaaacttacacagagaggttgacgacctaagcgagcgactctcccaactgctctctacaattgatgctgatagtccggaggccgctataattaggagcttgattatgcagtagatggatcatcaaatcacgagcaaccctaattatatatattggcgttttaattatatagttaattgtccttttatttgtttccaggttatagtacttcgtattgtatgtatttagagatgtcgtgtgtggttaattagagtgtttaataaattgaaggatttgcactgctactagcaagtcctattataaagaaaccctatttactttctcc

SEQ ID NO: 91 - protein, 91 aa, Malus x domestica
MSSRGSRQSGTPAIKDDQIIELVSKLRQLVPEIRDRRSNKVPASKVLQETCNYIRNLHTEVDDLSERLSQLLSTIDADSPEAAIIRSLITQ

SEQ ID NO: 92 - DNA, 587 bp, Malus x domestica
ttttaggctaatattctcgttcctaaaccctatatatataaagctccattgccaggctgatcattcatacatatatatattttgaaaacgataatataaaattagctacttgtctagctaggtaagtaaggatcatgtctagcagagggtcaaggcagtcaggtactccagcgatcaaagatgaccaaatcatcgaactggtgtccaaattacgtcaactggttcctgagattcgagataggcgctccaacaaggtgccagcatcgaaggtcctgcaagagacttgcaactatatcagaaacttacacacagaggttgacgacctaagcgagcgactctcccaactgctctctacaattgatgctgatagtccggaggccgctataattaggagcttgattacgcagtagatggatcatcagatcatgatgagaaaccctaatattatatatatatatatgtatgtatgtatatgtatatgtgtgtgtatgtgtgtctaagagcattttaatttatatagttaagtgtccttttactttctttccaggttagagtacttagtactgtatgtatttaaagatgtcatg

SEQ ID NO: 93 - protein, 92 aa, Malus x domestica
MSSRRSSSSSRTSKPSDDEIKELISKLQPLLPQLHHTRNAPVSASSILEETCSYIKRLHREVEDLSQRISQLLDSAGISDVDEELIRRLLQH

SEQ ID NO: 94 - DNA, 547 bp, Malus x domestica
atttgatagtgtaaagaagctaaaagctataagcagaaaataagcaaaagtgcttcttcaaccagccatgtcaagcagaagatcatcatcatcatcaagaacttctaaaccctcagatgatgagattaaggagctcatctcaaaattacaacctcttcttcctcagcttcatcatacgcgtaatgctccggtatcggcgtcgagcattttggaagaaacttgcagttacataaagaggctgcatagggaggtggaagatctgagccaaagaatatctcaactcctggattctgcaggcatctctgatgttgatgaagagcttattagaagacttttgcagcattaataatcgctctctctctctctaggctagcaattttaattacatgagttaagttttggttggactatccaaccagtgagagattatatatatgaggaatgcatatatatatatatatatctgtgtatatatgtttgtaatcttcagtgtaccttctgatatcttatgtgttgttaaaatggtatctagtcattgatctagctc

SEQ ID NO: 95 - protein, 92 aa, Medicago truncatula
MSSRRSRSRQTSSSRNITDDQIHDLVSKLQQLLPEIRNRSSDKVSASRVLQETCNYIRNLNREVDDLSERLSELLATTDTAQAAIIRNLLMQ

SEQ ID NO: 96 - DNA, 647 bp, Medicago truncatula
ctccaacatcactgtcccttccccattccttgttggaagctagtcactccttagtgcacctatctccatatcctatttcaatttgtctttgaaatttccatacattacattttctataccaaatcactaagattaaggtcacatatatatccaaaactaaagtagtatagtatatttgttttctaggattcacctaggcttgaaattattgaattttataaagatgtctagtaggaggtcgcggtcacggcaaacaagtagctcgaggaatatcaccgacgatcagatccatgatcttgtctccaagttgcagcaacttcttcctgagattcgcaataggagctctgacaaggtttcagcttcgagggtattgcaggagacttgcaactatattagaaacttgaacagggaagtcgacgacctaagcgagcgtttgtctgagctattggctacaacagacacagcacaagcagcaataattagaaatttacttatgcaatagatttttcttgtttcattatttttaattagcactcaaggactttgtgacctgatgtatttcttcttcattttcgtacttcagttttaaatttgtatttcctaatttcatccacttaatgcaagttttctagtttatgttttttttt

SEQ ID NO: 97 - protein, 93 aa, Medicago truncatula
MSSRRSRSRQSGGSSEITDAQITDLISKLQQLIPELHASRSNKVSATKVLQETCNYIKNLHREVDDLSDRLSQLLASTDSNSAQAAIIKSLLM

SEQ ID NO: 98 - DNA, 723 bp, Medicago truncatula
cttcaacccactcctcttcactctcttcgccaaatttaattttattatcataccttagcaacattacaaaaaacatataaccacttttatttatttctctcttaataaatactattcccttactctccatactcatttcacataacttctcttcactttcttcccttctttctctgacaaccaaccaacctcatttcatctctttctctttatttattttcttgtatcacaaattaatatttctgacatatagttactttacattaccactacctccttaattataacaatgtctagcagaagatctcgttccagacaatccggtggttcctctgagatcactgatgctcaaatcactgatctcatttccaagttacaacaacttatccccgaacttcacgctagccgctccaacaaggtttcagctactaaggtattgcaagagacttgcaactacataaaaaacttgcatagagaagttgatgatttaagtgacagattgtcacaacttttggcttctacagattccaacagtgctcaagcagctattattaagagcttacttatgtaatagtgtctagttatatatatatttataactactactactcttgcatttgtttgtcgtgtgcatgtccttggctcactttattggtattctcttatctggggaagggttaatttgggttggaaaccaaggcaaaagggttactatataccctt

SEQ ID NO: 99 - protein, 92 aa, Medicago truncatula
MSSRRSRQQSSSSRISDDQIIELVSKLRQLVPEIRHRRSDKVSASKVLQETCNYIRNLHREVDDLSERLSQLLVTIDADSPEANIIRSLINQ

SEQ ID NO: 100 - DNA, 795 bp, Medicago truncatula
tttaggctcatctctctccattttaattagcaacatcctacgccactgctttcccaatgcaattgtttgcttcaaatctaacatcattgtctatctaattgtaccctcttccttcatttatggtccctttctctccctttatatctctctcatcactttctccatttcttcattcatacctatagcttttaactctaccactcctaccattcctcctctccttttctccactacatagcttgtcttttgtttgttaatttccaacaaaataaaccaattaattcttgttactttcttactttatcctccctcatttgtatcttcttggttatattataaactatatatataaatagaaaaatacaccaattctaggtatatattatgtctagcagaaggtcaaggcaacaatcttcttcttcaagaatttctgatgatcaaatcatcgaacttgtttccaagttacgtcaacttgttcctgagattcgtcataggcgttcagacaaggtatcagcatctaaggtcctacaagagacttgtaactacataagaaacttacacagagaagttgatgatttaagtgaaagactgtctcagttattggtcacaattgatgctgatagtccagaggctaatattatcagaagcctaattaatcaataagtttagaaattaatctaattttcttaatttccattatgatgagaaatatatatttatatatctataagagtgtattttattttaaatgggctagctagagtgtagtttgaggtgtgcaattttcatatggcg

SEQ ID NO: 101 - protein, 92 aa, Nicotiana benthamiana
MSSRRSRQSSAGSSRISDDQIIDLVSKLQQLLPEIRTRRSNKASASKVLQETCNYIRNLNREVDDLSDRLSQLLSTIDADSPEAAIIRSLLM

SEQ ID NO: 102 - DNA, 587 bp, Nicotiana benthamiana
ctcgttctaaacaaataaaaggagacaaagattagttgtagacaagaacaaaaaagacaattaactttaatttaaggatgtcaagcagaaggtcaagacagtcatcggcaggttcctcaagaatttcagatgatcagataattgacctcgtatccaagctgcaacaacttcttccggaaattcgaacccgtcgttccaacaaggcatcggcatcaaaggtgctacaagaaacttgcaactatataagaaatttgaatagagaagtggatgatcttagtgatcgtctttctcaattactctcaaccattgatgctgatagtccagaagctgcaatcattcggagtttattaatgtagctattaattaatgtattatgagttttaaatattggagttatattttactgcgtatcaattaagttcttgttttttcttcttaattgctatagactcagctggtcactagctaggccaatcatgagttagaatttagaactgaaatattagtaatatcccccttcctatggcacacgcttgtaattatatatttcccttagtataattaataagatggagtacttgtgtttt

SEQ ID NO: 103 - protein, 93 aa, Nicotiana tabacum
MSGRRSRQSSEEGTSRISDDQIIELMSKLQQLLPEIPTRRTNKASASKVLQETCNYIRSLHKEVDDLSDRLSQLLSTIDADSPEAAIIRSLLM

SEQ ID NO: 104 - DNA, 650 bp, Nicotiana tabacum
ggttgcaggtctattttctaactaccagtacgccctcctctttactgctttactactactactactaccacttcccattgtgtaagctgccattcaaacaagttttgagccattaatttgtaccccaaaaagaaaaggagaaaaattactagtagttgaagtatgtcagggagaaggtcgaggcaatcatcagaggaggggacgtcgaggatttcagatgatcagatcattgaactgatgtccaagttgcaacaacttcttcctgaaattcccactcgtcgcaccaacaaggcatcagcatctaaagtgcttcaggaaacatgcaactacataagaagcttgcataaagaggtggatgatctcagtgatcgactttctcagttgttgtccaccattgatgctgacagtcctgaagctgcaatcattcgtagtttattaatgtaatcttcaatttgttcatcatatattgaatagacgtatactactacctagttcttcgtttcttcttcctctctaggctttgctggtcactactattagctaggccaatctcattagttagcatttataacttccccttgcttattatatgtacacgcgcttgtaaggatgtttccctagattaaataataataagatgtacttgtgct

SEQ ID NO: 105 - protein, 104 aa, Oryza sativa
MSSSRRSRSRRAGSSVPSSSSSSRTSISEDQIAELLSKLQALLPESQARNGAHRGSAARVLQETCSYIRSLHQEVDNLSETLAQLLASPDVTSDQAAVIRSLLM

SEQ ID NO: 106 - DNA, 315 bp, Oryza sativa
atgtcgagcagccggaggtcgcgctcacggcgagccgggagctcggtgccgtcgtcgtcgtcgtcgtcgaggacgtcgatctcggaggaccagatcgccgagcttctctccaagcttcaggccctgctcccggagtctcaggctcgcaatggcgcccataggggctcggcggcgagggttttgcaggagacgtgcagctacatcaggagcctgcaccaggaggtggacaacctcagcgagacgctcgctcagctgctcgcctcccccgacgtcaccagcgaccaggcggccgtcatcaggagcctcctcatgtga

SEQ ID NO: 107 - protein, 91 aa, Oryza sativa
MSSRRGGGGGGGRITDEEINELISKLQALLPESSRSRGASRSSASKLLKETCSYIKSLHREVDDLSDRLSELMSTMDNNSPQAEIIRSLLR

SEQ ID NO: 108 - DNA, 878 bp, Oryza sativa
atacaaccactccctggctccctgcttctactgaaacctataggctactagctagtgctctcactctcactcactcacactgtcacttccacattttctttgctctctcactgtccgtacgtcgtcggcttgatcaccaaccttgctgacggtggtcggtcgccggagttcgaggagaagaaggcagtagaggtgtggtggtgatattgcaggcggcgaccatgtcgagccgccgtggtggtggtggtggaggagggaggatcaccgacgaggagatcaacgagctcatctccaagcttcaggccctcctcccggagtcctcccgcagccgcggcgcaagccggtcgtcggcgtcgaagctgctcaaggagacgtgcagctacatcaagagcctgcaccgggaggtggacgacctgtccgaccggctgtcggagctcatgtcgacgatggacaacaacagcccgcaggccgagatcatccggagcctcctccggtgatcgatcctagctctatcagtagctgcagcgacgacgacgattgatggaggacgagcttgctagctagcgattgaggagggagacgacaagatttttttgctcttctttgtttctttcttcgatctctcctgaaaagggaaaagtgtttgtttctcaggtaatcaccgaaggcccctgcattgtttaggagaagaaaaagggttgtcttgtttggtatgtactgtaccagggctaatagagatcgatatatgtggatttctattagctgctagtagagttattagtagctagattagctacttaatttagcttgtagacgtactactactgtacttaagctgctttgataagcttcagagatgcatctagaggtgtttgttt

SEQ ID NO: 109 - protein, 88 aa, Oryza sativa
MSSRRSSRGSISEEEINELISKLQSLLPNSRRRGSSQASTTKLLKETCNYIKSLHREVDDLSDRLSDLMATMDHNSPGAEIIRSILRS

SEQ ID NO: 110 - DNA, 1071 bp, Oryza sativa
cacacacacttctcccatccagctagcttttgcagatcgagcgaaccttcaacacaaaccggccagcttgttcctctcctctctctctctctctctctctctctcggtcgcagccagctaacttcgagtgtgaagaacactcacgctagctagctttctcaaggccacatacgctagctccgcctccacgcgcggcgtacgtctagtttgattgaggctgaagagagtgcgtgtttggtagaagaggctagctgttgacgatgtcgagcagaaggtcgtcgcgtggctccatctcggaggaggaaatcaacgagctcatctccaagctccagtcgctgctccccaactcacgccgacgcggctccagccaggcgtcgacgacgaagctgctgaaggagacgtgcaactacatcaagagccttcaccgggaggtggatgacctcagcgaccggctgtccgacctcatggccaccatggatcacaacagccccggcgcggagatcatccgcagcatcctccgctcctgatcggcaagcagcagcagcaagcggcaccggccggccggccggcctatgtcgacgacgagataggccgggccggccgggcaacgcccgcggcgccaattaatcaagcgtacgtcctgccggcgccattctccggccaccagagagcatttagctagggtatatatatctacatatataaaatatttatgtcgtatgtccctccccaaatgtacgagccacgcttacacgcgcgtgtatcgatctctcgatctctctctctaagctccactcgaagtagggcattagcactaggggcctaagcagaggcttatcctcgctttagctcatattttcatcgcttgatgtgtcctctctgaacccccacatcttgtcttgtttggtttttccctctcccttccaatctatgtaaatttagctggtgtggtttggatcgagttgtgtcctctacagacaaccgaccgaccactactacctagaggaaattaatatcatcatgggtgtactgctccagctttaacttcaattcagttggctacgtactacg

SEQ ID NO: 111 - protein, 87 aa, Oryza sativa
MSSRRSSRSSVSEEEINELISKLQSLLPSSRRRGANQASTTKLLKETCSYIKSLHREVDDLSDRLSDLMAGMDHNSPGAEIIRSLLR

SEQ ID NO: 112 - DNA, 877 bp, Oryza sativa
ctgcttcaattcctacctcacacttctgcaacaagctttagctccagccaccggaacgagagatcgatacagctcgatcagctagccgcattcgccctcgccgccgccgacgatgtcgagccggaggtcgtcgcgctcctccgtgtcggaggaggagatcaacgagctcatctccaagctccagtccctcctccccagctcccgccgccgcggcgccaaccaggcgtcgacgacgaagctgctgaaggagacgtgcagctacatcaagagcctgcaccgggaggtggacgacctcagcgacaggctctccgacctcatggccggcatggatcacaacagcccaggcgccgagatcatccgcagcctcctccgctagctcatctcttctcatctgttcttcctcctcgatgatcgatcgatctcgtgttattctacgtacatgaaggatgatatgaatgcagctcgtgccctgtagctacgtataaatatacacatatgtatacatgcatacagtacatgatatgcatatgcctagatctgatctagctcagtagaaaatctactcatctcggtgtactactgatgatgatgatgattaaggcatgcagctggcttgatgatcggtactactactactacttcacttcagttcagtaggggcattagctagctagctagggccggctgcctaattaagtaattaagtaatttattatttgtagtagccagcttgatctcatctctcctcatgagccatatgacatgtggttaagttaattaatccttgatgcagtagagtcgtcccagctgtttcatcgatgatcgtcgtcatatgtgtgtatctcttgatctagctttgctgatctcctcttgtttc

SEQ ID NO: 113 - protein, 77 aa, Panicum virgatum
MSGRRGRISDDEINELISKLQALLPESSRRRNASRSSASKLLKETCTYIKSLHREVDDLSERLSGLMATMDNDSPQA

SEQ ID NO: 114 - DNA, 617 bp, Panicum virgatum
ccacgcgtccgctctggctctggctcctcccggttgcagttgcaagctgacctcgctgctgctgccaccactcactgctctgccagttctttccctcgacctcacacagtagcaagctattaagagcactccccggagttcaccagctaggtttaaggcattgaaaccctatccttttcaggtccctctcgccttttcttttcctcccttggctcctatttccgtttgcagaccggagctcgcacacacgtactactacatatatacccaagttctgcgagaggccgaggccgaggcagggacgacgacgaagaagaataatccaccaggagctgtatagtagcatcatccaagcacgtactctctgagctaggactcgccggagatgtcaggccgccgcggcaggatcagcgacgacgagatcaacgagctaatctccaagctccaggcgctcctcccggagtcctcacgccgccggaacgcgagccggtcgtcggcgtcgaagctcctgaaggagacgtgcacctacattaagagcctgcaccgggaggtggacgacctctcggagcggctgtcggggctcatggcgaccatggacaacgacagcccccaggccg

SEQ ID NO: 115 - protein, 92 aa, Petunia x hybrida
MSSRRSRQSSVGSSRISDDQIIELVSKLQQLLPEIRTRRSNKASASKVLQETCNYIRNLNREVDDLSDRLSQLLSTIDAESPEAAIIRSLLM

SEQ ID NO: 116 - DNA, 540 bp, Petunia x hybrida
cagaaaacttctcttttactagcttcttcctcattccaatttcttctactaccagttaattaattattgttcttaaattcctacgataaattcgaccatttgttataaacacttaagaggagacagacattagtcgagtacgtaaattgtagaaattaacaataagacatctttgtttaatttaaggatgtctagcagaaggtcaaggcaatcatcagtaggttcttcgaggatttcagatgatcaaatcattgaactcgtatccaaattgcaacaacttcttcctgagattcgcactcgtcgctccaacaaggcatcggcatcaaaggtgcttcaagaaacttgcaactacattaggaacttgaatagagaagtggatgatcttagtgatcgtctttctcagttactctccaccattgatgctgagagcccagaggctgcaatcatccgaagtttattaatgtgacaaatttattttgatttctaaatattggagctatatattttaagtaccaagttcttgttttttcttcttgctct

SEQ ID NO: 117 - protein, 92 aa, Petunia x hybrida
MSGRRSRQASEGSSRISDDQIIELMSKLQQLLPEIRSRRTNKEPASKVLQETCNYIRNLHKQVDDLSDRLSQLLSTIDADSPEAAIIRSLIM

SEQ ID NO: 118 - DNA, 539 bp, Petunia x hybrida
tagactagtgatccccgggctgaggaatcggcacggaggtatactaccggtaggcagtagttgctcttactactactactactactactacttcttgttccagttttaagtcgtgtaccatttaaattagttgaagaatgtcaggaagaaggtcgaggcaggcatcagaggggtcttcaagaatttcagatgatcagatcatagaattaatgtccaaattgcagcaacttcttcctgaaattcgcagtcgccgcaccaacaaggaaccagcatctaaggttctccaagagacatgcaactacattagaaatttgcataaacaggtggatgatctcagtgaccgactttctcagttattgtccaccattgatgctgacagtccagaagctgcaatcattcgtagtttaataatgtagtagtataacttttttctttattaaatgttggaggcattattctactccctgtttcttgtttaatttcttcttaatacctctaggctatgctggactggcgaggcaaatctgatcatgtgttag

SEQ ID NO: 119 - protein, 85 aa, Petunia x hybrida
MSSRRSSRISDDQIIELVSKLQQFLPEIRTRRSSKASASKVLQETCNYIRDLNREVDDLSDRLSQLLSTIDADSQEAAIIRSLIM

SEQ ID NO: 120 - DNA, 553 bp, Petunia x hybrida
gggatcccgggctgaggaattcggcacgagggttaccatatattttgaattatgtcaagtagaaggtcttcaaggatttcagatgatcaaattattgaacttgtgtccaagttgcagcaatttcttcctgaaattcggactcgtcgttccagcaaggcatcagcatcaaaggtgctccaagaaacttgcaactacattagagacttgaacagagaagtggatgaccttagtgatcgactttctcaattactatccactattgatgctgacagtcaagaagctgcaataattcgtagtttaataatgtaattatttttatttatatactaattgcaatgtactctaccacctaattgttgcttctttaacttcctctagtctctaagtactggtcattagctataggccaatcatgagttagaatttaactcgaaatatatgcaagtattggatctgatgtccccctagctaaggacacatgcttctgattatgttcccacccataaattaaataagatggtattgcattttaattaaaacaaccccaaacaaa

SEQ ID NO: 121 - protein, 92 aa, Phaseolus vulgaris
MSSRRSRQHSGSTRISDDQIIELVSKLRQLVPEIRSRRSDKVSASKVLQETCNYIRSLHREVSDLSERLSQLLTTIDADSAEAGIIRSLLNQ

SEQ ID NO: 122 - DNA, 692 bp, Phaseolus vulgaris
gtcctttctcttccttcattataactctctcatcactctctcctctctctcaaaacctccctcgtatccaacactcttctcttcttcctcctcctactcctatatatccccttccccttcttccactaactccatgtgttttatttctcacttcccaacccaaaaccattcattctttgcttccttctgcttctatatcactacctgctaaaacttatcatacatataaatacgcacacacatacctctgcttatagcaccaaaactaggctagcttagcttgcactttcaacaattatatacatacacaagtacaccaagctctacctagcaacctaccaacatgtctagccgaagatccagacaacattcagggtctacaaggatctccgatgaccaaatcatcgagcttgtttccaaattgcgccaacttgttcctgagattcgcagtaggcgatctgacaaggtttcagcgtccaaggtcctacaagaaacctgcaactacatcagaagcttgcatagagaggtgagtgacttgagtgagcgactgtctcagttgttgaccacaattgatgctgatagtgctgaagctggaatcattaggagcctacttaatcaataagcaatgagtgttatgattttttcattcaaagagtgctaattattatgagagtgtacttcattctaggtgt

SEQ ID NO: 123 - protein, 91 aa, Populus trichocarpa
MSSRRSRSRQSSSSRISDDQILDLVTKLQQLLPEIRNRRSDKVSAAKILQETCNYIKSLHREVGDLSERLSELLETTDTAQAAIIRNLLMQ

SEQ ID NO: 124 - DNA, 386 bp, Populus trichocarpa
cataattaagtgaatatcaatttcaagaacatgtctagccgaaggtcacgatcaaggcaatcaagtagttcaagaatcagtgatgatcagatccttgatcttgttacaaagttgcaacaacttcttcctgagattcgtaacaggcgttctgacaaggtttcggctgccaagatcttgcaggagacatgcaactatattaaaagcttgcatagagaggttggtgatcttagcgagcggctgtctgagctattggaaacaactgatacagcccaagctgcaataatcaggaacttacttatgcaatagagctaattaaggattaatactacttgcttggcttatgcagtcggatcatgtttcctctctcttccttaattttgttcttc

SEQ ID NO: 125 - protein, 91 aa, Populus trichocarpa
MSSRKSRSRQSGSSRINDDQILDLVTKLQQLLPETRNRRSEKVSAAKALQETCNYIKSLHREVDDLSERLSELLETTDTTQAAIIRNLLMQ

SEQ ID NO: 126 - DNA, 386 bp, Populus trichocarpa
attacaagttaatataaatttcaataagatatgtctagccggaagtcgcgatcaaggcaatcaggtagttcaagaatcaatgatgatcagatccttgatcttgttacaaagttgcaacaacttcttcccgagactcgaaatcggcgttctgaaaaggtctcagctgccaaggccttgcaggagacatgcaactatattaaaagcttgcacagagaggttgatgatcttagcgagcggctttctgagctattagagacaactgacactactcaagctgcgataattaggaatttgcttatgcaatagggctagttaaggattagtacttgttagcctatgcagtaggatcgtgtgcttgtttccctctcttcctttgtttttctcta

SEQ ID NO: 127 - protein, 91 aa, Prunus persica
MSSRRSRQSGTPTIKDDQIIELVSKLRQLVPEIRDRRSDKVSASKVLQETCSYIRNLHREVDDLSERLSQLLSTIDADSPEAAIIRSLITQ

SEQ ID NO: 128 - DNA, 742 bp, Prunus persica
aaaaaacaaaaaaaaaaaaaaaaaaaaaaaaactcagacctagttctctccctcgtgcccaaattggctcttgcgtacgtggccacttacctcttttaggttaatatttctccttcccagctagctatatatgtatatatattaatattaatatataaagccccgtagccagtctgatcatcgatctctttatatatatatatataaccagctgtagctaacagtgtagatatatagctagagatcatgtctagcagaaggtcgaggcagtctggaactccaacgatcaaagatgaccaaatcattgaacttgtctccaaattgcgccaactggttcctgagattcgtgataggcgctccgacaaggtatcagcatctaaggtcctacaagagacttgcagctacatcagaaacttacacagagaggttgacgacctaagtgagcggctctctcaactactctcgacaattgatgctgatagcccggaggccgccataattaggagcttgattacgcagtagatgatcagctagatgatgatcatcagacccttaattatatatttatatataattgtgctttgatgatgaatttatatagttaaattgtgttcatctaggttatctggtcacccgattattaaacaaggccagttagctagggtactcagcagtattgtatgtatttaaagatgtcctttcctgtcctgtcctgtgttgttaattagagcgaggccctagt

SEQ ID NO: 129 - protein, 91 aa, Prunus persica
MSSRRSRQSGTPTIKDDQIIELVSKLRQLVPEIRDKRSDKVSASNVLQETCSYIRNLHREVGDLSERLSQLLSDIDADSPEAAIIKSLITH

SEQ ID NO: 130 - DNA, 580 bp, Prunus persica
tcttctcctcctcctccttttgatctacttcttctccttctttttcctcctctcgagtactttttcctcacaaattggctcttgcgtacgtggccacttacctcttttaggttaatatttctccttcccagctagctatatatgtatatatattaatattaatatataaagccccgtagccagtctgatcatcgatctctttatatatatatatataaccagctgtagctaacagtgtagatatatagctagagatcatgtctagcagaaggtcgaggcagtctggaactccaacgatcaaagatgaccaaatcattgaacttgtctccaaattgcgccaactggttcctgagattcgtgataagcgctccgacaaggtatcagcatctaatgtcctacaagagacttgcagctacatcagaaacttacacagagaggttggcgacctaagtgagcggctctctcaactactctcggacattgatgctgatagcccggaggccgccataattaagagcttgatcacgcactagatgatcagctaaatgatgatcatcagacccctgattatatatctata

SEQ ID NO: 131 - protein, 93 aa, Prunus persica
MSSKVSRQSGTPTIKDDQIIELVYKLRQLVPDIRDKRSDKVSAYNVLQETCSSIRNLHKEVDDLSERFSQLLSTIDADSTEAALIKSLMSQEM

SEQ ID NO: 132 - DNA, 442 bp, Prunus persica
cttacctcttggatgttaatatttttccttgccagcttgctttatatgtatatatattaatattaatatataaagccccgtttctagtctgatcatcgatctctttatgtatatatatataaccatctgtaactaacagtgtatgatatatagctagacatcatgtctagcaaagtgtcaaggcagtctggaactccaacgatcaaagatgaccaaatcattgaacttgtctacaaattgcgccaactggtacctgatattcgtgataagcgctccgacaaggtatcagcatataacgtcctacaagagacttgcagctccatcagaaacttacacaaagaggtagacgacctaagtgagcggttctctcaactactctcgacaattgatgctgatagcacggaagccgccttaattaagagcttgatgtcgcaggaaatga

SEQ ID NO: 133 - protein, 91 aa, Prunus persica
MSSKRWRQFGIPTIKDDQIIEFVSKLRQLVPEICDRRSDKVSAFKVLQETCSYFRNLHREVDDLSEGLFQLLSTIDVDSPEAAIIRSLFTQ

SEQ ID NO: 134 - DNA, 612 bp, Prunus persica
taaagcccggtagccagtctgatcatggatctctttataaaaaaatatataaccagctgtagctaacagtgtagatatatatttagagatcatgtctagcaaaaggtggaggcagtttggaattccaacgatcaaagatgaccaaatcattgaatttgtttccaaattgcgccaactggttcctgagatttgtgataggcgttccgacaaggtatcagcatttaaggtcctacaagagacttgcagttacttcagaaacttacacagagaggttgacgacctaagtgaggggctttttcaactactctcgacaattgatgttgatagcccggaggccgccataattaggagcttgtttacgcagtagatgatcagctagatgatgatcatcagacccttaattatatatttatatataattgtgctttgatgatgaatttatatagttaaattgtgttcatttaggttatttggtcacccgatttttaaacaaggccagttagctagggtattcagcagtattgtatgtatttaaagatgtcttttcttgtcttgttttgtgttgttaattagagggaggcctttttaaagaatatttattaaagttttttt

SEQ ID NO: 135 - protein, 83 aa, Prunus persica
MSIKKSRQSGTPTIKDDQIIELVSKLRQLVPEIRDTRSNEVSASKVLQYTCSCITNLHREVDDLSERLSQLLSKLMLIARRPP

SEQ ID NO: 136 - DNA, 615 bp, Prunus persica
cttcttcttctcctcctcctcctttagatctacttcttctccttctttttcctcactctcaagtactttttcctcacaaattggctcttgcgtacgtggccacttacctctgttaggtcaatatttctccttaccagctagctatatatgtatatatattaatattaatatataaagccccgtcagccagtctgatcatcgatctctttatatatatatatataaccagctgtacctaacagtgtaaatatatacctagagatcatgtctatcaaaaagtctaggcaatctggaactccaacgatcaaagatgaccaaatcattgaacttgtctccaaattgcgccaactggttcctgagattcgtgatacgcgctccaacgaggtatcagcatctaaggtcctacaatatacttgcagctgcatcacaaacttacacagagaggttgacgacctaagtgagcggctctctcaactactctcgaaattgatgctgatagcccggaggccgccctaattaggagctcgattacgcagctgatgatcagctagatgatgatcatcagaccctggattatatatgcatatataattgtgcttcgatgatgaatttata

SEQ ID NO: 137 - protein, 90 aa, Ricinus communis
MSGRRSRQPSVPRITDDQIIDLVSKLRQLLPEIRQRRPDKVSASKVLQETCNYIRNLHREVDDLSERLSQLLATIDADSPEAAIIRSLIM

SEQ ID NO: 138 - DNA, 727 bp, Ricinus communis
aattcggcacgaggcttgtccttccctactcttgctctcttcttgctacttatatttttccatttccctttttgatttctttcctccatttttatgtttcttgagttatcgattctaacatattattaattaacacatacacaaccttcttgaattacttaaaaagaaaggatcatgtctggaagaaggtcgaggcagccaagtgttcctagaatcactgatgatcaaatcatcgaccttgtctccaagttacgccagcttcttcctgagattcgccaaaggcgtcccgataaggtatcagcttctaaggtcctacaagagacctgcaactacatcaggaacttgcacagggaggtggatgacttaagcgagcgattgtctcagcttttggctacaattgacgctgatagtcctgaagctgctataattaggagtttaattatgtaactagagcagagctccctgtgttaattaattaattaattaaaaaatatttttcccattgtaactttattagtagagagagtttgatcattatgtagatcagacaaaaagggtttttagaatgcattttaagatatatgttatatatatatatatatatatatacacacatatgcatgctatatggataagcatgcagtgtaattaggttgtataataaaaggactttgcacttagcaatgcctaatttatatggataatttatcttggttttacagttggt

SEQ ID NO: 139 - protein, 87 aa, Saccharum officinarum
MSSSGRRGRISDDEINELISKLQALLPESSRRRNASRSSASKLLKETCAYIKSLHREVDDLSERLSGLMSTMDNDSPQAEIIRSLLR

SEQ ID NO: 140 - DNA, 675 bp, Saccharum officinarum
  misc_feature (583)..(583): n is a, c, g, or t
cacgtacacactgcataaataggcgagctgcgagaggcagagacgacgaggacgaagaggaattaagccaacgaggtgctgtagcttccgagcgactggtgtctctcagcaagctacgaccgtcgtcaccagccggagatatgtcgtcgtcgggccgacgtggcaggatcagcgacgacgagatcaacgagctcatctccaagctccaggcgctcctcccggagtcctcacgccgccggaacgcgagccggtcgtcggcgtcgaagcttctgaaggagacgtgtgcctacatcaagagcttgcaccgggaggtggacgacctctcggaacggctgtcggggctcatgtcgaccatggacaacgacagcccccaggcggagatcatccggagcctcctccggtgacccggccgccccgccccggcgcgcgcggtccggcctcttctgcctgcgatctagctagctagctgcagaagacgacgacttccgggcgagcttgccttgctcgtttgctacggcgacgaccttaattatgttcctttgtcttttaatttcttcttcttcttcttccggtgtggtgtgtncgttcccgtgtttaattaattcaagagcaagagctctggctcccaagacaacgaccaaaggttatggatcttctcctggtacacggcaagca

SEQ ID NO: 141 - protein, 92 aa, Salvia miltiorrhiza
MSSRRSRSRASGSSRITDDQIADLVSKLQQLIPEIRSRRSDKASASKVLQETCNYIRNLHREVDDLSHRLSGLLESTDGDSAQAAIIRSLLL

SEQ ID NO: 142 - DNA, 509 bp, Salvia miltiorrhiza
aggaattcggcacgaggctctttctctctcttacacacacaacatccaacaaacacaacaactaattaaactcccttcactctcaccaccaccaccaccaccacaacttcttgtaacttaaaatgtctagcagaagatcgcgttcgagggcgtcgggatcctcgaggataaccgacgatcagatcgccgacctcgtctcgaaattgcagcaactcatccccgagatccgcagccgccgttccgacaaggcttcggcttcgaaggtgttgcaggagacgtgcaactacataaggaacttgcacagagaggtggatgatctgagccatcgattgtcggggctgctggaatcgacggacggcgacagcgctcaagccgccattattaggagcttgctattgtaatgttgttacagcgcatagatgtggttgtacgtttcgcttctgtagtcgtacgtctagggttaattttgccagtatatgtatgtagctatatggtttggtatgctatcc

SEQ ID NO: 143 - protein, 91 aa, Salvia miltiorrhiza
MSGRRSRPSTNTSRITDDQIIDLVSKLHQLLPEIRNNRRSNKASANKVLQETCNYIRNLHKEVDDLSERLSRLLSSIDADSPEAAIIRSLI

SEQ ID NO: 144 - DNA, 425 bp, Salvia miltiorrhiza
gcacgaggcttccttcaagctaactttatacacacacacacacatatactttaatttgaatttctggcattaaacatagatatttataatctttaagaaggatgtctgggagaagatcacggccgtcaaccaacacatcaagaatcacagacgaccagatcatcgatctcgtctccaaactgcatcaactcctccctgaaatccgcaacaaccgccgctccaacaaggcttctgcaaataaggtgcttcaagaaacttgcaactacatcagaaatttgcacaaagaggtggatgatttgagtgagaggctatcgcggctactgtcgtcgatagatgctgatagccctgaggcagccataattaggagcttaatatgagtgattaattatgtaataattatgattctatatataggatatgtggct

SEQ ID NO: 145 - protein, 88 aa, Secale cereale
MSSRRSSRGAISDEEINELMSKLQSLLPNSRRRGSSQASTTKLLKETCTYIKSLHREVDDLSDRLSELMSTMDHNSAGAEIIRSILRS

SEQ ID NO: 146 - DNA, 643 bp, Secale cereale
tcgcacgagctcgtgccgcgggagtctgcttgctccggcaccagcttgctcatcccggtcagccagtgacgtagcagcctctaggaggacgatgtcgagcagaaggtcgtcgcggggcgccatctccgacgaggagatcaacgagctcatgtccaagctccagtctctgctccccaactcacgccgccgcggctccagccaggcgtcgacgacgaagctgctcaaggagacgtgcacctacatcaagagcctccaccgggaggtggacgacctcagcgaccggctgtcggaactgatgtcgaccatggaccacaacagcgccggagcggagatcatccgcagcatcctccgctcgtgatcatactacagcgccggccggccgatcggagagagctcaaccgccaggacaattaagcggcggcggcgccatgggactctccggccagccggacacgtacgagagctttgcttagctagggtatatatatcgtcctccacatatttaaatatgtatctcttttcgcctccctttctgcctagatctgatcgtgtagatcgaaaaatgtactacgtgtctccaagcttcactccgtctgtactgcgtagggcattagcttagctagcgttgctaccttgagccaaa

SEQ ID NO: 147 - protein, 92 aa, Senecio squalidus
MSSRRSRQSSSGSSRITDDQIIQLISKLQQLLPGNRIQRSNKASASKVLQDTCNYVRSLHREVDDLSDRLSELLSTIDPNSPEASIIQSLIM

SEQ ID NO: 148 - DNA, 551 bp, Senecio squalidus
cgtatcataaataatcactctacatctgcgaccaactaacatcattaatttaatcacatcatatagtttgatcgattctattatgtcgagcagaagatcaagacaatcatcatcagggtcttcgagaatcaccgatgatcagatcatacaactcatctccaagttacaacaacttcttcctgggaatcgtatccaacgatctaacaaggcgtcagcgtcgaaggtgctacaagatacttgcaactatgttagaagcttgcatagagaggttgatgaccttagcgatcgactgtcagagttattatcgaccattgaccccaatagtcccgaagcgtccatcattcaaagtttaattatgtaaaatgcacttgtttacttttaaactattcattagcttcttgattaagcataattggagttccttaatctcttaattaacttcttaataatgtgtagggttaaaaatctaattaatgacccatgttaatgttacgtgtagtatcattatagttctttgtcgaataataataaataattaaaagttttctagt

SEQ ID NO: 149 - protein, 88 aa, Senecio vulgaris
MSSRRSGAPPRITDEQIIELVSKLQQLLPELRTRRSNKASASKVLQETCNYVRNLHKEVDDLSERLSRLLSTIDDNSPQASIIRSLID

SEQ ID NO: 150 - DNA, 369 bp, Senecio vulgaris
cttaaaaggattcaattctatttgatcataatgtcgagcagaaggtctggagcacctcctaggatcacggatgagcagatcattgaacttgtctccaagctgcaacaactacttccagagcttcgaactcgtcgttccaacaaggcatcagcttcaaaggtgttacaagagacgtgcaactatgtgagaaacttgcacaaggaggttgatgaccttagtgaacgtctctcccggttattgtccaccattgatgataacagccctcaagcttccatcattaggagtttaatcgattagtgaacgacaattatatatagataagcatttactcttttcaattaatcatatcgattgctagtgttgctcatc

SEQ ID NO: 151 - protein, 86 aa, Solanum lycopersicum
MASRRSRSRISDDQIADLVSKLQQLIPEIRNRRSDKVSASKVLQETCNYIRNLHREVDGLSERLSQLLESTDSDSAQAAIIRSLLM

SEQ ID NO: 152 - DNA, 532 bp, Solanum lycopersicum
  misc_feature (514)..(514): n is a, c, g, or t
tgttctttcttgaatttcattatatataatggcaagcagacgatcacgttccagaataagcgatgatcaaatcgctgatcttgtttccaagttgcaacaacttatccctgaaattcgtaatagacgttctgacaaggtttcagcttcaaaagtgcttcaagaaacttgcaactatataagaaatttacacagagaagtggatggattaagtgagagattatcacaacttttggaatcaactgatagtgatagtgctcaagctgctattattagaagcttacttatgtagtagctatttaattaaatagctcaaacttcattaaaatttctattattaaaaaaagatggagcatttatattattaattagggtttttttggccactataggtcaaaattaatttctatttttctgttttccaattttgaatagttctttttttttcttttactttgtgttgtattgttgtatccatctgttttaagagctgatgtttctttgatctcagcctttnttaagtatataagtattt

SEQ ID NO: 153 - protein, 92 aa, Solanum tuberosum
MSSRRSRQSSTGSSRISDDQIIELVSKLQQLLPEIRNRRSSKASASKVLQETCNYIRNLNRQVDDLSDRLSQLLSTIDADSPEAAIIRSLLM

SEQ ID NO: 154 - DNA, 766 bp, Solanum tuberosum
ccttaccacaacttcctctccttaacaagtttcatacacacacacaaaaaaaaatatcttctttcttttccttatccatatgtgttgtgtaagcaattaaataaaagaaaactacgctagcacagagccatatttgtaacgaaaagagagacatttttttgttgaatttaaggatgtcgagcagaaggtcgaggcaatcatcaacaggatcctcgaggatttcagatgatcagataattgaacttgtctcaaaattgcaacagcttctaccggagattcgcaatcgtcgctctagcaaggcatcggcatcgaaagtactgcaagaaacatgcaactacataagaaatttgaatagacaagtggatgatcttagtgatcgactttctcagttactctcaactattgatgctgatagtccagaagcagcaatcatcaggagtttattaatgtagtagccatatactcctatgaattaattaatgtattttcatttctaaatattggagctatatttatatataaatcttactaccaagttcttgattttctggttgttgttaagctctagtagactcagctggtcactatagctaggccaatcatgagttagaatttaattagaacttcaactatatagtagtgccgattgaaccaatgtgatccccctagctaagggcacacgtacgtacgtttgtatctatatttcctttactccttagttaaatataattaattaataagatgtacaaagacaagctaaaaaact

SEQ ID NO: 155 - protein, 86 aa, Solanum tuberosum
MSSRRSRSRISDDQIADLVSKLQQLIPEIRNRRSDKVSASKVLQETCNYIRNLHREVDGLSERLSQLLESTDSDSAQAAIIRSLLM

SEQ ID NO: 156 - DNA, 622 bp, Solanum tuberosum
gcagcgaggattctattctatcgtgacaactcactttaacaacaacaacaactctttctcccattgtttattttctctgccccttttgttctttcttcagtttcattatatataatgtcaagcagacgatcacgttccagaataagcgatgatcaaatcgctgatcttgtttccaagttgcaacaactaattcctgaaattcgcaatagacgttctgacaaggtttcagcttcaaaagtgctgcaagagacttgcaactatataagaaatttacacagagaagtggatggattaagtgagagattatcacaacttttggaatcaactgatagtgatagtgctcaagctgctattattagaagcttacttatgtagctatttaattaaatagctcaacttcattaaaatttctattattaaaaaagatggagtatttatattattgattagggttttttggccactataggtcaaaattaatttctatttttctgttttccaattttgaatagtttttttactttatgttgtattgttgtatccatctgttttaagagctgatgtttctttgatctcagcctttttttaagtatataagtatttagagatgtttgttatcgtt

SEQ ID NO: 157 - protein, 95 aa, Solanum tuberosum
MSNRRTRGSRQSSGASRISDDQIADLVSKLQLLIPESRSTRSSDKVEASKVLQETCNYIRSLHREVEDLSDRLSVLLESTESDSAQAAIIRSLFM

SEQ ID NO: 158 - DNA, 693 bp, Solanum tuberosum
catatggttctcttctctctttgcccataacttgtctctcaaatagttatcatcctcttctctttttaaactccccacaacacacacacaactctcacacatattcattcaaacaacacattctattactatatataacttctatcttgacaccttaattaacacaacttcttgcctacttaacacaatataatgtcaaatcgaagaacacgcggttcgagacaatcatcaggagcttctagaataagtgatgatcaaattgctgatctcgtatcaaagttacaattacttatccctgaaagccgcagtactaggagttccgataaggttgaagcttccaaagtgttgcaagaaacatgtaattacataagaagtttacacagagaagtggaagacttaagtgatagattatcagtccttttggaatctactgagagtgacagtgctcaagctgctattattagaagcctatttatgtgacaattattgccattatttgaagattactagttatgtaacaataatttcaatttactaggttctattttcatttgtaatttactttaattatcatcttgttattatttttcttcttctaagatgcaatagtacttatagttaattaattaattaagacattatatttgtattgagaaatttactaaatatttataaattgactatggattcagt

SEQ ID NO: 159 - protein, 92 aa, Sorghum bicolor
MSSRRSRSRQSGSSRITEEQISDLVSKLQDLLPEARLQSNARVPSARVLQETCNYIRSLHQEVDDLSERLSELLATSDMSSAQAAVIRSLLM

SEQ ID NO: 160 - DNA, 648 bp, Sorghum bicolor
cccatgcacttgtcttcctcctccaataagcttctctccagtccttgactaccatttcatcatctgtgtatctcaaacttgcttgcctcactcctaacatctcagtttgtttctcgatctcttgcaaaacttctttccccaatctcccagacaaccacatcaaccaagatgtcgagccggaggtcacggtctaggcagtctggttcgtcgaggatcactgaggagcaaatcagcgaccttgtatcaaagctgcaggacctcctccccgaagctcgccttcagagcaatgctagagtgccatctgcgagggtgttgcaggagacatgcaactacatcaggagcttgcaccaggaggtggacgacctgagcgagaggctgtcggagctgctggctacgtccgacatgagcagcgcgcaggcggctgtcatccgaagcctgctcatgtagctgagacatgcatctcgcagcagcgttcatagcctaagtagagtgatttttagtacttgttggagaggcaggtcaatcacctaattcgcccgtgtacttcgcctacgtgcattacgcactgttgtcgtctcgctacctgagccagaggccagagctatcttttttgtgtacttattaatcaatcctatcgttgttggcgcg

SEQ ID NO: 161 - protein, 90 aa, Sorghum bicolor
MSSRRSSSSRGNISEDEINELISKLQALLPSSRRRGSGQASTTKLLKETCSYIKSLHREVDDLSDRLSDLMSTMDHNSPGAEIIRSILRS

SEQ ID NO: 162 - DNA, 861 bp, Sorghum bicolor
aattcgcagagcgacccccagtccagcgcgcgcgggtagtccacacacacacaccttcgttcccagttcccaccaatacacagcgattgagccgcgcgccagtggacgcacacacgcggtagctttagcttcgttctcaagcttagcccggccgattctacgcgcgcagtgttcgtctcgttcgttccggcagcaggcggcgaagtacgacgacgacgacgagctagagagcgaggatgtcgagccgaaggtcgtcgtcgtcgcgtggcaacatctccgaggacgagatcaacgagctcatctccaagctccaggccctgctccccagctcccgccgccgcggctccggccaggcgtcgacgacgaagctgctgaaggagacctgcagctacatcaagagcctccaccgggaggtcgacgacctgagcgaccggctgtcggacctgatgtccactatggaccacaacagccccggcgcggaaatcatccgcagcatcctccgctcctgatcacgtacctcctactgcggcgccggcgccgggccgggggctgcgcgtgagagctagcgaggtcaacgccggcctcgtcgccgacgacgacgaccgcaaccgttctccgcctgatccggctggccacgtgcgggagctgagctcaattagctagggtatatatatatctttctctctacaagtatgtgtatctttctctgccttttacctgtctccctagatctgagatcatgtggctagctccatatcaaaatgtactagctacacgcacacgtatgatggtctctgctaagcttcacactgggtagggtactactagcgcactagggcctaagcaagcttatcccccggc

SEQ ID NO: 163 - protein, 90 aa, Spartina alterniflora
MSSRRSSRGGNISDEEINELISKLQALLPVSSRRRGSGQASTTKLLKETCSYIKSLHREVDDLSDRLSDLMSTMDQNSPGAEIIRSILRS

SEQ ID NO: 164 - DNA, 531 bp, Spartina alterniflora
ccaagcttgatttgactaatagatcgttcccagtcggcgacggcgaagcgaagcacgacgagcggcgagctagagagcatgtccagccggaggtcgtcgcgtggcggcaacatctccgacgaggagatcaacgagctcatctccaagcttcaggccctgctcccagtcagctctcgcagacgcggctccggccaggcgtcaacgacgaaactgctgaaggagacttgcagctacatcaagagcctccaccgggaggtggacgacctcagcgaccggctttcggacctcatgtccaccatggaccaaaacagccccggcgcggagatcatccgcagcattctccgctcatgatgacctgcggcaagcggtgtgtgtggctgtcaccgtcgaccaaccgccaacgacgaccggcctccacgccatgcattattccccgggccagattacgggagctccacattagctagggtatatgcacatatatatttctatctatccacaaatattgtgtactgtctcaacaaaaaaaaa

SEQ ID NO: 165 - protein, 91 aa, Triphysaria versicolor
MSSRRSRSRQSGTSRISEDQINELVSKLQQLLPELHNRRTDKRSATNVLQETCNYIRSLHREVDDLSERLSELLATTDTTQAALIRRLLSQ

SEQ ID NO: 166 - DNA, 597 bp, Triphysaria versicolor
tgtctcaccactgtttctaactctcaacttcacttcaaacttagttaatactcttgctttttaagttatttattacataattgttaaaaatgtcgagccgaagatcacggtcaagacaatccggaacttcgaggatctccgaagatcaaatcaacgagctcgtttccaagttacagcagcttcttcccgagttgcataaccgacgtaccgacaagagatcagcaacaaatgtgttgcaagagacatgtaactacataagaagcttgcatagagaggtggatgatttaagtgagagattgtctgaattgttggcaacaacagataccactcaagctgctctaattagaagactgctgtcacagtagtagacttaattgattttgctttcctttttaaaaataaaaaataaaacatttttgtttttattatttttcatttctcaagtaattgcaatattcgagtattattgttactagctagatctactttgtagacgaggatttaatgtactttgatgggtttttgagatgtttaatcatctttgctacctttcttatttcattttcgataatactaaatgaaaactacaatttcaaa

SEQ ID NO: 167 - protein, 88 aa, Triticum aestivum
MSSRRSSRGAISDEEVNELMSKLQSLLPNSRRRGSSQASTTKLLKETCSYIKSLHREVDDLSDRLSDLMSTMDHNSAEAEIIRGILRS

SEQ ID NO: 168 - DNA, 1096 bp, Triticum aestivum
  misc_feature (1068)..(1068): n is a, c, g, or t
  misc_feature (1083)..(1083): n is a, c, g, or t
  misc_feature (1086)..(1086): n is a, c, g, or t
gcacgaggcacaactagaagttatccagcgagtatcctgagcagctcgacgtcagcgagggccgcccttcgctcaccggccaaccgcccggcaccttttgagcgtacacactccgaagctttgttagccggcgggagtctgcagtctgcttgctccggcactagctagctagctcatcccggccggccagcgacgtagcagcggctaggaagaagatgtcgagcagaaggtcgtcgcgcggcgccatctccgacgaggaggtcaacgagctcatgtccaagctccagtctctgctccccaactctcgccgccgcggctccagccaggcgtcgacgacgaagctgctgaaggagacgtgcagctacatcaagagcctccaccgggaggtggacgacctcagcgaccggctgtcggacctcatgtcgaccatggaccacaatagcgccgaagcggagatcatccgcggcatcctccgctcgtgatcgtaccacagcgccggccggtcgatcggcgagagctcaaccgccaggacaattaagcggcagcggcgccatgggtctctccgccggccagccggacacgtacgagagctttgcttagctagggtatatatatcgtcctccacatatttaaatatgtaatgtctcttttctgctccctttctgcctagatctgatcgtgtagatcaaaaaatgtactgcgtgtctctaagcttcactccgtctgtactacgtagggcattagcttagctagcgttcctaccttgggccaaagcttatcctcgcgcgctggctgctgcttgagctaatctttcgatcgtctcctccgtgtgcttccctcgctagctcggagctggatagatagctccccccgtcctcctgtctgcctcttcccctcttttgttgtccctttcttgatctactactcgatctgtaaatttagttggtggcattggatcgagttgtgtcctctatagacaaccgaccgaccactactacggtactactactacctagagcaaattaatatcatcgtcatgttgtaccaccccagctttaacttattgtngaatacgtactacgnagnatcaaattaa

SEQ ID NO: 169 - protein, 85 aa, Triticum aestivum
MSSRRGRITDEEINELISKLQALVPESSRRRSASRSSASKLLKETCGYIKSLHQEVEDLSDRLSELMSTLDETSPQAEIIRGLLR

SEQ ID NO: 170 - DNA, 890 bp, Triticum aestivum
  misc_feature (8)..(8): n is a, c, g, or t
cacgcagnacgcttcatcggccgaatcacaggctaaggtaaagcatcaacatatacacgagcaagcaaggaaagccaagcgttagctgtctccgatcagagccggccggccgcagagagagagagggagggagatgtcgagccgccgtggcaggatcaccgacgaggagatcaacgagctcatctccaagctccaggcgctggtcccggaatcatcccgccgccgctccgcgagccggtcgtcggcgtcgaagctgctgaaggagacgtgcggatacatcaagagcctccaccaggaggtcgaggacctctccgacaggctctcggagctaatgtcgaccttggacgagaccagcccccaggccgagatcatccggggccttctccgctagccggattttatcctaccgattgagccagtaggcagtagctacatatatattagcttgaattcatcggccgaaggagggagacgaggaggcaagacaagagaagagaagacaacaagagaggcatagcattttttagctgctgcttgtttcttttcctgcctgctcccccgtctcctggtacgtcgtcgtccgtgtgcgtgtgtgtctttgtgaggccctcatttagcttccggtatactccactgtgtttcatttatgcaccaggttggtagaggttaataatatatatggtgcttctaccgattagccgagtgtattactactagcttgtagacgtgtgtgtttgtgctgttgtaggcctgtagcttctctcagactgagatgcatcctctggctatagagctgttgttttgtactactagtactatctattgctaggtttgtccctgttttccaacggacaagctagctaggttaacgacgagcgtggactacgggg

SEQ ID NO: 171 - protein, 88 aa, Triticum aestivum
MSGRRSRGSVSEEEINELISRLQTLLPTARRRGSSSSQASTTKMLKETCSYIKSLHREVDDLSDRLSDLMSTMDNNSPAAEIIRSLLR

SEQ ID NO: 172 - DNA, 894 bp, Triticum aestivum
  misc_feature (43)..(45): n is a, c, g, or t
gcacgagggcacttcgcagctagccgcccactctacttccgannnacagcctctccatcgaccgaccttcgttcgccctgcattactctgtgaagacgatgtctggcaggaggtcgcgcggctccgtgtcggaggaggagatcaacgagctcatctccaggctccagaccctgctccccaccgcgcgccgccgcggcagcagcagcagccaggcgtcgacgacgaaaatgctcaaggagacgtgcagctacatcaagagcctgcacagggaagtggacgacctcagcgaccgcctctccgacctcatgtccaccatggacaacaacagccccgccgccgagatcatccgcagcctcctccgctagctacctagctggctgactgctcatcatcatcatcgatcacctcctgctgattgtcctaagctagttcatcatctacgtacagctcgtgcagtcctagctaattaagcgcatatgatctacacatacagtacatggtatatatgtcgtccgtcgatctatgcaagcatatatatgcagatcgatcgatatctaattaaggaggactgcatgctaaggccggctggtctaatttactttggtagggcatccatcattagctagctagggccgccagctaagttctatagctgcttcattagctcacccttgcatgcggtttcctcacagtttccatcccccctccccctctctcttattttcatttgcttgtgtaaacttggttatttgcagtctggatcgagttgtttcccctagagacaaccggccgaccgctagctacccatccggtggtggtactgctaatatactactactgctactcagtatcaatcacccatgaatgtatgtactatgactgtcgggcttcggc

SEQ ID NO: 173 - protein, 91 aa, Vitis vinifera
MSSRRSRSRQSGGSRITDDQINDLVSKLQQLLPEIRGRHSDKVSAAKVLQETCNYIRSLNREVDDLSERLSELLATTDSAQAAIIRSLLTQ

SEQ ID NO: 174 - DNA, 276 bp, Vitis vinifera
atgtctagcagaagatcacgctcaaggcaatccggaggttccaggatcacggatgaccagatcaatgatctggtttccaagttgcaacagcttcttcctgagattcgaggcaggcactcggacaaggtctcggcagctaaggtcttacaggagacatgcaactatattagaagcctgaacagagaggttgatgacctaagtgagcgattgtctgagttattggcaacaacagactctgcccaggcagccattattaggagtctacttacgcaatag

SEQ ID NO: 175 - protein, 92 aa, Vitis vinifera
MSSRRSRSRQPGVSRISDDQIADLVSKLQQLIPEIRNRRSDKVSASKVLQETCNYIRNLHREVDDLSDRLSALLASTDTDSDQAAIIRSLLM

SEQ ID NO: 176 - DNA, 279 bp, Vitis vinifera
atgtcaagcaggagatcgcgttcaagacagccaggagtttcgaggataagtgacgatcagattgctgatctcgtgtccaagttacagcagcttattcctgagattcgcaataggcgctccgacaaggtatcggcttctaaagtcttgcaggagacttgcaactatattagaaatttgcatagagaggtggatgacctaagcgatcgattgtctgcgcttttggcttccaccgacacggatagcgatcaggctgccataattaggagcttactcatgtaa

SEQ ID NO: 177 - protein, 90 aa, Vitis vinifera
MSTQRARASRVTDDEINDLILKLQALLPHSNQRRTSTGASAWRILKETCSYIKRLHREVGDLSERLSQLLDSLDNINGVEVEQLRSLLQR

SEQ ID NO: 178 - DNA, 273 bp, Vitis vinifera
atgtctacccaaagagcaagagcttcacgagtcaccgacgatgagattaacgacctcatcctcaaactgcaggcgttgctacctcattcaaatcaaaggcgcacgtctacaggggcatcggcatggaggattctgaaagaaacgtgcagttacataaagaggctacacagagaggtgggcgacctgagtgagagactatcccagcttcttgattctcttgataatattaatggtgttgaggttgagcaacttagaagtttattgcagcgatag

SEQ ID NO: 179 - protein, 90 aa, Vitis vinifera
MSSRRSRQSGSSRISDDQIIELVSKLQQLLPEIRNRRSDKVSASKVLQETCNYIRSLHREVDDLSERLSRLLATVDADSPEAAIIRSLIM

SEQ ID NO: 180 - DNA, 273 bp, Vitis vinifera
atgtctagcagaaggtcgaggcagtcagggtcttcgaggatctcagatgatcagatcattgaacttgtgtccaagttgcagcaacttcttcctgagattcgcaataggcgttcagacaaggtgtcagcttccaaggtcctacaggagacctgcaactacattagaagcttacacagagaggtggatgacctaagcgaacgactgtccaggttactggctacagtcgatgctgatagtcctgaggctgcaataatcaggagtttaattatgtaa

SEQ ID NO: 181 - protein, 103 aa, Welwitschia mirabilis
MEGSSSSSRSRRSSGSSSRGASRHSCRVSEQQINDLLSKLQSLLPDVCESGDKMPASKVLQETCNYIKSLHREVDDLSERLAEILANVESDSVQAAIIRSLLT

SEQ ID NO: 182 - DNA, 657 bp, Welwitschia mirabilis
gtctggtttagtctcagttcagctcagtttactctccagctgagtttgtcggattgagtgtgtgaaaggaaagtcatagtgtgtggaatggaaggatcgtcgagctccagtagaagcagaaggtcttctggttcttcgtcgcgcggtgccagcaggcactcttgcagagtttccgaacagcaaatcaatgatcttctctcgaagcttcagtcgcttctgccggatgtttgtgaatccggagataagatgcctgcgtccaaagttctacaagaaacatgcaactacatcaagagtcttcacagagaggtggacgatttaagcgagcgcttagctgaaattctcgcaaacgtagagagcgacagcgtgcaggctgcaatcatcaggagccttctcacataaatgctcctgtttctttctaatttgtctacctcaggcccctttctacttctttgctttctgcttcttattttgtaacagacaagaagcacagttaagcataaactttagagtatcggcgatcttatgttgctcatgtcatatatatcataaaaaagaatttgcttttttactttgtttctcactcttgatgaacatcatctggtgagttgcagaatctaataattcacagacatacactgtgtatggatctgtattacg

SEQ ID NO: 183 - protein, 87 aa, Zea mays
MSSRRSRASTVSEEEINELISRLQTLLPSARRRGGSQASTTKLLKETCSYIKSLHREVDDLSDRLSDLMASMDHNSPGAEIIRSLLR

SEQ ID NO: 184 - DNA, 954 bp, Zea mays
ctcccctgagctcgatcggatcgtgcacagcccgaacagccgctccaccctcccgctcagctcgctcttcggcggtgctgctctcgatcgtcttctccgtcaccggcggccgcttagcctccgcgatcccggccggtggtcttcttcgccgcattatctctctctctctctctctctctctctaacacaaggacgatgtcgagccggaggtcccgcgcgtcgacggtctcggaggaggagatcaacgagcttatctcgaggctgcagacgctgctccccagcgcgcgccgccgtggcggcagccaggcgtcgacgacgaagctgctcaaggagacctgcagctacatcaagagcctgcaccgggaggtggacgatctgagcgaccgcctgtcggacctcatggccagcatggaccacaacagccccggcgccgagatcatccgcagcctcctccgctagcccaagtccgtccgtccggccggccggccacgcgcgccgcatatatgcagcatctgcgcgcgcgctgtctctctccatccatggacgacggccggcctctcgccatcgccagatctcagcgcattgccgagtgtgtgtgtgtacccatgcatatagcagctgaatatacccaaggacgacaggctaaggctggtttattaattggtagggcattattaagtactactccgtactattaactagggctgcctagcctaagtacggtttctctctctcttccactcttggttgtttgtttccttgctatactcctagtagcttagctccttttccatttgtttgtactcttggctgcttcgtcacatgttcttcctcgtcgtcgtcgtcgtcgtcgtaaacctatgtgtggtctggatcgagttgtttcctccttgacagacaaccgaccaaccgcgagtcgagctaccgatcgatctgtcaacttgtactacgtac

SEQ ID NO: 185 - protein, 89 aa, Zea mays
MSSRRPSSRGNISEDEINELISKLQALLPSSRRRGSGQASTTKLLKETCSYIKSLHREVDDLSDRLSDLMATMDHNSPGAEIIRSILRS

SEQ ID NO: 186 - DNA, 1489 bp, Zea mays
cccccgccccgagccagctcgctataaaggcacccctctccctttctgtctccctcgcagctcagttactcactccggcctccagcttttcctttgcgccccagggccggaagagcccacacaccaccaccaccactagatagccagcgacgatcgagccgcgcgccggtggacgcgcacatacacgcggtagcttccttctcaagcctagctagccccatcctgcgcgcaggcggcggcagcgagaattgacgtgcgacgacgagcttgtgcttgctagctagagagcgaggatgtcgagccggaggccgtcgtcgcgtggcaacatctccgaggacgagatcaacgagctcatctccaagctgcaggccctgctccccagctcccgccgccgcggctccggccaggcgtcgacgacgaagctgctcaaggagacctgcagctacatcaagagcctgcaccgggaggtggacgacctgagcgaccggctgtccgacctcatggccaccatggaccacaacagccccggcgcggagatcatccgcagcatcctccgctcctgatcgcccacgcgcctcctactgcggcggcgccggccggcgcgcgagagagcgaggtcggcgacgacctcgatcgccgacaacgacgaccgcgcgcgcccgcgccccccggcccatctgttctctccaccggctggccgccgggagccgagctcaattagctagggtatatctcaacctctctctctctctctctctcgctcgctctacatgtgtgtgtacctttctcttcctttcacctgcctgtctcctccctagatctgagatcatgtggctagctctcccatatcaaaatgtactagctacacgcgcacgtatggtggtctctgctaagcctaagcttcactgggtagtagggtactagtagcactaggggcttaagcaagctaagcttatcctcctcatcagaatcacattagctcgcaccgcactctctggcttctcgtcgatcgtcttcgtcgtcctcgctcctccgacccacatgatggctagctctccatgtgccgcttctcctctcgcacgtgccgcttctcctcttgtgttcgatatgttgtattgtttccatgtaaatttagtcggtggcctggatcgagttggctagctctctctacaggcaagagacaaccgaccgaccactactatcctagagcaaattgattatcgtcatgatggtgtactgttcaagcttttattaacgacttttaatttacttgctacgtacagcacgtacgtcctacgagaaatgcttttgtgttttttttttctttcactcagggtgccatggaccatggttaagagatgcaaccgctgtgtatgtactgtagtactagtatgcagcgaaaacgttgccctgcctttcatattggctgcttcgatcggtccacttatttggttccgtacggtggaacgtgaaacgacgcgtttacttc

SEQ ID NO: 187 - protein, 89 aa, Zea mays
MSSRRPSSRGNISEDEINELISKLRALLPSSRRRGSGQASTTNLLRETCIYIKSLHREVDDLSDRVSDLVATMDHNSPGAEIIRSILRS

SEQ ID NO: 188 - DNA, 606 bp, Zea mays
cagttactcactccggcctccagtttttcttttgcgccccagggccggaggagcccacccaccaccaccaccactaaatagccagcgacgatcgagccgcgcgccggtggacgcgcacatacacgcggtagtttctttctcaagcctagctagccccatcctgcgcgcaggggggggcagcgaaaattgacgtgcgacgacgagcttgtgcttgctatctagagagcgaggatgtcgagccggaggccgtcgtcgcgtggcaacatctccgaggacgagatcaacgagctcatctccaagctgcgggccctgctccccagctcccgccgccgcggctccggccaggcgtcgacgacgaatctgctcagggagacctgcatctacatcaagagcctgcaccgggaggtggacgacctgagcgaccgggtgtccgacctcgtggccaccatggaccacaacagccccggcgcggagatcatccgcagcatcctccgctcctgatcgcccacgcgcctcctactgcggcggccccggccggcgcgcgagagagcgaggtcggacacgacctcgatcgccgacaaagacgaccgcgcgcgcccgcgcccc

SEQ ID NO: 189 - protein, 90 aa, Zea mays
MSSRRSSSHGNISEDEMNELVSKLQALLPSSRRRRGSGQASTAKLLKETCSYIKSLQREVDDLSDRLSDLLSTMDHNSPAAEIIRSILRS

SEQ ID NO: 190 - DNA, 999 bp, Zea mays
  misc_feature (986)..(986): n is a, c, g, or t
gctacagctttaattttgcagagcgaccaccagtccaggtccagcgcgggacgcatcacatacacgcggtaccttcgttcgttctcaagcttatagcgcccgatcgaccctgcgcggagctagttcgttcgttccggcaggcggcggcagcgaagcagtgcgacgacgacgacgaggtacgtagagagcgagaggatagatgtcgagccgaaggtcgtcgtcgcacggcaacatctccgaggacgagatgaacgagctcgtctccaagctccaggccctgctccccagctcccgccgccgccgcggctccggccaggcgtcgacggcgaagctgctgaaggagacctgcagctacatcaagagcctccagcgggaggtggacgacctcagcgaccggctgtcggacctcttgtccaccatggaccacaacagccccgcggcggagatcatccgaagcatcctccgctcctgagcgcgcgcaagggcgaggtcaacgaacgccggcctccgatcgatcgccgacagcgcgcgctctccggccggctggtcacgtgcgggagctgagctcaattaggtagctagggtatatatacatataatatatatatctacatgtacgtgtatctaccttttcctttaccagtctccctagatctgagatcatgtggctagctccgtataaaaatgtactagccacacgctgacacgcacacgtatgcatgatggtctctgcgctaagcttcactgggtagtagggtatagcactagagcctaagcaagcttatcctcctcactttagtatagctcgcagcagcagcagtctctcgattcctcggcgatcttcggtggcttaattggatcgagctggctagtgcgctctctctctctctctcatctctagcaggcaggaaaagagacaaccgaccgaccactactagcctagcctagagcaaattgactgtcgttatgatgatgatgatgtactntaaagcttttatt

SEQ ID NO: 191 - protein, 105 aa, Zea mays
MSSGRRPSRTRRAGSSSLSSSSTSRSISDDQISELLSKLHALLAESQARNGGAHRGSAARVLHDTCSYIRSLHHEADNLTETLAELLTSADVTSDQPPVITSLFM

SEQ ID NO: 192 - DNA, 497 bp, Zea mays
ctattcgttggggaggaaaagacttaaagcagtctatttgtctactcgccgagagctctctttcccctcttctatagctactgcatgctctgctagtcgatcagctaggcagctaggtagctagctccgatccacatatataaaatcagatcgcacagctactagcggcaaccgacatgtccagcggccggaggccgtcacgcacgcggcgtgcggggagcagctcgctgtcgtcgtcgtcgacgtccaggtccatctcggatgaccagatctccgagctcctgtccaagcttcacgcgctgctcgcggagtctcaagctcgcaatggcggcgcacatagggggtccgcggcgagggtgctgcacgacacgtgcagctacatcaggagcctgcaccacgaggcggacaacctcaccgagacgctggccgagctgctcacctccgccgatgtcaccagcgaccagccccccgtcatcacgagcctgttcatgtgacca

SEQ ID NO: 193 - protein, 85 aa, Zingiber officinale
MSSRRNRVSEEEINELISKLQSLLPETRRRGAGRASAAKLLKETCSYIRSLNREVDDLSDRLSGLMATLDSNSAEAEIIRSLLPS

SEQ ID NO: 194 - DNA, 535 bp, Zingiber officinale
ttctagatttaagatgtcgagccggaggaacagggtctcggaggaggagatcaatgagctcatctccaaacttcagtctctcctcccggaaacccgccgccggggcgctggccgggcgtccgcggcgaagttgctgaaggagacgtgcagctacatcaggagcctgaacagggaggtggacgacctcagcgacaggctctcggggctcatggcgacgctggacagcaacagcgccgaggcggagatcatccggagcctgctcccctcctgattcaaattcctgatcgtcagttaggacttactccagacgtaaagatatatgtagtgtacgtacctctgttctgaaagcttaggagttaggacgacgaagctagcagcgattttcctttactcgaagcctgattcgagttgttttctctgtagacaaccgaccgacacatctgcttaaaaaaatcagtaatgcttcccatgtaatcgaggttcgagatgtagtgtcgtattttactatccactgtccgtctcttgttcttacgtg

SEQ ID NO: 195 - protein, 85 aa, Zingiber officinale
MSSQRGRITDKEIHELVSSLQALLPESRRRSTSRASSSKLLKETCSYIRSLQREVDDLSGRLAELMSTMDSDSPQAEIIRSIFRS

SEQ ID NO: 196 - DNA, 449 bp, Zingiber officinale
gcaccctttttcctctccgcaaacacatcctcagattactgcgcgcgctagaatgtcgagccagagaggaaggattactgacaaggaaatccacgagctcgtctcctcgctgcaggctcttctcccggagtctcgccgcaggagcacgagtagggcatcatcatccaagttgctgaaggagacatgcagctacatcaggagcttgcagcgggaggtggacgacctcagcggccggctcgccgagttgatgtcgacgatggactccgacagccctcaggctgagatcattaggagcatcttccggtcctaaataataactatatatagtcatctttgtaccatttcatcagcctattagctagtgttatatataagcatgcagaattaatactgctgctgctgcttcttcttcttgatgccatcgatgcgatctagcgagcaataaagtt

SEQ ID NO: 197 - DNA, 483 bp, Arabidopsis thaliana
atggctagtggaatcgctcgtggtcgtttagctgaagagaggaaatcgtggaggaagaatcatcctcatggttttgtggcaaagccggagacggggcaggatggaactgtgaatctaatggtgtggcattgcactatacctggtaaagctgggactgattgggaaggtggattctttccattaacgatgcacttcagtgaggattatccgagcaaacctccgaaatgtaaatttccacaagggtttttccaccctaatgtctatccatctggaactgtctgtctctctatccttaacgaggattatggatggagaccagccatcaccgtgaagcagattcttgttggtattcaggatttacttgacacaccgaatcccgctgaccctgcacagacagatggttatcatctcttctgtcaggatccagttgagtacaagaaaagggtgaagctgcagtccaagcagtatcctgctcttgtctaa

SEQ ID NO: 198 - protein, 160 aa, Arabidopsis thaliana
MASGIARGRLAEERKSWRKNHPHGFVAKPETGQDGTVNLMVWHCTIPGKAGTDWEGGFFPLTMHFSEDYPSKPPKCKFPQGFFHPNVYPSGTVCLSILNEDYGWRPAITVKQILVGIQDLLDTPNPADPAQTDGYHLFCQDPVEYKKRVKLQSKQYPALV

SEQ ID NO: 199 - DNA, 483 bp, Helianus annuus
atgtccggtggaattgctcgcggccgtctcaccgaggaacgcaaagcatggcgcaagaatcatcctcatggttttgtggcgaaaccggagactctacctggcggtacggttaatttgatgatttggagttgtatcatccctggtaagaatgggaccgactgggagggcggtttttacccccttaccctccacttcaccgaggattatccgagcaaaccaccaaagtgtaaattccctcaaggcttcttccaccccaatgtttacccttctgggaccgtttgtttgtccatccttaacgaagatagtggttggaggccagcaataacagttaaacaaattctagttggcatccaggacttgctggattcccccaatcccgctgatcccgcccagactgatggatatcatctctttatccaggacacggtggagtacaagagacgggtccgccagcaagcaaagcaatacccagcactcgtgtag

SEQ ID NO: 200 - protein, 160 aa, Helianus annuus
MSGGIARGRLTEERKAWRKNHPHGFVAKPETLPGGTVNLMIWSCIIPGKNGTDWEGGFYPLTLHFTEDYPSKPPKCKFPQGFFHPNVYPSGTVCLSILNEDSGWRPAITVKQILVGIQDLLDSPNPADPAQTDGYHLFIQDTVEYKRRVRQQAKQYPALV

SEQ ID NO: 201 - DNA, 1119 bp, Triticum aestivum
  misc_feature (873)..(873): n is a, c, g, or t
aaaaaaagtcgagaagacacaatcaaggagtcaagttcaaaagtatccacacatccatcattcaagcagaaggacctgatttttgtttggtcaacaccagaccacacgtcacaaatgtccacggtcacaatggaaagctctccacaataccccgctttcggttatatttagtcatacttcatattttcaggtgcggtaatttatgattttagactgctacatggatcctcaaaccagagcagggtactgcttggcctgcagccgaatgcgtctcttgtactctgctggatcctggataaagaggtgataaccatcagtctgggcaggatcagctggattaggttgatcaagcaaatcctgtattccaactagaatctgcttaacagtgatggcaggtctccaaccactatcctcattaagaatcgagaggcagaccgtccctgaaggatagacatttgggtggaaaaaaccctgcgggaacttgcacttgggaggtttgctagggtagtcctcactgaaatggagagtaagtgggtagtatccactttcccaatcagtcccctgctttccggggatggtgcagttccagatcatgagattcaccgacccgtcggccaccgtctccggcttcgcgacgaatccatgggggtggttcttgcgccaggccttgcgctcctccgcgaggcggccccgcgcgatccctcctcctgacatgggcggcggcggaggaggctgctggctgctctctgtcgctgagtctggggtttgggtttcggagtctgcgcagtttctaatccttcagtccatgtcagtgtcctcgtgctccagtcgcggacgcgttgggtcaagattttccgggaatttcgggaccgntaccagcctggtttttttgttacaaactggttctataggtgtcactaaataggcctaatggtcatacctggttcctgtgtgaaaatgttatcggcccgggggctaaggtaaagttcggaggttggatggtcccggggttttctatggaggtcaaaacagggtgattgggtttccggggaactgacggttactaaacgagcttgtttttttaaactccacgggtaacccttccgccctatttgttaa

SEQ ID NO: 202 - protein, 161 aa, Triticum aestivum
MSGGGIARGRLAEERKAWRKNHPHGFVAKPETVADGSVNLMIWNCTIPGKQGTDWESGYYPLTLHFSEDYPSKPPKCKFPQGFFHPNVYPSGTVCLSILNEDSGWRPAITVKQILVGIQDLLDQPNPADPAQTDGYHLFIQDPAEYKRRIRLQAKQYPALV

SEQ ID NO: 203 - DNA, 486 bp, Hordeum vulgare
atgtcaggaggagggatcgcccgcggccgcctcgcggaggagcgcaaggcctggcgcaagaaccacccccatggattcgtcgcgaagccggagacgatggccgacgggtcggtgaatctcatgatctggaactgcaccatccccggaaagcaggggactgattgggaaagtggatactacccacttaccctccatttcagtgaggactaccctagtaaacctcccaaatgcaagttcccgcagggttttttccacccaaatgtctatccttcagggacggtctgcctctcgattcttaatgaggatagcggttggagacctgccatcactgttaagcagatcctagttggaatacaggatttgcttgatcaacctaatccagctgatcctgcccagactgatggttatcacctctttatccaggatccagcagagtataggaggcgtattcggctgcaggctaagcagtaccctgctctggtttga

SEQ ID NO: 204 - protein, 161 aa, Hordeum vulgare
MSGGGIARGRLAEERKAWRKNHPHGFVAKPETMADGSVNLMIWNCTIPGKQGTDWESGYYPLTLHFSEDYPSKPPKCKFPQGFFHPNVYPSGTVCLSILNEDSGWRPAITVKQILVGIQDLLDQPNPADPAQTDGYHLFIQDPAEYRRRIRLQAKQYPALV

SEQ ID NO: 205 - DNA, 480 bp, Glycine max
atgtctggtatcgcccgtggacgtcttgccgaggagcgaaagtcatggcgcaaaaaccaccctcatggtttcgttgccaagcccgagactctccctgatgccaccgttaatttgatggtctggcattgcactattcctggcaaggctgggactgattgggagggtggatatttcccactgacaatgcacttcagtgaagattaccctagcaagcctcccaagtgcaaattccctcaaggtttctttcaccccaatgtgtatccatctggaactgtttgcttgtctatacttaatgaagatagtggatggagaccagctataacagtgaagcaaattcttgtgggcatccaggacctgcttgatcaaccaaatcctgctgaccctgcccagacggagggttatcatctattcatccaggatgcagctgagtacaagagaagagtccgacagcagtcaaagcaatatccacctcttgtctag

SEQ ID NO: 206 - protein, 159 aa, Glycine max
MSGIARGRLAEERKSWRKNHPHGFVAKPETLPDATVNLMVWHCTIPGKAGTDWEGGYFPLTMHFSEDYPSKPPKCKFPQGFFHPNVYPSGTVCLSILNEDSGWRPAITVKQILVGIQDLLDQPNPADPAQTEGYHLFIQDAAEYKRRVRQQSKQYPPLV

SEQ ID NO: 207 - DNA, 483 bp, Zea mays
atgtcgggaggaatcgcgcgcggccgcctcgccgaggagcgcaaggcctggcgcaagaaccacccccacggtttcgtggcgaggccggaaacgctggccgacgggtcggcgaacctcatggtctggagctgtaccatccccggcaagcaggggactgattgggaaagtgggtactacccacttacccttcatttcagtgaagattacccaagcaagcctcccaaatgcaagttcccacagggttttttccacccaaatgtttatccttcaggaacagtctgcctctcaattctcaatgaggatagtggttggagacctgctatcactgttaagcagattctagttggaatacaagacttgcttgatcagcccaatccagctgatcctgcccaaactgatggttatcacctattcatccaggatccaacagaatataagcgacgtgttcgtctgcaggccaagcaatatcctgctctggtctga

SEQ ID NO: 208 - protein, 160 aa, Zea mays
MSGGIARGRLAEERKAWRKNHPHGFVARPETLADGSANLMVWSCTIPGKQGTDWESGYYPLTLHFSEDYPSKPPKCKFPQGFFHPNVYPSGTVCLSILNEDSGWRPAITVKQILVGIQDLLDQPNPADPAQTDGYHLFIQDPTEYKRRVRLQAKQYPALV

SEQ ID NO: 209 - DNA, 483 bp, Zea mays
atgtctggagggatcgcgcgcggccgcctcgccgaggagcgcaaggcctggcgcaagaatcacccccacggtttcgtggcgaggccggagtcgctgaccgacgggtccgtgaacctcatggtctggaactgtaccatccccggcaagcacgggaccgattgggaaggtgggtactacccacttacccttcatttcagtgaagattacccaagcaaacctcccaaatgcaagtttccacagggttttttccatccaaatgtttatccatcaggaacagtctgcctctcaattctgaacgaggatagcgattggagacctgctatcactgttaagcagattctagttggaatacaggacttgcttgatcagcccaatccagctgatcctgctcagactgatggttatcacctattcatccaggatcctgcagaatataagcgacgtgttcgtctgcaggccaagcaatatcctgctctggtctga

SEQ ID NO: 210 - protein, 160 aa, Zea mays
MSGGIARGRLAEERKAWRKNHPHGFVARPESLTDGSVNLMVWNCTIPGKHGTDWEGGYYPLTLHFSEDYPSKPPKCKFPQGFFHPNVYPSGTVCLSILNEDSDWRPAITVKQILVGIQDLLDQPNPADPAQTDGYHLFIQDPAEYKRRVRLQAKQYPALV

SEQ ID NO: 211 - DNA, 483 bp, Zea mays
atgtctgggggaatcgcccgcggccgcctcgccgaggagcgcaaggcctggcgcaagaaccacccgcacggtttcgtcgcgaagccggagtcgctgcccgacgggacggtgaacctgatgatctggcagtgcaccatccccggcaagcaagggactgactgggaaggtggatatttccctctcacccttcattttagtgaggattaccctagcaagcctcccaagtgcaagttccctcagggtttcttccacccaaatgtgtatccttctggaacagtctgtctttcgatccttaatgaagatagtggttggagaccagctattactgttaagcagattctcgtcgggatccaggacttgctagatcagccaaatcctgctgatcctgctcaaacggatggctatcacctttttatccaggatcctacagaatataagaggcgtgttaaactgcaggcgaagcagtatcccgcgttggtctga

SEQ ID NO: 212 - protein, 160 aa, Zea mays
MSGGIARGRLAEERKAWRKNHPHGFVAKPESLPDGTVNLMIWQCTIPGKQGTDWEGGYFPLTLHFSEDYPSKPPKCKFPQGFFHPNVYPSGTVCLSILNEDSGWRPAITVKQILVGIQDLLDQPNPADPAQTDGYHLFIQDPTEYKRRVKLQAKQYPALV

SEQ ID NO: 213 - DNA, 483 bp, Oryza sativa
atgtcggggggaatcgcgcgcggccgcctcgcggaggagcggaaggcgtggcggaagaaccacccacacggtttcgtcgccaagccggagacgttggccgacgggacggtcaacctcatgatctggcactgcacaatccccggcaagcaagggactgattgggaaggtggatactttcctctcactcttcatttcagtgaggattaccctagcaaacctcccaagtgcaagttcccacagggtttcttccacccaaatgtctatccttcagggacagtctgcctttcaattcttaatgaagacagcggttggagacctgctattaccgtcaagcaaattcttgttggaatccaggacttgcttgatcagcctaatcctgctgatcctgctcagaccgatggttaccatctttttatccaggatcctacggaatacaagaggcgtgttcggctgcaggccaagcagtatcctccgattgtctga

SEQ ID NO: 214 - protein, 160 aa, Oryza sativa
MSGGIARGRLAEERKAWRKNHPHGFVAKPETLADGTVNLMIWHCTIPGKQGTDWEGGYFPLTLHFSEDYPSKPPKCKFPQGFFHPNVYPSGTVCLSILNEDSGWRPAITVKQILVGIQDLLDQPNPADPAQTDGYHLFIQDPTEYKRRVRLQAKQYPPIV

SEQ ID NO: 215 - DNA, 483 bp, Oryza sativa
atgtcgggagggatcgcacgcggccgcctcgcggaggagcgcaaggcctggcggaagaaccaccctcacgggttcgtggcgaagccggagacgatggccgacgggtcggcgaacctcatgatctggcactgcaccatccccggcaagcaggggaccgattgggaaggtgggtactaccctcttacccttcacttcagtgaggactatcctagcaaaccacccaagtgcaagttcccacagggctttttccacccaaatgtctatccttcaggaacagtgtgcctctcaattcttaatgaggatagtggctggagacctgctatcactgtaaagcagatccttgttggaatacaggacttgcttgatcagccaaatcctgctgatcctgcacagactgacggttatcacatttttatacaggacaaaccagaatataagaggcgtgttcgtgttcaggccaagcagtaccctgctttgctttga

SEQ ID NO: 216 - protein, 160 aa, Oryza sativa
MSGGIARGRLAEERKAWRKNHPHGFVAKPETMADGSANLMIWHCTIPGKQGTDWEGGYYPLTLHFSEDYPSKPPKCKFPQGFFHPNVYPSGTVCLSILNEDSGWRPAITVKQILVGIQDLLDQPNPADPAQTDGYHIFIQDKPEYKRRVRVQAKQYPALL

SEQ ID NO: 217 - DNA, 402 bp, Oryza sativa
atggtctggcgatgcatcatccccggcaaagaagggactgattgggagggtggatatttcccacttactatgcaattcactgaagactatccaaccaacgctccttcttgcaagttcccatcgggtttcttccacatcaatgtctatgactctggggcagtatgcctatcaatcttgagtaccgcatggaaaccttcaattacagtgaggcaaattcttataggcatccaggaattgtttgatgatccaaaccctaactctgctgcacagaatataagctatgagctttataggacatggaggagtacaggaaacgcgttcgtcagcaggctaagaagtatccttcagctctgtagccgcgcaatgcctgcaggattctggcagctaaaacattttgattga

SEQ ID NO: 218 - protein, 133 aa, Oryza sativa
MVWRCIIPGKEGTDWEGGYFPLTMQFTEDYPTNAPSCKFPSGFFHINVYDSGAVCLSILSTAWKPSITVRQILIGIQELFDDPNPNSAAQNISYELYRTWRSTGNAFVSRLRSILQLCSRAMPAGFWQLKHFD

SEQ ID NO: 219 - DNA, 483 bp, Vitis vinifera
atgtcaggaggcatcgcgcgtggtcgtctcgccgaggagcgaaaagcctggcgtaagaatcatccccatggtttcgtggctaagccagagactggtccggacggttctgtcaatttgatggtgtggcattgcaccatccctggtaaggctgggactgattgggaagggggctacttcccacttactttgcacttcagtgaggactaccctagcaaacccccaaagtgcaagttccctcaaggtttcttccaccctaatgtctacccatctggaactgtatgtctctcgatcctcaatgaagacagtggttggagacctgccattacagtgaaacaaattctagtgggcattcaagacttgctggaccagcccaatcctgcagatccagcacaaactgatgggtatcagctcttcatccaggaacccgcagagtataaaagaagggtgcggcaacaggccaagcaatatccacctcttgtctaa

SEQ ID NO: 220 - protein, 160 aa, Vitis vinifera
MSGGIARGRLAEERKAWRKNHPHGFVAKPETGPDGSVNLMVWHCTIPGKAGTDWEGGYFPLTLHFSEDYPSKPPKCKFPQGFFHPNVYPSGTVCLSILNEDSGWRPAITVKQILVGIQDLLDQPNPADPAQTDGYQLFIQEPAEYKRRVRQQAKQYPPLV

SEQ ID NO: 221 - DNA, 483 bp, Nicotiana benthamiana
atgtcaggaggtatagcccgtggccgtcttgcagaggagcgcaaagcttggcgcaaaaatcacccccatgggtttgtagcaaagccagagacgctttcggatgggtcagttaacttgatggtttggcactgcagtattcctggtaaagcaggaacggactgggaaggcggtttttatccggttacgatacacttcagtgaagattatcctagcaaaccacctaagtgcaaattcccacaaggcttcttccatccgaatgtctatccatcaggaacagtttgcttgtcgatcctcaacgaagatagcggttggagacctgccattacagtgaaacagatactggttggtatccaagacttgttagatcagccaaaccctgctgatcctgcccaaaccgaagggtatcatctctttattcaggatgctattgagtacaagaagcgggttaggctgcaggccaagcagtatcctcctctggtgtag

SEQ ID NO: 222 - protein, 160 aa, Nicotiana benthamiana
MSGGIARGRLAEERKAWRKNHPHGFVAKPETLSDGSVNLMVWHCSIPGKAGTDWEGGFYPVTIHFSEDYPSKPPKCKFPQGFFHPNVYPSGTVCLSILNEDSGWRPAITVKQILVGIQDLLDQPNPADPAQTEGYHLFIQDAIEYKKRVRLQAKQYPPLV

SEQ ID NO: 223 - DNA, 483 bp, Populus x canadensis
atgtcaggtggcatcgcacgtggtcgtcttgctgaggaaaggaagtcctggcgcaaaaaccaccctcatggttttgtggcgaaaccagagacacagccagatggaacagtaaatttgatggtctggcattgcacaatccctggaaaacttggtactgattgggaaggtggttattttcctcttacactcaacttcagtgaagattatcctagcaagccaccaaagtgtaaatttcctcagggtttcttccaccctaatgtatatccatctggaactgtttgcttgtcaatccttaacgaggacagtggatggagaccagccatcacagtgaagcagattcttgtgggtatccaggacttgctggaccagccaaatcctgctgatcctgcccaaactgaaggttatcatctgtttatccaggatgctgcagagtacaagaaaagagttcgccagcaagctaagcaatacccttctcttgtctaa

SEQ ID NO: 224 - protein, 160 aa, Populus x canadensis
MSGGIARGRLAEERKSWRKNHPHGFVAKPETQPDGTVNLMVWHCTIPGKLGTDWEGGYFPLTLNFSEDYPSKPPKCKFPQGFFHPNVYPSGTVCLSILNEDSGWRPAITVKQILVGIQDLLDQPNPADPAQTEGYHLFIQDAAEYKKRVRQQAKQYPSLV

SEQ ID NO: 225 - DNA, 486 bp, Triticum turgidum
atgtcttccggtgggatcgcgcgcggccgcctcgcggaggagcgcaaggcctggcggaagaaccacccccacggcttcgtcgccaagccggagacgctgggcgacggcacggtcaacctcatggtctggcactgcaccatccccggcaagcaagggactgattgggaaggtggatacttccctctcacccttcatttcagcgaggattaccccagcaagcctcccaagtgcaagttccctacaaatttcttccacccgaatgtctatccttcggggacagtctgcctttcaatcctcaatgaggacagcggctggagacctgctattactgtgaagcaaatccttgttggaattcaggacttgcttgatcagcccaacccggctgaccctgctcagactgatggttatcaccttttcatccaggatccagctgagtacaagaggcgtgttcgggcgcaggcaaagcagtatcccgcattggtctga

SEQ ID NO: 226 - protein, 161 aa, Triticum turgidum
MSSGGIARGRLAEERKAWRKNHPHGFVAKPETLGDGTVNLMVWHCTIPGKQGTDWEGGYFPLTLHFSEDYPSKPPKCKFPTNFFHPNVYPSGTVCLSILNEDSGWRPAITVKQILVGIQDLLDQPNPADPAQTDGYHLFIQDPAEYKRRVRAQAKQYPALV

SEQ ID NO: 227 - DNA, 486 bp, Populus trichocarpa
atgtcaggaggaggcatagctcgtggtcgtcttgctgaagagagaaagtcatggcgtaagaatcatccccacggttttgtggctaagcctgataatgcacaagatggttctcttgatttgatggtgtggaagtgcatcatacctggcaaacccgggacagattgggagggtggcttcttccccctctcgcttcatttcagtgaggactacccaagcaaacctccaaagtgcaagttcccccaaggtttcttccaccctaatgtctacccttcaggaactgtgtgcttatctattctcaatgaggactatggctggagaccagccattactgtgaagcaaattttagttggcattcaggatttgcttgatcaaccaaatccttctgatcctgcgcaaactgatggctatcagctttttgtccaggacccgactgagtacaggagaagggtgcgccaacaagccaagcaatatccacctgcgctctga

SEQ ID NO: 228 - protein, 161 aa, Populus trichocarpa
MSGGGIARGRLAEERKSWRKNHPHGFVAKPDNAQDGSLDLMVWKCIIPGKPGTDWEGGFFPLSLHFSEDYPSKPPKCKFPQGFFHPNVYPSGTVCLSILNEDYGWRPAITVKQILVGIQDLLDQPNPSDPAQTDGYQLFVQDPTEYRRRVRQQAKQYPPAL

SEQ ID NO: 229 - DNA, 486 bp, Populus trichocarpa
atgtcaggaggaggcatagctcgtggtcgtcttgctgaagagagaaaggcatggcgtaagaatcaccctcacggttttgtggctaagcctgataatgctccagatggttctctagatttgatgatgtggaagtgcattatacctggcaaacccgggactgattgggagggtggctacttccccctcactcttcatttcagtgaggactacccaagcaaacctccaaagtgcaagttcccccaaggtttcttccaccctaatgtctacccttcaggaactgtgtgcttatctatcctcaatgaggactatggctggagaccagccattacagtgaagcaaatattaattggcattcaggatttgcttgatcaaccaaatccttctgatcctgcacaaactgatggctatcagctttttgtccaggaccctgctgagtacaggagaagggtgcgccaacaagccaagctatacccacctacgctctga

SEQ ID NO: 230 - protein, 161 aa, Populus trichocarpa
MSGGGIARGRLAEERKAWRKNHPHGFVAKPDNAPDGSLDLMMWKCIIPGKPGTDWEGGYFPLTLHFSEDYPSKPPKCKFPQGFFHPNVYPSGTVCLSILNEDYGWRPAITVKQILIGIQDLLDQPNPSDPAQTDGYQLFVQDPAEYRRRVRQQAKLYPPTL

SEQ ID NO: 231 - DNA, 483 bp, Physcomitrella patens
atgtcaggaggaatcgcacgcggtcgtcttgcggaggagcgcaaggcctggcgcaagaatcatcctcatggatttgtagcgaggccggagacaggtgcagatggagctctaaatttgatggtttggcagtgcactttgcctggaaaagttgggacggactgggaaggtggattttaccctgtagcaattcacttcagtgaggattatcccagcaagccccccaagtgcaagtttccacagggttttttccaccccaacgtttatccttcaggcacagtttgcctatccatccttaatgaagattctggttggagaccagctatcactgtaaaacagatccttgtgggtattcaggagcttctcgacgctccgaacccagcagatcccgctcaaaccgaagcctatcagctttttattcaagatccagttgaatacaagcgtcgtgttaggcagcaagccaagcagtacccaccgccaatttaa

SEQ ID NO: 232 - protein, 160 aa, Physcomitrella patens
MSGGIARGRLAEERKAWRKNHPHGFVARPETGADGALNLMVWQCTLPGKVGTDWEGGFYPVAIHFSEDYPSKPPKCKFPQGFFHPNVYPSGTVCLSILNEDSGWRPAITVKQILVGIQELLDAPNPADPAQTEAYQLFIQDPVEYKRRVRQQAKQYPPPI

SEQ ID NO: 233 - DNA, 483 bp, Physcomitrella patens
atgtcgggaggaattgcgcggggtcgacttgcggaggagcgcaaggcctggcggaagaatcatcctcatgggtttgtggctaggcctgagacatgtgcagatggagctcttaatttgatggtttggcagtgtactttacctggaaaagttgggaccgactgggaaggtggattctatcctgtagcaattcactttactgaagattatcccagcaagcctcctaagtgcaaattcccacagggtttcttccaccccaacgtgtatccttcaggcacagtttgcctctccatcctgaatgaagattcgggttggagaccagctatcaccgtgaagcagatcctcgtcggtatccaggagctgctcgacgctccaaacccagcagatcccgctcagactgaagcttatcagctttttattcaggatccagttgaatacaagcgacgagtaaggcagcaagccaagcaatacccaccaccaatctaa

SEQ ID NO: 234 - protein, 160 aa, Physcomitrella patens
MSGGIARGRLAEERKAWRKNHPHGFVARPETCADGALNLMVWQCTLPGKVGTDWEGGFYPVAIHFTEDYPSKPPKCKFPQGFFHPNVYPSGTVCLSILNEDSGWRPAITVKQILVGIQELLDAPNPADPAQTEAYQLFIQDPVEYKRRVRQQAKQYPPPI

SEQ ID NO: 235 - DNA, 480 bp, Chlamydomonas reinhardtii
atgtctggcgtcgcacgctcacgcttgcaagaggagcggaaagcctggcggagggataagccgttcggcttccatgctcgaccagaaaccgcagacgacgggagcgtgaacctgatgaagtggaagtgccacatccccgggaaacaaggcacggactgggagggcggcttctacccgctcaccatggagttcagcgaggactaccccaccaagccgcccaagtgcaagttccccgcgggcttcttccaccccaacatctacccctccggtaccgtgtgcctcagcatcctcaacgaggacgagggctggcggccctccatcaccatcaagcagctgctgttgggcatccaggagctgctggacacgcccaaccccggcagccccgcccagtccgacgccttcgtgctgttcacgcagcagaaggccgagtacgtcaagaaggtgaagcgccaggcgctcaactacccgccaccctcgtga

SEQ ID NO: 236 - protein, 159 aa, Chlamydomonas reinhardtii
MSGVARSRLQEERKAWRRDKPFGFHARPETADDGSVNLMKWKCHIPGKQGTDWEGGFYPLTMEFSEDYPTKPPKCKFPAGFFHPNIYPSGTVCLSILNEDEGWRPSITIKQLLLGIQELLDTPNPGSPAQSDAFVLFTQQKAEYVKKVKRQALNYPPPS

SEQ ID NO: 237 - DNA, 381 bp, Prunus armeniaca
gggacggtgaatttgatggtgtggcattgcacgattcctggcaagaccggtactgactgggaggggggttttttcccacttacccttcacttcagtgaagactaccctagcaagcctccaaagtgtaaattcccaccaggtttcttccacccaaatgtatatccatctggaactgtttgtctatcaattcttaatgaggacagtggttggagaccagcaataaccgtgaagcaaattcttgtgggcattcaggatttactggatcagccaaatcctgctgatcctgcacagacagaagggtatcacctcttcattcaggatgccacggagtacaagaaaagggttcggcagcaggccaagcaatacccacctctagtttaa

SEQ ID NO: 238 - protein, 126 aa, Prunus armeniaca
GTVNLMVWHCTIPGKTGTDWEGGFFPLTLHFSEDYPSKPPKCKFPPGFFHPNVYPSGTVCLSILNEDSGWRPAITVKQILVGIQDLLDQPNPADPAQTEGYHLFIQDATEYKKRVRQQAKQYPPLV

SEQ ID NO: 239 - DNA, 480 bp, Ostreococus tauri
atggcgtcggtcgccctcgcgcgcctgggcgaggagcgccgaaactggcgtcgcgaccacccgcccggattcttcgcgcgtcccgagaaaacggcgagcggggagacgaatctgtttcgatggcggtgctcgataccgggcgcgcgcgggacgcgctgggagggcgcgttcgtgccgctgacgatggagttcccgagggagtacccggccaagccgatgaagtgtaaatttcctgcgggattttatcacccgaacgtgtacccgagcgggacggtgtgcctgagcattctgaacgaggacgaggggtggcggccgagcgtgacggtgaagcaggtggcgctggggatacaggaactgctggataatccgaacgagaagtcgccggcgcagagcgatgcgtacgtgacgtacacgacggatagggcgaagtacgagcggagggtgagggaggaggtggcgaagtatccgccgccggagtag

SEQ ID NO: 240 - protein, 159 aa, Ostreococus tauri
MASVALARLGEERRNWRRDHPPGFFARPEKTASGETNLFRWRCSIPGARGTRWEGAFVPLTMEFPREYPAKPMKCKFPAGFYHPNVYPSGTVCLSILNEDEGWRPSVTVKQVALGIQELLDNPNEKSPAQSDAYVTYTTDRAKYERRVREEVAKYPPPE

SEQ ID NO: 241 - DNA, 483 bp, Picea sitchensis
atgtctggaggcatagctcgaggtcgacttgctgaggagcggaaggcctggcgtaagaaccatccccatggtttcgtagctagacctgacactcagccagatggttcccttaacttaatggtgtggcaatgcatcattcctggcaaatctgggacggattgggagggtggttactttcctctaacaattaatttcagtgaggattacccaagtaaacctccaaaatgcaagttccctcaagggtttttccatcctaatgtttatccatcaggaactgtttgtctgtctatccttaatgaggattctgggtggcggccagccattactgtgaagcaaatacttataggtattcaagaccttttagatcagccaaatccaggtgatcctgcacaaacggatggctaccatctttttatccaagacctcacagaatacaagcggagagttcgacaacaggcaaaacaatacccacctctggtgtga

SEQ ID NO: 242 - protein, 160 aa, Picea sitchensis
MSGGIARGRLAEERKAWRKNHPHGFVARPDTQPDGSLNLMVWQCIIPGKSGTDWEGGYFPLTINFSEDYPSKPPKCKFPQGFFHPNVYPSGTVCLSILNEDSGWRPAITVKQILIGIQDLLDQPNPGDPAQTDGYHLFIQDLTEYKRRVRQQAKQYPPLV

SEQ ID NO: 243 - DNA, 54 bp, Artificial sequence
  primer 1
ggggacaagtttgtacaaaaaagcaggcttaaacaatggctagtggaatcgctc

SEQ ID NO: 244 - DNA, 49 bp, Artificial sequence
  primer 2
ggggaccactttgtacaagaaagctgggtatcagttttggtgcgttctc

SEQ ID NO: 245 - DNA, 2194 bp, Oryza sativa; identical to SEQ ID NO: 5

SEQ ID NO: 246 - DNA, 2196 bp, Lycopersicon esculentum
atggatgcttatgaagctacaaaaattgtttttcaaaggattcaaagtttggatcctgaaaatgcatcaaaaattatggggattcttctgatgcaagaccatggtgagaaagaaatgattcgattagcttttggtccagaagctttagttcactcggtgattcttaaagcaagaaaggagcttggtgtttcttcaaactcaccttctacaccttcaactccttcttcaccttcaccttttggtggttcaatgtgtttttcaaggcagaattcttcttcttcagctacttctggtaggattcttgggggtcttagccttccttcacctcttagcataactagtaacaacaaccactcttcaaatgtttctgcttcttggagtaccagtcctagtttctctgagtttcaagaagctgatcttgttagtcctagtgcttccaacatctcatatactgctgctactactactaatggaatgaccaattccaccatgaattcctcagctcctcccttttattgcaatggtgaagtagacttgatagatgagtttcaactacaggaccagctttctttcttgaatgatgggtcaccaaccttggggcctaagaatcctgatgtttattaccagcaacagcagcaacaacaagatttagcctcaagtccaagtggggattccatgcttttctcttcatataactggggtggtggttgcaactcagtcaacggcctctctcatagaaggagctgctctgtgagtgatgtatgcttgggggctgatgacccaagtggaggacttggctggaaaccttgtctctattttgccagagggtattgcaagaatggaagtagctgtaggttccttcatggtgctgggcctggtgaaggtgaagttgggtcaccaaacaagtttgagatgatggaacattgccaagaacttctcagatctaagtctgctcaccagcaaagactagccacagcttctcagctcgtggcttcttctaactttcctctctctcccatggctgctaacaaatgcatgaactttcttcagcagcaacagttgcagtctgctgaaagcccaagggcagctgctgcattgatgatgggtgatgacatgcataaattgagcagaagtcgttttgaaagaggggattttggactgaatggtggagttggaatagcaaatccaggttcaaggcaaatttacttgacatttccagctgatagtactttcaaagaagaggatgtttccaattatttcagcacttatgggcctgttcaagatgtgaggattccatatcagcaaaagaggatgtttggttttgttacatttgtttatccagagactgtgaagaccattcttgccaaaggaaatcctcattttgtatgtgatgctagggtgcttgtcaagccttacaaagagaagggcaaagtcccagagaagtttaggaagcaacaccaacagcagatggagaggggagaattcactggatgcggtagtcctactggtctggactccagtgatccttatgatcttcagcttggtgcaagaatgttttacaacactcaagatgcgctgtggaggagaaaattggaggaacaagctgatctgcaacaggcaattgagctccaaagcaggagattgctgaatttacagcttcttgatgtcaaaaggagcaaccatcatcgtgccctttccatgagtgctgttatcccatccccaccgcattctccaggcttcttcaatcagaatatggttcgctccacagactttggcagccgagaagagaatggttttgcaccaaaaatggccaattttgctgctgttactgctgagcaaaagaatgcaaatcttactgccaaggagagagaatgcttcacaggtaaagatgaaaatagcagtggcaaagaaagttccaagaaggaagcaagtgattttcaagaaagcttggagcataatctcccagatagtccatttgcatcacctaaagcagttggggacttcatcacaactttctcaaatgaagctgctggagatgttgacaaaggtgctggattaaatgcatcatcctctgctaacaataatatgatcccttcttcctccttgtcaactagtactctagacatgactcctttcaaatcatgttacttccaagtgcctaggttcccttccggacatggcgccattggaatgtag

SEQ ID NO: 247 - protein, 731 aa, Lycopersicon esculentum
MDAYEATKIVFQRIQSLDPENASKIMGILLMQDHGEKEMIRLAFGPEALVHSVILKARKELGVSSNSPSTPSTPSSPSPFGGSMCFSRQNSSSSATSGRILGGLSLPSPLSITSNNNHSSNVSASWSTSPSFSEFQEADLVSPSASNISYTAATTTNGMTNSTMNSSAPPFYCNGEVDLIDEFQLQDQLSFLNDGSPTLGPKNPDVYYQQQQQQQDLASSPSGDSMLFSSYNWGGGCNSVNGLSHRRSCSVSDVCLGADDPSGGLGWKPCLYFARGYCKNGSSCRFLHGAGPGEGEVGSPNKFEMMEHCQELLRSKSAHQQRLATASQLVASSNFPLSPMAANKCMNFLQQQQLQSAESPRAAAALMMGDDMHKLSRSRFERGDFGLNGGVGIANPGSRQIYLTFPADSTFKEEDVSNYFSTYGPVQDVRIPYQQKRMFGFVTFVYPETVKTILAKGNPHFVCDARVLVKPYKEKGKVPEKFRKQHQQQMERGEFTGCGSPTGLDSSDPYDLQLGARMFYNTQDALWRRKLEEQADLQQAIELQSRRLLNLQLLDVKRSNHHRALSMSAVIPSPPHSPGFFNQNMVRSTDFGSREENGFAPKMANFAAVTAEQKNANLTAKERECFTGKDENSSGKESSKKEASDFQESLEHNLPDSPFASPKAVGDFITTFSNEAAGDVDKGAGLNASSSANNNMIPSSSLSTSTLDMTPFKSCYFQVPRFPSGHGAIGM

SEQ ID NO: 248 - DNA, 1866 bp, Pinus radiata
atggatgcctatgaagctacaaggattgtgttctccaggatccagagcttagagccagaaaatgtgtctaaaattattgggtacttgttattacaagaccatggtgaacaggaaatgattaggttggctttcagtcctgattccttgattcagtccatgatcatcaaggttaagaaagatctaggtttgatgcaacagcaaggtactccagctccaactgtttcatcttacctatccagaatcaatcgtctccccaacttgccactgcagtctgctcagatttctcaatccagagctttctcttctccaaccgccctttcacctcatgcagctccatggggatctcatatttctcaacagactaggcctttatccaacaattttaactcgatcttgaatgagatacagactaacactagtacttctagtactataaattctactagcaatggctatcttaatttcagtctgccatccatgccagatcaagctaatagccttccttacagcgagcatcctggccttgtagatgaatttcaattgcaagatcagcttccctttctcaatgattctccagagtctgcccaatctcatgctaattatctcaactatcctgagatgttgcaggcatattgcaatggcaatcagccattgactatagaccatgtaagcccaacagcagctaataatagctatcctggtactactcatatacccaagcagcctagttcaatttcagatatttacaatctaacttcagaatctgcacctgggtcagccttggcctggaagccatgcatgtactttgctaggggttactgcaagaatgggagcaattgcaggtttcttcatggtaactatggtggtcatgtaaggtcagagagcaataatgaccacagtgaaaagttcatgggatccagtagtggcccattggaaaaactggagttagaattgaaggaattgctcagaggaagagggtctccagtttcagttgcttctctacctcagttctatagtgagaggcttgggaaggctcttcaggccgagaggtttacaaggtatagaagtgaacgagattcatctgatcacttggccagttctgcttccaattctggatctcgccaaatatacttgacttttcctgcagagagtactttcagggaggaggacgtatcaaattatttcagcatttttggacccgtgcaggatgtaaggattccttatcagcagaagaggatgtttgggtttgtgacatttgtatatcaagagactgttaagattattttggcaaaaggcaatcctcattatgtctgtgatgcccgtgttcttgtcaaaccatacaaagaaaaaggatccaaacccgcagagagaatgaagtataccgactgtaggggcgattattcaggatatgtgacaactcacaatcttgatatcaaggacagcaatttgcaacttggccctcccagatttgttgaaaacagcttagacctggtgacaagaaggcagttggaggaggagcaggatcatgtagagcaagccgttgagcttcaaacaaaacgacttgcagagctgcagcttggtgacagaaagagaccacagcttgtaccttcagatcctcaagtttctatggcttcaacaaactccggcccggctcagcattatcaaaatcagttctcaaatggacccaataatcattcagaagaggacgcaacaacctcagaagattttagcagttctacattagcagaacattttggttatgtgctacaggttttagatagtgaatctgtctatgaggaacacccaaaacctgtcaaccatcaccatgacaggcttcctattactaatggtgcaatgagactgtaa

SEQ ID NO: 249 - protein, 621 aa, Pinus radiata
MDAYEATRIVFSRIQSLEPENVSKIIGYLLLQDHGEQEMIRLAFSPDSLIQSMIIKVKKDLGLMQQQGTPAPTVSSYLSRINRLPNLPLQSAQISQSRAFSSPTALSPHAAPWGSHISQQTRPLSNNFNSILNEIQTNTSTSSTINSTSNGYLNFSLPSMPDQANSLPYSEHPGLVDEFQLQDQLPFLNDSPESAQSHANYLNYPEMLQAYCNGNQPLTIDHVSPTAANNSYPGTTHIPKQPSSISDIYNLTSESAPGSALAWKPCMYFARGYCKNGSNCRFLHGNYGGHVRSESNNDHSEKFMGSSSGPLEKLELELKELLRGRGSPVSVASLPQFYSERLGKALQAERFTRYRSERDSSDHLASSASNSGSRQIYLTFPAESTFREEDVSNYFSIFGPVQDVRIPYQQKRMFGFVTFVYQETVKIILAKGNPHYVCDARVLVKPYKEKGSKPAERMKYTDCRGDYSGYVTTHNLDIKDSNLQLGPPRFVENSLDLVTRRQLEEEQDHVEQAVELQTKRLAELQLGDRKRPQLVPSDPQVSMASTNSGPAQHYQNQFSNGPNNHSEEDATTSEDFSSSTLAEHFGYVLQVLDSESVYEEHPKPVNHHHDRLPITNGAMRL

SEQ ID NO: 250 - DNA, 2274 bp, Eucalyptus grandis
atggacgcatatgaagccacaaggattgtcttctcaagaatccaaagtttagaccctgagaatgcctccaagatcatgggtctcctcctcatccaagatcatggtgagaaggagatgatcaggctggctcttggaccggagactctgcttcactcagtggtcctcaaggcaaggaaggacataatccttccgtcaaactctccctcaacgccctccacaccttcttctccctctcctttcatgtctaccaaccccatctccatctcctccaggcctaaaggaagcaacttttcgccatcttctctctcaaatatccccagcccatcttcttgggggggtggtggtggtggtggtggttctttctctgatctctcaagtggagatgatttgatcaattcttcctcttgtttgtatggaaatggaggcagtgacaccatgattgatgagcttcagctccaagaccagctttccttcctcaacgataactccccaccccttggacccaacagcaaccctgatatgttctgcccccagcaggacttgctgtccagtcccaccgccgtatacggcggagcggccgcgggctggggcgccccggtgcaccggaggagctgctcggtcagcgatgtgtgctcgggttcctcagaagacccatcttgtggagtcgggtggaggccatgcttgtattatgctagagggtactgcaagaatgggatcagctgcaggttcttgcacagtggtggacttggcgatgccgcttctgtggtcggcagcccggacggcagtgcgtccgcggtggtcggctccccgagtaaagtggacatgatgggccagtgccatgaagctgttctgaggtccaaatctgctcagcagcagagactagctgctgcttctcagctcataggttctgcaaccttcccttacactcccaaatccatgaatttacttctccatcaccagcaaaatgatgctcatagggctgctgctgctgctgcgctgatgatgggtgatgacttttacaagtatggcagatcaaggctagaaaggagtgatttttcagtgaatggttgtgtgaatcctgcttctaggcagatttacttgactttcccagccgacagtactttcaaggaggaagatgtttccaactatttcagcaactttgggccggtgcaagatgtgagaattccttaccagcagaagaggatgtttggctttgttacatttgtttacccagaaacggtgaagctcattttggccaaagggaaccctcattttgtttgtgatgctagagttctcgtcaagccttacaaagagaagggaaaagtgccagacaagttcaggaagcagtcccagctggtggaaaggggtgatttttcgccttgtggaactccaactgggttggattcgagggggggaccatttgacctcaacctcggagcgaggccgttttacaattctcaggacatgctgtggaggaggagattcgaggagcaagctgatcttcaacaagcccttgaataccagagtcaacggctgatgagtctgcagcttctagatgtcaagaagcatcatcatcagagggctctctcaactggctcccccattccatctcctgctcaatcgcctactttgttcaacaatccaaccttcctcaacattccttcggttcgcagcctgggcgtcacagaagagaatggttctagccctggcttatccgacagtcagcccttgaactaccagtctgtgattgtctctgctgggaaagatttgactggaagtgacaagagtaatgggaatgacaaggaaagctcccatactgaagataaaggcttggctgaaagtttggagcataaccttcctgatagtccctttgcatctcctactaaagcctccgcagagcacttctcttccttaaccaatgtagtcagcgaggctgaaaaggatggtgtaggttcggcctcatcttctcccaacagcaataacccagtctcttcacccttgatcccgggcacctccgccatggacatggcttcattcacgtctttcaactgccagattcctgccataggaatgtatgccggtgctggagggccaacatgcccagtgggtatatagctagctcttccttgactgagggagattaacaacaataaccaaacaaacccgttatcaaagaccagatctgtagagaatccgtaccactaccatcacctccaccactacctcgattatccatactatactactggataccatacaaaaatgcatacgtaa

SEQ ID NO: 251 - protein, 755 aa, Eucalyptus grandis
MDAYEATRIVFSRIQSLDPENASKIMGLLLIQDHGEKEMIRLALGPETLLHSVVLKARKDIILPSNSPSTPSTPSSPSPFMSTNPISISSRPKGSNFSPSSLSNIPSPSSWGGGGGGGGSFSDLSSGDDLINSSSCLYGNGGSDTMIDELQLQDQLSFLNDNSPPLGPNSNPDMFCPQQDLLSSPTAVYGGAAAGWGAPVHRRSCSVSDVCSGSSEDPSCGVGWRPCLYYARGYCKNGISCRFLHSGGLGDAASVVGSPDGSASAVVGSPSKVDMMGQCHEAVLRSKSAQQQRLAAASQLIGSATFPYTPKSMNLLLHHQQNDAHRAAAAAALMMGDDFYKYGRSRLERSDFSVNGCVNPASRQIYLTFPADSTFKEEDVSNYFSNFGPVQDVRIPYQQKRMFGFVTFVYPETVKLILAKGNPHFVCDARVLVKPYKEKGKVPDKFRKQSQLVERGDFSPCGTPTGLDSRGGPFDLNLGARPFYNSQDMLWRRRFEEQADLQQALEYQSQRLMSLQLLDVKKHHHQRALSTGSPIPSPAQSPTLFNNPTFLNIPSVRSLGVTEENGSSPGLSDSQPLNYQSVIVSAGKDLTGSDKSNGNDKESSHTEDKGLAESLEHNLPDSPFASPTKASAEHFSSLTNVVSEAEKDGVGSASSSPNSNNPVSSPLIPGTSAMDMASFTSFNCQIPAIGMYAGAGGPTCPVGILALPLREINNNNQTNPLSKTRSVENPYHYHHLHHYLDYPYYTTGYHTKMHT

SEQ ID NO: 252 - DNA, 1806 bp, Pinus radiata
atggatacatatgaagcaacaaggattgtgttcacaaggattcagagcatagaaccagagaatgtgtccaagatcattgggtatttgcttcttcaagacctgggagatcaagaaatgattcgcctggcatttgggcctaatacactcttacagtccatgatttccaaggccaagacagagcttggtttatcatctccgtcccctcttcagtcacctctgcgctacactcagttttctaatttgttgtctcgctcattctcttctccagcacccaatggctttgatgattgtcagctccaagatcatctcttctatcccactgagaatcttgattcctacagcctaccaaatgatagtcatgtctatacagagatgctctctttcttgaatggaagcaagacaggattgaatcacaggcgatcttactccatgacagacgtttctgtaagctgtgagccagtttcttggaaaccatgcctgtattttgccaggggatattgcaagcatggatccagctgccgttttactcacagttattcacgctctgacaacgtttcgagttcaattcccttggatccccgcttcgaagaagctttctctgtggaatcattggaaagattagagttagaacttcaagaattattgagaggaagacgggcccctgtttccattgcttccctacctcaactctattacgagagatttgggaaaaccctgcaagccgagggatacttgactgagagtcagaggcatgggaaagcaggatacagcttgacaaatctactagcacgcttgaagaatacagtaagcctcattgataggcctcatggtcagcacgccattgttttggcagaggatgctaacaggttcacaacttataggccaagtgaacgagatccctactatttgagtggtgtcagctctggatcccgtcaaatttacatgacatttcctgctgaaagcaccttcacggaggaggatgtttccaactatttcaggatatacggacctgtagaggatgtgagaattccataccaacagaagcgtatgtttggatttgtaacatatgttttcccagagactgtcaaattgatactggctaaaggaaatcctcactatgtctgcggtgctcgtgttctggttaagccatacaaggagagaaacaagcatggagacaggaaaaatggcgatagaggagaacaatatgcaagatacttgctgccatcttacaatgtagattcaaaggactatgatctatgtccagctcctaggatgtttcagaattccgaactgatcagaaggcatatagaagagcaagagcaagccatcgaattggaaagactacgcctgacagagttgcatctggctgaccgtgcgcagagaactcaaaataatgccatcactctgcaacaacaaaattctcattctaatgggctactcaatgtagaggaagaagaaactcaggtttcagaagagctaaacagctttgatccgcccacggatcactttggttatctgctggatgtgctggatagtgagcagaaccctgaagaagagcctaaacaacagaaagccgacaatgacgaagaatgcaacgggcacaaccttcctgatagcccttttgggttttctcattccattaaaacaacattgccccatcccgagaaaacgaacaacttttcctttttcgacaccagcccagcacaagaatcatccacttccatcatgacaaaggaagggacttgttccatgtgcctcgattccattgtggagcaggtgcggttagagtgcaagcatgtgaggtga

SEQ ID NO: 253 - protein, 601 aa, Pinus radiata
MDTYEATRIVFTRIQSIEPENVSKIIGYLLLQDLGDQEMIRLAFGPNTLLQSMISKAKTELGLSSPSPLQSPLRYTQFSNLLSRSFSSPAPNGFDDCQLQDHLFYPTENLDSYSLPNDSHVYTEMLSFLNGSKTGLNHRRSYSMTDVSVSCEPVSWKPCLYFARGYCKHGSSCRFTHSYSRSDNVSSSIPLDPRFEEAFSVESLERLELELQELLRGRRAPVSIASLPQLYYERFGKTLQAEGYLTESQRHGKAGYSLTNLLARLKNTVSLIDRPHGQHAIVLAEDANRFTTYRPSERDPYYLSGVSSGSRQIYMTFPAESTFTEEDVSNYFRIYGPVEDVRIPYQQKRMFGFVTYVFPETVKLILAKGNPHYVCGARVLVKPYKERNKHGDRKNGDRGEQYARYLLPSYNVDSKDYDLCPAPRMFQNSELIRRHIEEQEQAIELERLRLTELHLADRAQRTQNNAITLQQQNSHSNGLLNVEEEETQVSEELNSFDPPTDHFGYLLDVLDSEQNPEEEPKQQKADNDEECNGHNLPDSPFGFSHSIKTTLPHPEKTNNFSFFDTSPAQESSTSIMTKEGTCSMCLDSIVEQVRLECKHVR

SEQ ID NO: 254 - DNA, 2124 bp, Populus trichocarpa
atggatggttatgaagcaacaagaatagttttctcgagaatccaaaacctagacccagaaaatgcttcaaaaatcatgggtcttcttttgattcaggaccatggtgaaaaggaaatgattaggttagcttttggaccagaagcacttgttcactcagtaatccttaaagcgaggaaagaactaggactttgctctccaacaaacccttctaaaagtccttcgcccccttcgcctctatattcaagcaacccaataaccatctctagacagaattcatcttcttcaacttcaagacttgggtttaacatcccaccttcacttactatcccaaacccttcatcaaatttttcttcttcttggagtgaccttccaaaccctgatgacttgattagtcctaatggtagttcactcaatcctgcttctgctcctttctatgctaatggagtaagaggtggaggagagtctgatttgatggatgagtttcagctccaagaccagctttcattcttgaatgacaattcagcaaatcttggtccaaaaagctcagatcttttttactctcaactggatgctttatcaagtccaactggtgctagtgattctgtgatgtttccttcttactggggtgggtctgtgcacagaaggagctgttctgtcagtgatgttttggggtctgaggatccaaattcaggctttgggtggagaccttgcctttactttgctagagggtactgtaagaatggaagtaactgtaggtttgttcacggtgggctcggagaatctgatggtgcaggtgttgttgtgggttcacctaatggtaacaacaagattgatatgatggaccagtgccatgagttgcttagatccaagtctgctcaacagcaaaggttagctgctgcttctcagctcatgggtggctctgctgcttcttttccttactctcctaaaagcatgaactttcttcttcaacaacagcaaaatgatagccagagggctgctgctgctttgatgatgggggaggacatgcacaaatttgcaagatctaggcttgataggaatgatttgattaatcctgcttccaggcagatctacttgactttccctgctgatagcacttttagagaggaagatgtgtcaaattacttcagtatttatgggccagtgcaagatgtgaggattccttatcagcagaagaggatgtttggatttgttacctttttgtatccagagaccgtgaagataatattggccaaagggaaccctcattttgtttgtgatgcaagggtgcttgttaagccttacaaagagaaaggcaaagtcccagacaagaagcaacagcagcaacaagttgagaggggtgagttctcaccatgtggtactcctactggccttgattcaagagatccatttgatctccaacttggtgcaagaatgttttacaacacacaagacatgttgtggaggaggaagctagaggagcaagctgatttgcagcaagcccttgagcttcaaagtagaagattgatgagtttgcagcttcttgatgtcaagaaacatcatcatagggctctttccactggcagccctgtcccctccccaactcactctccaaatattttcaatcaatctcttgcctttcctccactccacagcaacacagaagttccacaagagaattgttctagcccaatgccagccatttcagtggctgccccaactgaaaaacagatatcaaatgctaattctgggaaagaatgtactagcagtgaagagaatggcagtggtaaagagagctcccatggtgaagacagcgatttacaagaaagtttggagcacaacctccctgatagtccctttgcatctcctaccaaaggctccggggactactactctgccttcatccatggagttcctgacctctcccatgagaaggatgctaacatcccggcttcatcttctgctaacaatagtttggtcactacaagtctaatctctcctaattcttcactagaaatggcatccttcaagtccttcaattgccaaatgcccaggttttcatccgggcatggagcaatagggatgtatgccaacacagatggacctacctgccctgttggaatttag

SEQ ID NO: 255 - DNA, 2196 bp, Lycopersicon esculentum; identical to SEQ ID NO: 246

SEQ ID NO: 256 - protein, 731 aa, Lycopersicon esculentum; identical to SEQ ID NO: 247

SEQ ID NO: 257 - DNA, 1866 bp, Pinus radiata; identical to SEQ ID NO: 248

SEQ ID NO: 258 - protein, 621 aa, Pinus radiata; identical to SEQ ID NO: 249

SEQ ID NO: 259 - DNA, 2274 bp, Eucalyptus grandis; identical to SEQ ID NO: 250

SEQ ID NO: 260 - protein, 755 aa, Eucalyptus grandis; identical to SEQ ID NO: 251

SEQ ID NO: 261 - DNA, 1806 bp, Pinus radiata; identical to SEQ ID NO: 252

SEQ ID NO: 262 - protein, 601 aa, Pinus radiata; identical to SEQ ID NO: 253

SEQ ID NO: 263 - DNA, 2124 bp, Populus trichocarpa; identical to SEQ ID NO: 254

SEQ ID NO: 264 - protein, 707 aa, Populus trichocarpa
MDGYEATRIVFSRIQNLDPENASKIMGLLLIQDHGEKEMIRLAFGPEALVHSVILKARKELGLCSPTNPSKSPSPPSPLYSSNPITISRQNSSSSTSRLGFNIPPSLTIPNPSSNFSSSWSDLPNPDDLISPNGSSLNPASAPFYANGVRGGGESDLMDEFQLQDQLSFLNDNSANLGPKSSDLFYSQLDALSSPTGASDSVMFPSYWGGSVHRRSCSVSDVLGSEDPNSGFGWRPCLYFARGYCKNGSNCRFVHGGLGESDGAGVVVGSPNGNNKIDMMDQCHELLRSKSAQQQRLAAASQLMGGSAASFPYSPKSMNFLLQQQQNDSQRAAAALMMGEDMHKFARSRLDRNDLINPASRQIYLTFPADSTFREEDVSNYFSIYGPVQDVRIPYQQKRMFGFVTFLYPETVKIILAKGNPHFVCDARVLVKPYKEKGKVPDKKQQQQQVERGEFSPCGTPTGLDSRDPFDLQLGARMFYNTQDMLWRRKLEEQADLQQALELQSRRLMSLQLLDVKKHHHRALSTGSPVPSPTHSPNIFNQSLAFPPLHSNTEVPQENCSSPMPAISVAAPTEKQISNANSGKECTSSEENGSGKESSHGEDSDLQESLEHNLPDSPFASPTKGSGDYYSAFIHGVPDLSHEKDANIPASSSANNSLVTTSLISPNSSLEMASFKSFNCQMPRFSSGHGAIGMYANTDGPTCPVGI

SEQ ID NO: 265 - DNA, 2199 bp, Populus trichocarpa
atggatgcttatgaagcaacaagaatagttttctcaagaatccaaaatctagacccagaaaatgcttcaaagatcatgggtcttcttttgattcaagaccatggtgaaaaggaaatgattaggttagcttttggaccagaagcacttgttcactcagtgatccttaaagccaggaaagaactaggactaagctctccgacaaacctttctacaagtccttcctctccttctcctctttactcaagcaacccaatagccatctctagacaaaatagttcttcaacttcaagacttgggtttaatatcccaccttcacttgctatcccaaacccttcatcaaataattcttcttcttggagtgaccttccaaacccagatgacttgatgattagtcctaatgatagctcactcaatcctgcttcagtgcctttctatgctaatggagtaagaggtggagagtctgatttgatggatgagtttcagctccaagaccagctttcattcttaaatgataattcacaaaatctcggtccaaaaagttcagatcttttttaccctcagcttgatgctctatcaagtccaactggtgctagtgattctatgatgtttccttcttactggggtgggtctgtgcacagaaggagctgctctgtcagtgatgttttggggtctgaggatccaaattcagggtttggatggagaccatgtctttactttgctagagggtactgtaagaatggaagtaattgtaggtttgttcatggtgggcttggagaactagatggtgcaggtgttgtcggttcacccaatagcaacaacaagattgatatgatggaccagtgccatgagttgcttagatctaagtctgctcaccagcaaaggttagctgctgcttctcagctcatgagtagctctgctgcttcttttccttactctcctaaaagcatgaactttcttcttcaacagcagcaaaatgatagccagagggctgctgctactgctttgatgatgggggaggacatgcacaaatttggaagatctaggcttgacaggaatgatttggttaatcctgcttcaaggcagatctacttgactttcccagcggatagcacttttagagaggaagacgtgtcaaattatttcagtatttatgggccagtgcaagatgtgaggattccttatcagcagaagaggatgtttggatttgttacctttgtttatccagagacggtgaagataatcttggccaaagggaatcctcattttgtttgtgatgcaagggtgcttgttaagccatacaaagagaaaggcaaagtcccagacaagaagcaacagcagcaacaagttgagaggggtgagttctcaccttgcggtactcctactggtcttgattcaagagatccctttgatctccagcttggtgcaagaatgttttacaatactcaagacatgctgtggaggaggaagctagaggagcaagctgatttgcagcaagcccttgagcttcaaagtagaagattaatgagcttgcagcttcttgatgtcaagaaacatcatcacagggctctttccaatggcagccctgtcccctctcctactcactctcccaatattttcaatcactctcttgccttccctccactccacagcagcaccgaagttccacagggtatggctgtctctttgttactctactctatacaggtgaaaattgagctttacaacctaactttggattgttttgtttcagagaattgttctagctcaatgccagccacgtcagtgactgccccgcctgaaaaacagatatcaaatgctacttctggtaaagaatatactagcagtgaagagaacggcagtggaaaagagagctcccatggtgaagacagtgatttacaagaaagtttggagcacaacctccctgacagtccctttgcatctccaacaaaaggcaccggggactattactctgccttcatcaatggacttactgaggcccgtgagaaggatgctagcatcccaacttcaacttctgctaacaataatttggtcccctcaagtctaatctctcctaattcttcactggaaatggcatccttcaaatccttcaattgccaaatccctaggttttcatctgggcatggagcaattgggatgtatgccagcacagatggacctacctgtcccgttggaatttag

SEQ ID NO: 266 - protein, 732 aa, Populus trichocarpa
MDAYEATRIVFSRIQNLDPENASKIMGLLLIQDHGEKEMIRLAFGPEALVHSVILKARKELGLSSPTNLSTSPSSPSPLYSSNPIAISRQNSSSTSRLGFNIPPSLAIPNPSSNNSSSWSDLPNPDDLMISPNDSSLNPASVPFYANGVRGGESDLMDEFQLQDQLSFLNDNSQNLGPKSSDLFYPQLDALSSPTGASDSMMFPSYWGGSVHRRSCSVSDVLGSEDPNSGFGWRPCLYFARGYCKNGSNCRFVHGGLGELDGAGVVGSPNSNNKIDMMDQCHELLRSKSAHQQRLAAASQLMSSSAASFPYSPKSMNFLLQQQQNDSQRAAATALMMGEDMHKFGRSRLDRNDLVNPASRQIYLTFPADSTFREEDVSNYFSIYGPVQDVRIPYQQKRMFGFVTFVYPETVKIILAKGNPHFVCDARVLVKPYKEKGKVPDKKQQQQQVERGEFSPCGTPTGLDSRDPFDLQLGARMFYNTQDMLWRRKLEEQADLQQALELQSRRLMSLQLLDVKKHHHRALSNGSPVPSPTHSPNIFNHSLAFPPLHSSTEVPQGMAVSLLLYSIQVKIELYNLTLDCFVSENCSSSMPATSVTAPPEKQISNATSGKEYTSSEENGSGKESSHGEDSDLQESLEHNLPDSPFASPTKGTGDYYSAFINGLTEAREKDASIPTSTSANNNLVPSSLISPNSSLEMASFKSFNCQIPRFSSGHGAIGMYASTDGPTCPVGI

SEQ ID NO: 267 - DNA, 1623 bp, Arabidopsis thaliana
atggatggatatgaagctactaggattgtgctctctagaatccaaagcttagaccctgaaaacgcatcaaagatcatgggtcttctccttcttcaagatcacggtgaaaaagagatgataaggctagcttttggtccagagactcttgttcactctgttatagtgaaagccaagaaagagttaggtctcatgaactgttcaaggtctccgtggagtcatcaagatgagttgattagccctaagaacaaccgtggctcttcactcaatccagcttctttgcccttttacgctaatggaggaagatcttctagggatttaaccaacgatttcgagctcatggatgatatgaactccagaagtactgattttttgggctctgtgcatgcgagaagcggtagctgcgttttggacggtttagggtatggtggtgattctgatttagggtttggaggtgtgccctgttcttacttcgctagaggcttctgcaaaaacggagctagctgcagattcgtccacagtgatggaggagctgatttggttggctccccaagcagaatcgagcttcttaggtctaactcggtacccccaagacttgctcaccacttcatgactcgctcttctctcccttctttttcaactaaaggtgttaacttgcagcaaaacgatgttcaaagagctgctgctgctttgatgataggagatgaattgcagaagcttggaagatggagacctgaaaggattgatctttctgctatggcttgtccagcttcaagacagatctatctgacattccctgccgacagtaggttcagggaggaagatgtgtccaattacttcagtacttttggaccagttcaagatgtgaggataccatatcagcaaaagagaatgtttggttttgtgacatttgtgtaccctgagactgttaagagcattctcgccaaagggaatcctcactttgtgtgtgattccagagttcttgtcaagccttacaaggagaaaggcaaagtccctgacaaatacagaactaaccaaacaacagagcgagaactgtccccaacaggccttgattctagccctagggacgttctaggagggagagggttttataacaacactcaagatgtgttgtggaggagtaagtttgaagaagagattcttgaacttcagagcagaaggctgatgaatctgcagcttcttgacgtcaagaagcatttccaactcaattcccctaccaacattcactctccgaatcctttcagccaatcacttatatctccacgcccattgtccgtgatcaagagagagtatgatggaggagagaaagggaaaggaagttctaaagaaggatctgatgatgatacaatgaatctaccagagaggttggaggatagcttgccagatagtccgtttgcatcgcccgctcatcatttgcttctgtttgctgattctgctgacaataacggatcggatctgtggtcgccttcttctgataatgatgataattctactccttctacactctccgactccttcaactctttcaactaccaaatgccaaggttaccggcgattggaatgttacccggtaggggtggaccaacctgtcgtgttgggatataa

SEQ ID NO: 268 - protein, 540 aa, Arabidopsis thaliana
MDGYEATRIVLSRIQSLDPENASKIMGLLLLQDHGEKEMIRLAFGPETLVHSVIVKAKKELGLMNCSRSPWSHQDELISPKNNRGSSLNPASLPFYANGGRSSRDLTNDFELMDDMNSRSTDFLGSVHARSGSCVLDGLGYGGDSDLGFGGVPCSYFARGFCKNGASCRFVHSDGGADLVGSPSRIELLRSNSVPPRLAHHFMTRSSLPSFSTKGVNLQQNDVQRAAAALMIGDELQKLGRWRPERIDLSAMACPASRQIYLTFPADSRFREEDVSNYFSTFGPVQDVRIPYQQKRMFGFVTFVYPETVKSILAKGNPHFVCDSRVLVKPYKEKGKVPDKYRTNQTTERELSPTGLDSSPRDVLGGRGFYNNTQDVLWRSKFEEEILELQSRRLMNLQLLDVKKHFQLNSPTNIHSPNPFSQSLISPRPLSVIKREYDGGEKGKGSSKEGSDDDTMNLPERLEDSLPDSPFASPAHHLLLFADSADNNGSDLWSPSSDNDDNSTPSTLSDSFNSFNYQMPRLPAIGMLPGRGGPTCRVGI

SEQ ID NO: 269 - DNA, 1611 bp, Arabidopsis thaliana
atgaatttcacagaatcaatgaacgttgtgcacgccagaatccaacaacttgaaccagagaatgcagcaaagatctttggttatctcttgttgatgcaagaaaatggcaaccgcgacatgatccgtctcgcgttctgtcctgattctgttatgtgttctgtcatcaattgcgttaaatacgagttagctaggaattctcatcattaccacagccctccttctgatcacattcctactcccaaatttggatcattcaccggttcatcgcctctttcggtttcggtttctcctcccatgaaaaccggtttttgggagaattcaaccgagatggataccttgcagaacaatcttcagttcttgaattttgaggatcctttgaccagccctgaattctctaacgggttcttctctcaagaacgtcaatgtttgcctttgcgaactagccgaagatccccgagtttacccgagttcccggtaaaaatctgccattacttcaacaaagggttctgcaaacacggcaacaactgtaggtacttccacgggcagattataccggagagggagagttttgctcagatgtttaatccaaacaacaacctaagtgacgaagagcatgttgtttcccctgtatctcttgagaagctagaaggtgagatcattgagttactcaagttaagaagaggagctccaatctccatagcttcattgccaatgatgtactacgaaaaatacggtaggactcttcaagctgaaggatatctcactgagtcacaaagacatggcaaagctggctatagtctcaccaagcttcttgctcgcttgaagaacacgattcgcctcgtcgacaggcctcatgggcaacattcagttatattagcagaggatgcatcaaagtttgtggaatacactggagagagaaatgaacatggagcaatccttgctggttctagacagatttacttaacgtttccggcagagagtagtttcactgaacatgatgtctcaatctacttcacctcatatggacatgtggaagatgtgaggattccttgccagcagaaaagaatgtatggatttgtaacatttgcttcctcagaaacagttaaacacattcttgctaaaggcaatcctcatttcatttgcaatgcacgtgttctagtcaagccttaccgggaaaaatcacgctctagtcgatacctcgacaattacaagcctctacacggcatgcgatatggctctaaatttattgagagagacatagagatgaacacattgccaccgcgggttagtgagagctcaagaatgaggaagccatttcttagtgagcctgaacaatcagtttctaagtccttacctactaattactcctacctcggcttctcctcggatgacttcaagttaacatcaaatgcggagcaagaggaacaagcagaacggttgagctacctactggactatttgaacacggaagataatgtcatgaacataaccactaactacagagacaatgatcggagaactcattgtgaatcgttggacagtcaagtcctgaatctacccgagagtccgttttcttccctttcggggaaggagatttcaacggttacatag

SEQ ID NO: 270 - protein, 536 aa, Arabidopsis thaliana
MNFTESMNVVHARIQQLEPENAAKIFGYLLLMQENGNRDMIRLAFCPDSVMCSVINCVKYELARNSHHYHSPPSDHIPTPKFGSFTGSSPLSVSVSPPMKTGFWENSTEMDTLQNNLQFLNFEDPLTSPEFSNGFFSQERQCLPLRTSRRSPSLPEFPVKICHYFNKGFCKHGNNCRYFHGQIIPERESFAQMFNPNNNLSDEEHVVSPVSLEKLEGEIIELLKLRRGAPISIASLPMMYYEKYGRTLQAEGYLTESQRHGKAGYSLTKLLARLKNTIRLVDRPHGQHSVILAEDASKFVEYTGERNEHGAILAGSRQIYLTFPAESSFTEHDVSIYFTSYGHVEDVRIPCQQKRMYGFVTFASSETVKHILAKGNPHFICNARVLVKPYREKSRSSRYLDNYKPLHGMRYGSKFIERDIEMNTLPPRVSESSRMRKPFLSEPEQSVSKSLPTNYSYLGFSSDDFKLTSNAEQEEQAERLSYLLDYLNTEDNVMNITTNYRDNDRRTHCESLDSQVLNLPESPFSSLSGKEISTVT

SEQ ID NO: 271 - DNA, 2034 bp, Oryza sativa
atggatgcatacgaggcgaccaaggtggtgttctcccggatccaggcgctggaccctgaccacgccgccaagatcatgggcctcctgctcatccaggaccacggcgacaaggagatgatacgcctcgccttcggccccgaggcgctgctccacagcgtcatggcgcaggcgcgcaaggagctcgccctcctgccgccgcctcaggcggcgtcgtcgtcgcccaccgtgccggcagcccactcgccgttcctgctgtcgaggcagaactccggccgctgccccgcgccgtcgccgtcgtcgtgggcgcaggcgcagccgttctcgaggagtaacagcatgggcaatggcggcgccgcggatgagatggtcggcgctggggaggagctaatgagccccttgaacggcgggggaggcgccgcggcgaacgccccgcccttctttcctcggggtggggacgcgctcctggacgacttcgagctgcaggagcagctcgcgttcctgcacgacggagccgggggcgtgaaccccgggcatgctctccaggcgttcgacggagcggagtgccggagccccggccccggcgagagcggcgggatgctcccctacggcctcgcctgggccaacggcggccctgggcaccgccgcagcgcgtcggtgaacgagctctgcctcggcggcgacggcttcgggtggaaaccttgcctgtactacgcgcgcgggttctgcaagaacggcagcacctgcaggttcgtccatggcggcctctccgacgacgccgccatggacgcaaccaccgccgaacagcagcagtgccaggatttcctcctccgctccaagagccagcgcctcggccccgccgccttcccgttcacccccacaggctctctccctgcctcgccatccgccaccagcaagtgcctcagcctcctcctgcagcagcagcagcagcacaacgacaaccaaagagccgccgcggccgcgctgatgctcgccggcggcgacgaggcgcacaagttcatggggcggccgcgcctggaccgcgtcgatttcgccagcatgatgaaccccgggtcgcgccagatttacctcaccttcccggccgacagcacgttccgcgaggaggacgtctccaactacttcagcatctacgggccggtgcacgacgtgcgcatcccgtaccagcagaagcgcatgttcgggttcgtcaccttcgtttacccggagacggtgaagctgatcttggccaagggcaacccgcacttcatctgcgacgcccgcgtgctcgtcaagccctacaaggagaagggcaaggtccccgacaagtacaggaagcagcagcaaggcgatttctgctgcatgtcgcccacggggttagacgccagggacccctttgattttcaccagctcggtgcaaggatgctgcagcactccaacagcgcgaacgagctaatgctgcggcggaagctcgaggagcagcaacaggcggcggagctgcagcaggctattgatctccatagccgccgcctcattggcctccagctgctcgacctcaagtcttctgccgctgtccatgcggcggagacgacaacaatgtctctgcccactccgatcaccaatgccttcacctccggccaacccggtgccaccacaatcgtcgagtcaccgcctagctctactgggcaactgatggcgagctgcggctctccatcggaggggaaagttgtcaatggtggtaataaggcggattctgccggtgaggttacccgcaatgccgatagtgaccaaagtggtgagcacaacttgccagacagcccgtttgcttcctctaccaagtcgaccgcattctttaccgcaaccgctgccactgccattggtagcgagggagatttcaccaccggtagtagctgcaatattggtggcagtgcggtcggcagcgccaaccctctccggcctcccacattggacataccttcgccaaggacctgcttcttccccatgcccaggctgtccgagcacggggcgatcgggatgtaa

SEQ ID NO: 272 - protein, 677 aa, Oryza sativa
MDAYEATKVVFSRIQALDPDHAAKIMGLLLIQDHGDKEMIRLAFGPEALLHSVMAQARKELALLPPPQAASSSPTVPAAHSPFLLSRQNSGRCPAPSPSSWAQAQPFSRSNSMGNGGAADEMVGAGEELMSPLNGGGGAAANAPPFFPRGGDALLDDFELQEQLAFLHDGAGGVNPGHALQAFDGAECRSPGPGESGGMLPYGLAWANGGPGHRRSASVNELCLGGDGFGWKPCLYYARGFCKNGSTCRFVHGGLSDDAAMDATTAEQQQCQDFLLRSKSQRLGPAAFPFTPTGSLPASPSATSKCLSLLLQQQQQHNDNQRAAAAALMLAGGDEAHKFMGRPRLDRVDFASMMNPGSRQIYLTFPADSTFREEDVSNYFSIYGPVHDVRIPYQQKRMFGFVTFVYPETVKLILAKGNPHFICDARVLVKPYKEKGKVPDKYRKQQQGDFCCMSPTGLDARDPFDFHQLGARMLQHSNSANELMLRRKLEEQQQAAELQQAIDLHSRRLIGLQLLDLKSSAAVHAAETTTMSLPTPITNAFTSGQPGATTIVESPPSSTGQLMASCGSPSEGKVVNGGNKADSAGEVTRNADSDQSGEHNLPDSPFASSTKSTAFFTATAATAIGSEGDFTTGSSCNIGGSAVGSANPLRPPTLDIPSPRTCFFPMPRLSEHGAIGM

SEQ ID NO: 273 - DNA, 2049 bp, Oryza sativa
atggacgcctacgaggcgaccaaggtggtgttctcgcggatccaggcgctcgacccggaccacgcagccaagatcatgggcttcctcctcatccaggaccatggcgagaaggagatgatacgcctcgctttcggccccgaggcgctgctccacaccgtcatggccaaggccaggaaggagctcggcctcctcccggcgtccgggcccgggacgcccacctccgtggcggcggccgcggccgccgcccactcgcccttcatgctctcgcggcagaactccgggcgctgcggcaccgcgccctcgccgctctcggtgtcctccccctcctcgtgggcgcctcccccggtgttttcgaggaacaacagcatcagcaatggcgccggggaggagatggtcggcctcggcgacgagctcatcagcccggccaacggcgggggcccgccatcgcccttcttcggcggcgacccgctcatggacgagctccagctgcaggaccagctcgcgttcctcaacgagggcggcgtccccgcggggcaccagatgcccatgttcgacggcggcgagtgccggagccccggcggaggcgacggcggccttttctcctacaacctagggtgggcgaacggtggccccggacaccgccggagcgcgtcggtcagcgagctctgcctcggcggcgccgacggcctcggctggaagccgtgcctctactacgcgcgcggctactgcaagaacggcagcgcttgccggttcgtccacggcggcctccccgacgacgccgccggcaagatggacccctccgccgtggagcagcagtgtcaggacttcctcatccgctccaagtcccagcgcctcgccgccgccgccttcccctactcgcccaccggctcactccccggctcgccatccgcagccaccaagtgtttgagcttgctcctccagcagcagcagcagcagaacgagagccagagggcggcggcagcggcggcgctgatgctaggcggcgacgaggcgcacaagttcatggggcggccgcggctggagcgcgccgacttcgcgagcatgatgaaccccggctcgcgccagatttacctcaccttcccggcggatagcaccttccgcgaggaggacgtctccaactacttcagcatctacggccccgtccacgacgttcgcatcccgtaccagcagaagcgcatgttcggcttcgtcaccttcgtctacccggagacggtgaagctgattctcgccaagggcaacccgcacttcatctgcgacgcccgcgtgctcgtcaagccatacaaggagaagggcaaggtccccgacaagtacaggaagcagcaccagccgggcgagagggtggacttctccagctgcactactccaactggactcgatgccagagaccccttcgacatgcaccagctcggtgcgagaatgctgcagcactccaacagcgcgaatgagatgctgctgaggaggaagctggaggagcagcagcaggccgccgagctgcagcaggcgatcgagctccacagccgccgcctcatggggctgcagctgctagacttcaagtcgcgcgccgccgcggcgccgacccccattggcaatcctttcagcgcttctcagaccgccgccaacgcgaccggcgagtcgcctcccgattccggggagctcggtaagggaagcggcttccttcttgctcacaagaaggcggtcaacggagccgataaggaggaatccaccggagagtcctccagccctaacacagacagtgaccaaagtgtggagcataatctgccggacagcccgtttgcgtcgccgaccaagtccgcgggatttgctcgcgatcctttcgctcccactgaggcggagatctccgccaccgcgtcgactggttgtagcgccacctatgttggcatcaacaatggcgccagcaatggcggcactaaccatctcctaccttctgccttggacatgccctcaccaaaaccttatttcttccccatgtccaggctggcctccgatcacggcgcgatcggaatgtaa

SEQ ID NO: 274 - protein, 682 aa, Oryza sativa
MDAYEATKVVFSRIQALDPDHAAKIMGFLLIQDHGEKEMIRLAFGPEALLHTVMAKARKELGLLPASGPGTPTSVAAAAAAAHSPFMLSRQNSGRCGTAPSPLSVSSPSSWAPPPVFSRNNSISNGAGEEMVGLGDELISPANGGGPPSPFFGGDPLMDELQLQDQLAFLNEGGVPAGHQMPMFDGGECRSPGGGDGGLFSYNLGWANGGPGHRRSASVSELCLGGADGLGWKPCLYYARGYCKNGSACRFVHGGLPDDAAGKMDPSAVEQQCQDFLIRSKSQRLAAAAFPYSPTGSLPGSPSAATKCLSLLLQQQQQQNESQRAAAAAALMLGGDEAHKFMGRPRLERADFASMMNPGSRQIYLTFPADSTFREEDVSNYFSIYGPVHDVRIPYQQKRMFGFVTFVYPETVKLILAKGNPHFICDARVLVKPYKEKGKVPDKYRKQHQPGERVDFSSCTTPTGLDARDPFDMHQLGARMLQHSNSANEMLLRRKLEEQQQAAELQQAIELHSRRLMGLQLLDFKSRAAAAPTPIGNPFSASQTAANATGESPPDSGELGKGSGFLLAHKKAVNGADKEESTGESSSPNTDSDQSVEHNLPDSPFASPTKSAGFARDPFAPTEAEISATASTGCSATYVGINNGASNGGTNHLLPSALDMPSPKPYFFPMSRLASDHGAIGM

SEQ ID NO: 275 - DNA, 2067 bp, Oryza sativa
atggacgcctacgaggcgaccaaggtggtgttctcccggatccaggcgctggaccctgaccacgccgccaagatcatgggcctcctgctcatccaggaccacggcgacaaggagatgatacgcctcgccttcggccccgaggcgctgctccacagcgtcatggcgcaggctcgcaaggagctcgccctcctcccgccgccgccgccgccgtcgtcgtcgtcgcccaccgtgccggcggcccactcgccgttcctgctgtcgcggcagaactccggccgcggccccgcgccgtcgccgtcgccgctgtccgcgtcctcgccgtcctcgtgggcgcaggcgcagccgttctcgaggagcaatgggtccgtggatgaggtggtgggcgctggggaggagctaatcagcccggcgaacagcgggggaggcgccgcggcgaacgcgccgcccttctttcctcggggtggggacgtgctcctggacgacttccagctgcaggagcagctcgcgttcctcaacgaggggggcgtcaaccccagccaccctctccaggggttcgatggagcggagtgccggagccccggtcccggcgagggcggcgggatgttcccgtacggtctcggctgggcaaacggtggccctgggcaccgccggagcgcgtcggtcaacgagctctgcctcggcggcggcagcagcgacggcttcgggtggaagccttgcctgtactacgcgcgcgggttctgcaagaacggcagcagctgcaggttcgtccacggcgacgacgccgccgctctgacgggcgccgccatggacgcggccaccgccgagcagcagcagtgccaggatttcctcctccgttccaagagccagcgcctcggccctgctgccttcccctattcacccacagggtcgctccccggctcgccatccgccgccaccaagtgcctcagcctcttgctgcagcagcagcacaacgacaaccaaagagccgcggcggcggcggcgctgatgctcggcggcagcgacgaggcgcacaagttcatggggcggccgcgcctggaccgcgtcgactttgccagcatgatgaaccccggatcgcgccagatttacctcaccttcccggccgacagcacgttccgcgaggaggacgtctccaactacttcagcatctacggtccggtgcacgacgtgcgcatcccgtaccagcagaagcgcatgttcgggttcgtcaccttcgtgtaccctgagacggtgaagctgatcttggccaagggcaatccgcatttcatctgcgacgcccgcgtgctcgtcaagccttacaaggagaagggcaaggtccccgacaagtacaggaaacatcaaggtgacttctccggctgcacgacccccactggcctggacggcagagacccattcgatctgcatcagctcggtgcgaggatgctgcagcactccaatagcacaaacgagatgatgcttcgtaggaaactggaggagcagcagcaggctgccgagctgcaacaggccatcgaactccacagtcgccgtctcatggacctccagctgctcgacctcaagaatagagccgcagctgctgtcacaacagcaatggcgatgacaattcccaccgccaatgccttcggttccagtcagcctcttgccaccaccatggtcgagtcaccgcctgattctggcgagcagctcaagggaacaggttacttcacagaagagaggaaaatggtcaacggaggaggtgataaggaagaatctgctggtgaggcgagcctgaatgctgatagcgaccaaagcttggagcacaatttgccggacagcccgtttgcttcgccgactaagtcctctgtctcagctcaccaaagtttcaccaccactgataccggtgtcgtcgcgacaagtagctgcagtgcatctcatgtaggcatcagcgcgggcactaatgctggaggtggaatcaaccatctgcggccctctactttggatataccttcgccaagagacttcttctcggtttccagcaggctggcctctgatcacggagcgattgggatgtaa

SEQ ID NO: 276 - protein, 688 aa, Oryza sativa
MDAYEATKVVFSRIQALDPDHAAKIMGLLLIQDHGDKEMIRLAFGPEALLHSVMAQARKELALLPPPPPPSSSSPTVPAAHSPFLLSRQNSGRGPAPSPSPLSASSPSSWAQAQPFSRSNGSVDEVVGAGEELISPANSGGGAAANAPPFFPRGGDVLLDDFQLQEQLAFLNEGGVNPSHPLQGFDGAECRSPGPGEGGGMFPYGLGWANGGPGHRRSASVNELCLGGGSSDGFGWKPCLYYARGFCKNGSSCRFVHGDDAAALTGAAMDAATAEQQQCQDFLLRSKSQRLGPAAFPYSPTGSLPGSPSAATKCLSLLLQQQHNDNQRAAAAAALMLGGSDEAHKFMGRPRLDRVDFASMMNPGSRQIYLTFPADSTFREEDVSNYFSIYGPVHDVRIPYQQKRMFGFVTFVYPETVKLILAKGNPHFICDARVLVKPYKEKGKVPDKYRKHQGDFSGCTTPTGLDGRDPFDLHQLGARMLQHSNSTNEMMLRRKLEEQQQAAELQQAIELHSRRLMDLQLLDLKNRAAAAVTTAMAMTIPTANAFGSSQPLATTMVESPPDSGEQLKGTGYFTEERKMVNGGGDKEESAGEASLNADSDQSLEHNLPDSPFASPTKSSVSAHQSFTTTDTGVVATSSCSASHVGISAGTNAGGGINHLRPSTLDIPSPRDFFSVSSRLASDHGAIGM

SEQ ID NO: 277 - DNA, 2037 bp, Zea mays
atggacgcctacgaagccaccaaggtggtgttctcgcggatccaggcgctggacccggaccacgccgccaagatcatgggcttcctgctcatccaggaccacggcgagaaggagatgatacgcctcgccttcggccccgaggcgctgctgcacaccgtcatggccaaggcgcgcaaggacctgggcctgctcccgtcgccaggcccggggacgcccacatccgtcaccgccgccgcaacgcactcgccgttcctcctctcgcgccagaactccgggcgctgcggcgccggcaccgcgccgtcgccgctctcggtgtcgtcgccctcgtcgtgggcgccgccgccccacttctccaggaccaacagcgtcgtcagcaatggcgcgccggcggaggccttggccgccgacctcatgagcccggccgccgccgggaacgcgccgccgtcgcccttctttgccgccggggaaccgctcctcgacgagctccagctgcaggagcagctcgcgttcctcagcgacgccgccgccggcggccaccagctgcccctgttcgacgccagcgagtgccggagccccggctcaggagacgccgccgggttcttcccgtacggcgccctcgggtgggccaacggcggcccggggcaccgccggagctcgtcggtcagcgagctctgcctcggcggggccgacgggctcggctggaagccctgcctgtactacgcccgcgggtactgcaagaacggcagcgcttgccggttcgtgcacggcggcctcaccgacgacgccaccgctaagatggacaccgccaccttggagcagcagtgccaggacatcctgctccgctccaagtcccagcgccttgccgccttcccctactccccgaccggctcggtcccgggctccccgtccgcggccaccaagtgcctgagcttgctcttgcaccagcagcagcagcagaacgagaaccagagggtggcagctgcagcggccgccgcggcgctgatgctgggcggcgacgacgcgcacaagttcattgggcggccgcggctggaccgcgccgacttggcgagcttggtgaacccgggctcgcgccagatttacctcaccttcccggcagacagcaccttccgcgaggaggacgtgtccaactacttcagcatctacggccccgtccacgacgtgcgcatcccgtaccagcagaagcgcatgttcgggttcgtcaccttcgtgtacccggagacggtgaagctgatcctggccaagggcaacccgcacttcatctgcgacgcgcgcgtgctcgtgaagccctacaaggagaagggcaaggtccccgacaagtacaggaagcagcagctgcaaggcgagagggcggtggatttcttctccaacgggttagacggcagagaaaaccacttggatctgcaccagctgggtgcgaggatgctccagcactcgcacagcgcgaacgagatgctgctgaggaggaagctggaggagcagcagcaggccgctgctgccgagctgcagcaggccatggagctccagagccgccgcctgatgaggctgcagctgctggacctgaagccgcgcgcgtcgccgagccccatcggcagcatgcccctgggccccacccaaagggccgtcgactcgccgcccgattccggcagggaggagtcgtccgccggcgacgcgagcccgaacgcggacagcgaccaaagcgccgagcacaacctgccggacagcccgttcgcgtcgccgacgaggtccgcggccttggctcgcgaccctttcgcggccatcgaccgggagatggctgcctcgcccggtcgtcgaaacggcgccggttccttcgctggcatcagcagcagcagcggcgtcctcgccggccatctgaggccgtcggctctggacatcccctccccgttcttccccatgtcgatgaccaggctgtcctccgatcacggcgccgggcgcgatcgggatgtaaagttatatagtcctcgctgctacttgcctaatcatagaatacttaactag

SEQ ID NO: 278 - protein, 678 aa, Zea mays
MDAYEATKVVFSRIQALDPDHAAKIMGFLLIQDHGEKEMIRLAFGPEALLHTVMAKARKDLGLLPSPGPGTPTSVTAAATHSPFLLSRQNSGRCGAGTAPSPLSVSSPSSWAPPPHFSRTNSVVSNGAPAEALAADLMSPAAAGNAPPSPFFAAGEPLLDELQLQEQLAFLSDAAAGGHQLPLFDASECRSPGSGDAAGFFPYGALGWANGGPGHRRSSSVSELCLGGADGLGWKPCLYYARGYCKNGSACRFVHGGLTDDATAKMDTATLEQQCQDILLRSKSQRLAAFPYSPTGSVPGSPSAATKCLSLLLHQQQQQNENQRVAAAAAAAALMLGGDDAHKFIGRPRLDRADLASLVNPGSRQIYLTFPADSTFREEDVSNYFSIYGPVHDVRIPYQQKRMFGFVTFVYPETVKLILAKGNPHFICDARVLVKPYKEKGKVPDKYRKQQLQGERAVDFFSNGLDGRENHLDLHQLGARMLQHSHSANEMLLRRKLEEQQQAAAAELQQAMELQSRRLMRLQLLDLKPRASPSPIGSMPLGPTQRAVDSPPDSGREESSAGDASPNADSDQSAEHNLPDSPFASPTRSAALARDPFAAIDREMAASPGRRNGAGSFAGISSSSGVLAGHLRPSALDIPSPFFPMSMTRLSSDHGAGRDRDVKLYSPRCYLPNHRILN

SEQ ID NO: 279 - DNA, 1671 bp, Vitis vinifera
atggatttttctgagtccacagcagttgttttcaatagaattcaaaaactagaaccagagaatgtttcaaagattatagggtatcttcttgttaagggttttagtaagggagaaatgattcggttggcttttggtcccgataaggcgattcgtatgataattgaaggggtcaaaatagagcttggtttgattccaaatccaccatctccaatctctcctcacttccctcctctctctccttcaactggttcttggttcccttcatcttctccgtctcccgtgaaccgatatctccaacatgctacagagcaactaccaaaagattatagtctccaaagtcagcctttgggtttagaggaacagttagaacaggtcaacccagcaattttaggagtttccggtgattactattacccggagacggcagtggaaaatttgagtgtgaggacgggtccaagatctctgattggttcggaatttccagttaaggtttgtcactatttcaacaaggggttttgtaagcatggaaataattgtcgatacttgcatgcacaagtctttcctgaggttttaagccctattgcaaatgatcttgctaatgatgatcatattttctcacctggttcaatcgagaagttggaattggagttaacagagctcctgaaatcaagaagaggcaatcctgtctccattgcctctctgcctatgatgtattatgagagatatggtaggggtcttcaggctgaagggtacctcactgagagccaacgacatgggaaagctggttatagtttgacaaagcttcttgctcggttgagaaccattcgtctaattgacaggcctcatgggcagcattcagtaattttggcagaagacgtgccaaaatacatggaaagccggagtgagaggagtgaccctggtccaattgtaagtggttcccggcagatatatctgacatttccagctgagagtacttttactgaagaagatgtctctgactacttcagcacctttggactggttgaggatgtgaggattccctgccagcagaaacggatgtttgggtttgtaacctttgacagttctgataccgtgaagagcattttggccaagggaagtccacattatgtttgtggggctcgtgttcttgtgaaaccttacagagaaaagccaaggactggtgataggaaatattcagagaaatttgagtcttcaatgtattatcctttgcaatatgcagacatggattccgagcttcacatgatgcctagaggaatggagacctcaagattgctcagaaagcagattatggaagagcaagagtttgcacaggatcttgaatttgagacaaggcgtctctccaagctgcagctagcacgaaatcctctggccaatcagctccaccatggctattccttggatgaactaaaagtcttggaagccattattccatcattctggaattgttctctctcttctgacttgacgctttttcttggtcttgttcaaaccatagcacatgcaaatcattccaagttcccgactgttgtccattccaattatccattggatgtttcaaacaatggctctacaagtgatgataagccatggcgtgcagtcaacaaccccatcgatcataagaggtataaatgcatttctattagtgattag

SEQ ID NO: 280 - protein, 556 aa, Vitis vinifera
MDFSESTAVVFNRIQKLEPENVSKIIGYLLVKGFSKGEMIRLAFGPDKAIRMIIEGVKIELGLIPNPPSPISPHFPPLSPSTGSWFPSSSPSPVNRYLQHATEQLPKDYSLQSQPLGLEEQLEQVNPAILGVSGDYYYPETAVENLSVRTGPRSLIGSEFPVKVCHYFNKGFCKHGNNCRYLHAQVFPEVLSPIANDLANDDHIFSPGSIEKLELELTELLKSRRGNPVSIASLPMMYYERYGRGLQAEGYLTESQRHGKAGYSLTKLLARLRTIRLIDRPHGQHSVILAEDVPKYMESRSERSDPGPIVSGSRQIYLTFPAESTFTEEDVSDYFSTFGLVEDVRIPCQQKRMFGFVTFDSSDTVKSILAKGSPHYVCGARVLVKPYREKPRTGDRKYSEKFESSMYYPLQYADMDSELHMMPRGMETSRLLRKQIMEEQEFAQDLEFETRRLSKLQLARNPLANQLHHGYSLDELKVLEAIIPSFWNCSLSSDLTLFLGLVQTIAHANHSKFPTVVHSNYPLDVSNNGSTSDDKPWRAVNNPIDHKRYKCISISD

SEQ ID NO: 281 - DNA, 4830 bp, Vitis vinifera
atggcttctgcttctttgatctatgctgttaattttatcaaccctccaaaatgtattttgcattcgaaaagggattatttttctttcaatatgagttatacttgtacaaaatccaaaacctcacaaagacagatgagaaataagcaatcaagaatggctacaattgctgctggaaatcaagaaatggacttcactgatccctgttggaagaccaaatttcaagaggatttcgagttgagatttaatttgcctcaccttaaggatgtattgcccataaagccaaggcctacgacgttttccctgaaaaatagaaatgctcaattagtgaatggcgccaatgtgcttgaggatcggaaaaatggttatgttaatgaggatgatagagcacttctaaaggttatcagatattcttcaccaacttctgctggagctgagtgcattgatcctgattgcagctgggtggagcaatgggtacatcgtgctgggccacgtgaggagatattctttgagcctggggaagtgaaagctggaattgttacctgtggagggctctgtcctggtctcaatgatgtcattagacagattgttttcactctggaactctatggggttaagaagattgttggaatccagtatggttatcgtggactttttgatcggggcttagctgaaatagagcttttccgtgaagtggttcaaaacattaatcttgccggtggaagtctgcttggagtttcccgtggaggtgctgatatcagtgagattgtagatagcatacaggccaggggaattgatatgattttcatacttgggggcaatggtacacatgcaggagcaaatgcaatacacaacgagtgccgcaggaggaagatgaaagtatcggttatatgtgttccaaaaacaattgataatgatattctgttaatggataaaacctttggatttgatactgctgtagaagaagctcaaagggctattaattctgcatatattgagtgcatatcatggcatcgggcttgtaaaactgatgggaagaagcagcggctttatagcaatgcacgcttcgctttcaagcggtaccatttcaaattgagggaccttataggtgtcttacgacatttagagcatctcatagagactaaagggtcagctgtgctctgtgtggctgaaggagcaggacaagattttgtagaaaagacgaattcgacggatgcatctgggaatgcgagacttggagacattggtgtttatctccaacagcagatcaagaaacattttaggaggattggcgttccagctgatgttaaatacattgatcccacttatatgattcgagcgtgtcgagcaaatgcatctgatgctgttctttgcactgttcttggccagaatgctgtccatggagcatttgcagggttcagtggaatcactgttggaatatgtaacagccactacgtctacttaccaatcccggaagtgatcgcctctccaagagtcgtcgatccagacagccggatgtggcaccgtaactgccgctttcatggtgttgccataatactattgcaggaagtggcaaggttcacgttcccagagctatttagctctcttccgaaatgcgtctattcttcatctaccagtgaagattcacaatctccaaccgacgatgagtttccattgtttggattcgaattttttgactctgcgcaaatagagaagagcctcatcaaatcaatctggagggatgacagagctcagtttctaactcaatcaaagcgactctttcagaaccctgagaatcgagagagcttggaagacgacgaaaacagagactcaccagaactactggaccgaagcattgttgcaaagcttctgcagtggtgttgcaggtttgattcagtggactgcgccacagctttgctaaatggagcggttggaatgttgcctctcatcaacgaaatggatgaaaayggacgaacggcactgcatacggcggcggaggctcacgcggccagatgcgtcgagcttctcctacgcaaacgcgctcgtacggacctcaagtccaaggacggttgctcacagcttgctttggagctgtctctgtctagcagaaggatggatgtactgtggaatccagatracgccattgaagacttgatcgttctgcttcgtgaaaaggacctaacggcggtaaaatttctgacggagaaaacaaaagatatcgcggaagtcgcttacgtgaccgccatggaggggcgtgtgattgcgttagctgctctgctagtggttgccgcagacaaggttaacgcttcgatactggtgttgcaaagcgacggggacttgagttccaaggagaagaccagcatttacgaatgcgtggttaaagaggccttatctctgggccggacggagacctcaaagtcgaccacatgcaaatggaaaagcgaggaagtggagaagagggcactcctgctatgcgagatggagctgcttcagctcttcggagctgtagctcacaacagttgtacggaaaggaaggtgacgtcacctctcattcgtgcagcccaggctggagatgaagctgtaatcaatttacttctgaagacgagtatagaagttgacgacacagatgcagaaggaaactctgctcttcaatgttcccttaagacgtccatggcctcagttgctaagcagataagaattgtatggctccttctaaagcatggtgctcgagtgagccacagaaataaattggggctaaatgcaatccatattgctgccgcaaatggtaattcagaggccctccatttgcttctactggaagagccagacggtgtgaatgcaacaactgaaatgaaagaaaccccactattttttgcagtaaagaacaattatatggactgtgctgagcttcttttgcgctggggagcaaatagccaagtcctcaacttacgtagacaaagacctattgacttggcaaagtcgcaagagatgcggttcatgctaagtccaaccaatattggccttaggcaccgagctttccccatgcaacggaaatttactacttacttccataaccatgagatgatttcagaaacctgtgagtcactcccaaacgtgatagaagaaggcacccttcctgagagctctagaacttgctcgatcgcgaagacaggaatctgcagatactttgaatctccaggaggttgtgtgcgaggggctaagtgcttctatgcacatggggaagatgagcttcggcggctgaagcagggaacaagaacacagcactcaagtacaattgaggagcttaaaaggaaaatttttgtaggaggcctgccctcttcactggacactgtcacctttgacgaatgtctttttctctatcaatcttgtgaagcagactcattggctaagatttttgaagaacaatttggttcagtagaagaagccatagtcatgggtgatcaaatgggcgaccaaatacactctcgaggatttggtttcgtcatctttaaacatgagaaatccgcctcagatgctgttcaagcgcactacattaccattatgggcaagcaagttgagataaaaagtgctgttccaaaatgcatattatttgcagagctccaaacactaccacctcaacaagaagatcaggaacaaggacaaattattcagtttcagccacaagcagcaacacctgatgagaagaatactgatgatggtgaacctaggcagatgtcttgggttgataaattactccagaatccgccaaatacatgtttcagtgaacctcagattcttcttaattctacaactccaaaccaaagcatgcctaaatgggtcagaattttcaagaggtggcttcccagcttcttaaatgatgtttcaaagcgtcttaaggagggagagtggtaccccctctcttctctaaaagctgattttagggctacatgtggccaagaactggatcacacctctctgggctatcccaagcttagtgacttcatgagatctttccctggcctatgtcgcatgaagattgtccctgtaggtggacgaggacctgccactcacatggtcctccagcctaaccatcagcaacagacccaaccacttccaatgcggtgtttttctcccaccccttcaccccttgatgactatgatgatgatggttccattgatttgaagtctcttggtgaatttctaccagtttcttatgataatgctggctcccttggtggcagctttgaggatggggactcactccatgggactcttgaagagagtcctgctcacaaggatgcaaaacatggtgtccacccatggttcttggaatttttaaagccagatacactcctgggtcaaccatggtttaggaatgaaaatgcagctgcaggagatgattataaaagcaaggagctcaggcaacaaaaaaggcacctggttctagaggcccttgcaagagaaaaaaacaacacctctgtcttcttcttgcgtgagtttgatttttatgagaattacaagtcaagtgtggctcagggaaagtgctttgcatgcaatagaagtgaaatgttctgggctaattttccgtgcaaacacttgctgtggtgcggcaactgtaagatacatgctattcaggcagccagcattttggagcacaaatgcgtggtgtgtgatgctcaagtgcagaatattggtccactcccatggactgagaaatatcagcaaatctgtgatgtccccaacaacgacttccctcctttcgaccctaaccctataagaatgtacgcccattccatgcctacattttcccacaagtgtatgatcgtttcatag

SEQ ID NO: 282 - protein, 697 aa, Vitis vinifera
  UNSURE (542)..(542): Xaa can be any naturally occurring amino acid
  UNSURE (564)..(564): Xaa can be any naturally occurring amino acid
MDSYEATRIVFSRIQALDPENASKIMGYILLIQDHGEKEMIRLAFGPETLLHNLILKAKTQLGILSNTPSTPTSPSPFNPISKPTRLPTNNGFNPSSSWPVSGFSDLRSPNSTTAQLSYAAVVNGATNVSDLGTVSSSPASIPYYNNCSGSNNSSVVCNDNVMDDYQLQDHLSFLNDASKPEDLFDPRLELAMSPSFGETQLHRRSYSFNDACYGSDDGASGFGWKPCLYFARGFCKNGNTCKFLHGGFADSVEASSAASAAIVGSPGKLDGFEQEMLRSQQQRLAVASQLMAGLNFPYNKCMNFFMQQNETQRSAAAALMMGEELHKFGRCRPERNDFSGMGLGGAVNPGSRQIYLTFPADSTFREEDVSNYFSIFGPVQDVRIPYQQKRMFGFVTFVYPETVKLILAKGNPHFVCDSRVLVKPYKEKGKVPEKKQQHQQQQQQQQQQQQLERGEYSTCSSPSGIDPREPYDLHLGARMFYNTQEMLLRRKLEEQADLQQAIELQGRRLMNLQLLDLKNHQHQHQHHLHNLSGGAPVASPXQSSIHNNQSLGLPSDGNNQEVXEENSSSPAATTSPTAAADKPLRQEVNISCNSNSGNDSGNNSTEESSNPADFDLHESLEHILPDSLFASPTKSAGDRSVFSTASASVDESTTISITPASNNNPVLPGTTLNMASLKSCFFEMPRFPSGHGAIEM

SEQ ID NO: 283 - protein, 20 aa, Artificial sequence
  C3H consensus
  UNSURE (2)..(8): Xaa can be any naturally occurring amino acid
  UNSURE (9)..(9): Xaa can be any naturally occurring amino acid or no amino acid
  UNSURE (11)..(15): Xaa can be any naturally occurring amino acid
  UNSURE (17)..(19): Xaa can be any naturally occurring amino acid
CXXXXXXXXCXXXXXCXXXH

SEQ ID NO: 284 - protein, 5 aa, Artificial sequence
  Motif I
MIRLA

SEQ ID NO: 285 - protein, 17 aa, Artificial sequence
  Motif II
  VARIANT (16)..(16): / repalce = “Lys”
  VARIANT (17)..(17): / repalce = “Ala”
ESLEHNLPDSPFASPTK

SEQ ID NO: 286 - DNA, 56 bp, Artificial sequence
  primer 1
ggggacaagtttgtacaaaaaagcaggcttaaacaatggatgcttatgaagctaca

SEQ ID NO: 287 - DNA, 50 bp, Artificial sequence
  primer 2
ggggaccactttgtacaagaaagctgggtacgtaacataacatgctgtcc

SEQ ID NO: 288 - DNA, 2194 bp, Oryza sativa; identical to SEQ ID NO: 5

SEQ ID NO: 289 - DNA, 309 bp, Arabidopsis thaliana
atggatatgataacgaagatggtgatggagagaccggtggtgatttacagcaagagctcttgctgtatgtctcacacgatcaagactttgctctgcgatttcggagcaaatccagcggtttacgagctggatgagatatctagagggagggagatcgagcaggcgttgttgcggctcgggtgtagccccgcagttccgggcgttttcattggtggagagttggtcggtggagccaacgaggtcatgagtctacatcttaacggatccttgattcccatgcttaagcgggctggtgcattgtgggtttga

SEQ ID NO: 290 - protein, 102 aa, Arabidopsis thaliana
MDMITKMVMERPVVIYSKSSCCMSHTIKTLLCDFGANPAVYELDEISRGREIEQALLRLGCSPAVPGVFIGGELVGGANEVMSLHLNGSLIPMLKRAGALWV

SEQ ID NO: 291 - DNA, 309 bp, Arabidopsis thaliana
atggagaagatatcaaatttgttagaagacaagcccgtggtgatattcagcaagacgtcctgctgtatgagtcactcgatcaagtcgcttatatctggttacggtgcgaattcaacagtgtatgagctagacgaaatgtctaatggaccagagatcgaacgagcacttgtagagcttgggtgcaaaccgactgtgccagctgtctttatagggcaagagctcgtaggtggtgcaaatcaacttatgtctcttcaagtcaggaaccaactagcttcgttgctccgaagagctggagccatatggatttaa

SEQ ID NO: 292 - protein, 102 aa, Arabidopsis thaliana
MEKISNLLEDKPVVIFSKTSCCMSHSIKSLISGYGANSTVYELDEMSNGPEIERALVELGCKPTVPAVFIGQELVGGANQLMSLQVRNQLASLLRRAGAIWI

SEQ ID NO: 293 - DNA, 887 bp, Arabidopsis thaliana
aaacgaacccaactacacaagtctctctctcttttcccatttctccctctctctctttgtctctctatcatcaattatgcaaaaagcaattcgaccatacgagtcaccgtggacgaagaccgtgccgggcaatagcattttccttttaaagaatgaagataaaccatcatcatcatcatcatcattatcatggttaacatcaggatcaccaaagccaacatctataagcaataagagatcaagcaacctagttgtgatggagaatgctgtggtggtgtttgcaaggagaggctgttgtttgggacacgtggcaaaacggctgctactgacacatggcgtgaatccagtggtggttgagattggtgaagaagacaacaacaactacgacaatatcgtaagtgataaagagaaattacctatgatgtacataggaggaaagttgtttggaggattggaaaatctgatggctgctcatattaatggccactccatcaagattcgaaccgatacatggtcgtcgttctcagtcgccaccgtcgaccgaatccgctggtgagaatagcgtaagaagcatccacggtaacgaatcaacaagaagggttaaattagaagagaccataactaaatcacgataagagaatgctttcctccgctcaaatcttacggataggttctgagatgaagaacgaagttgtttcagagataatttagtaatggatcatcatcggagatcttaaaattcgtagataaaatatggatttattttttgtcgtttagaagaagaagaatagcaaattttcgagtatttccttttttccgactaggttacgaaaaaggaaatttcattaattatgctaaaaacaaaaaaatatggaaattttctcaca

SEQ ID NO: 294 - protein, 150 aa, Arabidopsis thaliana
MQKAIRPYESPWTKTVPGNSIFLLKNEDKPSSSSSSLSWLTSGSPKPTSISNKRSSNLVVMENAVVVFARRGCCLGHVAKRLLLTHGVNPVVVEIGEEDNNNYDNIVSDKEKLPMMYIGGKLFGGLENLMAAHINGDLVPTLRQAGALWL

SEQ ID NO: 295 - DNA, 505 bp, Arabidopsis thaliana
aatccatatacttcttcttcttcaccttatgcaagataatggacaaagttatgagaatgtcgtccgaaaaaggggtggttatatttaccaagagctcctgttgtttgtcctatgcggttcaagttctcttccaagatcttggtgttaaccctaagatccacgagattgataaggaccctgaatgccgagagatagagaaggctcttatgaggctagggtgttcaaagccggtcccagccgtcttcattggtggcaagctcgttggttcgaccaacgaagtaatgtccatgcacctaagcagctcgctcgttcccctagtgaagccatatttatgttaaacaacaacgaaggagtatttatgatattaattagctatgtatatgttattcaataaggaacaaaattgagccaaatctttgtaatgtgttttttggtattattattggttgtataacattgggaaagtgtacgtataattataagactgttatattgattcgaaggt

SEQ ID NO: 296 - protein, 99 aa, Arabidopsis thaliana
MDKVMRMSSEKGVVIFTKSSCCLSYAVQVLFQDLGVNPKIHEIDKDPECREIEKALMRLGCSKPVPAVFIGGKLVGSTNEVMSMHLSSSLVPLVKPYLC

SEQ ID NO: 297 - DNA, 600 bp, Arabidopsis thaliana
atctatctttaaaaacatacttgaaaatgcaaggaacgatttcttgtgcaagaaattataacatgacgacaaccgtcggggaatctctgcggccgctatcgcttaaaacgcagggaaacggcgagagagttcggatggtggtggaggagaacgcggtgattgtgattggacggagaggatgttgcatgtgtcatgtggtgaggaggctgcttcttggacttggagtgaatccggcggtccttgagattgatgaggagagggaagatgaagttttgagtgagttggagaatattggagttcaaggcggcggaggtacggtgaagttaccggcggtttatgtaggagggaggttgtttggagggttagatagggttatggctactcatatctccggtgagttagttccaattcttaaggaagttggggctctgtggttgtgattgtaaattaataatttaaaattatttttttttcttttaattaagaatcttgattggtaattgttgtttacggtttataattgaatcgtttcatatatatgtatataaagaaataaataaaagaaaagtctcaagttgaaatttgctagagattgtaccc

SEQ ID NO: 298 - protein, 137 aa, Arabidopsis thaliana
MQGTISCARNYNMTTTVGESLRPLSLKTQGNGERVRMVVEENAVIVIGRRGCCMCHVVRRLLLGLGVNPAVLEIDEEREDEVLSELENIGVQGGGGTVKLPAVYVGGRLFGGLDRVMATHISGELVPILKEVGALWL

SEQ ID NO: 299 - DNA, 680 bp, Arabidopsis thaliana
ctcaggcaacactgagcttaatactgtagtacacacacacacacacacacacacacacacaaaaccctcttttcttcaaacaggaaccccaaaagcgaggtttaatctcaggcgttttcagttctaaatctctttcaatccacaagagaaggaagattttgtttcttcttctccaagaaacaatccctagattgatcgagacctccttcaagaaaccatggacaaagttgtgagaatgtcgtcagagaaaggagtggttattttcagcaagagctcgtgttgcatgtcctatgcggtccaagtacttttccaagaccttggggttcacccaacagtccatgagatcgataaagaccctgaatgtcgtgagatcgagaaagccctaatgaggttagggtgttccacgccggtcccagccatctttgtgggtgggaagctcattggttcgaccaatgaagtcatgtcgcttcacttaagcggctcgctggttccgctagttaagccgtttcaagccaatctatgttaaaaaggtgttctaattttctatactacaaaaatgtatttcaataagaaacacaaatcttatagcctatgcaacctttgtaatgtagctttatatatatattgtttgttctgtaatttcaggtaatatccaataataattgactaatttctactca

SEQ ID NO: 300 - protein, 102 aa, Arabidopsis thaliana
MDKVVRMSSEKGVVIFSKSSCCMSYAVQVLFQDLGVHPTVHEIDKDPECREIEKALMRLGCSTPVPAIFVGGKLIGSTNEVMSLHLSGSLVPLVKPFQANLC

SEQ ID NO: 301 - DNA, 312 bp, Arabidopsis thaliana
atggaacgagtaagagatttggcatcggagaaggcggctgtgatattcacgaagagctcgtgttgcatgtgtcatagcatcaagactctcttctacgaactcggggcgagtcctgccatccatgagcttgacaaggacccgcaaggccctgacatggaacgggccctcttccgggtattcgggtctaaccctgctgtccctgcggttttcgtaggaggaaggtacgtcggctcagctaaagacgtcatctccttccacgtggatggctccctcaagcagatgttaaaggcctctaacgccatatggttgtga

SEQ ID NO: 302 - protein, 103 aa, Arabidopsis thaliana
MERVRDLASEKAAVIFTKSSCCMCHSIKTLFYELGASPAIHELDKDPQGPDMERALFRVFGSNPAVPAVFVGGRYVGSAKDVISFHVDGSLKQMLKASNAIWL

SEQ ID NO: 303 - DNA, 632 bp, Arabidopsis thaliana
atactcaaaacaaaacaaaacatacatcaaaacgctaaagtttaaacccctagccatcatcagatcttcagacttctgaggatcatggacaaagtgatgagaatgtcttcagagaaaggagtggtgatcttcacgaagagctcatgttgtctctgctacgccgttcaaatcctgttccgtgaccttagggttcaaccaaccatccacgagatcgacaacgacccggactgccgtgagatcgagaaggctcttctccggctcggctgttccacggcggttccagctgtctttgtcggaggcaagcttgttggctccaccaatgaagtcatgtcccttcaccttagtggctctcttgtcccattgatcaaaccctatcagtccatcctttactagcaaaattaaaccaactcaatatataatatctaattattagctagtgagaataaacacagttacagctagagtgtgagctagctagatattcagtgaggacttcgtctgaattaatgtttatcgtttgtatgttctattgtttagcttctctcgtgtttcagtttagttaatcaactggtgtatgttgatgtatgactctctgtttatgctaatgaaaatagtattgaaacttttacatt

SEQ ID NO: 304 - protein, 102 aa, Arabidopsis thaliana
MDKVMRMSSEKGVVIFTKSSCCLCYAVQILFRDLRVQPTIHEIDNDPDCREIEKALLRLGCSTAVPAVFVGGKLVGSTNEVMSLHLSGSLVPLIKPYQSILY

SEQ ID NO: 305 - DNA, 748 bp, Arabidopsis thaliana
ccaacaaactttagccaatccctctttctctcttattcgtgtttaatctagtttctttccaaacaaaagtgtaacaagagaaagagaagagatatcaaagatgcaataccagacagaatcgtggggatcatacaagatgagcagcttaggattcggcggtttggggatggtggctgacactggtctgcttcgtatagagtccctggcttctgagagcgcggtggtgatcttcagcgtgagcacgtgctgcatgtgccacgccgtgaagggtctcttcagaggaatgggcgtcagccccgccgtccacgagctcgacctccacccctacggcggcgacatccagcgagccctcattcgtctcctcggctgctccggctcctcttctccagggtctctcccggtcgtcttcatcggcggtaaactggttggagctatggacagagtcatggcttctcacataaacggctctctcgttcctcttctcaaagacgccggcgctctctggctctgatcccttcctctgctttcttttttcttttctatttgaagttttcttgtaagagaatgtggtggaggaagattaggaaactagtcaatggctgtaatgacaggttttagattatagtttgtaattagagagagagttgttttaagctcacctttctctgtcttcctcttcttcatctcttattgatctttcgaatgctctcattaaatcataatagtaaacactttgcatctttattaa

SEQ ID NO: 306 - protein, 136 aa, Arabidopsis thaliana
MQYQTESWGSYKMSSLGFGGLGMVADTGLLRIESLASESAVVIFSVSTCCMCHAVKGLFRGMGVSPAVHELDLHPYGGDIQRALIRLLGCSGSSSPGSLPVVFIGGKLVGAMDRVMASHINGSLVPLLKDAGALWL

SEQ ID NO: 307 - DNA, 813 bp, Arabidopsis thaliana
atggaggaattaaggcaagcaacgaacaattttgacatggataatactattgggcatggaagtcacggaacagtttacactgggctgatcaaggacatgcccgttgtgatcaaaagggagtggtgtagtccccaacctagattcttggatgaggtacacacaaacaaaaaattggtttcttacatccaacataaaaatttggttaatctgctcggttattgttgcgacgacgagaaccaatcgctcgtctttgagtatatggtcaacggtagcgtccgagattatctagagtctagcgacttaacgttcaagaaaagaatatctatagctcttgatgcagccaaagggttactacacttgcacaacttagatcctccagtacaacacaagagatttacggcgagtaaagttttgctcgatgccaacctcaatgccaaggtatcagatggggcaatgttgggactgcttcaagcaagtgatattcatctcgctaatccaagaggtggttcagaggagacaattgatgtgtatagcttcgggttgttccttctagagcttatcacctgtcaaaaccctggactgttacaatcagactatgaagttttacaatggaatatacctgcagaaacattcacgagaccatcgttccaagcttatctgataattacgtcggaatgcttggaatatcccccgataaatcgcccaaagatggatgtggtcgtgaccgagcttgagacgatttaccttaatgtcatcagtgagaaccatgaggtttctctaggaagtgagctttttaacataaccattgagtaa

SEQ ID NO: 308 - protein, 100 aa, Arabidopsis thaliana
MDVVARLASQRAVVIFSKSTCCMSHAIKRLFYEQGVSPAIVEIDQDMYGKDIEWALARLGCSPTVPAVFVGGKFVGTANTVMTLHLNGSLKILLKEAGAL

SEQ ID NO: 309 - DNA, 309 bp, Arabidopsis thaliana
atggatgtggtagcaagattagcgtcgcaaagagcggtggtgatattcagcaagagtacgtgttgcatgtctcatgcaattaaacggttgttttacgagcaaggtgtgagcccggcaattgtagagatcgaccaagacatgtatgggaaagatatcgagtgggccttggcccgattaggctgtagccctacggttcctgcggtttttgttggagggaaattcgtaggaacggccaatactgtcatgactcttcatctcaatggatcattgaaaatattgctcaaggaggctggtgctttgtggctttag

SEQ ID NO: 310 - protein, 102 aa, Arabidopsis thaliana
MDVVARLASQRAVVIFSKSTCCMSHAIKRLFYEQGVSPAIVEIDQDMYGKDIEWALARLGCSPTVPAVFVGGKFVGTANTVMTLHLNGSLKILLKEAGALWL

SEQ ID NO: 311 - DNA, 486 bp, Arabidopsis thaliana
aaagcaaaaacaaacataaagatccttgagttcccactcacatagtcacactacatttgtatcacatttatatacatagaaatggaaagcgttagaagtttagttgaagacaaaccagtggtgatattcagcaaaagctcttgctgcatgagccactcaattcaaacactgatctcagggtttggggcaaagatgacggtctacgagctagaccaattctcaaacggtcaggagatcgagaaggcattggtacagatggggtgtaaacccagtgtaccagctgtgttcatagggcaacaattcatcggtggtgctaaccaagtaatgactcttcaggtcaagaaccagctagccgcaatgctaagaagagccggagccatatgggtgtaacagaagacaagagagtcaaatgcaaactaggatagaataagatgagaatgatacatatcgttgttttgcttcttctttaattttcggggtttcgag

SEQ ID NO: 312 - protein, 102 aa, Arabidopsis thaliana
MESVRSLVEDKPVVIFSKSSCCMSHSIQTLISGFGAKMTVYELDQFSNGQEIEKALVQMGCKPSVPAVFIGQQFIGGANQVMTLQVKNQLAAMLRRAGAIWV

SEQ ID NO: 313 - DNA, 686 bp, Arabidopsis thaliana
ctcattacaaaaaaaacaacacttattgatcactaactttcttgaccatcgcaaatggagagaataagagatttgtcgtcgaagaaagcggcggtgatattcacaaagagctcatgttgtatgtgccatagcatcaagacgctattctacgaactaggcgctagtccggcgatccatgagctcgacaaagaccctgaaggccgtgaaatggaacgggccctacgtgccctcggctcatcgaacccggcggttccagctgttttcgttggaggaaggtacatcggatcagccaaagacatcatctcattccacgtggacgggtcactcaagcagatgcttaaagacgctaaggccatttggttatagcgtccgatcatcgtacatgtatgtgtatatatctatatatatatatatatatgtatgcacatgtaattgtgtcaataatcgatgtgctaagagcgcatagatgataaatataatcgggagggagcatagatgtattaaaatgctttgctttcttcggatggatcgtagttattggataataatttatatacatgtatagatgtatatatgtatgttgacttacgtacattgtacggatgaaaagacaagtgtacctttaagcactgttgtataacattactgttcgaattccacgtaaagagtaaactcaaatcttaaaa

SEQ ID NO: 314 - protein, 103 aa, Arabidopsis thaliana
MERIRDLSSKKAAVIFTKSSCCMCHSIKTLFYELGASPAIHELDKDPEGREMERALRALGSSNPAVPAVFVGGRYIGSAKDIISFHVDGSLKQMLKDAKAIWL

SEQ ID NO: 315 - DNA, 615 bp, Arabidopsis thaliana
atcaaagctacatatactaaaagatatataatttctcgattgtcctgagccctcagaccaaaatctgaccatggacaaggttatgagaatgtcatcggagaaaggagtcgtgatcttcaccaaaagttcatgttgtctctgctacgccgtgcaaatccttttccgtgatcttagggttcaaccaacaatccacgagatcgacaacgatcctgactgccgtgagatcgagaaggccttagttcgtcttggctgcgccaacgcggttcctgctgtctttgtaagtggcaagctcgtgggttcgaccaacgatgtcatgtcgcttcacctaagtggctccctcgttcccttgatcaagccgtatcagtcatttcataactagaaaaacaatatcgatgcttaagaaagataattagtatatactattaattggacagtgagaataatgatggaattagataacacgtaaatgtgtatcaggttcttatttatagctagtgcttatatcttgtttagcttatgtctaagaaattgatgagctagctttgtatttccagcttaactaatcggtggatgtactgatgtgtactatctatttaatggaaaaattattgagca

SEQ ID NO: 316 - protein, 102 aa, Arabidopsis thaliana
MDKVMRMSSEKGVVIFTKSSCCLCYAVQILFRDLRVQPTIHEIDNDPDCREIEKALVRLGCANAVPAVFVSGKLVGSTNDVMSLHLSGSLVPLIKPYQSFHN

SEQ ID NO: 317 - DNA, 505 bp, Arabidopsis thaliana
cagtctccttgattgtctctaattttccaatctttcatttctgattttgcattgtaatcgaacatacccactaatatcctttgcaagccgcttttcaaacaacctattacattataacaccaatggagaagatacaaaagatgatctccgagaagtcggtagtaatatttagcaataactcttgttgcatgtcacacacaatcaagactctcttcttagaccttggcgtgaacccgacaatctatgagctagacgagatcaacagaggaaaagagatagagtatgcattggctcagcttggctgcagcccgactgtgccagtggtgttcataggagggcagcttgttggtggagccaatcaagtcatgagtctccatctcaaccgttctctcattccaatgcttaaacgctttggggctttatggctttgataaaatataaaagaatagttacttattgatcgtagcttggtaataatgatgtcatgaaaccaatcaaggatgac

SEQ ID NO: 318 - protein, 102 aa, Arabidopsis thaliana
MEKIQKMTSEKSLVIFSKNSCCMSHTIKTLFLDLGVNPTIYELDEINRGKEIEQALAQLGCSPTVPVVFIGGQLVGGANQVMSLHLNRSLIPMLKRFGALWL

SEQ ID NO: 319 - DNA, 587 bp, Arabidopsis thaliana
cataacctatttcatcctcctcaagtcacttttcaaacacactttcaaattaacaaaaatggagaagctacagaagatgacctcggagaagtcgttagtgatatttagcaaaaactcatgctgcatgtcgcacacaatcaagactctcttcttagaccttggcgtaaatccgacgatttatgagctagatgagatcaacagaggaaaagagatagagcaagcattggctcagcttggctgcagcccgaccgtgccagtggtgttcataggagggcagcttgtcggtggagccaatcaagtcatgagtctccatctcaaccgttctctcattccaatgcttaaacgcgttggggcgttatggctttgacaaaatataaagaaatagttacttattgattgtagattggtaataataatgtattgaaaccaatcatatacatatgaatgttttgtgtctatctaagttttgaaaggatagattatgaggcagggtactgatttcgtaacgttcttggtatacttagtgttgtatttctattttcagtttttatgaatacaaacatgcatttaagaaaagtgtatgccat

SEQ ID NO: 320 - protein, 102 aa, Arabidopsis thaliana
MEKIQKMISEKSVVIFSNNSCCMSHTIKTLFLDLGVNPTIYELDEINRGKEIEYALAQLGCSPTVPVVFIGGQLVGGANQVMSLHLNRSLIPMLKRVGALWL

SEQ ID NO: 321 - DNA, 644 bp, Arabidopsis thaliana
atcaaaaacaaactctaagtcatctcttatatatcgtcagccaaagacttcaagttctctagcttaccaatttcaccccttttctctcatttcaataaaaaaaactacccatttcatcctcggcgagtcgctcttcgaacacatttcacattataataccaatggataagctacagaagatgatctccgagaagtcggtagtgatctttagcaaaaactcatgttgcatgtctcacactatcaagactctcttcatagactttggcgtgaatccaacgatctatgagctagatgagatcaacagaggaaaggagatagagcaagcattggctcagcttggctgcagcccaaccgtgcctgtggtgtttattggagggcagcttgttggtggagccaatcaagtcatgagtctccatctcaatcgctctctggttcctatgctaaagagggttggagcactatggctttgatttcaagataaggggatatttacgttatgaacgtagcatggtaataataatgtaatgaaagtaatcaaagatgacaaacaaaaacacatatacatgtatgttgtctatctaagttttggaaggatagactatgactataggatacctattttgtaacgtgtgggtaaactttat

SEQ ID NO: 322 - protein, 102 aa, Arabidopsis thaliana
MDKLQKMISEKSVVIFSNNSCCMSHTIKTLFIDFGVNPTIYELDEINRGKEIEQALAQLGCSPTVPVVFIGGQLVGGANQVMSLHLNRSLIPMLKRVGALWL

SEQ ID NO: 323 - DNA, 598 bp, Arabidopsis thaliana
atcttcacccaaagattataagctctctacacttttttgtagtatagtctcttatttcagtcaaaagacacacatttgcatcctccgtgaatcactttcttcagaaaatttacaaaaacaatggagaacctacagaagatgatctctgagaagtcggtagtaatttttagcaagaactcatgctgcatgtctcatacaattaagactctcttcttagactttggcgtgaacccgactatctatgagctcgacgagatcaacataggaagggagatagagcaagcattggctcagctcggatgcagcccgaccgttccggtggtgttcattggagggcagcttgttggtggagccaatcaagtcatgagtctccatctcaaccgctcccttgttcctatgcttaaacgtgctggagcattatggctttaacttcaaaataaatgtaatttcaaatatataatgcaattaagttactataattaaagtgaacaaagaaaacatacacaagaatgtatgtatgagttaatgctatgtctatctaagttttaaaaagatagattatccggttgcctattttgtaaacactggttctattata

SEQ ID NO: 324 - protein, 102 aa, Arabidopsis thaliana
MENLQKMISEKSVVIFSKNSCCMSHTIKTLFIDFGVNPTIYELDEINRGKEIEQALAQLGCSPTVPVVFIGGQLVGGANQVMSLHLNRSLIPMLKRFGALWL

SEQ ID NO: 325 - DNA, 500 bp, Arabidopsis thaliana
gtcacttctcattttcacccaaatatatcaataatctctctataagttatctagttttcccatattcatctctaatctagcattttgaccaaacacacctatttttctcctttgcaagacagttttcaaatatctatcacgttatattactaatggagaatctacaaaagatgatctccgagaagtcggtagtgatctttagcaagaactcttgctgcatgtctcacacaatcaagactctcttcttagaccttggcgtgaacccgacgatctatgaactcgatgagattagcagaggaaaggagatagagcatgcattggctcagctcgggtgcagcccgacagtgccagtggtgttcataggagggcagcttgttggtggagccaatcaagtcatgagtctccatctcaaccgctcccttgttccaatgcttaagcgcgctggagctttatggctttgacttcaaaataaatggaattgcaaatgacttaggttctga

SEQ ID NO: 326 - protein, 102 aa, Arabidopsis thaliana
MENLQKMISEKSVVIFSKNSCCMSHTIKTLFLDLGVNPTIYELDEISRGKEIEHALAQLGCSPTVPVVFIGGQLVGGANQVMSLHLNRSLVPMLKRAGALWL

SEQ ID NO: 327 - DNA, 770 bp, Arabidopsis thaliana
gattccttcttttgttttaaacttctttttgattctttctatatatacaaatacaaaatctcctcttcttcttgaaaaagctctttacgtttctctctctctctctaattgcctgctatgatgcaagaattaggcttacaacgtttctcaaacgacgtcgttcgcttagacctcactcctccttctcaaacctcatctacttctctttccatcgacgaagaggaatcaacggaagccaagatccgacggctgatatcggagcatcctgtgatcatcttcagtagatcttcatgttgcatgtgccacgtcatgaagagactcttagcaacgatcggcgtaatccccaccgtcatcgagctcgatgatcacgaggtttcctctcttcccacggctctacaagatgaatattccggtggcgtctccgtcgttggtcctccgccggcggttttcattggccgtgagtgcgtcggaggtcttgagtcccttgtcgctcttcacttaagtggtcaacttgttcctaagcttgtccaagttggagctctttgggtatgattgtaattttcatcgtcttcttacttgtgttaaaatcaataggcttttgcagtatcaaaagaaaacaaaaattagggtttcacttcaattggtcatgaaagccaaattctgattatcatcagatgatcataattaaacacccatgtagaaaatgaattatgaataattaaagatgtgtaattagtaaattttatatgatttgcttcttttgataaagt

SEQ ID NO: 328 - protein, 144 aa, Arabidopsis thaliana
MMQELGLQRFSNDVVRLDLTPPSQTSSTSLSIDEEESTEAKIRRLISEHPVIIFSRSSCCMCHVMKRLLATIGVIPTVIELDDHEVSSLPTALQDEYSGGVSVVGPPPAVFIGRECVGGLESLVALHLSGQLVPKLVQVGALWV

SEQ ID NO: 329 - DNA, 706 bp, Arabidopsis thaliana
ctctctctccctctctaagtactctttctctctaatgaagacgatgcgaggtttacgaaactgctcaaacgacgccgtaacgctagacctgacggttcatcctcctcctcctcctcctcttcctcctccagcaccatcaacagtctcctcctccaccgcctcgacgagcctgtcgttcgatgaagaggaaacatcagagtcaaagatcggacggctgatatcagagcatccagtcatcatattcactcgattttcctcatgttgcatgtgccacgtcatgaagaagcttctatcgaccgttggagttcacccaacagtgatcgagatcgacgacggagaaattgcttacctcgccgttgaagccgctccggtgcttttcatcggtggtacttgcgtcggtggcttcgagtcacttgtagcacttcacctaagtggtcagcttattcctagactcgtcgaggttggagccttatgggcataattgtaattttctgtatttttctttctttctttcaagtctagcctatttataaccttaagaaattctgataataaaatccattgtaaaattttccattaatccactctttctttctgaaacacaaaaatgtttttttttctttatcttttgccagtcggtaagtattgtttagatcatcaagtgtaagatttgttccatcataattattacaatttttgtgaatga

SEQ ID NO: 330 - protein, 145 aa, Arabidopsis thaliana
MRGLRNCSNDAVTLDLTVHPPPPPPLPPPAPSTVSSSTASTSLSFDEEETSESKIGRLISEHPVIIFTRFSSCCMCHVMKKLLSTVGVHPTVIEIDDGEIAYLAVEAAPVLFIGGTCVGGFESLVALHLSGQLIPRLVEVGALWA

SEQ ID NO: 331 - DNA, 679 bp, Arabidopsis thaliana
caaccaactctcacacaaattctctcgctctctcactctcttattctctttctctttttctctaagaatacaaaaaaagatgcaatacaaaacagaaactcgagggtcgttgtcctacaacaacaacagtaaggtgatgaacaacatgaatgtgtttccgtcggagacactggcgaagatagagtcgatggcggcagagaatgcggtggttatattcagcgtgagcacttgctgcatgtgccatgccatcaagcgtctcttccgtggaatgggcgtcagccccgccgtccacgagctcgacctcctcccttacggagttgaaatccaccgagctctcctccgtctccttggctgttccagcggtggcgccacatctccgggggcacttccggtggtgttcatcggagggaagatggtaggagcaatggagagagtgatggcttcacatatcaatggctcactcgtccctcttctcaaagatgctggcgctctttggctctgatgagtgctaatctcatcctccaaatatcaacctttggtttatctttggtttttaaggacagaagaaataggttaatcccagtgttgaattagagacagtgagagagaagagtgatgcgtttgttttaagcttagctctttgtctatcttaaatcacactaatatataaatgttaact

SEQ ID NO: 332 - protein, 140 aa, Arabidopsis thaliana
MQYKTETRGSLSYNNNSKVMNNMNVFPSETLAKIESMAAENAVVIFSVSTCCMCHAIKRLFRGMGVSPAVHELDLLPYGVEIHRALLRLLGCSSGGATSPGALPVVFIGGKMVGAMERVMASHINGSLVPLLKDAGALWL

SEQ ID NO: 333 - DNA, 505 bp, Brassica napus
ggcaagcaaaaaccaaactccaactcccgctttggtttcaggaacctccttcattttcttgaagacagctaaaaccaagaagaaaatggacaaggttctgagaatgtcatcggaaaaaggagtggttatattcagcaagagctcgtgttgcttgtcctacgcggttcaagtcctcttccaagatcttggggttagccctaagatccacgagatcgacaaggaccccgaatgccgagagatggagaaggcactgatgaagctaggctgctcaaagccagttccagccgtcttcattggtggtaagctcgttggttccaccaacgaagtcatgtccatgcacctaagcagctccttggttcccttagtgaagccatatctatgttaaaagaaaaggtcggaatgtatctcaataaggaaacaaatgtgagccaaatcttcgtaatgtgttttagtaattatattggctgtgtaaccttaaaagttatataaaatgtcttttcgtcca

SEQ ID NO: 334 - protein, 99 aa, Brassica napus
MDKVLRMSSEKGVVIFSKSSCCLSYAVQVLFQDLGVSPKIHEIDKDPECREMEKALMKLGCSKPVPAVFIGGKLVGSTNEVMSMHLSSSLVPLVKPYLC

SEQ ID NO: 335 - DNA, 603 bp, Brassica napus
cggcacgaggcaacataatatctcgaccgttgggagccgcaagatcagaaactgatcatggacaaggttatgagaatgtcatcagggaaaggagttgtgatcttcaccaaaaactcatgttgtctgtgctacgccgtgcagatactttttcgtgaccttagggttcaaccaacaatccacgagattgacaacgatcctgactgcctcgagatcgagaaggccttagtccgtcttggctgccccaacgcagttcctgctgtttttgtaagtggtaagctggtgggttctaccaatgaagtcatgtcgcttcacctaagtggctctctcgttcccttgatcaagccgtatcagttatttcataactagaaataaatggatctttaaggaaaagaaagataattgttgtatgttgagattggatagtaaataatgatggaaagattacacttgaatgtgtatcatgttatatatatagctgattttatattttgtttcgctcatgtccaagaaattaatttgctatctttgtattttccagcttaactaatcagtagatgtactgctgtattatctaatatctatagtaatgaagaaaattatact

SEQ ID NO: 336 - protein, 102 aa, Brassica napus
MDKVMRMSSGKGVVIFTKNSCCLCYAVQILFRDLRVQPTIHEIDNDPDCLEIEKALVRLGCPNAVPAVFVSGKLVGSTNEVMSLHLSGSLVPLIKPYQLFHN

SEQ ID NO: 337 - DNA, 833 bp, Brassica napus
cggcacgagggtttcttttctctctctgattgcaactatgatgcaagaattgggcttagaacgtttctccaacgatgtctttcccttagacctcactcctccttctcaaacctcatctacttctctttccatcgacgaagaggaatcttcggaggccaagatccggcggctgatatcggagcatccagtgatcatcttcagcagatcttcttgttgcatgtgccacgtcatgaaaagcctcctttcaacaatcggagtcgtccccaccgtcatcgagcttgatgaccacgaggtttcctctctccccatggctcttgaagaagaatattccggcggccgctccgtcgttgttcctccgccggcggttttcattggccgtgagtatgtcggtggtcttgagtcccttgttgctcttcatctaagtagtcacttggtccctaagcttgtccaagttggagctctttggttatgacttgctttttaatagtatcaaaaagcagaaacttagggtttttttctgattattatccgatgatcagaaccaaacacccatgtataaaatgaattatgagaaataataattaaagatgtgtaagtaaaaaaaaaaaaaaaaaaactcgagcgtcgaggaggataaagacctaaaaggagaaatggtgcagcgccttgtataccgttcgcgtcacagctacgccaccaagtccaaccagcacaggatcgtcaaaaccccaggtggtaaattgacataccagaccactaagaagcgtgcaagtggaccaaaatgccccgttaccggcaagcgtatccaggggatccctcacttgaggcctgct

SEQ ID NO: 338 - protein, 144 aa, Brassica napus
MMQELGLERFSNDVFPLDLTPPSQTSSTSLSIDEEESSEAKIRRLISEHPVIIFSRSSCCMCHVMKSLLSTIGVVPTVIELDDHEVSSLPMALEEEYSGGRSVVVPPPAVFIGREYVGGLESLVALHLSSHLVPKLVQVGALWL

SEQ ID NO: 339 - DNA, 665 bp, Brassica napus
actttctctcctctctctcctcccttctctctcatacaacaattatgcaaaaagcagtaagaccctacgagtcatcgtggacgaagaccataccggggaatagcattttccgtccagagaatgaagataaaccatcatcatccttatcatggttaacatcatcaccacaaaaaccatcatctttaagcatcaagaaaccaaacaacgtattggtgatggagaatgctgcggtagtgtttgccaggaaaggttgttgtatgggacatgtagccaaacggttgttactgacacatggagtgaacccattggtagttgagattgatgaaggagacaacaacggtgacaatatcatcatgagtgagctgggtaataacgtgattagtaaagagaaattaccagtcatgttcattggagggaagttgtttggaggattagagaatctgatggctgctcatattaatggtgatttaggacctactctcagacgagctggggctttatggctttgattcatcattgcaattctcatataaaagtttatttagttgccttttgatttttttttttctctttgttgcatttgcttgttgatattgtatgcaattttttaaacctatgtgaatttgtgattggttgttatgtgattggtttgatcataaacaaca

SEQ ID NO: 340 - protein, 154 aa, Brassica napus
MQKAVRPYESSWTKTIPGNSIFRPENEDKPSSSLSWLTSSPQKPSSLSIKKPNNVLVMENAAVVFARKGCCMGHVAKRLLLTHGVNPLVVEIDEGDNNGDNIIMSELGNNVISKEKLPVMFIGGKLFGGLENLMAAHINGDLGPTLRRAGALWL

SEQ ID NO: 341 - DNA, 666 bp, Brassica napus
gaacaaacctctctacgtttctctctctctctaattgttgcaatgatgcaagaattaggcttagaacgtttctccaacgacgtcgtttccttagacctcactcttccttctcaaacctcatccacctctctctccatcgacgaagaggaatcatctgaggccaagatccgacggctcataaccgagcatcccgtgatcatattcagcagatcttcttgttgcatgtgccacgtcatgaaaagactcttggcaacgatcggtgtcatccccaccgtcatcgagctcgatgatcacgaggtttcctctctccccttggctcttggagaagaatattccggcggtggctccggcgttgttcctcctccggcggttttcattggccgtgagtgtgtaggaggtctcgagtccctcgtggcgctccatctaagtggtcaccttgtccctaagcttgtccaagttggagctctttgggtatgatattgtaattttacttctttgagtttcaatagtattttgcagtatcaaaagcataaatttagggtttctcttttctggttattatctgatgatcataattaaacacccatgtactatatatgaataataataataattaaagaatgtgtaagtagtaaattttatataatttgcttcctctcggaggag

SEQ ID NO: 342 - protein, 144 aa, Brassica napus
MMQELGLERFSNDVVSLDLTLPSQTSSTSLSIDEEESSEAKIRRLITEHPVIIFSRSSCCMCHVMKRLLATIGVIPTVIELDDHEVSSLPLALGEEYSGGGSGVVPPPAVFIGRECVGGLESLVALHLSGHLVPKLVQVGALWV

SEQ ID NO: 343 - DNA, 596 bp, Brassica napus
tttctctctctctaaatgaagacgatgcaaggtttacggaacttcacaaacgacaatgtttcgctagacctgacgtttcctcccccagcaccaccacccatctcctcctccaccgcctccacgagcctatccttcgatgaggaggagacgtcagcgtcgaagatcgaacggctgatatctgagcacccggtcatcatattcactagatcatccacctgctgcatgtgtcacgtcatgaagaagcttctatcaaccgttggagtccacccaacagtgatcgagatcgacgaggaagagatcgcttgcctcgccgttcaagccgctccggtgctcttcataggtggtgcttgtgtcggtgggtttgagtctcttgtggcacttcaccttagtggtcatcttattcctagactcgtcgaggttggagccttgtgggaataattgtgtatgtttaacttataactatttaagaaaatctggtaataatatccattgtaaatctcatgatctactactattttctgttcttgattctgaaacataaaaatgtatttttcattttcccttgtgtacttttttttaatatctttcaccacttg

SEQ ID NO: 344 - protein, 140 aa, Brassica napus
MKTMQGLRNFTNDNVSLDLTFPPPAPPPISSSTASTSLSFDEEETSASKIERLISEHPVIIFTRSSTCCMCHVMKKLLSTVGVHPTVIEIDEEEIACLAVQAAPVLFIGGACVGGFESLVALHLSGHLIPRLVEVGALWE

SEQ ID NO: 345 - DNA, 808 bp, Medicago truncatula
ggacaacacaatccaactatatcacaaagaaaaaaccacacgttccttcttctctcatcactcaacaaacaaccatgcaacaagcaattccttataggtcatggacacacaacacttccaccactcacttcaatgttatcaaaccacacatattaactacaactaaaatccacaatacaattgatgagtcttctcataggccctcctcctttaattttaatgaagaggacaaaacaatgtttcataacatggtatcagagaacgcagttatagtctttgctagacgtggatgttgtatgagccatgtcgtgaagcgcttgcttctcggtcttggtgttaatcctgctgtacatgaggttgaggagaaagatgaagttggtttggttaaagaattggaatcaattgcaaatgaagagaaggttcaatttccagcagtgtttataggtggaaatttgtttggaggactggatcgaattatggccactcatatttctggtgaattggtccccattcttaaacaagcaggagctttatggctttgactcattaatatcatatttttttccactaaatttttcatttccggatatattgattccatcctttggaatttgtagagaaaaatatcaggagtgtttttcaaaaattattcatgtctttattggatgcaaatttttggctcgactatgtcaagttgttaagagtccaacactgaaaaaaatatgatttccgtaaatttataaatgagagatatcatcaccatagtaactgattttgtaaggtcgtattagactcgatttaaatctaa

SEQ ID NO: 346 - protein, 155 aa, Medicago truncatula
MQQAIPYRSWTHNTSTTHFNVIKPHILTTTKIHNTIDESSHRPSSFNFNEEDKTMFHNMVSENAVIVFARRGCCMSHVVKRLLLGLGVNPAVHEVEEKDEVGLVKELESIANEEKVQFPAVFIGGNLFGGLDRIMATHISGELVPILKQAGALWL

SEQ ID NO: 347 - DNA, 378 bp, Oryza sativa
atgcaggcggtggcggcggcggcggggatgatgcggcgggggagcctgacgatagacccggcgggggaggaggaggcgccggccgagagggtggggcggctggtgcgggagagccccgtggtggtgttcgcgcggcgggggtgctacatggcgcacgtcatgaggcgcctcctcgccgccgtcggcgcgcacgccaccgtcatcgagctggagggcggcgcggcggaggaggaggaggcggcgctgggcggcggcgccgcgctccccgcgctcttcgtcggcggcgaccccgtcggcggcctcgagggcctcatgggcctccacctcagcggccgcctcgtcccgcgcctcagagaggtcggcgccctctgcacctag

SEQ ID NO: 348 - protein, 125 aa, Oryza sativa
MQAVAAAAGMMRRGSLTIDPAGEEEAPAERVGRLVRESPVVVFARRGCYMAHVMRRLLAAVGAHATVIELEGGAAEEEEAALGGGAALPALFVGGDPVGGLEGLMGLHLSGRLVPRLREVGALCT

SEQ ID NO: 349 - DNA, 411 bp, Oryza sativa
atgcaaggagcaaggtcggcggcggcgatggcggcggcggcggccgacgaggagagggaggtgcggagggcggtggaggagaagccggttgtggtggtggggcggcgcgggtgctgcatggcgcacgtcgcgcggcgcctgctgctggggcagggcgcgaacccggcggtgctcgaggtcggcgacgacgccgacccggcggcgctcgtcgacgccgcgctgcaggcccgccggcgcaaggacggcggcgacaaggctgcggcgggcgacggaggcggcggagcggcggtggcattcccggcggtgttcatcggcgggaggctggtgggcgggctcgatcggctcatggccatgcacatggccggcgagctcgtgccggtcttgaagcaggcaggagccctgtggctctga

SEQ ID NO: 350 - protein, 136 aa, Oryza sativa
MQGARSAAAMAAAAADEEREVRRAVEEKPVVVVGRRGCCMAHVARRLLLGQGANPAVLEVGDDADPAALVDAALQARRRKDGGDKAAAGDGGGGAAVAFPAVFIGGRLVGGLDRLMAMHMAGELVPVLKQAGALWL

SEQ ID NO: 351 - DNA, 312 bp, Oryza sativa
atggacagggtgaacaggctggcggcgcagcgggcggtggtgatcttcagcatgagctcgtgctgcatgtgccacaccgtgacgcgcctcttctgcgagctcggggtgaacccgacggtggtggagctggacgaggacccgagggggaaggagatggagaaggcgctggcgaggctcctcggccgcagccccgccgtgccggcggtgttcatcggcgggaggctcgtcggctccaccgacaaggtcatgtcgctgcacctcagcggcaaccttgtcccgctgcttcgcaatgcgggtgccctctgggtgtag

SEQ ID NO: 352 - protein, 103 aa, Oryza sativa
MDRVNRLAAQRAVVIFSMSSCCMCHTVTRLFCELGVNPTVVELDEDPRGKEMEKALARLLGRSPAVPAVFIGGRLVGSTDKVMSLHLSGNLVPLLRNAGALWV

SEQ ID NO: 353 - DNA, 444 bp, Oryza sativa
atgtaccaggcgatcccgtacagcagcacccggccgtggctcaggccggagccggcggcgagcgtggtcgacgtcgtgaaggtggagacgacgacggccgtcgcgggtcggggcggtgaggcggaggtcgtgggggaggaggaggcggcggaggtgcggagggcggtggcggagagcccggtgctggtggtggggaggcgcgggtgctgcctcatccacgtggtgaagcggctgctgcaggggctcggggtcaacccggccgtgcacgaggtcgccggcgaggccgcgctcaagggggttgtgccggccggtggggaggccgcggcgctccccgccgtgttcgtcggggggaagctcctcggcgggctcgaccgcctcatggccgtccacatctccggcgagctcgtgcccatcctcaagaaggccggtgccctctggctttaa

SEQ ID NO: 354 - protein, 147 aa, Oryza sativa
MYQAIPYSSTRPWLRPEPAASVVDVVKVETTTAVAGRGGEAEVVGEEEAAEVRRAVAESPVLVVGRRGCCLIHVVKRLLQGLGVNPAVHEVAGEAALKGVVPAGGEAAALPAVFVGGKLLGGLDRLMAVHISGELVPILKKAGALWL

SEQ ID NO: 355 - DNA, 378 bp, Oryza sativa
atggcggagagggtggcgcggctgtcgtcgcagagggcggtggtgatcttcggggcgagcaactgcttcatgtgccacgtggtgaagacgctcttctcggagctcggggtgagctgggcggtgcacgaggtggacaaggaccccaacggcaaggacgtcgagagggcgctcgccggaatggtcggccggacgccgccggtgccggccgtcttcatcggcggcaagctcgtcgggcccaccgaccaggtcatgtcgctccacctcgccggcaagctcgtcccgctcctccgcgaagccggcgccctctggctcagagatacgaagtactcctatatactaccagctaatcaattaattaactatcgatcaattaattaa

SEQ ID NO: 356 - protein, 125 aa, Oryza sativa
MAERVARLSSQRAVVIFGASNCFMCHVVKTLFSELGVSWAVHEVDKDPNGKDVERALAGMVGRTPPVPAVFIGGKLVGPTDQVMSLHLAGKLVPLLREAGALWLRDTKYSYILPANQLINYRSIN

SEQ ID NO: 357 - DNA, 408 bp, Oryza sativa
atgcagtacggagcggcggccgagcaggcgtggtacatgccggcggcggcgccggcaccgatggtggagagcgcggtggcgcgggtggagcggctggcgtcggagagcgcggtggtggtgttcagcgtgagcagctgctgcatgtgccacgccgtgaagcgcctcttctgcggcatgggggtgcacccgacggtgcacgagctggacctcgacccgcgcggccgcgagctggagcgcgccctggcgcgcctcgtcgggtacggcggccccgccgccgcgtcgccgcccgtcgtccccgtcgtcttcatcggcggcaagctcgtcggcgccatggaccgcgtcatggccgcgcacatcaacggctccctcgtccccctcctcaaggaggccggcgcgctctggctctag

SEQ ID NO: 358 - protein, 135 aa, Oryza sativa
MQYGAAAEQAWYMPAAAPAPMVESAVARVERLASESAVVVFSVSSCCMCHAVKRLFCGMGVHPTVHELDLDPRGRELERALARLVGYGGPAAASPPVVPVVFIGGKLVGAMDRVMAAHINGSLVPLLKEAGALWL

SEQ ID NO: 359 - DNA, 408 bp, Oryza sativa
atgcagtacggcgcggcggcggcggagcaggcgtggtacatgccggcggcggcgatggtggtggcagcggcggcggagacggcggcggagcgggtggagaggctggcgtcggagagcgcggtggtggtgttcagcgtgagcagctgctgcatgtgccacgccgtgaagcgcctcttctgcggcatgggcgtgcacccggcggtgcacgagctggacctcgacccgcgcggccgcgacctggagcgcgccctggcgcgcctcgtcggcgccggcggcgccgccgctgccgccgtgcccgtcgtgttcatcggcggcaagctggtcggcgccatggaccgcgtcatggccgcgcacatcaacggctccctcgtgccgctgctcaaggaggccggcgcgctctggctttag

SEQ ID NO: 360 - protein, 135 aa, Oryza sativa
MQYGAAAAEQAWYMPAAAMVVAAAAETAAERVERLASESAVVVFSVSSCCMCHAVKRLFCGMGVHPAVHELDLDPRGRDLERALARLVGAGGAAAAAVPVVFIGGKLVGAMDRVMAAHINGSLVPLLKEAGALWL

SEQ ID NO: 361 - DNA, 345 bp, Oryza sativa
atggacagggtgacaaggctggcgtcgcagaaggcggtggtggtgttcagcaagagctcgtgcggcatgtcccacgccgtgacgcgcctgctccgggagctcggcgtcgacgcccgcgtggtggagctcgacgaggagcccgccggcgccgacatggagaacgcgctcgccgggatgttgctcgccggcaccgccgccaacggcggtggccgcggccgcggcgtcgtggtgccgacagtgttcatcggcggcaggctcgtcggctccaccgaccgggtcatgtcgctccacgtcgccggcggccttgtcccgctcctccgcgacgccggcgcgctctgggtgtag

SEQ ID NO: 362 - protein, 114 aa, Oryza sativa
MDRVTRLASQKAVVVFSKSSCGMSHAVTRLLRELGVDARVVELDEEPAGADMENALAGMLLAGTAANGGGRGRGVVVPTVFIGGRLVGSTDRVMSLHVAGGLVPLLRDAGALWV

SEQ ID NO: 363 - DNA, 417 bp, Oryza sativa
atgcaaggaggaggcggcgtgagctgcgcggtggccggggacgcgccgtcgtcgacgagggggggaggcggcggagggatgctggggctgacgctgttcgacccgccgggaggggagcagccggcggagaggatcgggaggctggtgcgggagagccccgtggtgatcttcgcgaggagggggtgctgcatgtgccacgtcatgcgccgcctcctggccgccgtgggggcgcacgccaccgtcatcgagctcgacgaggccgccgaggaggccgcggcgtccgcggccgccgccgccgccgtcccggcgctcttcgtcggcggcgccccagtcggcggcctcgacggcctcatgggcctccacctcagcggccgcctcgtcccccgcctcagggaggtcggcgccctctgcggctag

SEQ ID NO: 364 - protein, 138 aa, Oryza sativa
MQGGGGVSCAVAGDAPSSTRGGGGGGMLGLTLFDPPGGEQPAERIGRLVRESPVVIFARRGCCMCHVMRRLLAAVGAHATVIELDEAAEEAAASAAAAAAVPALFVGGAPVGGLDGLMGLHLSGRLVPRLREVGALCG

SEQ ID NO: 365 - DNA, 399 bp, Oryza sativa
atgtaccaggcgatcccgtacaacgcgaaccgggcttggccggcggcgagccggccggcgacggcggcggcggcgccgccgccgccgccgccgcgtggcgaggaggaggaggtgaggagggcggtggcggagtgcccggtggtggtggtgggtcggagcgggtgctgcctgagccacgtcgtgaagcggctgctgcaggggctcggggtcaacccggcggtgcacgaggtcgccggcgaggccgagctcgccggggtggtcgccggcggcggcggcgtcgcgctgccggcggtgttcgtcggcgggaggctcctcggcgggctcgaccggctcatggccgtgcacatctccggcgagctcgtgcccattctgaaggaggccggtgcactctggctctga

SEQ ID NO: 366 - protein, 132 aa, Oryza sativa
MYQAIPYNANRAWPAASRPATAAAAPPPPPPRGEEEEVRRAVAECPVVVVGRSGCCLSHVVKRLLQGLGVNPAVHEVAGEAELAGVVAGGGGVALPAVFVGGRLLGGLDRLMAVHISGELVPILKEAGALWL

SEQ ID NO: 367 - DNA, 579 bp, Oryza sativa
atgtcagagcgtgtgttcgccgagctcgcgaccatccactaccaaaaaagccttccatgtcgccactcctttgacccccctcgcaccacaccaattctccatctatatatcatccaccttcttcttcctcctctcattgccattgtgtgtttgtgttacattgcaatcgtgccatttgaagaagaggaggagaggatgaggatgcaggtggtggagacggcggcggtggaggaggaggaggcggcggcggcgatgatgtcggtgtacgagagggtggcgaggatggcgagcgggaacgcggtggtggtgttcagcgcgagcgggtgctgcatgtgccacgtcgtcaagcgcctcctcctcggcctcggcgtcggccccgccgtctacgagctcgaccagctcgccgccgccgccgacatccaggccgcgctgtcgcagctcctcccgccgggccagccgccggtgcccgtcgtgttcgtcggcggcaggctcctcggcggcgtcgagaaggtgatggcgtgccacatcaatggcaccctcgtccccctcctcaagcaggccggcgccctctggctctga

SEQ ID NO: 368 - protein, 192 aa, Oryza sativa
MSERVFAELATIHYQKSLPCRHSFDPPRTTPILHLYIIHLLLPPLIAIVCLCYIAIVPFEEEEERMRMQVVETAAVEEEEAAAAMMSVYERVARMASGNAVVVFSASGCCMCHVVKRLLLGLGVGPAVYELDQLAAAADIQAALSQLLPPGQPPVPVVFVGGRLLGGVEKVMACHINGTLVPLLKQAGALWL

SEQ ID NO: 369 - DNA, 327 bp, Oryza sativa
atggagagggtggcgaagctggcgtcggagagggcggtggtggtgttcacggcgagcaactgcggcatgtgccacgccgtgacgagcctcctcgtcggcgagctgggcgtcaacgccgccgtgcacgagctcgacaaggacccccgcggcagggacatggagagggagctcgccaggaggctcaacggcggcggcggcggcggccgcgccctgccggcggtgttcgtcggaggcaacctcgtcggcggcgccaaccgggtcatgtcgctccacctcgccggcgagctcgtccccatgctcaagaacgccggcgcgctctggctctag

SEQ ID NO: 370 - protein, 108 aa, Oryza sativa
MERVAKLASERAVVVFTASNCGMCHAVTSLLVGELGVNAAVHELDKDPRGRDMERELARRLNGGGGGGRALPAVFVGGNLVGGANRVMSLHLAGELVPMLKNAGALWL

SEQ ID NO: 371 - DNA, 330 bp, Oryza sativa
atggcggagatggtggcgaggctggcgtcggagagggcggtggtggtgttcaccaagagcggctgctgcatgtgcaccgcggtgacgacgctgctcggcgagctcgccgtcagcgccgccgtgcacgagctcgacagggacccgctcgggaaggagatggagaaggagctcgccaggaggctctacggctccagcggccgcggcgggcccgccgtgccggcggtgttcatcggcgggagcctcgtcggcggcaccagcaaggtgatggccatgcacctcaagggcgagctcgtgcccttgctcaagagcgccggtgcactgtggctttga

SEQ ID NO: 372 - protein, 109 aa, Oryza sativa
MAEMVARLASERAVVVFTKSGCCMCTAVTTLLGELAVSAAVHELDRDPLGKEMEKELARRLYGSSGRGGPAVPAVFIGGSLVGGTSKVMAMHLKGELVPLLKSAGALWL

SEQ ID NO: 373 - DNA, 330 bp, Oryza sativa
atggcggagatggtggcgaggctggcgtcggagagggcggtggtggtgttcaccaagagcggctgctgcatgtgcaccgcggtgacgacgctgctcggcgagctcgccgtcagcgccgccgtgcacgagctcgacagggagccgctcgggaaggagatggagagggagctcgccaggaggctctacggctccggcggccgcggcgggcccgccgtgccggcggtgttcatcggcgggagcctcgtcggcggcaccagcaaggtgatgaccgtgcacctcaagggggagctcgtgccaatgctcaagagtgccggtgcactgtggctttga

SEQ ID NO: 374 - protein, 109 aa, Oryza sativa
MAEMVARLASERAVVVFTKSGCCMCTAVTTLLGELAVSAAVHELDREPLGKEMERELARRLYGSGGRGGPAVPAVFIGGSLVGGTSKVMTVHLKGELVPMLKSAGALWL

SEQ ID NO: 375 - DNA, 330 bp, Oryza sativa
atggcggagatggtggcgaggctggcgtcggagagggcggtggtggtgttcaccaagagcggctgctgcatgtgcaccgcggtgacgacgctgctcggcgagctcgccgtcagcgccgccgtgcacgagctcgacagggagccgctcgggaaggagatggagagggagctcgccaggaggctctacggctccggcggccgcggcgggcccgccgtgccggcggtgttcatcggcgggagcctcgtcggcagcaccagcaaggtgatggccatgcatctcaagggggagctcgtgccaatgctcaagaacgccggtgcactgtggctttaa

SEQ ID NO: 376 - protein, 109 aa, Oryza sativa
MAEMVARLASERAVVVFTKSGCCMCTAVTTLLGELAVSAAVHELDREPLGKEMERELARRLYGSGGRGGPAVPAVFIGGSLVGSTSKVMAMHLKGELVPMLKNAGALWL

SEQ ID NO: 377 - DNA, 312 bp, Oryza sativa
atggaccgtgtgatgaagctagcatccgagcgtgcggtggtgatcttcaccttgagctcatgctgcatgtgccacaccgtgacacgcctcttctgtgatctcggtgtcaacgcgctagtgcatgagctggaccaagaccctaggggcaaggagatggagagggcactcctcaagctgctcggaagggggccgcctgtgccggtggtgttcattggtgggaagctcgttgggggaaccaacaagatcatgtccctccaccttggaggtgagctgatccccatgctcaagaatgcgggagccctctggctgtag

SEQ ID NO: 378 - protein, 103 aa, Oryza sativa
MDRVMKLASERAVVIFTLSSCCMCHTVTRLFCDLGVNALVHELDQDPRGKEMERALLKLLGRGPPVPVVFIGGKLVGGTNKIMSLHLGGELIPMLKNAGALWL

SEQ ID NO: 379 - DNA, 315 bp, Oryza sativa
atggagagggtggcgaagctgtcgacggagaaggcggtggtgatcttcacggcgagcaactgcccgatgtgccacacggtggtgagcctcttctccgacctcggcgtcggcgccgccgtccacgagctcgaccgcgacccgctccacggccgggacatggagcgcgacctcgcccgccgcctcggccgctccccgcccgtccccgccgtcttcatcgccggcaagctcgtcggctccaccgaccgcgtcatgtcgctccacctcgccggcaagctcgtccccatgctcaaggccgccggcgccatttggctctag

SEQ ID NO: 380 - protein, 104 aa, Oryza sativa
MERVAKLSTEKAVVIFTASNCPMCHTVVSLFSDLGVGAAVHELDRDPLHGRDMERDLARRLGRSPPVPAVFIAGKLVGSTDRVMSLHLAGKLVPMLKAAGAIWL

SEQ ID NO: 381 - DNA, 892 bp, Picea abies
cgatacagtagttctgccgattccgacgtcacagtgggcgacgacgcagcgtgcaagcttcggcctgcaaagcagctagagctcatgggctgcaatctgttaatgaaaactgttgtcgagtctcccgtggacaagattcgcaggcttactacggagaacgccgtgctggtattcagcatgacttcttgctgcatgtgccatgtcgtgaagcggctcttgtgcagcctgggcgtacatcctactgtttgtgaattggacgaggaagaagaaggagtggagatggagaagatactgcgggcgttggtgggagcacagaaatcgtcggtgcccgctgtgttcatcggagggaatctcataggcggcctggaccgggtcatggccatgcacatcgaaggcgatttggtaccgaaattgaaagaagccaaagctctatggctttgatgagctgttagggtaaggtaatcatgttatgtccatagttaaaaatccttttctagtgaggcggtgtttctggagcagattttctttggttgaagttcgagtccggctgaatcttgttggattaccagctgttattggtcaatcgtgttgtcgtcttccatctggacattctaaaaaagagggattttgcaagattttccttgttgattgcctctatgtcgcctcctcctcctccagagacatctttatatgtaattttctaaccgatggcgtcgggtttttcgactttgcgatcttttattttgcagctttcaatccagggtttgtatattcagatgatcatcatgaagaaactacgctgtaggtttctgaattctgtaaagctagaattctactttgattgtcaaatcaaagcccgtgtcgcagcttttaatatattgcatttttaacc

SEQ ID NO: 382 - protein, 118 aa, Picea abies
MGCNLLMKTVVESPVDKIRRLTTENAVLVFSMTSCCMCHVVKRLLCSLGVHPTVCELDEEEEGVEMEKILRALVGAQKSSVPAVFIGGNLIGGLDRVMAMHIEGDLVPKLKEAKALWL

SEQ ID NO: 383 - DNA, 761 bp, Picea abies
tctttttttttccatgcgtaacgacgctctgccttgtcttatagtcactcgttcttcctgagcgagcttccgcaagcttccgcgagcttcagatttcctttccttacattccaagctcgaatctccgctaccctacttgtgtttttcttccttcctgcatatatacaatatgcagtatcacgctcgggcctacaatcagttcggcgagggttcgtatggtgagcgaacgtctctacatctggcaagtcagtcgggttcgtcctccagcagcctcatgtctccggtggaaagaatccatcagttggcctccgagagcgccgtggtggtcttcagcataagctcctgctgcatgtgccatgttgtcaagaggctcttctgtggcctgggagtcaatcccaccgtctacgaactggacgaggagcacggcggcaaggagatcgagaaggccctgctcaggctgctgggcggaagcccagccgttcccgccgtctttgtaggcggaaagcttgtgggaggactggaccgcgtaatggcttctcatataaacggctctctcgtgcctctcttgaaggaagctggagcactgtggctctgagccctttttcataatctgcgggcattttcattaattccttgtcgtgtctacttttttgtatatcgtcaatttcgtctctggtcatgctcagggtctccttttttgtaatatcggagattcactatatatatataaaatataagtaacgctgtagatttttcttggc

SEQ ID NO: 384 - protein, 141 aa, Picea abies
MQYHARAYNQFGEGSYGERTSLHLASQSGSSSSSLMSPVERIHQLASESAVVVFSISSCCMCHVVKRLFCGLGVNPTVYELDEEHGGKEIEKALLRLLGGSPAVPAVFVGGKLVGGLDRVMASHINGSLVPLLKEAGALWL

SEQ ID NO: 385 - DNA, 788 bp, Picea abies
tcgtccctcatcggtgctggggaaagggggaacgttgtcttcatcgttgtcatcatgatgcagggtttacaatacagggccggcggatcgtctgctccaggcaccgtggcctccgcggcctgcaggaggcctgcagccgccacgctgcatctggggcagagctccgtggaggacgacgaagagacgatgacgaggacggccatgatgagtatgagccctttggaaagagtgcagcgcctggcctccgataacgcggtgctcgtattcagcgtcacttcctgctgcatgtgccacgttgttaagcgcctcttctgcggccttggggttaatccggcggtgttcgagctcgacgaggaaggggaaggtactggaagctcagatatggagaaggttcttgtcagattggtcggcaaaaagcccgcggtgcctgcggttttcatcggcggagagctcgtcggcggcctcgatcgcctcatggccgcgcacatcagcggcgagctcgtgcccaaattgaagcaagccggagctctgtggctttaaagaatcagaattcgcccgcctcatttttgctttcattgtgcgacagatcaatgaattaatcaccatctctcctgcatagaaggcgagtatataattaattaatatttgttgggggtcttgacaattagaattctgttcttcttgttctgtaaatattctctcggctttacagagataattataacatataaatagtgaaaatctctcacgccgatacaattttgaatagattataaatgctattctcc

SEQ ID NO: 386 - protein, 161 aa, Picea abies
MMQGLQYRAGGSSAPGTVASAACRRPAAATLHLGQSSVEDDEETMTRTAMMSMSPLERVQRLASDNAVLVFSVTSCCMCHVVKRLFCGLGVNPAVFELDEEGEGTGSSDMEKVLVRLVGKKPAVPAVFIGGELVGGLDRLMAAHISGELVPKLKQAGALWL

SEQ ID NO: 387 - DNA, 727 bp, Picea abies
aatcataaataacctacaggagagcatatttccaatcatatacattgaggaattgcaggcccagaggggattggattgggttcattgatgcagggcctccagtacaggccgggcgcggccggttccgcgccatgcaagaagaaaagcgcagcgcttcaattgaataagccgttacagaggttggagtctccgctggaggcggtgcaaaggctcgcctcagaaaacgccgtgctcgttttcagcatgagttcctgctgcatgtgccatgtggttaagcggctcctgtgcagcctcggggtcaacccggccgtgtgcgagctggacgaggaagatcaagaggaggaggaggagacccacggaaaattgcgcgcccacagggatgatatagagaaggcgctcttcagattagtcgggcagaggccgcccgtgccggcggtttttataggcggacagctcgtgggtggcctggaccagctcatggccgcacatatcagcggagagcttgtgcctagattgaaagaggccggcgctctctggctttaataaagctcaattgcttcgtttgtctggggaatttttaggtttatttgtttggggttcgagggtttgatcagttcttcttgtatattgttctttgtaatagctttgatttcaaaagcagtgggtagagtcgagttcatttattgttctcgttcttttcaatatagaatgagatttttagggatcg

SEQ ID NO: 388 - protein, 151 aa, Picea abies
MQGLQYRPGAAGSAPCKKKSAALQLNKPLQRLESPLEAVQRLASENAVLVFSMSSCCMCHVVKRLLCSLGVNPAVCELDEEDQEEEEETHGKLRAHRDDIEKALFRLVGQRPPVPAVFIGGQLVGGLDQLMAAHISGELVPRLKEAGALWL

SEQ ID NO: 389 - DNA, 309 bp, Physcomitrella patens
atgcaggagatagagaagctggtgcaggaaaatgctgtggttgtgttcagccagagcgggtgctgcatgtgtcatgtggtaaagcgtctcttctgcagtctgggagtggggccaactgtgcacgaactcgatgaacggaaggaaggtggcgacatggagaaggcattgctgcgcctcaacaacaaagttgcgcttcctaccgtgtttgtgggcggcaaactggtggggggcgtcgatgctgtcatggctgcccacgtgagtgggaaccttgtcccccgcttgaaggaagccggagctctttggctgtag

SEQ ID NO: 390 - protein, 102 aa, Physcomitrella patens
MQEIEKLVQENAVVVFSQSGCCMCHVVKRLFCSLGVGPTVHELDERKEGGDMEKALLRLNNKVALPTVFVGGKLVGGVDAVMAAHVSGNLVPRLKEAGALWL

SEQ ID NO: 391 - DNA, 705 bp, Pinus taeda
catttcttatcttcttcttcttcgtctcgttttcttcttcttctttttttcttgtttcgtggaagatgcagtaccatcatcccgcagcagaggcgtggggaggctcctacggcggcgacgacatgtcttcggggcggaggacgtccctacacatgccggcggggggccaatctggctcgatatcgccgctggagcgggtggagagacttgcttctgagaacgcagtggtggtcttcagcatgagctcatgttgtatgtgccatgtgatcaagcgcctcttctgcagcctcggcgtcaatccgactgtgtacgaactggacgaggagcatcacggcaaagacatggagaaggctctgcagagactggtaggcggtggccaggccgtccctgccgtgttcgtcggtggaaaattcttgggaggaatggaccgcgtaatggcctcccacatcaatggcacgctcgtccctcttttgaaggaagcgggagccttgtggctctgaatcattccggccgcagatacttcttgttttctcggttttccgctgatcttgtggctcaaaactgcgaccgagcctctgtaaccgtggttccttcattttgcattatgtccagctttagaattttctttgcggaattattagttagcgtgttgtcgaagctctgataaatgtgtatacatgctttatctttctttctcatggagct

SEQ ID NO: 392 - protein, 144 aa, Pinus taeda
MQYHHPAAEAWGGSYGGDDMSSGRRTSLHMPAGGQSGSISPLERVERLASENAVVVFSMSSCCMCHVIKRLFCSLGVNPTVYELDEEHHGKDMEKALQRLVGGGQAVPAVFVGGKFLGGMDRVMASHINGTLVPLLKEAGALWL

SEQ ID NO: 393 - DNA, 707 bp, Pinus taeda
gcacgagcgaggtttcataatccctcaccattgctgggaagcaggaatcgttgccttcatctttgtcatcatgatgcagggtttgcaatacagggccggcggatcggcggctccaggcaccgtggcctccgcggcctgcaggaggcccgccgctgctacgctgcagttggggcggagctccgtggacgacgccgaggaggaaacgatgaccacgaagacagccacgatgagcctgagccctttggaaagagtgcagcgcctcgcctccgataacgcggtgctcgtattcagcgtcacttcctgctgcatgtgccacgtggtcaagcgcctcttctgcggcctcggggttaatccggccgtcttcgagctcgacgaggaaggggaaggcgccgggaactctgatatggagaaggttctggtgagattggtcggcaaaaagcctgctgtgccggcggttttcatcggcggagagctcgtcggcggcctcgatcgcctcatggccgcgcacatcagcggcgagctcgtgcccaaattgaagcaagccggggctctgtggctttagagaatcagaattttcgccggcctcaatttttgctgctttctttggcgatagatcaattaattaatcaccatttctcgtggcatagaaggcgagtatataattaatgaatatttgttgggggtctcaacaattagaatgaaaaaaa

SEQ ID NO: 394 - protein, 163 aa, Pinus taeda
MMQGLQYRAGGSAAPGTVASAACRRPAAATLQLGRSSVDDAEEETMTTKTATMSLSPLERVQRLASDNAVLVFSVTSCCMCHVVKRLFCGLGVNPAVFELDEEGEGAGNSDMEKVLVRLVGKKPAVPAVFIGGELVGGLDRLMAAHISGELVPKLKQAGALWL

SEQ ID NO: 395 - DNA, 896 bp, Pinus taeda
cggccggggacacaccacattctcattccctctttatttttcatgcgtaacgacgctctcctctcccttgtcttctagtctcagaccttcgttcttgtttcagcttgcgcgcgccccccttagcagtccgcccccccgtccccccactatacaatatgcagtatcacgctcgggcctataatcagttcggcgaaggctcctatggtgagcgaacgtctctgcatctggcgagccagtcgggttcgtccaccagcagcttgatgtcgcctgtggaaagaatccatcagctggccgccgagagcgccgtggtggttttcagtataagttcttgctgcatgtgccatgttgtcaagaggctcttctgtggtctgggagtcaatcctactgtctacgaactcgacgaggagcacggcggtaaggagattgagaaagccctgctcaggttgctgggcgggagcccatcggttcctgctgtttttgtcggcggaaagcttgtgggaggactggaccgcgtaatggcttctcatataaatggctcgctcgttcctctcttgaaggaagccggagcactgtggctctgagccccctttttccttaaggggctgtcattttcattaattccatgtcgcgtcgcgtctcctttttgtatatcgttaattctgtctcgggtcaagtcgggtctcctttttgtaatatcggagattcaatctctctatatataaaaatatatatataaactatgctgtagatttttgttggcaactttagagcttatgggttttcattttctgcacttgcaattttgtaaagaatcttgcgtttggggtaagaagttgcctctgttcgggaaatttcttcatgcccaacgagtaccgaatacaagtgttctgcgcact

SEQ ID NO: 396 - protein, 141 aa, Pinus taeda
MQYHARAYNQFGEGSYGERTSLHLASQSGSSTSSLMSPVERIHQLAAESAVVVFSISSCCMCHVVKRLFCGLGVNPTVYELDEEHGGKEIEKALLRLLGGSPSVPAVFVGGKLVGGLDRVMASHINGSLVPLLKEAGALWL

SEQ ID NO: 397 - DNA, 427 bp, Populus trichocarpa
tttgtacaaaaaagcaggcttaaacaatgtatcaaaccgagtcatgggggtcttacatgccagcaagaaccaaccttggtgacccattggaacgcataggaaggctagcctcagagaatgcagtggtgatctttagcataagctcatgttgcatgtgtcatgctattaagaggctcttttgtggcatgggagtgaacccaacagtgtacgagctggatgaagacccaagaggtaaagaaatggagaaggctctcatgaggcttctcggtagctcctctgctgttcctgttgttttcatcggtggcaagcttgtgggtgccatggatagagtcatggcttctcatattaacggtactcttgtccctcttctcaaggaagccggtgctctttggctttaattgatgctacccagctttcttgtacaaag

SEQ ID NO: 398 - protein, 123 aa, Populus trichocarpa
MYQTESWGSYMPARTNLGDPLERIGRLASENAVVIFSISSCCMCHAIKRLFCGMGVNPTVYELDEDPRGKEMEKALMRLLGSSSAVPVVFIGGKLVGAMDRVMASHINGTLVPLLKEAGALWL

SEQ ID NO: 399 - DNA, 416 bp, Populus trichocarpa
ggcattagtttcttgctcgttgatttggaaatggagagggtgacaaatttggcatccgagagaccagttgtgatcttcagcaagagcacttgctgtatgtgccacaccatcaagactctcttcaatgagtttggggtgaacgtggctgtccatgagctcgatgagatgcctagaggaagggaaattgagcaagcactctcaaggtttggatgcccaacattgcctgccgtgttcattggtggtgaacttgtgggtggagccaatgaggtgatgagccttcaccttaatcgttccttaatcccaatgcttaaacgtgctggcgcgttatgggtttgatccatcgatgaactagcttagctacgcatcaggcactaaccaaataaattatgaaacatgttctggttgctcataacata

SEQ ID NO: 400 - protein, 101 aa, Populus trichocarpa
MERVTNLASERPVVIFSKSTCCMCHTIKTLFNEFGVNVAVHELDEMPRGREIEQALSRFGCPTLPAVFIGGELVGGANEVMSLHLNRSLIPMLKRAGALWV

SEQ ID NO: 401 - DNA, 491 bp, Populus trichocarpa
ggttagtgtgtaaattacaaggaaattaagatgcaatatcatcaggctgagtcatggggttaccatgtgccaacgaggacctgcatggcatcagacccattggagaaggttgcgaggctggcatcggagagtgctgttgtggtatttagtatcagcagctgttgcatgtgtcatgccgtgaagagactcttttgtgggatgggtgtgaacccaactgtttatgagctcgaccatgacccaagaggggaagagattgagaaggcattaatgaggctgcttgggaattcaacttctgtgcctgttgtattcattggagggaagttaattggtgccatggaacgagtcatggcttcccatatcagtggaactctcgtgcccctcctcaaggaagctggagcactctggctttgattttgatcatcgatcaacaaagttaacatcacaaactgggtccaaaacatggaaattatcattacaaggaaaaaagagga

SEQ ID NO: 402 - protein, 126 aa, Populus trichocarpa
MQYHQAESWGYHVPTRTCMASDPLEKVARLASESAVVVFSISSCCMCHAVKRLFCGMGVNPTVYELDHDPRGEEIEKALMRLLGNSTSVPVVFIGGKLIGAMERVMASHISGTLVPLLKEAGALWL

SEQ ID NO: 403 - DNA, 419 bp, Populus trichocarpa
accaactctttgaacgctctgtgacattaaatggatgtgttaaatgtgatgatacaagaaaagccagtggtgattttcagcaagagttcttgctgcatgagccactccatcaagtcgcttatacgtggatttggagcaaatccaacagtttatgagctcgacaggattccaaatggacaacaaattgaaagggcactagtgcagctggggtttggacagagcgtacctgctgtgttcataggccaacggctggttggtaatgaaaggcaggtgatgagcctccacgtccagaaccagctggtcccattgcttatacaagccggtgctatttggatttagaataagtagtttctcactgacataggacatggatcattgtcatgcatctcaaaatttaggtaagttgctgctgctagcta

SEQ ID NO: 404 - protein, 102 aa, Populus trichocarpa
MDVLNVMIQEKPVVIFSKSSCCMSHSIKSLIRGFGANPTVYELDRIPNGQQIERALVQLGFGQSVPAVFIGQRLVGNERQVMSLHVQNQLVPLLIQAGAIWI

SEQ ID NO: 405 - DNA, 428 bp, Populus trichocarpa
ttcaaactctttcaatcgatcttcgtgaaaatggacgtggtgaatgtaatgattcaaggaaagcctgtcgtgattttcaggaagagttcttgctgcatgagccactccgtcgagtcacttatacgtggatttggagcaaatctaactatttatgagctcgacagaataacaaatggacaacaaattgaaagggcactagtgcaactggggtttcgacagagcttacccgctgtgttcataggccagcagctggttggcaatgaaaggcaggtgatgagcctccacgtccagaaccagctggtaccattgcttatacaagcaggtgctatatggatgtggaataagtagtttctcactgacatatatagggcacggatcaatatcatgctgctgaacattttgataagctactacttttgttttttgtt

SEQ ID NO: 406 - protein, 105 aa, Populus trichocarpa
MDVVNVMIQGKPVVIFRKSSCCMSHSVESLIRGFGANLTIYELDRITNGQQIERALVQLGFRQSLPAVFIGQQLVGNERQVMSLHVQNQLVPLLIQAGAIWMWNK

SEQ ID NO: 407 - DNA, 422 bp, Populus trichocarpa
tcactttgccaacttctattcttcgtagacatggatatggtgaacaggttggttgctgacaggccagtggtggtctttagcaggagcacttgttgcatgagccactccattaagacacttatatctagctttggggcaaatcctacagtttatgagctggatcaaataccaaatgggaagcaaattgaaaaggcattagtgcagcagctaggatgtcagccaagtgtaccagctgttttcataggccaagagtttgttggtggtgataagcaagtcatgagcttacaagtcaggaatgagctagccccattgctcaggaaggcgggagctatatggatttgatcaaatggcagtcattagtcaaatttgcacttgctttaattcagtactgtttgctagtgctcgaattttgatgggcttga

SEQ ID NO: 408 - protein, 103 aa, Populus trichocarpa
MDMVNRLVADRPVVVFSRSTCCMSHSIKTLISSFGANPTVYELDQIPNGKQIEKALVQQLGCQPSVPAVFIGQEFVGGDKQVMSLQVRNELAPLLRKAGAIWI

SEQ ID NO: 409 - DNA, 419 bp, Populus trichocarpa
tcttatcaacaccaggaaaaggttttagaaatggataaggttagagatttggcatctaggaacgctgcagtgatcttcaccaagagctcatgctgcatgtgccacagcatcaagacacttttttatgaactaggtgcaagccccgcaattcatgagctagaccgtgaagccaatggtaaggaaatggagtgggctttacgtgggctagggtgcaaccccaccgtcccagctgtcttcataggtggaaaatgggtaggatcagccaaagatgtgctatccctgcacctggatgggtctttgaaacaaatgctcatggaagccaaggcaatctggttctagctatagtacctctcaaataagccacacagtactttagtttaacgctgtatgatatggaaatagctcttatgctatgttga

SEQ ID NO: 410 - protein, 102 aa, Populus trichocarpa
MDKVRDLASRNAAVIFTKSSCCMCHSIKTLFYELGASPAIHELDREANGKEMEWALRGLGCNPTVPAVFIGGKWVGSAKDVLSLHLDGSLKQMLMEAKAIWF

SEQ ID NO: 411 - DNA, 606 bp, Triticum aestivum
gctagctcaagtgaagtgagctgatcgattgagatacagaggagagatagctaggcaattcagcaatggcggagagggtgaccgcgattgcgtcgcagggcacagtggtgatctttggggcgagctgctgctgcatgtcccacaccatgacgaggctctttgcggagctgggtgttttttctacggtgcacgagctggacaaggacccccagagggaggacctggagagggcgctcgctggcatggtgggccagagcccggcggtgccggcagtattcatccgtggcgcgcttgtcggtggcaccaggcaggtcatgcagctgcatctcggcggccatctcgtgccgctgctccgccaagctggtgccctgtggtcctgaggcattattaataataatggctgatgcgtacttgcatagtaatctacgtgtgatcataacctcaaaagctgtagctagtgatcgacaatacagtgtgtgccgctacttaatgttgaacaatgcttctcgccggacattgaacccaactctcaggcttcttgccagatagatatctgtctcactatgatgatctggcaagaaatataggacatgattgttaggaatc

SEQ ID NO: 412 - protein, 104 aa, Triticum aestivum
MAERVTAIASQGTVVIFGASCCCMSHTMTRLFAELGVFSTVHELDKDPQREDLERALAGMVGQSPAVPAVFIRGALVGGTRQVMQLHLGGHLVPLLRQAGALWS

SEQ ID NO: 413 - DNA, 448 bp, Triticum aestivum
ttcggcacgaggcacaagctcactatagcagctacaagctactcaggagctgaccaacacccttccaagagcctccttccatcactttgccgccgagctcgaccactgcgaggttacatcacaagatggaccgtgtgatgaagctagcatctgagcgtgccgtggtggtgttcaccctgagtccctgctgcatgtgccacacagtggagcgtctgtttcgtgaccagcttggggtcaatgcgctggtgcacgagctcgaccaggaccctaggggcaaggagatggagagagccctcctcaagatgctcggcagggggccgtcggtgccggtcgtcttcatcggcgggaagcttgttggtggcaccaacaggatcatgtctctacacctcggtggcgagctagtccccatgctcaagagtgccggtgctctctggctatagagagctag

SEQ ID NO: 414 - protein, 104 aa, Triticum aestivum
MDRVMKLASERAVVVFTLSPCCMCHTVERLFRDQLGVNALVHELDQDPRGKEMERALLKMLGRGPSVPVVFIGGKLVGGTNRIMSLHLGGELVPMLKSAGALWL

SEQ ID NO: 415 - DNA, 714 bp, Triticum aestivum
cgtacacgtagcactgaaagacttctacatcaacagcattgttcttttgagttggtggtgaagatcaaggtcgggtgagcttgatggagcaggtgacgaagctggcggggcagcgggcggtggtgattttcagcatgagctcctgctgcatgtgccacacggtggcgcgactcttccgggatctcggggcgaacccggcggaggtggatctcgacgaggaccctagggggaaggagatggagaaggcgctggcgaggcttctcggtcggaacccggccgtgccggcggtgttcatcggcggcaggctcgtcggatccaccgacaaggtcatgtcccttcacctcagcggcaagcttgtaccgctgcttcgtaacgcaggtgctgtctgggtctagtgcgcgggagatggttttttaggtgcctgatgctagagaataatgtaatacacgtatgcctacgtgttctgttttctctatcaggtgatgatcaaagataaaaaaaggaaaccatgcaggtcgttttattttagcactgcagagggggagtacgattctactccctctgttccgaattactccatttcaacgacgagtaatttggaacggagggagtatgactaaagttgtgactggcatagtggcatgtggaccaatttgatgggggccccatgtcctttatcttaaagtcgagagatttaagtctaaggatctcagt

SEQ ID NO: 416 - protein, 103 aa, Triticum aestivum
MEQVTKLAGQRAVVIFSMSSCCMCHTVARLFRDLGANPAEVDLDEDPRGKEMEKALARLLGRNPAVPAVFIGGRLVGSTDKVMSLHLSGKLVPLLRNAGAVWV

SEQ ID NO: 417 - DNA, 661 bp, Triticum aestivum
ccacgcgtccgggcaaaccactaagtaacactagctttttttctctgagaaaaacactagatttctttccagagaaaaacaccagctttccaactaggtcctttcacaatccatcaagaagatcgacgagaaaatatggagagggtgacgaggctatcgacggagaaggcagtggtgatcttcacgccgagcaacgactgcccaatgagctacacagtgacgaccctcttctctggcctcggcgtttgcgcggccgtgcacgagctggacaaggacccccggggccgtgacatggagcgcgacctcgcccgtcgcctgggccgcacgccggccgtcccggccgtcttcatcggcggcaagctcgtcggttccaccgacagggtcatgtcgctgcaccttggtgggaagctggtgcccatgctcaaggccgccggggcgatctggctctgagcctgagaggctgagagaagcatcaagttaactcccacgtataagtaccgtttataatcaaaattttacactgcatgcatgcgcgcgtagagaggtttggtttgtattttgcctactaaacatcctcacatgagtacgtaatgaagacgggagcaccagaagttttggcctctatcgataagggaatgaccaagctgcgtatctttatgag

SEQ ID NO: 418 - protein, 104 aa, Triticum aestivum
MERVTRLSTEKAVVIFTPSNDCPMSYTVTTLFSGLGVCAAVHELDKDPRGRDMERDLARRLGRTPAVPAVFIGGKLVGSTDRVMSLHLGGKLVPMLKAAGAIWL

SEQ ID NO: 419 - DNA, 453 bp, Vitis vinifera
atgcagttcctcctccttcgggcgtataccatcacattttggacacccagggcactgatattcaccagacactttgatattgcataccttgttggtgtattttgggactacattgttagttgctcagtcatggcggatccgctggagcgagtggcgaggctggcgtcggagaatgcggtggtgatcttcagcctcagctcctgctgcatgtgccatgccgtgaagaggctcttctgcggaatgggcgtgaacccgactgtgtacgagctggaccaggaccccagagggaaggagattgagcgggcgttgatgaggctgctggggaactctccggcggtgcctgtggtgttcatcggggggaagctcgtggggtcaatggacagtgtcatggcttcacatatcaatgggactctggtccctctcctcaaggaagccggagctctctggctctga

SEQ ID NO: 420 - protein, 150 aa, Vitis vinifera
MQFLLLRAYTITFWTPRALIFTRHFDIAYLVGVFWDYIVSCSVMADPLERVARLASENAVVIFSLSSCCMCHAVKRLFCGMGVNPTVYELDQDPRGKEIERALMRLLGNSPAVPVVFIGGKLVGSMDSVMASHINGTLVPLLKEAGALWL

SEQ ID NO: 421 - DNA, 312 bp, Vitis vinifera
atggagagggcagtggcaaggttggcatcagagaggccagtggtgatattcagcaagagctcatgctgcatgtgccacaccatcaagacccttttctcggactttggagttaacccggccgtccacgagctggatgaaatgccgagagggcgtgaaattgagcaggccttggccaggcttgggtgcaaccccacggtgccaacagtgttcattggtggtgaacgggtgggtggaaccaatgagatcatgacccttcacctcaatagatccttaatccccatgctcaagagggctggtgccttatgggtctga

SEQ ID NO: 422 - protein, 103 aa, Vitis vinifera
MERAVARLASERPVVIFSKSSCCMCHTIKTLFSDFGVNPAVHELDEMPRGREIEQALARLGCNPTVPTVFIGGERVGGTNEIMTLHLNRSLIPMLKRAGALWV

SEQ ID NO: 423 - DNA, 309 bp, Vitis vinifera
atggatcgtgttgggaagttggcgtcgcaaaaggcagtggtgatcttcagtaagagttcctgttgcatgagccatgccatcaagagattgttctatgaacaaggggttagtccggcaatccacgagctcgacgaggactccagagggaaagaaatggagtgggctctgatgaggctagggtgcaacccctcagttccggctgtgttcattggggggaaatttgtgggctctgcaaatactgtgatgacccttcatctcaatggctcacttaagaaaatgctgaaagaagccggagctatatggctttag

SEQ ID NO: 424 - protein, 102 aa, Vitis vinifera
MDRVGKLASQKAVVIFSKSSCCMSHAIKRLFYEQGVSPAIHELDEDSRGKEMEWALMRLGCNPSVPAVFIGGKFVGSANTVMTLHLNGSLKKMLKEAGAIWL

SEQ ID NO: 425 - DNA, 321 bp, Vitis vinifera
atggattcggtgatggcattggggactgcaaagccagtggtgatctttaccaagaactcattatgttgcatgagccacagcattaagacactcataagcagctatggggccagtcctacggtctatgagcttgatgaaatgccaaacggagagcaaatggaaaaggccctaccaatactagggtgtccgaatttaccagcggtattcataggtaaaaagctggtgggtggggctcgtgagataatgagcctgcaagtcaggggcgagctctctgatctgctcatagaggcaaaagctatatttttttggaacaaaaaatag

SEQ ID NO: 426 - protein, 106 aa, Vitis vinifera
MDSVMALGTAKPVVIFTKNSLCCMSHSIKTLISSYGASPTVYELDEMPNGEQMEKALPILGCPNLPAVFIGKKLVGGAREIMSLQVRGELSDLLIEAKAIFFWNKK

SEQ ID NO: 427 - DNA, 309 bp, Vitis vinifera
atggatagggtggaggagttggcgaggaagaatgctgcagtgattttcaccaagagctcatgctgcatgtgccacagcatcaagacactgttctacgatctgggtgcgagccctgcgattcatgagctcgataaggacgctagaggaagggaaatggagtgggctttgcggcggatagggtgcaacccctccgtccctgctgtgtttgtaggcggaaaatttgttgggtctgctaaagatgtgattaccagccatgttgatgggtctctaaagcaaatgctcattgcagccagagccatctggttctag

SEQ ID NO: 428 - protein, 102 aa, Vitis vinifera
MDRVEELARKNAAVIFTKSSCCMCHSIKTLFYDLGASPAIHELDKDARGREMEWALRRIGCNPSVPAVFVGGKFVGSAKDVITSHVDGSLKQMLIAARAIWF

SEQ ID NO: 429 - DNA, 309 bp, Vitis vinifera
atggataaggtgacgagattggcttcagagaaaggggtggtgatcttcagcaagagctcatgctgcctgtgctatgctgtcaacattctgtttcaagaacttggggtcactcccacggttcatgaaatcgaccaagaccccgatggaagggaaatggagaaagccctcttgaggctgggatgcaatgctcctgtcccagctgtgttcataggtggaaagctcgtgggatccaccaacgaagtcatgtcccgtcacctaagcggctccctcattcccctgctgaagccatatcagcagactttatcttaa

SEQ ID NO: 430 - protein, 102 aa, Vitis vinifera
MDKVTRLASEKGVVIFSKSSCCLCYAVNILFQELGVTPTVHEIDQDPDGREMEKALLRLGCNAPVPAVFIGGKLVGSTNEVMSRHLSGSLIPLLKPYQQTLS

SEQ ID NO: 431 - DNA, 372 bp, Vitis vinifera
atgcattatcagacggaatcgtggggctcctacatgccaacaaggagcgttggagacccattggagcgcatagagagactggcctcagagaatgcggtggtgatattcagcatcagttcttgctgtatgtgccacgccattaagaggctcttctgcgggatgggcgtgaaccccacggtctacgagctcgacgaggaccccagagggaaggagatggagaaggccttgatgaggcttctgggtagttcctcggcggttccggtggtcttcatcggcgggaagctcgtcggagcaatggacagagtcatggcctcccatattaatgggacgctggtgcctctcctcaaggacgccggtgctctctggctttaa

SEQ ID NO: 432 - protein, 123 aa, Vitis vinifera
MHYQTESWGSYMPTRSVGDPLERIERLASENAVVIFSISSCCMCHAIKRLFCGMGVNPTVYELDEDPRGKEMEKALMRLLGSSSAVPVVFIGGKLVGAMDRVMASHINGTLVPLLKDAGALWL

SEQ ID NO: 433 - DNA, 404 bp, Zea mays
  misc_feature (47)..(47): n is a, c, g, or t
cgaagagccggtaaagaagttaatatcgtctagcaatggcccgggtnacgaaactggcttctcagcgcccggtggtcatcttcagcccgagctcctgctgtatgtgccccccggtgacgcgcctgttccgcgagctcggggtgaacccgccggtggtggagctggccgaggaccccagggggaaggagatggagaaggccctggcgaggctgctcgggcgcaaccccgctgtgccggcagtgttcatcggcggcaggctcgtcggctccaccgacaaggtcatgtcgcttcacctgagcggcaacctcgttccgcttctgcgcaatgccggcgcgctctgggtgtacccgtgttgcatatatatgtagctacccgttttccattccatatgcatgctatctatc

SEQ ID NO: 434 - protein, 110 aa, Zea mays
MARVTKLASQRPVVIFSPSSCCMCPPVTRLFRELGVNPPVVELAEDPRGKEMEKALARLLGRNPAVPAVFIGGRLVGSTDKVMSLHLSGNLVPLLRNAGALWVYPCCIYM

SEQ ID NO: 435 - DNA, 595 bp, Zea mays
aagtagtcaaagcatctagctcgttacaagtggctcttgtcgcctacaattttgccttcttccaagtcttgtttgaagttgctctctgcgaagagccggtagagaagttaatatcgtctagcaatggaccgggtgacgaaactggcttctcagcgcgcggtggtcatcttcagcacgagctcctgctgtatgtgccacacggtgacgcgcctgttccgcgagctcggggtgaacccgacggtggtggagctggacgaggaccccagggggaaggagatggagaaggcactggcgaggctgctcgggcgcaaccccgctgtgccggcagtgttcatcggcggcaggctcgtcggctccaccgacaaggtcatgtcgcttcacctgagcggcaacctcgttccgcttctgcgcaatgccggcgcgctctgggtgtagccgtgttgcatatatatgtagctagccgtgttccattccatatgcatgctatctatcaggagaggagacacacggagggaagtacgtagtaataattaattaagcaggcctttttatctgcaccgtaataattttccatagaggacggagtgtaaaaaaa

SEQ ID NO: 436 - protein, 103 aa, Zea mays
MDRVTKLASQRAVVIFSTSSCCMCHTVTRLFRELGVNPTVVELDEDPRGKEMEKALARLLGRNPAVPAVFIGGRLVGSTDKVMSLHLSGNLVPLLRNAGALWV

SEQ ID NO: 437 - DNA, 579 bp, Zea mays
aaacaagcagcagcagagcagaggatggcaatggaccacgtggcgaggctggcgtcggagcgcgcggtggtggtgttcacggcgagcaactgcagcatgggcgacgtggtgacgtcgctgctgagcagcctgggcgtcaacgcggcggtgcacgacctggacagggacccccggggcatggagatggagcgggagctggccaggaggctcggcggcggcggcgggcgcggcacgacgacgacccccaccgtgccggcggtgttcgtgggcggtgacctcgtcggcgggaccaacagggtcatggctctgcacctctccggcgagctcgtgcccatgctcaggaaagccggcgccctctggttgtagtaaattgagaacgcccgccgatcgaccgcgcgcatgcaagcatggctgatgagctttttacatcagctgtgtgtatatgtatgtgaataaaaaagaagcggtcaaagaaggcaaagagatggagagagaaggatccacccacgtactaagagtacgtgatgaatgcgaggtttttgttgtgtccttggaataaatgtcacctccgttttcact

SEQ ID NO: 438 - protein, 113 aa, Zea mays
MAMDHVARLASERAVVVFTASNCSMGDVVTSLLSSLGVNAAVHDLDRDPRGMEMERELARRLGGGGGRGTTTTPTVPAVFVGGDLVGGTNRVMALHLSGELVPMLRKAGALWL

SEQ ID NO: 439 - DNA, 622 bp, Zea mays
ccgggatctcaagcaacacgacgcattgcactagctagaccttgggctcgcaaacctcacacacccttccgcttaacctgcagcccttcgatcatcatcagcactcagcagtagcataggcatcaaggtagagaccccgacaatgcagtacggcgcggcggcggcggagcaggcgtggtcgtacatgccggtggtggcaccgtcgtcggccgtggagacggcggcggagcgcgtggagcggctggcgtcggagagcgcggtggtggtgttcagcgtgagcacctgctgcatgtgccacgccgtgaagcgcctcttctgcggcatgggcgtgcacccgacggtgcacgagctggaccacgacccgcggggccgcgagctggagcgcgccctggcctgcctcctcggcgcctccggagcctcggcggcgggcgcgccggtcgtgcccgtcgtgttcatcggcggcaggctggtcggcgccatggaccgcgtcatggccgcgcacatcaacggcaccctcgtgccgctgctcaaggacgccggcgcgctctggctgtgatctcatcgccggccgctagctactagccattgcccgcgttgcagctgttcaatcggggggctagct

SEQ ID NO: 440 - protein, 137 aa, Zea mays
MQYGAAAAEQAWSYMPVVAPSSAVETAAERVERLASESAVVVFSVSTCCMCHAVKRLFCGMGVHPTVHELDHDPRGRELERALACLLGASGASAAGAPVVPVVFIGGRLVGAMDRVMAAHINGTLVPLLKDAGALWL

SEQ ID NO: 441 - DNA, 997 bp, Zea mays
ctccaagcactcacctcctcagctcattgccagcctttggttccttccagctagcaaccggacggccagcaagccagcagactagtagctcttctcagttctcacacctactgtgtctgtgtgtgctcgcctttagcggcaagcgacccaccccccttcggtagcgagcgacaatgcagtacgcggcggcggagcaggcgtggtacatgccggcgaccacgacgatgatggcggagagcgcggtggcgcgcgtggagcggctggcgtcggagagcgcggtggtggtgttcagcgtgagcagctgctgcatgtgccacgccgtgaagcggctcttctgcggcatgggcgtgcacccgacggtgcacgagctggacctggacccgcgcggacgagagctggagcacgccctcgcccgcctcatcggctacggccccgccggcgcgcccgtcgtccccgtcgtcttcatcggcgggaagctggtcggcgccatggaccgggtcatggccgcgcatatcaacggctccctcgtcccacttctcaaggaggccggcgcgctctggctctgaactgtaggcaggccgcgcgccattgctggtgactcgtgtgccaacaggctacgcagcttcttccgttgtctaagttagttccttgttgctgtagaacctgatgtcacttgctgcaagctaatcaaactacgtactaagctacggtgataggaggatacatccatggacagggccggatggtggtagatcagtgatgcatcggattgggcttaatgtgtgtgagtagtgtgtgcaagagagaagagaggctggtaccgtgtgtgtgcttttttctcactacctactgcctccgtcggtgtttgtgtgtgcgtgcatgtggtgtttgacgcatgcgctggatttgctttgcttggggtgctacactgttaccaccactgttgtttaatttgatattcctttaattttaataccttgtttcctctcaaaaaaaa

SEQ ID NO: 442 - protein, 130 aa, Zea mays
MQYAAAEQAWYMPATTTMMAESAVARVERLASESAVVVFSVSSCCMCHAVKRLFCGMGVHPTVHELDLDPRGRELEHALARLIGYGPAGAPVVPVVFIGGKLVGAMDRVMAAHINGSLVPLLKEAGALWL

SEQ ID NO: 443 - DNA, 1568 bp, Oryza sativa
cccacgcgtccgcccacgcgtccgggacaccagaaacatagtacacttgagctcactccaaactcaaacactcacaccaatggctctccaagttcaggccgcactcctgccctctgctctctctgtccccaagaagggtaacttgagcgcggtggtgaaggagccggggttccttagcgtgagcagaaggccaagaagccgtcgctggtggtgagggcggtggcgacgcggcgggccggtggcgagccccggcgcgggcacgtcgaaggcggacgggaagaagacgctgcggcagggggtggtggtgatcaccggcgcgtcgtcggggctcgggctcgcggcggcgaaggcgcttggcggagacggggaagtggcacgtggtgatggcgttccgcgactttcctgaaggcggcgacggcggcgaaggcggcggggatggcggcggggagctacaccgtcatgcacctggacctcgcctccctcgacagcgtccgccagttcgtggacaacttccggcgctccggcatgccgctcgacgcgctggtgtgcaacgccgcacatctaccggccgacggcgcggcaaccgacgttcaacgccgacgggtacgagatgagcgtcggggtgaaccacctgggccacttcctcctcgcccgcctcatgctcgacgacctcaagaaatccgactacccgtcgcggcggctcatcatcctcggctccatcaccggcaacaccaacaccttcgccggcaacgtccctcccaaggccgggctaggcgacctccgggggctcgccggcgggctccgcgggcagaacgggtcggcgatgatcgacggcgcggagagcttcgacggcgccaaggcgtacaaggacagcaagatctgtaacatgctgacgatgcaggagttccaccggagattccacgaggagaccgggatcacgttcgcgtcgctgtacccggggtgcatcgcgacgacgggcttgttccgcgagcacatcccgctgttccggctgctgttcccgccgttccagcggttcgtgacgaaggggttcgtgtcggaggcggagtccgggaagcggctggcgcaggtggtgggcgacccgagcctgaccaagtccggcgtgtactggagctggaacaaggactcggcgtcgttcgagaaccagctctcgcaggaggccagcgacccggagaaggccaggaagctctgggacctcagcgagaagctcgtcggcctcgtctgagtttattatttacccattcgtttcaactgttaatttcttcggggtttagggggtttcagctttcagtgagagaggcctgtcaagtgatgtacaattagtaatttttttttacccgacaaatcatgcaataaaaccacaggcttacattatcgatttgtccacctaaattaagtttcaactgttaatttcttcggggtttagggggtttcagctttcagtgagagaggcctgtcaagtgatgtacaattagtaatttttttttacccgacaaatcatgcaataaaaccacaggcttacattatcgatttgtccacctaaattaagt

SEQ ID NO: 444 - DNA, 56 bp, Artificial sequence
  primer: prm09053
ggggacaagtttgtacaaaaaagcaggcttaaacaatggatatgataacgaagatg

SEQ ID NO: 445 - DNA, 50 bp, Artificial sequence
  primer: prm09054
ggggaccactttgtacaagaaagctgggtaaaaacatgataagtcaaacc

SEQ ID NO: 446 - DNA, 799 bp, Arabidopsis thaliana
ttgattgagatacttgagatccaagataaatatgtctttaagtcgtagagatcctcttgtggtcggcagtgttgttggagatgttcttgatcctttcacgaggttggtctctcttaaggtcacttatggccatagagaggttactaatggcttggatctaaggccttctcaagttctgaacaaaccaatagtggagattggaggagacgacttcagaaatttctacaccttggttatggtggatccagatgtgccgagtccaagcaaccctcaccaacgagaatatctccactggttggtgactgatatacctgccaccactggaaatgcctttggcaatgaggtggtgtgctacgagagtccacgtcccccctcgggaattcatcgtattgtgttggtattgttccggcaactcggaagacaaacggtttatgcaccggggtggcgccaacagttcaacactcgtgagtttgctgagatctacaatcttggtcttcctgtggctgcctcttacttcaactgccagagggagaatggctgtgggggaagaagaacgtagatgcgtacctacttacgttaactaataatctaatcgtataatattcccttaatgaagtatttaagcatctatgtcaatgtaataagaatttaaagatacgagctaaaaaaaatgatgcatatgctgacatcgatgtaaagtagtttacacttttaatgtaataactaggttttaacccgcggtacaccgcgagactattttgtttttttaagaataaaaatataatttgtttagtcgatt

SEQ ID NO: 447 - protein, 175 aa, Arabidopsis thaliana
MSLSRRDPLVVGSVVGDVLDPFTRLVSLKVTYGHREVTNGLDLRPSQVLNKPIVEIGGDDFRNFYTLVMVDPDVPSPSNPHQREYLHWLVTDIPATTGNAFGNEVVCYESPRPPSGIHRIVLVLFRQLGRQTVYAPGWRQQFNTREFAEIYNLGLPVAASYFNCQRENGCGGRRT

SEQ ID NO: 448 - DNA, 2194 bp, Oryza sativa; identical to SEQ ID NO: 5

SEQ ID NO: 449 - DNA, 58 bp, Artificial sequence
  primer: prm4759
ggggacaagtttgtacaaaaaagcaggcttaaacaatgtctttaagtcgtagagatcc

SEQ ID NO: 450 - DNA, 50 bp, Artificial sequence
  primer: prm4760
ggggaccactttgtacaagaaagctgggtgtacgcatctacgttcttctt

(19) United States
(12) Patent Application Publication
(10) Pub. No.: US 2011/0209241 A1
(43) Pub. Date: Aug. 25, 2011

(54) Plants Having Enhanced Yield-Related Traits and a Method for Making the Same

(75) Inventors: Yves Hatzfeld, Lille (FR); Valerie Frankard, Waterloo (BE); Christophe Reuzeau, Tocan Saint Apre (FR); Ana Isabel Sanz Molinero, Gentbrugge (BE); Steven Vandenabeele, Oudenaarde (BE)

(73) Assignee: BASF Plant Science GmbH, Ludwigshafen (DE)

(21) Appl. No.: 12/863,800

(22) PCT Filed: Jan. 23, 2009
(86) PCT No.: PCT/EP2009/050735

§ 371 Date: Jul. 21, 2010

Related U.S. Application Data
(60) Provisional application No. 61/031,546, filed on Feb. 26, 2008.
(60) Provisional application No. 61/031,444, filed on Feb. 26, 2008.
(60) Provisional application No. 61/031,736, filed on Feb. 27, 2008.
(60) Provisional application No. 61/031,713, filed on Feb. 27, 2008.
(60) Provisional application No. 61/031,716, filed on Feb. 27, 2008.
(60) Provisional application No. 61/031,723, filed on Feb. 27, 2008.

(30) Foreign Application Priority Data
Jan. 25, 2008 (EP) 08150637.0
Jan. 31, 2008 (EP) 08150893.9
Jan. 31, 2008 (EP) 08150897.0
Jan. 31, 2008 (EP) 08150912.7
Jan. 31, 2008 (EP) 08150913.5

(51) Int. Cl. A01H 5/00; C12N 15/82; C12N 5/10; A01H 5/12; A01H 5/10; C07H 21/04

(52) U.S. Cl. 800/278; 800/298; 435/320.1; 435/419; 435/412; 800/320.1; 800/320.2; 800/320.3; 800/320; 536/23.6

(57) ABSTRACT

The present invention relates generally to the field of molecular biology and concerns a method for improving various plant growth characteristics by modulating expression in a plant of a nucleic acid encoding a PRE-like (Paclobutrazol REsistance) polypeptide. The present invention also concerns plants having modulated expression of a nucleic acid encoding a PRE-like polypeptide, which plants have improved growth characteristics relative to corresponding wild type plants or other control plants. The invention also provides constructs useful in the methods of the invention. In another embodiment, the present invention relates generally to the field of molecular biology and concerns a method for enhancing various yield-related traits by modulating expression in a plant of a nucleic acid encoding an SCE1 (SUMO Conjugating Enzyme 1). The present invention also concerns plants having modulated expression of a nucleic acid encoding an SCE1, which plants have enhanced yield-related traits relative to corresponding wild type plants or other control plants. The invention also provides hitherto unknown SCE1-encoding nucleic acids, and constructs comprising the same, useful in performing the methods of the invention. In yet another embodiment, M the present invention relates generally to the field of molecular biology and concerns a method for enhancing various yield related-traits by modulating expression in a plant of a nucleic acid encoding a YEF1 (Yield Enhancing Factor 1). The present invention also concerns plants having modulated expression of a nucleic acid encoding a YEF1, which plants have enhanced yield related traits relative to corresponding wild type plants or other control plants. The invention also provides constructs useful in the methods of the invention. In yet another embodiment, the present invention relates generally to the field of molecular biology and concerns a method for enhancing various yield-related traits by modulating expression in a plant of a nucleic acid encoding a subgroup III glutaredoxin (Grx). The present invention also concerns plants having modulated expression of a nucleic acid encoding a subgroup III Grx, which plants have enhanced yield-related traits relative to corresponding wild type plants or other control plants. The invention also provides constructs useful in the methods of the invention. In a further embodiment, the present invention relates generally to the field of molecular biology and concerns a method for altering the ratio of roots to shoots in plants by modulating expression in a plant of a nucleic acid encoding a Sister of FT protein or a homologue thereof. The present invention also concerns plants having modulated expression of a nucleic acid encoding a Sister of FT protein or a homologue thereof, which plants have altered root to shoot ratio relative to corresponding wild type plants or other control plants. The invention also provides constructs useful in the methods of the invention.

The present invention relates generally to the field of molecular biology and concerns a method for improving various plant growth characteristics by modulating expression in a plant of a nucleic acid encoding a PRE-like (Paclobutrazol REsistance) polypeptide. The present invention also concerns plants having modulated expression of a nucleic acid encoding a PRE-like polypeptide, which plants have improved growth characteristics relative to corresponding wild type plants or other control plants. The invention also provides constructs useful in the methods of the invention.

In another embodiment, the present invention relates generally to the field of molecular biology and concerns a method for enhancing various yield-related traits by modulating expression in a plant of a nucleic acid encoding an SCE1 (SUMO Conjugating Enzyme 1). The present invention also concerns plants having modulated expression of a nucleic acid encoding an SCE1, which plants have enhanced yield-related traits relative to corresponding wild type plants or other control plants. The invention also provides hitherto unknown SCE1-encoding nucleic acids, and constructs comprising the same, useful in performing the methods of the invention.

In yet another embodiment, the present invention relates generally to the field of molecular biology and concerns a method for enhancing various yield related-traits by modulating expression in a plant of a nucleic acid encoding a YEF1 (Yield Enhancing Factor 1). The present invention also concerns plants having modulated expression of a nucleic acid encoding a YEF1, which plants have enhanced yield related traits relative to corresponding wild type plants or other control plants. The invention also provides constructs useful in the methods of the invention.

In yet another embodiment, the present invention relates generally to the field of molecular biology and concerns a method for enhancing various yield-related traits by modulating expression in a plant of a nucleic acid encoding a subgroup III glutaredoxin (Grx). The present invention also concerns plants having modulated expression of a nucleic acid encoding a subgroup III Grx, which plants have enhanced yield-related traits relative to corresponding wild type plants or other control plants. The invention also provides constructs useful in the methods of the invention.

In a further embodiment, the present invention relates generally to the field of molecular biology and concerns a method for altering the ratio of roots to shoots in plants by modulating expression in a plant of a nucleic acid encoding a Sister of FT protein or a homologue thereof. The present invention also concerns plants having modulated expression of a nucleic acid encoding a Sister of FT protein or a homologue thereof, which plants have altered root to shoot ratio relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigour has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

Another trait of particular agricultural interest is altered root:shoot ratio. Plants having a decreased aboveground plant area whilst retaining a sufficient root biomass would be particularly suited to cultivation in exposed areas. This would allow for the cultivation of crops during adverse conditions and in territories where cultivation of crops may not otherwise be possible. It has now been found that plant root:shoot ratio may be improved by modulating expression in a plant of a nucleic acid encoding a Sister of FT protein or a homologue thereof. One approach to increasing yield (seed yield and/or biomass) in plants may be through modification of the inherent growth mechanisms of a plant, such as the cell cycle or various signalling pathways involved in plant growth or in defense mechanisms.

It has now been found that various growth characteristics may be improved in plants by modulating expression in a plant of a nucleic acid encoding a PRE-like (Paclobutrazol REsistance) polypeptide.

In another embodiment has now been found that various yield-related traits may be improved in plants by modulating expression in a plant of a nucleic acid encoding an SCE1 (SUMO Conjugating Enzyme 1), or a YEF1 (Yield Enhancing Factor 1), or encoding a subgroup III glutaredoxin or Grx.

### BACKGROUND

### PRE-Like

### Paclobutrazol REsistance

Gibberellins are a group of structurally related compounds in angiosperms, gymnosperms, ferns, possibly also in mosses and algae, and at least in a few fungi. They interfere in diverse aspects of plant growth and development, including germination, stem elongation, leaf expansion, flowering and fruit development (Holey, Plant Mol. Biol. 26, 1529-1555, 1994). Recently PRE1, a HLH transcription regulator, was shown to be involved in gibberellin signalling (Lee et al., Plant Cell Physiol. 47, 591-600). It is induced by gibberellins, and under the control of GAI and SPY, which are upstream negative regulators of gibberellin signalling. PRE1 is not a bHLH transcription factor, as it lacks the basic domain in front of the HLH domain. It has nuclear localisation. Overexpression or activation-tagging of PRE1 in Arabidopsis results in a shorter life cycle, and early flowering, both under short and long day conditions. PRE1 reportedly had no effect on germination frequency, but seedlings overexpressing PRE1 had increased hypocotyl length. No effects on primary inflorescences were observed.

PRE1 belongs to a small gene family, Lee et al. (2006) report 6 members in Arabidopsis, all being similar in sequence and length. Overexpression in transgenic plants gave similar effects, implying that PRE genes may be functionally redundant (Lee et al., 2006). The PRE-like polypeptides show little sequence homology with the Id proteins. These proteins are about 120-150 amino acids long, and also have an HLH domain without a basic domain. The Id proteins bind to the ubiquitous bHLH protein E, thereby preventing the binding of the E protein to other bHLHs, which on their turn can no longer bind to their target promoters, and thus inactivate the expression of the bHLH target genes. Id proteins are expressed at low levels in normal cells but they play a role in many tumor types (progression of the cell cycle, invasiveness of tumor, tumor angiogenesis).

WO2005/072100 describes the identification of a PRE-like polypeptide from Arabidopsis, which, when overexpressed in Arabidopsis, caused an increase in the seed oil content. No other phenotypic effects were reported.

### SCE1 (SUMO Conjugating Enzyme 1)

Eukaryotic protein function is regulated in part by posttranslational processes such as the covalent attachment of small polypeptides. The most frequent and best characterized is the modification by ubiquitin and ubiquitin-like proteins. SUMO, the small ubiquitin-like modifier is similar to ubiquitin in tertiary structure but differs in primary sequence. SUMO conjugation to target proteins, a process referred to as sumoylation, involves the sequential action of a number of enzymes, namely, activating (E1), conjugating (E2 or SUMO E2) and ligase (E3). The process is reversible, and desumoylation, that is, removal of SUMO from the substrate, is mediated by SUMO proteases. Mechanistically sumoylation comprises distinct phases. Initially the E1 enzyme complex activates SUMO by binding to it via a highly reactive sulfhydryl bond. Activated SUMO is then transferred to the E2 conjugating enzyme via trans-sterification reaction, involving a conserved cysteine residue in the E2 enzyme. Residue cysteine 94 is the conjugated residue in the Arabidopsis thaliana E2 enzyme, also named AtSCE1 protein. In the last step, SUMO is transferred to the substrate via an isopeptide bond.

While protein modification by ubiquitin often results in protein degradation, sumoylation, i.e. conjugation of SUMO to proteins, is often associated with protein stabilization. Sumoylation function is best understood in yeast and animals where it plays a role in signal transduction, cell cycle DNA repair, transcriptional regulation, nuclear import and subsequent localization and in viral pathogenesis. In plants, sumoylation has been implicated in regulation of gene expression in response to development, hormonal and environmental changes (Miura et al. 2007. Current Opinion in Plant Biol. 10, 495-502).

Protein components of the sumoylation pathway are encoded in the genome of eukaryotes. In yeast and mammals there is a single SUMO E2 conjugating enzyme described. Although initially in Arabidopsis thaliana only a single SUMO E2, AtSCE1a, was found (Lois et al. 2003. The Plant Cell 15, 1347-1359), some plants may have multiple isoforms, as is the case for rice, for which three genes encoding E2 enzymes have been described (Miura et al. 2007). The AtSCE1a protein is characterized by the presence of a UBC domain and of an active site cysteine amino acid residue. In Arabidopsis thaliana there are more than 40 UBC domain-containing proteins, of which the great majority are thought to act as ubiquitin conjugating enzymes, and only four of them are predicted or shown to function on conjugation of ubiquitin-like proteins. Of the latter only AtSCE1a (At3g57870) and a truncated SCE1b protein (At5g02240) thought to be encoded by a pseudogene are proposed to act as SUMO E2 conjugating enzymes (Kraft et al. Plant Phys 2005, 1597-1611). In comparison to other UBC proteins, SCE1a protein has higher amino acid identity to human UBC12 and UBC9. Phylogenetic analysis revealed that Arabidopsis proteins with a UBC domain and an active site cysteine amino acid residue can be divided into 16 groups, with group I functioning in SUMO conjugation pathway (Kraft et al. 2005).

Functional characterization of a Nicotiana SCE1 protein showed that it can activate SUMO in vitro and it can complement a yeast SUMO E2 mutant (Castilo et al. 2004. J. virology 78: 2758-2769). Arabidopsis thaliana transgenic plants overexpressing a modified AtSCE1a by a histidine tag were used to demonstrate nuclear colocalization of AtSCE1a and SUMO1/2 (Lois et al 2003). The authors showed altered behaviour of the transgenic plant response to specific stresses such as salt and the hormone ABA, but not the hormone Auxin. However the authors failed to state any growth difference between the control and the transgenic plants grown on control medium lacking the factor causing the stress.

### YEF1 (Yield Enhancing Factor 1)

Interactions between proteins and RNAs underlie many aspects of plant development and function. Accordingly, plants and other eukaryotes encode hundreds of proteins containing domains that interact with nucleic acids such as RNA (ribonucleic acid) and DNA (deoxyribonucleic acid). Examples of protein domains present in proteins that interact with nucleic acids are the CCCH Zinc Finger (C3H Znf) domain and the RRM (RNA recognition motif) domain.

The CCCH domain has been found in proteins involved in cell cycle or growth phase-related regulation e.g. human TIS11B (butyrate response factor 1) and the human splicing factor U2AF 35 kD subunit, which plays a critical role in both constitutive and enhancer-dependent splicing by mediating essential protein-protein interactions and protein-RNA interactions required for 3′ splice site selection. Zinc-binding domains are stable structures, and they rarely undergo conformational changes upon binding their target. It has been proposed that Zinc finger domains in proteins are stable scaffolds that have evolved specialized functions. For example, Znf-domains function in gene transcription, translation, mRNA trafficking, cytoskeleton organization, epithelial development, cell adhesion, protein folding, chromatin remodeling and zinc sensing. It has been shown that different CCCH-type Znf proteins interact with the 3′-untranslated region of various mRNA (Carballo et al. 1998 Science 281 1001-1005). The CCCH domain can be represented by sequence C-x8-C-x5-C-x3-H, where the conserved cysteine and histidine residues are proposed to coordinate Zn ions (Brown 2005. Curr. Opin. Struct. Biol. 15 94-8).

RNA recognition motifs or RRMs are typically present in a large variety of RNA-binding proteins involved in post-transcriptional events, whereby the number of RRMs per protein varies from one up to several copies. The RRM is a region of around eighty amino acids containing several well conserved residues, some of which cluster into two short submotifs, RNP-1 (octamer) and RNP-2 (hexamer) (Birney et al., Nucleic Acids Research, 1993, Vol. 21, No. 25, 5803-5816). Examples of RRM domain containing proteins include heterogeneous nuclear ribonucleoproteins (hnRNPs), proteins implicated in regulation of alternative splicing (SR, U2AF, Sxl), protein components of small nuclear ribonucleoproteins (U1 and U2 snRNPs), and proteins that regulate RNA stability and translation (PABP, La, Hu) 5REF). The motif also appears in a few single stranded DNA binding proteins. The typical RRM domain consists of four anti-parallel beta-strands and two alpha-helices arranged in a beta-alpha-beta-beta-alpha-beta fold with side chains that stack with RNA bases. Specificity of RNA binding is determined by multiple contacts with surrounding amino acids. A third helix is present during RNA binding in some cases (Birney E. et al. 1993; Maris C. et al. 2005 FEBS J 272 2118-31).

Several databases have catalogues of proteins comprising RRM domains, such as Plant RBP (Walker, et al. 2007. Nucleic Acids Res, 35, D852-D856); pfam (Bateman et al. 2002. Nucleic Acids Research 30(1): 276-280) and InterPro (Mulder et al., (2003) Nucl. Acids. Res. 31, 315-318). The accession number of the RRM domain and CCCH in InterPro are IPR000504, IPR000571 respectively.

Mining of protein and protein domain databases such as IntrePro and pfam reveals that only a small number of eukaryotic proteins comprise in addition to the CCCH, and the RRM domains, a well conserved domain which is typically found at the N-terminus and that resembles the histone fold domain (InterPro accession number IPR0009072). An example of such a protein is the Le_YEF1_{—}1, a tomato protein hereafter described. The histone-fold domain consists of a core of three helices, where the long middle helix is flanked at each end by shorter ones. Proteins displaying this structure include the nucleosome core histones and the TATA-box binding protein (TBP)-associated factors (TAF), where the histone fold is a common motif for mediating TAF-TAF interactions. The TAF proteins are a component of transcription factor IID (TFIID). TFIID forms part of the pre-initiation complex on core promoter elements required for RNA polymerase II-dependent transcription.

### Subgroup III Glutaredoxin (Grx)

The redox chemistry that living cells experience in their normal environment is dominated by oxygen. The cytosol of living cells however is a very reducing environment and reducing conditions are essential for its proper function. Oxygen and reactive derivatives of molecular oxygen are a constant threat to biological systems. The only significantly redox active component of generic proteins is the amino acid cysteine, which under normal atmospheric conditions will oxidize completely to form a disulfide bond. While disulfide cross-links are important for the structure and stability of many secretory proteins, they are essentially absent from cytosolic proteins. Should they arise from spontaneous oxidation by molecular oxygen or reactive oxygen species, living cells have two major pathways that deal with reduction of disulfide bonds in the cytosol: the thioredoxin and the glutaredoxin pathways. The key players are small enzymes of similar structure (thioredoxin and glutaredoxin (Grx)) that employ reactive thiol-disulfide relay systems in CysXaaXaaCys sequence motifs (where Xaa can be a number of different amino acid residues). Glutaredoxin (Grx) catalyses the reduction of disulfide bonds in proteins converting glutathione (GSH) to glutathione disulfide (GSSG). GSSG is in turn recycled to GSH by the enzyme glutathione reductase at the expense of NADPH. During the reaction cycle it is thought that a cysteine pair in the active site of glutaredoxin is converted to a disulfide.

When submitted to adverse environmental conditions (biotic or abiotic stresses), plants very often react by generating oxidative bursts. To avoid biological damage, the concentration of the oxidizing species must be kept under control. One of the most documented functions of glutaredoxins (Grxs) in plants is their involvement in the oxidative stress response. They are implicated in many different ways, for example by directly reducing peroxides or dehydroascorbate (DHA), by reducing peroxiredoxins (Prx), and also by protecting thiol groups on other enzymes via gluathionylation/deglutathionylation mechanisms. Grxs need to be reduced in order to function, the reducing system being composed of an NADPH dependent pyridine nucleotide oxidoreductase called glutathione reductase (GR) and the small tripeptide, glutathione. Rouhier et al., 2006, Journal of Experimental Botany, 23 May.

Grx polypeptides have been divided into three subgroups based on sequence alignments, active site sequences and construction of unrooted phylogenetic trees (see Rouhier et al., 2006).

Rouhier et al., 2006 report that subgroup I contains Grxs with CPYC, CGYC, CPFC, and CSY[C/S] active sites. This group comprises five different classes of Grx (Grx C1-04 and S12) which differ in their active site sequences. The nomenclature used (C or S) is based on the presence of a cysteine or a serine in the fourth position of the active site (CxxC or CxxS). They report that proteins of subgroup II possess CGFS active sites, but they differ in the number of repeated modules. Proteins of subgroup III are reported to mainly possess active sites of the CC[M/L][C/S] form.

### Sister of FT

The FLOWERING LOCUS T (FT) gene plays a central role in integrating flowering signals in Arabidopsis because its expression is regulated antagonistically by the photoperiod and vernalization pathways. FT belongs to a family of six genes characterized by a phosphatidylethanolamine-binding protein (PEBP) domain. In Arabidopsis, FTencodes a protein similar to a phosphatidylethanolamine-binding protein (PEBP). FT is a member of a small gene family, which includes five other genes: TERMINAL FLOWER 1 (TFL1), TWIN SISTER OF FT (TSF), ARABIDOPSIS THALIANA CENTRORADIALIS (ATC), BROTHER OF FT AND TFL1 (BFT), and MOTHER OF FT AND TFL1 (MFT). BFT has not been implicated in flowering, but constitutive expression of FT, TSF, and, to a lesser extent, MFT accelerates flowering. Faure et al., 2007, Genetics 176: 599-609.

### SUMMARY

Surprisingly, it has now been found that modulating expression of a nucleic acid encoding a PRE-like polypeptide gives plants having enhanced yield-related traits relative to control plants, in particular increased seed yield relative to control plants, provided that the increased seed yield does not encompass increased oil content of seeds.

According to one embodiment, there is provided a method for improving yield-related traits of a plant relative to control plants, comprising modulating expression of a nucleic acid encoding a PRE-like polypeptide in a plant. The improved yield related traits comprise increased seed yield.

Also surprisingly, it has now been found that modulating expression of a nucleic acid encoding an SCE1 polypeptide gives plants having enhanced yield-related traits relative to control plants.

According one embodiment, there is provided a method for enhancing yield related traits of a plant relative to control plants, comprising modulating expression of a nucleic acid encoding an SCE1 polypeptide in a plant. The enhanced yield related traits comprise increased shoot and root biomass and increase number of panicles and of seeds of a plant.

Furthermore, surprisingly, it has now been found that modulating expression of a nucleic acid encoding a YEF1 polypeptide gives plants having enhanced yield-related traits in particular increased yield relative to control plants.

According to one embodiment, there is provided a method for enhancing yield related traits of a plant relative to control plants, comprising modulating expression of a nucleic acid encoding a YEF1 polypeptide in a plant and optionally selecting for plants having enhanced yield-related traits.

Furthermore, surprisingly, it has now been found that modulating expression of a nucleic acid encoding a subgroup III Grx polypeptide gives plants having enhanced yield-related traits, in particular (increased yield) relative to control plants.

Furthermore, surprisingly, it has now been found that modulating expression of a nucleic acid encoding a Sister of FT protein or a homologue thereof gives plants having an altered root:shoot ratio relative to control plants.

According one embodiment, there is provided a method for altering the root:shoot ratio of plants, comprising modulating expression in a plant of a nucleic acid encoding a Sister of FT protein or a homologue thereof.

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### Homoloque(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| Residue | Conservative Substitutions | Residue | Conservative Substitutions |
|---|---|---|---|
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Ortholoque(s)/Paraloque(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

Motif/Consensus sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Lᵢ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ, is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]−0.61×% formamide

2) DNA-RNA or RNA-RNA hybrids:

Tm=79.8+18.5(log₁₀[Na⁺]^{a})+0.58(%G/C^{b})+11.8(%G/C^{b})²−820/L^{c}

3) oligo-DNA or oligo-RNA^{d} hybrids:
For <20 nucleotides: Tₘ=2 (Iₙ)
For 20-35 nucleotides: Tₘ=22+1.46 (Iₙ)
   ^{a}or for other monovalent cation, but only accurate in the 0.01-0.4 M range.
   ^{b}only accurate for % GC in the 30% to 75% range.
   ^{c}L=length of duplex in base pairs.
   ^{d} oligo, oligonucleotide; Iₙ,=effective length of primer=2×(no. of G/C)+(no. of NT).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCI and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGP | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, WO 2004/065596 |
| Ubiquitin | Christensen et al, Plant Mol. Biol. 18: 675-689, 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small subunit | U.S. Pat. No. 4,962,028 |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. 12(20): 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

Examples of root-specific promoters are listed in Table 2b below:

TABLE 2b
Examples of root-specific promoters

| Gene Source | Reference |
|---|---|
| RCc3 | Plant Mol Biol. 1995 Jan; 27(2): 237-48 |
| Arabidopsis PHT1 | Kovama et al., 2005; Mudge et al. (2002, Plant J. 31: 341) |
| Medicago phosphate transporter | Xiao et al., 2006 |
| Arabidopsis Pyk10 | Nitz et al. (2001) Plant Sci 161(2): 337-346 |
| root-expressible genes | Tingey et al., EMBO J. 6: 1, 1987. |
| tobacco auxin-inducible gene | Van der Zaal et al., Plant Mol. Biol. 16, 983, 1991. |
| β-tubulin | Oppenheimer, et al., Gene 63: 87, 1988. |
| tobacco root-specific genes | Conkling, et al., Plant Physiol. 93: 1203, 1990. |
| B. napus G1-3b gene | U.S. Pat. No. 5,401,836 |
| SbPRP1 | Suzuki et al., Plant Mol. Biol. 21: 109-119, 1993. |
| LRX1 | Baumberger et al. 2001, Genes & Dev. 15: 1128 |
| BTG-26 Brassica napus | US 20050044585 |
| LeAMT1 (tomato) | Lauter et al. (1996, PNAS 3: 8139) |
| The LeNRT1-1 (tomato) | Lauter et al. (1996, PNAS 3: 8139) |
| class I patatin gene (potato) | Liu et al., Plant Mol. Biol. 153: 386-395, 1991. |
| KDC1 (Daucus carota) | Downey et al. (2000, J. Biol. Chem. 275: 39420) |
| TobRB7 gene | W Song (1997) PhD Thesis, North Carolina State |
| | University, Raleigh, NC USA |
| OsRAB5a (rice) | Wang et al. 2002, Plant Sci. 163: 273 |
| ALF5 (Arabidopsis) | Diener et al. (2001, Plant Cell 13: 1625) |
| NRT2;1Np (N. plumbaginifolia) | Quesada et al. (1997, Plant Mol. Biol. 34: 265) |

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm and/or aleurone and/or embryo-specific. Examples of seed-specific promoters (endosperm/aleurone/embryo specific) are shown in Tables 2c-f below. Further examples of seed-specific promoters are given in Qing Qu and Takaiwa (Plant Biotechnol. J. 2, 113-125, 2004), which disclosure is incorporated by reference herein as if fully set forth.

TABLE 2c
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW glutenin-1 | Mol Gen Genet 216: 81-90, 1989; NAR 17: 461-2, 1989 |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98:1253-62, 1999; Plant J 4: 343-55, |
| | 1993; Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice a-globulin Glb-1 | Wu et al, Plant Cell Physiology 39(8) 885-889, 1998 |
| rice OSH1 | Sato et al, Proc. Natl. Acad. Sci. USA, 93: 8117-8122, |
| | 1996 |
| rice α-globulin REB/OHP-1 | Nakase et al. Plant Mol. Biol. 33: 513-522, 1997 |
| rice ADP-glucose | Trans Res 6:157-68, 1997 |
| pyrophosphorylase | |
| maize ESR gene family | Plant J 12:235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2d
examples of endosperm-specific promoters

| Gene source | Reference |
|---|---|
| glutelin (rice) | Takaiwa et al. (1986) Mol Gen Genet 208: 15-22; |
| | Takaiwa et al. (1987) FEBS Letts. 221: 43-47 |
| zein | Matzke et al., (1990) Plant Mol Biol 14(3): 323-32 |
| wheat LMW and HMW glutenin-1 | Colot et al. (1989) Mol Gen Genet 216: 81-90, |
| | Anderson et al. (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, hordein | Cho et al. (1999) Theor Appl Genet 98: 1253-62; |
| | Muller et al. (1993) Plant J 4: 343-55; |
| | Sorenson et al. (1996) Mol Gen Genet 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin REB/OHP-1 | Nakase et al. (1997) Plant Molec Biol 33: 513-522 |
| rice ADP-glucose pyrophosphorylase | Russell et al. (1997) Trans Res 6: 157-68 |
| maize ESR gene family | Opsahl-Ferstad et al. (1997) Plant J 12: 235-46 |
| sorghum kafirin | DeRose et al. (1996) Plant Mol Biol 32: 1029-35 |

TABLE 2e
Examples of embryo specific promoters:

| Gene source | Reference |
|---|---|
| rice OSH1 | Sato et al, Proc. Natl. Acad. Sci. USA, 93: 8117-8122, 1996 |
| KNOX | Postma-Haarsma et al, Plant Mol. Biol. 39: 257-71, 1999 |
| PRO0151 | WO 2004/070039 |
| PRO0175 | WO 2004/070039 |
| PRO005 | WO 2004/070039 |
| PRO0095 | WO 2004/070039 |

TABLE 2f
Examples of aleurone-specific promoters:

| Gene source | Reference |
|---|---|
| α-amylase (Amy32b) | Lanahan et al, Plant Cell 4: 203-211, 1992; |
| | Skriver et al, Proc Natl Acad Sci |
| | USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38,1998 |

A green tissue-specific promoter as defined herein is a promoter that is transcriptionally active predominantly in green tissue, substantially to the exclusion of any other parts of a plant, whilst still allowing for any leaky expression in these other plant parts.

Examples of green tissue-specific promoters which may be used to perform the methods of the invention are shown in Table 2g below.

TABLE 2g
Examples of green tissue-specific promoters

| Gene | Expression | Reference |
|---|---|---|
| Maize Orthophosphate dikinase | Leaf specific | Fukavama et al., 2001 |
| Maize Phosphoenolpyruvate | Leaf specific | Kausch et al., 2001 |
| carboxylase | | |
| Rice Phosphoenolpyruvate | Leaf specific | Liu et al., 2003 |
| carboxylase | | |
| Rice small subunit Rubisco | Leaf specific | Nomura et al., 2000 |
| rice beta expansin EXBP9 | Shoot specific | WO 2004/070039 |
| Pigeonpea small subunit Rubisco | Leaf specific | Panguluri et al., 2005 |
| Pea RBCS3A | Leaf specific | |

Another example of a tissue-specific promoter is a meristem-specific promoter, which is transcriptionally active predominantly in meristematic tissue, substantially to the exclusion of any other parts of a plant, whilst still allowing for any leaky expression in these other plant parts. Examples of green meristem-specific promoters which may be used to perform the methods of the invention are shown in Table 2h below.

TABLE 2h
Examples of meristem-specific promoters

| Gene source | Expression pattern | Reference |
|---|---|---|
| Brice OSH1 | Shoot apical meristem, from | Sato et al. (1996) |
| | embryo globular stage to | Proc. Natl. Acad. |
| | seedling stage | Sci. USA, 93: |
| | | 8117-8122 |
| Rice metallothionein | Meristem specific | BAD87835.1 |
| WAK1 & WAK 2 | Shoot and root apical | Wagner & Kohorn |
| | meristems, and in expanding | (2001) Plant Cell |
| | leaves and sepals | 13(2): 303-318 |

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adh1-5 intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene. The isolated gene may be isolated from an organism or may be manmade, for example by chemical synthesis.

### Decreased Expression

Reference herein to “decreased expression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants. Examples of various methods for the reduction or substantial elimination of expression in a plant of an endogenous gene, or for lowering levels and/or activity of a protein, are known to the skilled in the art. The person skilled in the art is aware of the different approaches that allow a reduction or substantial elimination of expression, such as, but not limited to gene silencing, RNA-mediated silencing, co-suppression or insertion mutagenesis. Methods for decreasing expression are known in the art and the skilled person would readily be able to adapt the known methods for silencing so as to achieve reduction of expression of an endogenous gene in a whole plant or in parts thereof through the use of an appropriate promoter, for example.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest, preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, the polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15, 1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved. Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. MiRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. The person skilled in the art would readily be able to adapt the aforementioned methods for silencing so as to achieve reduction of expression of an endogenous gene in a whole plant or in parts thereof through the use of an appropriate promoter. The skilled is also aware of the different approaches that allow a reduction or substantial elimination of expression, such as, but not limited to gene silencing, RNA-mediated silencing, co-suppression or insertion mutagenesis.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transqenic/Transqene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen Genet 202: 179-185); DNA or RNA-coated particle bombardment (Klein TM et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): β-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### Tilling

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei GP and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8), and approaches exist that are generally applicable regardless of the target organism (Miller et al, Nature Biotechnol. 25, 778-785, 2007).

### Yield

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), and g) increased number of primary panicles, which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased seed yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elate, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticale sp., Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

Surprisingly, it has now been found that modulating expression in a plant of a nucleic acid encoding a PRE-like polypeptide, or an SCE1 polypeptide, or a YEF1 polypeptide, or a subgroup III Grx polypeptide, gives plants having enhanced yield-related traits relative to control plants. According to a first embodiment, the present invention provides a method for enhancing yield-related traits in plants relative to control plants, comprising modulating expression in a plant of a nucleic acid encoding a PRE-like polypeptide, or an SCE1 polypeptide, or a YEF1 polypeptide, or a subgroup III Grx polypeptide.

Surprisingly, it has now been found that modulating expression in a plant of a nucleic acid encoding a Sister of FT protein or a homologue thereof gives plants having an altered root:shoot ratio relative to control plants. According to a first embodiment, the present invention provides a method for altering the root:shoot ratio of plants relative to control plants, comprising modulating expression in a plant of a nucleic acid encoding a Sister of FT protein or a homologue thereof.

A preferred method for modulating (preferably, increasing) expression of a nucleic acid encoding a PRE-like polypeptide, or an SCE1 polypeptide, or a YEF1 polypeptide, or a subgroup III Grx polypeptide, or a Sister of FT protein is by introducing and expressing in a plant a nucleic acid encoding a PRE-like polypeptide, or an SCE1 polypeptide, or a YEF1 polypeptide, or a subgroup III Grx polypeptide, or a Sister of FT protein.

Concerning PRE-like polypeptides/genes, any reference hereinafter to a “protein useful in the methods of the invention” is taken to mean a PRE-like polypeptide as defined herein. Any reference hereinafter to a “nucleic acid useful in the methods of the invention” is taken to mean a nucleic acid capable of encoding such a PRE-like polypeptide. The nucleic acid to be introduced into a plant (and therefore useful in performing the methods of the invention) is any nucleic acid encoding the type of protein which will now be described, hereinafter also named “PRE-like nucleic acid” or “PRE-like gene”.

Regarding SCE1 polypeptides/genes, any reference hereinafter to a “protein useful in the methods of the invention” is taken to mean an SCE1 polypeptide as defined herein. Any reference hereinafter to a “nucleic acid useful in the methods of the invention” is taken to mean a nucleic acid capable of encoding such an SCE1 polypeptide. The nucleic acid to be introduced into a plant (and therefore useful in performing the methods of the invention) is any nucleic acid encoding the type of protein which will now be described, herein+after also named “SCE1 nucleic acid” or “SCE1 gene”.

Concerning YEF1 polypeptides/genes, any reference hereinafter to a “protein or polypeptide useful in the methods of the invention” is taken to mean a YEF1 polypeptide as defined herein. Any reference hereinafter to a “nucleic acid useful in the methods of the invention” is taken to mean a nucleic acid capable of encoding such a YEF1 polypeptide. The nucleic acid to be introduced into a plant (and therefore useful in performing the methods of the invention) is any nucleic acid encoding the type of protein which will now be described, hereinafter also named “YEF1 nucleic acid” or “YEF1 gene”.

Regarding subgroup III Grx polypeptides/genes, any reference hereinafter to a “protein useful in the methods of the invention” is taken to mean a subgroup III Grx polypeptide as defined herein. Any reference hereinafter to a “nucleic acid useful in the methods of the invention” is taken to mean a nucleic acid capable of encoding such a subgroup III Grx polypeptide. The nucleic acid to be introduced into a plant (and therefore useful in performing the methods of the invention) is any nucleic acid encoding the type of protein which will now be described, hereinafter also named “subgroup III Grx nucleic acid” or “subgroup III Grx gene”.

Concerning Sister of FT polypeptides/genes, any reference hereinafter to a “protein useful in the methods of the invention” is taken to mean a Sister of FT protein or a homologue thereof as defined herein. Any reference hereinafter to a “nucleic acid useful in the methods of the invention” is taken to mean a nucleic acid capable of encoding such a Sister of FT protein or a homologue thereof. The nucleic acid to be introduced into a plant (and therefore useful in performing the methods of the invention) is any nucleic acid encoding the type of protein which will now be described, hereinafter also named “Sister of FT nucleic acid” or “Sister of FT” gene”.

A “PRE-like polypeptide” as defined herein refers to the protein presented by SEQ ID NO: 2 and orthologues and paralogues thereof. Preferably, the PRE-like polypeptide sequence comprises at least one of the motifs 1, 2 or 3:

Motif 1 (SEQ ID NO: 7):
(E/D/N)X₁(E/Q)(I/V/M)X₂(E/D/Q/A/N)(L/F/I)
(I/V/L/M)(S/I/T/L/Y)X₃L(Q/R/H)X₄(L/F/I/S)
(L/V/I)(P/A)

Wherein X₁ can be any amino acid, but preferably one of E, D, K, N, A, Q; more preferably X₁ is E or D, and
Wherein X₂ can be any amino acid, but preferably one of N, I, A, T, S, G, H, L, M, K; more preferably X₂ is one of N, I, A, T, S, and
Wherein X₃ can be any amino acid, but preferably one of K, R, S, Q, E, T; more preferably X₃ is K, and
Wherein X₄ can be any amino acid, but preferably one of Q, A, D, S, T, R, H, L, P; more preferably X₄ is one of Q, A, D, S.

Preferably, motif 1 is
(E/D)(E/D)(E/Q)I(N/I/A/T/S)(E/D/Q)L(I/V)SKL(Q/R)
(Q/A/D/S)L(L/V/I)P
Motif 2 (SEQ ID NO: 8):
(A/T/S)X(K/R/N/S)(V/L/I/M/A)L(Q/K/R/E/H)(E/D/Y/Q)
TC(N/S/T/I/A)(Y/S/C)(I/F/V)(R/K/G)(S/N/D/T/R)
(L/S)(H/Q/N/S)

Wherein X can be any amino acid, but preferably one of S, T, A, G, F, Y, N, W; more preferably one of S, T, A.

Preferably, motif 2 is
(A/T/S)(S/T/A)(K/R)(V/L)L(Q/K)ETC(N/S/T)YI(R/K)
(S/N)LH
Motif 3 (SEQ ID NO: 9): (E/Q) A (A/E) IIRSL

Further preferably, the PRE-like polypeptide also comprises one or more of the following motifs:

Motif 4 (SEQ ID NO: 10):
MS(S/G)R(R/K)SRSRQ(S/T)
at the N-terminus
Motif 5 (SEQ ID NO: 11):
(K/Q)L(Q/H)(D/Q/R)LLPE
Motif 6 (SEQ ID NO: 12):
LQ(E/D)TC(T/N/S)YI
Motif 7 (SEQ ID NO: 13):
EV(D/G)DLSERLS(E/Q)LL
Motif 8 (SEQ ID NO: 14):
QAA(I/V/L)IR(S/N/R)LL
at the C-terminus

Typically, PRE-like polypeptides comprise a Helix-Loop-Helix DNA binding domain (InterPro IPR011598, Superfamily SSF47459, SMART SM00353, Profile PS50888) but do not comprise a basic domain; in this aspect, they differ from bHLH transcription factors.

Alternatively, the homologue of a PRE-like protein has in increasing order of preference at least 45%, 46%, 47%, 48%, 49%, 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2, provided that the homologous protein comprises the conserved motifs as outlined above. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered.

Preferably, the polypeptide sequence which when used in the construction of a phylogenetic tree, such as the one depicted in FIG. 3, clusters with the group of PRE-like polypeptides comprising the amino acid sequence represented by SEQ ID NO: 2 rather than with any other group.

A “SCE1 polypeptide” as defined herein refers to any polypeptide comprising a Ubiquitin-conjugating domain (UBC domain) and preferably having SUMO E2 conjugating activity.

The conserved UBC domain is approximately 140 to 150 amino acids long and corresponds to the entry with accession number IPR000608 in the InterPro database (InterPro (Mulder et al., (2003) Nucl. Acids. Res. 31, 315-318).

Examples of SCE1 polypeptides useful in the methods of the invention SCE1 polypeptides are given in Table A2 of Example 1 herein. Table C2 in Example 4 describes the UBC domains as present in the SCE1 polypeptides of Table A1.

A preferred SCE1 polypeptide useful in the methods of the invention comprises an amino acid sequence having, in increasing order of preference, at least 50%, 55%, 60%, 65%, 70%, 75%, 80%, 85%, 90%, 95%, 96%, 97%, 98%, 99% or more sequence identity to the amino acid sequence of any of the UBC domains as set forth in Table C2 of Example 4.

Further preferably, the SCE1 polypeptide mentioned above is a polypeptide having, in increasing order of preference, at least 50%, 55%, 60%, 65%, 70%, 75%, 80%, 85%, 90%, 95%, 96%, 97%, 98%, 99% or more sequence identity to the amino acid sequence of any of the polypeptides of Table A2. Most preferably, the SCE1 polypeptide is one of the polypeptides of Table A2.

Alternatively, the homologue of an SCE1 protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 200. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered.

Alternatively, the sequence of the SCE1 polypeptide useful in the methods of the invention when used in the construction of a phylogenetic tree, such as the one depicted in FIG. 6 of Kraft et al. 2005, clusters with the group I comprising the amino acid sequence of AtSCE1a rather than with any other group.

A “YEF1 polypeptide” as defined herein refers to any polypeptide comprising an NPD1 domain (novel protein domain 1), an RRM (RNA recognition motif) domain and optionally a CCCH (C3H Zinc Finger) domain.

An NDP1 domain resembles the histone fold domain (InterPro accession number IPR009072). An IPR009072 domain folds into alpha helices. Example 4 gives the amino acid coordinates of the NPD1 domains as present in the polypeptides of Table A3.

Preferred YEF1 polypeptides useful in the methods of the invention comprise an NPD1 domain or a protein domain having in increasing order of preference at least 50%, 55%, 60%, 65%, 70%, 75%, 80%, 85%, 90%, 95% or more sequence identity to any of the NPD1 domains as set forth in Table C of Example 4. Most preferably the abovementioned YEF1 polypeptides comprise an NPD1 domain as represented by the amino acid sequences specified in Table C3 of Example 4.

Furthermore, RRM domains are well known in the art and consist of around 90 amino acids; they have a structure consisting of four strands and two helices arranged in an alpha/beta sandwich, with a third helix sometimes being present during RNA binding. RRM domain-containing proteins have a modular structure. RRM domains may be identified for example by using the tool SMART (Schultz et al. PNAS, 95, 5857-5864 (1998); Letunic et al., (Nucleic Acids Res. 30(1), 242-244).

Preferred YEF1 polypeptides useful in the methods of the invention comprise an RRM domain or a protein domain having in increasing order of preference at least 50%, 55%, 60%, 65%, 70%, 75%, 80%, 85%, 90%, 95% or more sequence identity to any of the RRM domains as set forth in Table C3 of Example 4; Most preferably the YEF1 polypeptides above-mentioned comprise an RRM domain as represented by the amino acid sequences specified in Table C3 of Example 4.

CCCH (C3H) Zinc finger domains are well known in the art and consist of about 20 amino acids comprising three cysteine (Cys) and one histidine (Hys) capable of coordinating of a zinc ion. The Cys and His residues are arranged in a sequence as follows: C-X(7-8)-C-X5-C-X3-H, where X represents and the digit number behind the X indicates the number times that X occurs (SEQ ID NO: 276). CCCH domains occurring in a polypeptide may be readily identified for example by simply reading the amino acid sequence or by searching in databases of conserved amino acids domains in proteins such as InterPro and Pfam. CCCH has accession number IPR000504 in InterPro and PF0642 in Pfam. Example 4 gives the amino acid coordinates of the CCCH domains as present in the polypeptides of Table A3. Preferred YEF1 polypeptides useful in the methods of the invention comprise a CCCH domain or a domain having or a domain having in increasing order of preference at least 50%, 55%, 60%, 65%, 70%, 75%, 80%, 85%, 90%, 95% or more sequence identity to any of the CCCH domains as set forth in Table C3 of Example 4.

Typically NDP1 domains are located at the N-terminus, while RRM domains are located at the C-terminus of YEF1 polypeptides. CCCH domains are typically located upstream, at the N-terminus, of the RRM domains.

YEF1 polypeptides may comprise a multiplicity of NDP1, RRM and/or CCCH domains. Preferably the NPD1 and the RRM domains occur in the YEF1 polypeptides useful in the methods of the invention in increasing order of preference one, two, three, four, up to ten times.

Additionally YEF1 polypeptides may comprise one or more of the conserved amino acid motifs as follows:

(SEQ ID NO: 277)
(i) Motif I: MIRLA
(SEQ ID NO: 278)
(ii) Motif II: ESLEHNLPDSPFASPTK

A further preferred YEF1 protein useful in the methods of the invention comprises a motif having at least 75%, 80%, 85%, 90% or 95% sequence identity to SEQ ID NO: 277 (Motif I) and/or a motif having at least 50%, 55%, 60%, 65%, 70%, 75%, 80%, 85%, 90% or 95% sequence identity to SEQ ID NO: 278 (Motif II).

A person skilled in the art will readily be able to identify motifs having at least 75%, 80%, 85%, 90% or 95% sequence identity to Motif I and/or motifs having at least 50%, 55%, 60%, 65%, 70%, 75%, 80%, 85% or 90% sequence identity to Motif II. This may easily be achieved by making a protein sequence alignment and searching for homologous regions.

Further preferred YEF1 polypeptides useful in the methods of the invention are orthologues or paralogues of any one of the amino acid sequences given in Table A3. More preferably the YEF1 polypeptide abovementioned is any of the polypeptide of Table A3. Most preferably is SEQ ID NO: 249.

Alternatively, the YEF1 protein has in increasing order of preference at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 249. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered.

Preferably, the polypeptide sequence which when used in the construction of a phylogenetic tree, such as the one depicted in FIG. 11, clusters with any polypeptide comprised in the YEF1 group which comprises the amino acid sequence represented by SEQ ID NO: 249 rather than with any other group.

A “subgroup III Grx polypeptide” as defined herein refers to any polypeptide sequence which when used in the construction of a phylogenetic tree, such as the ones depicted in FIGS. 16 to 18, clusters with members of subgroup III Grx polypeptides (which comprise the amino acid sequence represented by SEQ ID NO: 283) rather than with members of subgroup I or subgroup II.

Preferably, the sequence of the active site of the subgroup III Grx is: CCxx, where x can be any amino acid.

Further preferably, the sequence of the active site of the subgroup III Grx is CCxS, where x is any amino acid.

Most preferably, the sequence of the active site of the subgroup III Grx is CCMS, where x is any amino acid.

In A. thaliana, all the proteins of subgroup III possess active sites of the CC[M/L][C/S] form, except one with a CCLG active site (At1g03850). The situation is almost similar in P. trichocarpa; only one sequence is divergent, with a CYMS active site. In O. sativa, the active site sequences vary compared with A. thaliana or P. trichocarpa. Some atypical active sites, differing in the second or fourth position or both, such as CFMC or CPMC, CGMC, CGMS, CCMA, CCLI, and CYMA, are found in O. sativa [respective accession numbers Os01g70990, Os12g35340, Os11g43520, Os05g05730, Os01g13950, Os01g47760, and Os01g09830 of The Institute of Genome Research (TIGR)]. These sequences are not restricted to O. sativa, since similar active site sequences are mostly present in Poaceae such as Hordeum vulgare, Triticum aestivum or Zea mays. See Rouhier et al., 2006.

In contrast, subgroup I contains Grxs with CPYC, CGYC, CPFC, and CSY[C/S] active sites. This group comprises five different classes of Grx (Grx C1-04 and S12) which differ in their active site sequences. The nomenclature used (C or S) is based on the presence of a cysteine or a serine in the fourth position of the active site (CxxC or CxxS).

The proteins of subgroup II possess CGFS active sites, but they differ in the number of repeated modules (one in Grx S14, S15 and S16, and three in Grx S17) and thus in their size, ranging from 170 to 492 amino acids.

Subgroup III Grxs are typically located in the cytosol.

The subgroup III Grx typically has in increasing order of preference at least 50%, 51%, 52%, 53%, 54%, 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 283. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered.

A “Sister of FT protein or a homologue thereof” as defined herein refers to any polypeptide having in increasing order of preference at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 440. The overall sequence identity is determined using a global alignment algorithm, such as the Needleman Wunsch algorithm in the program GAP (GCG Wisconsin Package, Accelrys), preferably with default parameters. Compared to overall sequence identity, the sequence identity will generally be higher when only conserved domains or motifs are considered.

Preferably, polypeptide sequence useful in the methods of the invention, and nucleic acids encoding the same, when used in the construction of a phylogenetic tree of FT sequences, cluster with the group comprising the amino acid sequence represented by SEQ ID NO: 440 rather than with any other group.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

Furthermore, PRE-like polypeptides (at least in their native form) may have DNA-binding activity. This has already been shown for PRE-like proteins of animal origin, and tools and techniques for measuring DNA-binding activity are well known in the art.

In addition, PRE-like polypeptides, when expressed in rice according to the methods of the present invention as outlined in Examples 7 and 8, give plants having increased yield related traits, in particular increased seed size.

Furthermore, SCE1 polypeptides typically have sumoylation activity. Tools and techniques for measuring sumoylation activity are well known in the art. Further details are provided in Example 6.2.

In addition, SCE1 polypeptides, when expressed in rice according to the methods of the present invention as outlined in Examples 6 and 7, give plants having increased yield related traits, in particular increased shoot and/or root biomass.

Furthermore, YEF1 polypeptides typically have RNA-binding activity. Tools and techniques for measuring RNA-binding activity are well known in the art. For example, RNA-binding activity may readily be determined in vitro or in vivo using techniques well known in the art. Examples of in vitro assays include: nucleic acid binding assays using North-Western and/or South-Western analysis (Suzuki et al. Plant Cell Physiol. 41 (3): 282-288 (2000)); RNA binding assays using UV cross linking; Electrophoretic Mobility Shift Assay for RNA Binding Proteins (Smith, RNA-Protein Interactions—A Practical Approach 1998, University of Cambridge). Examples of in vivo assays include: TRAP (translational repression assay procedure) (Paraskeva E, Atzberger A, Hentze M W: A translational repression assay procedure (TRAP) for RNA-protein interactions in vivo. PNAS 1998 Feb. 3; 95(3): 951-6.).

In addition, YEF1 polypeptides, when expressed in rice according to the methods of the present invention as outlined in Examples 6 and 7, give plants having increased yield related traits, in particular increased total weight of the seeds per plant. Further details are provided in the example section.

Furthermore, subgroup III Grx polypeptides (at least in their native form) typically catalyse the reduction of disulfide bonds in proteins converting glutathione (GSH) to glutathione disulfide (GSSG). GSSG is in turn recycled to GSH by the enzyme glutathione reductase at the expense of NADPH. During the reaction cycle it is thought that a cysteine pair in the active site of glutaredoxin is converted to a disulfide.

In addition, subgroup III Grx polypeptides, when expressed in rice according to the methods of the present invention as outlined in the Examples section herein, give plants having enhanced yield related traits, in particular increased aboveground area, emergence vigour, total seeds weight, total number of seeds, number of filled seeds, fill rate, number of flowers per panicle, harvest index and TKW, each relative to control plants.

In addition, Sister of FT proteins or homologues thereof, when expressed in rice according to the methods of the present invention as outlined in the Examples section, give plants having an altered root:shoot ratio relative to control plants.

Concerning PRE-like sequences, the present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any PRE-like-encoding nucleic acid or PRE-like polypeptide as defined herein.

Concerning PRE-like sequences, examples of nucleic acids encoding PRE-like polypeptides are given in Table A1 of Example 1 herein. Such nucleic acids are useful in performing the methods of the invention. The amino acid sequences given in Table A1 of Example 1 are example sequences of orthologues and paralogues of the PRE-like polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A1 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1 or SEQ ID NO: 2, the second BLAST would therefore be against Triticum aestivum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning SCE1 sequences, the present invention is illustrated by transforming plants with the nucleic acid sequence represented by SEQ ID NO: 199, encoding the polypeptide sequence of SEQ ID NO: 200. However, performance of the invention is not restricted to these sequences; the methods of the invention may advantageously be performed using any SCE1-encoding nucleic acid or SCE1 polypeptide as defined herein.

Concerning SCE1 sequences, examples of nucleic acids encoding SCE1 polypeptides are given in Table A2 of Example 1 herein. Such nucleic acids are useful in performing the methods of the invention. The amino acid sequences given in Table A2 of Example 1 are example sequences of orthologues and paralogues of the SCE1 polypeptide represented by SEQ ID NO: 200, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A2 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 199 or SEQ ID NO: 200, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning YEF1 sequences, the present invention is illustrated by transforming plants with the nucleic acid sequence represented by SEQ ID NO: 248, encoding the polypeptide sequence of SEQ ID NO: 249. However, performance of the invention is not restricted to these sequences; the methods of the invention may advantageously be performed using any YEF1-encoding nucleic acid or YEF1 polypeptide as defined herein.

Concerning YEF1 sequences, examples of nucleic acids encoding YEF1 polypeptides are given in Table A3 of Example 1 herein. Such nucleic acids are useful in performing the methods of the invention. The amino acid sequences given in Table A3 of Example 1 are example sequences of orthologues and paralogues of the YEF1 polypeptide represented by SEQ ID NO: 249, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A3 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 248 or SEQ ID NO: 249, the second BLAST would therefore be against Lycopersicum esculentum sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning subgroup III Grx sequences, the present invention is illustrated by transforming plants with the nucleic acid sequence represented by SEQ ID NO: 282, encoding the polypeptide sequence of SEQ ID NO: 283. However, performance of the invention is not restricted to these sequences; the methods of the invention may advantageously be performed using any subgroup III Grx-encoding nucleic acid or subgroup III Grx polypeptide as defined herein.

Concerning subgroup III Grx sequences, examples of nucleic acids encoding subgroup III Grx polypeptides are given in Table A4 of Example 1 herein. Such nucleic acids are useful in performing the methods of the invention. The amino acid sequences given in Table A4 of Example 1 are example sequences of orthologues and paralogues of the subgroup III Grx polypeptide represented by SEQ ID NO: 283, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A4 of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 282 or SEQ ID NO: 283, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

Concerning Sister of FT sequences, the present invention is illustrated by transforming plants with the nucleic acid sequence represented by SEQ ID NO: 439, encoding the polypeptide sequence of SEQ ID NO: 440. However, performance of the invention is not restricted to these sequences; the methods of the invention may advantageously be performed using any Sister of FT-encoding nucleic acid or Sister of FT protein or homologue thereof as defined herein.

Concerning Sister of FT sequences, orthologues and paralogues of the sequence represented by SEQ ID NO: 440 are also useful in performing methods of the invention, the terms “orthologues” and “paralogues” being as defined herein. Orthologues and paralogues may readily be identified by performing a so-called reciprocal blast search. Typically, this involves a first BLAST involving BLASTing a query sequence (for example using SEQ ID NO: 439 or SEQ ID NO: 440) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 439 or SEQ ID NO: 440, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A1-A4 of Example 1, the terms “homologue” and “derivative” being as defined herein. Also useful in the methods of the invention are nucleic acids encoding homologues and derivatives of orthologues or paralogues of any one of the amino acid sequences given in Table A1-A4 of Example 1. Homologues and derivatives useful in the methods of the present invention have substantially the same biological and functional activity as the unmodified protein from which they are derived.

Nucleic acid variants may also be useful in practising the methods of the invention. Examples of such variants include nucleic acids encoding homologues and derivatives of a Sister of FT as defined herein or nucleic acids encoding homologues and derivatives of SEQ ID NO: 2, the terms “homologue” and “derivative” being as defined herein. Also useful in the methods of the invention are nucleic acids encoding homologues and derivatives of orthologues or paralogues of SEQ ID NO: 2. Homologues and derivatives useful in the methods of the present invention have substantially the same biological and functional activity as the unmodified protein from which they are derived.

Nucleic acid variants useful in practising the methods of the invention include portions of nucleic acids encoding PRE-like polypeptides, or SCE1, or YEF1, or subgroup III Grx, or Sister of FT polypeptides, nucleic acids hybridising to nucleic acids encoding PRE-like polypeptides, or SCE1, or YEF1, or subgroup III Grx, or Sister of FT polypeptides, splice variants of nucleic acids encoding PRE-like polypeptides, or SCE1, or YEF1, or subgroup III Grx, or Sister of FT polypeptides, allelic variants of nucleic acids encoding PRE-like polypeptides and variants of nucleic acids encoding PRE-like polypeptides, or SCE1, or YEF1, or subgroup III Grx, or Sister of FT polypeptides obtained by gene shuffling. The terms hybridising sequence, splice variant, allelic variant and gene shuffling are as described herein.

Nucleic acids encoding PRE-like polypeptides, or SCE1, or YEF1, or subgroup III Grx, or Sister of FT polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in any of Table A1 to A4 of Example 1, or a portion of a nucleic acid encoding an orthologue, paralogue or homologue of any of the amino acid sequences given in Table A1 to A4 of Example 1.

Nucleic acids encoding Sister of FT proteins or homologues thereof need not be full-length nucleic acids, since performance of the methods of the invention does not rely on the use of full-length nucleic acid sequences. According to the present invention, there is provided a method for altering the root:shoot ratio in plants, comprising introducing and expressing in a plant a portion of a nucleic acid sequence of SEQ ID NO: 1, or a portion of a nucleic acid encoding an orthologue, paralogue or homologue of the amino acid sequence of SEQ ID NO: 2.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Concerning PRE-like sequences, portions useful in the methods of the invention, encode a PRE-like polypeptide as defined herein, and have substantially the same biological activity as the amino acid sequences given in Table A1 of Example 1. Preferably, the portion is a portion of any one of the nucleic acids given in Table A1 of Example 1, or is a portion of a nucleic acid encoding an orthologue or paralogue of any one of the amino acid sequences given in Table A1 of Example 1. Preferably the portion is at least 100, 150, 200, 250, 300, 350 consecutive nucleotides in length, the consecutive nucleotides being of any one of the nucleic acid sequences given in Table A1 of Example 1, or of a nucleic acid encoding an orthologue or paralogue of any one of the amino acid sequences given in Table A1 of Example 1. Most preferably the portion is a portion of the nucleic acid of Table A1 of Example 1. Preferably, the portion encodes a fragment of an amino acid sequence which, when used in the construction of a phylogenetic tree, such as the one depicted in FIG. 3, clusters with the group of PRE-like polypeptides comprising the amino acid sequence represented by SEQ ID NO: 2 rather than with any other group.

Concerning SCE1 sequences, portions useful in the methods of the invention, encode an SCE1 polypeptide as defined herein, and have substantially the same biological activity as the amino acid sequences given in Table A2 of Example 1. Preferably, the portion is a portion of any one of the nucleic acids given in Table A2 of Example 1, or is a portion of a nucleic acid encoding an orthologue or paralogue of any one of the amino acid sequences given in Table A2 of Example 1. Preferably the portion is at least 100, 200, 300, 400, 500, 600, 700, 800, 900, 1000 consecutive nucleotides in length, the consecutive nucleotides being of any one of the nucleic acid sequences given in Table A2 of Example 1, or of a nucleic acid encoding an orthologue or paralogue of any one of the amino acid sequences given in Table A2 of Example 1. Most preferably the portion is a portion of the nucleic acid of SEQ ID NO: 199. Preferably, the portion encodes a fragment of an amino acid sequence which, when used in the construction of a phylogenetic tree, such as the one depicted in FIG. 6 of Kraft et al. 2005, clusters with the group I comprising the amino acid sequence of AtSCE1a rather than with any other group.

Concerning YEF1 sequences, portions useful in the methods of the invention, encode a YEF1 polypeptide as defined herein, and have substantially the same biological activity as the amino acid sequences given in Table A3 of Example 1. Preferably, the portion is a portion of any one of the nucleic acids given in Table A3 of Example 1, or is a portion of a nucleic acid encoding an orthologue or paralogue of any one of the amino acid sequences given in Table A3 of Example 1. Preferably the portion is at least 500, 550, 600, 650, 700, 750, 800, 850, 900, 950, 1000, 1500, 2000 consecutive nucleotides in length, the consecutive nucleotides being of any one of the nucleic acid sequences given in Table A3 of Example 1, or of a nucleic acid encoding an orthologue or paralogue of any one of the amino acid sequences given in Table A3 of Example 1. Most preferably the portion is a portion of the nucleic acid of SEQ ID NO: 248. Preferably, the portion encodes a fragment of an amino acid sequence which, when used in the construction of a phylogenetic tree, such as the one depicted in FIG. 11, clusters with any polypeptide comprised in the YEF1 group which comprises the amino acid sequence represented by SEQ ID NO: 249 rather than with any other group.

Concerning subgroup III Grx sequences, portions useful in the methods of the invention, encode a subgroup III Grx polypeptide as defined herein, and have substantially the same biological activity as the amino acid sequences given in Table A4 of Example 1. Preferably, the portion is a portion of any one of the nucleic acids given in Table A4 of Example 1, or is a portion of a nucleic acid encoding an orthologue or paralogue of any one of the amino acid sequences given in Table A4 of Example 1. Preferably the portion is at least 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, consecutive nucleotides in length, the consecutive nucleotides being of any one of the nucleic acid sequences given in Table A4 of Example 1, or of a nucleic acid encoding an orthologue or paralogue of any one of the amino acid sequences given in Table A4 of Example 1. Most preferably the portion is a portion of the nucleic acid of SEQ ID NO: 282.

Preferably, the portion encodes a polypeptide with a CCxx active site, where x can be any amino acid.

Further preferably, the portion encodes a polypeptide with a CCxS active site, where x is any amino acid.

Most preferably, the portion encodes a polypeptide with a CCMS active site.

Concerning Sister of FT sequences, portions useful in the methods of the invention, encode a Sister of FT protein or a homologue thereof as defined herein, and have substantially the same biological activity as the amino acid sequence of SEQ ID NO: 440. Preferably, the portion is a portion of the nucleic acid represented by SEQ ID NO: 439, or is a portion of a nucleic acid encoding an orthologue or paralogue of the amino acid sequence of SEQ ID NO: 440. Preferably the portion is at least 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, consecutive nucleotides in length, the consecutive nucleotides being of SEQ ID NO: 439, or of a nucleic acid encoding an orthologue or paralogue of the amino acid sequence of SEQ ID NO: 440. Most preferably the portion is a portion of the nucleic acid of SEQ ID NO: 439.

Another nucleic acid variant useful in the methods of the invention is a nucleic acid capable of hybridising, under reduced stringency conditions, preferably under stringent conditions, with a nucleic acid encoding a PRE-like polypeptides, or SCE1, or YEF1, or subgroup III Grx polypeptide, or a Sister of FT protein or a homologue thereof as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A1-A4 of Example 1, or comprising introducing and expressing in a plant a nucleic acid capable of hybridising to a nucleic acid encoding an orthologue, paralogue or homologue of any of the nucleic acid sequences given in Table A1-A4 of Example 1.

Concerning Sister of FT, according to the present invention, there is provided a method for altering the root:shoot ratio in plants, comprising introducing and expressing in a plant a nucleic acid capable of hybridizing to SEQ ID NO: 439, or comprising introducing and expressing in a plant a nucleic acid capable of hybridising to a nucleic acid encoding an orthologue, paralogue or homologue of SEQ ID NO: 440. Hybridising sequences useful in the methods of the invention encode a Sister of FT protein or a homologue thereof as defined herein, having substantially the same biological activity as the amino acid sequence of SEQ ID NO: 440.

Another nucleic acid variant useful in the methods of the invention is a nucleic acid capable of hybridising, under reduced stringency conditions, preferably under stringent conditions, with a nucleic acid encoding a Sister of FT protein or a homologue thereof as defined herein, or with a portion as defined herein.

Concerning PRE-like sequences, hybridising sequences useful in the methods of the invention encode a PRE-like polypeptide as defined herein, having substantially the same biological activity as the amino acid sequences given in Table A1 of Example 1. Preferably, the hybridising sequence is capable of hybridising to any one of the nucleic acids given in Table A1 of Example 1, or to a portion of any of these sequences, a portion being as defined above, or the hybridising sequence is capable of hybridising to a nucleic acid encoding an orthologue or paralogue of any one of the amino acid sequences given in Table A1 of Example 1. Most preferably, the hybridising sequence is capable of hybridising to a nucleic acid as represented by SEQ ID NO: 1 or to a portion thereof.

Concerning SCE1 sequences, hybridising sequences useful in the methods of the invention encode an SCE1 polypeptide as defined herein, having substantially the same biological activity as the amino acid sequences given in Table A2 of Example 1. Preferably, the hybridising sequence is capable of hybridising to any one of the nucleic acids given in Table A2 of Example 1, or to a portion of any of these sequences, a portion being as defined above, or the hybridising sequence is capable of hybridising to a nucleic acid encoding an orthologue or paralogue of any one of the amino acid sequences given in Table A2 of Example 1. Most preferably, the hybridising sequence is capable of hybridising to a nucleic acid as represented by SEQ ID NO: 199 or to a portion thereof.

Concerning YEF1 sequences, hybridising sequences useful in the methods of the invention encode a YEF1 polypeptide as defined herein, having substantially the same biological activity as the amino acid sequences given in Table A3 of Example 1. Preferably, the hybridising sequence is capable of hybridising to any one of the nucleic acids given in Table A3 of Example 1, or to a portion of any of these sequences, a portion being as defined above, or the hybridising sequence is capable of hybridising to a nucleic acid encoding an orthologue or paralogue of any one of the amino acid sequences given in Table A3 of Example 1. Most preferably, the hybridising sequence is capable of hybridising to a nucleic acid as represented by SEQ ID NO: 248 or to a portion thereof.

Concerning subgroup III Grx sequences, hybridising sequences useful in the methods of the invention encode a polypeptide as defined herein, having substantially the same biological activity as the amino acid sequences given in Table A4 of Example 1. Preferably, the hybridising sequence is capable of hybridising to any one of the nucleic acids given in Table A4 of Example 1, or to a portion of any of these sequences, a portion being as defined above, or the hybridising sequence is capable of hybridising to a nucleic acid encoding an orthologue or paralogue of any one of the amino acid sequences given in Table A4 of Example 1. Most preferably, the hybridising sequence is capable of hybridising to a nucleic acid as represented by SEQ ID NO: 282 or to a portion thereof.

Concerning Sister of FT sequences, according to the present invention, there is provided a method for altering the root:shoot ratio in plants, comprising introducing and expressing in a plant a nucleic acid capable of hybridizing to SEQ ID NO: 439, or comprising introducing and expressing in a plant a nucleic acid capable of hybridising to a nucleic acid encoding an orthologue, paralogue or homologue of SEQ ID NO: 440. Hybridising sequences useful in the methods of the invention encode a Sister of FT protein or a homologue thereof as defined herein, having substantially the same biological activity as the amino acid sequence of SEQ ID NO: 440.

Concerning PRE-like sequences, preferably, the hybridising sequence encodes a polypeptide with an amino acid sequence which, when full-length and used in the construction of a phylogenetic tree, such as the one depicted in FIG. 3, clusters with the group of PRE-like polypeptides comprising the amino acid sequence represented by SEQ ID NO: 2 rather than with any other group.

Concerning SCE1 sequences, preferably, the hybridising sequence encodes a polypeptide with an amino acid sequence which, when used in the construction of a phylogenetic tree, such as the one depicted in FIG. 6 from Kraft et al. 2005, clusters with the group I comprising the amino acid sequence of AtSCE1a rather than with any other group.

Concerning YEF1 sequences, preferably, the hybridising sequence encodes a polypeptide with an amino acid sequence which, when full-length and used in the construction of a phylogenetic tree, such as the one depicted in FIG. 11, clusters with any polypeptide comprised in the YEF1 group which comprises the amino acid sequence represented by SEQ ID NO: 249 rather than with any other group.

Concerning subgroup III Grx sequences, the hybridising sequence encodes a polypeptide sequence which when used in the construction of a phylogenetic tree, such as the ones depicted in FIGS. 16 to 18, clusters with members of subgroup III Grx polypeptides (which comprise the amino acid sequence represented by SEQ ID NO: 283) rather than with members of subgroup I or subgroup II.

Preferably, the hybridizing sequence encodes a polypeptide with a CCxx active site, where x can be any amino acid.

Further preferably, the hybridizing sequence encodes a polypeptide with a CCxS active site, where x is any amino acid.

Most preferably, the hybridizing sequence encodes a polypeptide with a CCMS active site.

Another nucleic acid variant useful in the methods of the invention is a splice variant encoding PRE-like polypeptides, or SCE1, or YEF1, or subgroup III Grx polypeptide, or a Sister of FT protein or a homologue thereof as defined hereinabove, a splice variant being as defined herein.

Concerning PRE-like polypeptides, or SCE1, or YEF1, or subgroup III Grx sequences, according to the present invention, there is provided a method for enhancing yield-related traits in plants, comprising introducing and expressing in a plant a splice variant of any one of the nucleic acid sequences given in Table A1-A4 of Example 1, or a splice variant of a nucleic acid encoding an orthologue, paralogue or homologue of any of the amino acid sequences given in Table A1-A4 of Example 1.

Concerning Sister of FT sequences, according to the present invention, there is provided a method for altering root:shoot ratio in plants, comprising introducing and expressing in a plant a splice variant of SEQ ID NO: 439, or a splice variant of a nucleic acid encoding an orthologue, paralogue or homologue of SEQ ID NO: 440.

Concerning PRE-like sequences, preferred splice variants are splice variants of a nucleic acid represented by SEQ ID NO: 1, or a splice variant of a nucleic acid encoding an orthologue or paralogue of SEQ ID NO: 2. Preferably, the amino acid sequence encoded by the splice variant, when used in the construction of a phylogenetic tree, such as the one depicted in FIG. 3, clusters with the group of PRE-like polypeptides comprising the amino acid sequence represented by SEQ ID NO: 2 rather than with any other group.

Concerning SCE1 sequences, preferred splice variants are splice variants of a nucleic acid represented by SEQ ID NO: 199, or a splice variant of a nucleic acid encoding an orthologue or paralogue of SEQ ID NO: 200. Preferably, the amino acid sequence encoded by the splice variant, when used in the construction of a phylogenetic tree, such as the one depicted in FIG. 6 from Kraft et al. 2005, clusters with the group I comprising the amino acid sequence of AtSCE1a rather than with any other group.

Concerning YEF1 sequences, preferred splice variants are splice variants of a nucleic acid represented by SEQ ID NO: 248, or a splice variant of a nucleic acid encoding an orthologue or paralogue of SEQ ID NO: 249. Preferably, the amino acid sequence encoded by the splice variant, when used in the construction of a phylogenetic tree, such as the one depicted in FIG. 11, clusters with any polypeptide comprised in the YEF1 group which comprises the amino acid sequence represented by SEQ ID NO: 249 rather than with any other group.

Concerning subgroup III Grx sequences, preferred splice variants are splice variants of a nucleic acid represented by SEQ ID NO: 282, or a splice variant of a nucleic acid encoding an orthologue or paralogue of SEQ ID NO: 283.

The splice variant encodes a polypeptide sequence which when used in the construction of a phylogenetic tree, such as the ones depicted in FIGS. 16 to 18, clusters with members of subgroup III Grx polypeptides (which comprise the amino acid sequence represented by SEQ ID NO: 283) rather than with members of subgroup I or subgroup II.

Preferably, the splice variant encodes a polypeptide with a CCxx active site, where x can be any amino acid.

Further preferably, the splice variant encodes a polypeptide with a CCxS active site, where x is any amino acid.

Most preferably, the splice variant encodes a polypeptide with a CCMS active site.

Another nucleic acid variant useful in performing the methods of the invention is an allelic variant of a nucleic acid PRE-like polypeptides, or SCE1, or YEF1, or subgroup III Grx polypeptide, or a Sister of FT protein or a homologue thereof as defined hereinabove, an allelic variant being as defined herein.

Concerning PRE-like polypeptides, or SCE1, or YEF1, or subgroup III Grx sequences, according to the present invention, there is provided a method for enhancing yield-related traits in plants, comprising introducing and expressing in a plant an allelic variant of any one of the nucleic acids given in Table A1-A4 of Example 1, or comprising introducing and expressing in a plant an allelic variant of a nucleic acid encoding an orthologue, paralogue or homologue of any of the amino acid sequences given in Table A1-A4 of Example 1.

Concerning Sister of FT sequences, according to the present invention, there is provided a method for altering root:shoot ratio in plants, comprising introducing and expressing in a plant a splice variant of SEQ ID NO: 439, or a splice variant of a nucleic acid encoding an orthologue, paralogue or homologue of SEQ ID NO: 440.

Another nucleic acid variant useful in performing the methods of the invention is an allelic variant of a nucleic acid encoding a Sister of FT protein or a homologue thereof as defined hereinabove, an allelic variant being as defined herein.

According to the present invention, there is provided a method for altering the root:shoot ratio in plants, comprising introducing and expressing in a plant an allelic variant of SEQ ID NO: 439, or comprising introducing and expressing in a plant an allelic variant of a nucleic acid encoding an orthologue, paralogue or homologue of SEQ ID NO: 440.

Concerning PRE-like sequences, the allelic variants useful in the methods of the present invention have substantially the same biological activity as the PRE-like polypeptide of SEQ ID NO: 2 and any of the amino acids depicted in Table A1 of Example 1. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2. Preferably, the amino acid sequence encoded by the allelic variant, when used in the construction of a phylogenetic tree, such as the one depicted in FIG. 3, clusters with the PRE-like polypeptides comprising the amino acid sequence represented by SEQ ID NO: 2 rather than with any other group.

Concerning SCE1 sequences, the allelic variants useful in the methods of the present invention have substantially the same biological activity as the SCE1 polypeptide of SEQ ID NO: 200 and any of the amino acids depicted in Table A2 of Example 1. Allelic variants exist in nature, and encompassed within the methods of the present invention is the use of these natural alleles. Preferably, the allelic variant is an allelic variant of SEQ ID NO: 199 or an allelic variant of a nucleic acid encoding an orthologue or paralogue of SEQ ID NO: 200. Preferably, the amino acid sequence encoded by the allelic variant when used in the construction of a phylogenetic tree, such as the one depicted in FIG. 6 from Kraft et al. 2005, clusters with the group I comprising the amino acid sequence of AtSCE1a rather than with any other group.

Concerning YEF1 sequences, the allelic variants useful in the methods of the present invention have substantially the same biological activity as the YEF1 polypeptide of SEQ ID NO: 249 and any of the amino acids depicted in Table A3 of Example 1. Allelic variants exist in nature, and encompassed within the methods of the present invention is the use of these natural alleles. Preferably, the allelic variant is an allelic variant of SEQ ID NO: 248 or an allelic variant of a nucleic acid encoding an orthologue or paralogue of SEQ ID NO: 249. Preferably, the amino acid sequence encoded by the allelic variant, when used in the construction of a phylogenetic tree such as the one depicted in FIG. 11, clusters with any polypeptide comprised in the YEF1 group which comprises the amino acid sequence represented by SEQ ID NO: 249 rather than with any other group.

Concerning subgroup III Grx sequences, the polypeptides encoded by allelic variants useful in the methods of the present invention have substantially the same biological activity as the subgroup III Grx polypeptide of SEQ ID NO: 283 and any of the amino acids depicted in Table A4 of Example 1. Allelic variants exist in nature, and encompassed within the methods of the present invention is the use of these natural alleles. Preferably, the allelic variant is an allelic variant of SEQ ID NO: 282 or an allelic variant of a nucleic acid encoding an orthologue or paralogue of SEQ ID NO: 283.

The allelic variant encodes a polypeptide sequence which when used in the construction of a phylogenetic tree, such as the ones depicted in FIGS. 16 to 18, clusters with members of subgroup III Grx polypeptides (which comprise the amino acid sequence represented by SEQ ID NO: 283) rather than with members of subgroup I or subgroup II.

Preferably, the allelic variant encodes a polypeptide with a CCxx active site, where x can be any amino acid.

Further preferably, the allelic variant encodes a polypeptide with a CCxS active site, where x is any amino acid.

Most preferably, the allelic variant encodes a polypeptide with a CCMS active site.

Concerning Sister of FT sequences, the allelic variants useful in the methods of the present invention have substantially the same biological activity as the Sister of FT protein or a homologue thereof of SEQ ID NO: 440. Allelic variants exist in nature, and encompassed within the methods of the present invention is the use of these natural alleles. Preferably, the allelic variant is an allelic variant of SEQ ID NO: 439 or an allelic variant of a nucleic acid encoding an orthologue or paralogue of SEQ ID NO: 440.

Gene shuffling or directed evolution may also be used to generate variants of nucleic acids encoding PRE-like polypeptides, or SCE1, or YEF1, or subgroup III Grx polypeptides, or Sister of FT proteins or homologues thereof as defined above; the term “gene shuffling” being as defined herein.

Concerning PRE-like polypeptides, or SCE1, or YEF1, or subgroup III Grx sequences, according to the present invention, there is provided a method for enhancing yield-related traits in plants, comprising introducing and expressing in a plant a variant of any one of the nucleic acid sequences given in Table A1 to A4 of Example 1, or comprising introducing and expressing in a plant a variant of a nucleic acid encoding an orthologue, paralogue or homologue of any of the amino acid sequences given in Table A1 to A4 of Example 1, which variant nucleic acid is obtained by gene shuffling.

Concerning Sister of FT sequences, according to the present invention, there is provided a method for altering the root:shoot ratio of plants, comprising introducing and expressing in a plant a variant of the nucleic acid sequences of SEQ ID NO: 439, or comprising introducing and expressing in a plant a variant of a nucleic acid encoding an orthologue, paralogue or homologue of any of the amino acid sequences of SEQ ID NO: 440, which variant nucleic acid is obtained by gene shuffling.

Concerning PRE-like sequences, preferably, the amino acid sequence encoded by the variant nucleic acid obtained by gene shuffling, when used in the construction of a phylogenetic tree such as the one depicted in FIG. 3, clusters with the group of PRE-like polypeptides comprising the amino acid sequence represented by SEQ ID NO: 2 rather than with any other group.

Concerning SCE1 sequences, preferably, the amino acid sequence encoded by the variant nucleic acid obtained by gene shuffling, when used in the construction of a phylogenetic tree, such as the one depicted in FIG. 6 of Kraft et al. 2005, clusters with the group I comprising the amino acid sequence of AtSCE1a rather than with any other group.

Concerning SCE1 sequences, preferably, the amino acid sequence encoded by the variant nucleic acid obtained by gene shuffling, when used in the construction of a phylogenetic tree such as the one depicted in FIG. 11, clusters with any polypeptide comprised in the YEF1 group which comprises the amino acid sequence represented by SEQ ID NO: 249 rather than with any other group.

Concerning subgroup III Grx sequences, the amino acid sequence encoded by the variant nucleic acid obtained by gene shuffling, when used in the construction of a phylogenetic tree, such as the ones depicted in FIGS. 16 to 18, clusters with members of subgroup III Grx polypeptides (which comprise the amino acid sequence represented by SEQ ID NO: 283) rather than with members of subgroup I or subgroup II.

Preferably, the variant nucleic acid obtained by gene shuffling encodes a polypeptide with a CCxx active site, where x can be any amino acid.

Further preferably, the variant nucleic acid obtained by gene shuffling encodes a polypeptide with a CCxS active site, where x is any amino acid.

Most preferably, the variant nucleic acid obtained by gene shuffling encodes a polypeptide with a CCMS active site.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding PRE-like polypeptides may be derived from any natural or artificial source. The nucleic acid may be modified from its native form in composition and/or genomic environment through deliberate human manipulation. Preferably the PRE-like polypeptide-encoding nucleic acid is from a plant, further preferably from a monocotyledonous plant, more preferably from the family Poaceae, most preferably the nucleic acid is from Triticum aetivum.

Nucleic acids encoding SCE1 polypeptides may be derived from any natural or artificial source. The nucleic acid may be modified from its native form in composition and/or genomic environment through deliberate human manipulation. Preferably the SCE1 polypeptide-encoding nucleic acid is from a plant, further preferably from a dicotyledonous plant, more preferably from the family brasicaceae, most preferably the nucleic acid is from Arabidopsis thaliana.

Advantageously, the present invention provides hitherto unknown SCE1 nucleic acid and polypeptide sequences.

According to a further embodiment of the present invention, there is provided an isolated nucleic acid molecule comprising:
(i) a nucleic acid represented by SEQ ID NO: 3; SEQ ID NO: 5; SEQ ID NO: 7; SEQ ID NO: 9; SEQ ID NO: 11; SEQ ID NO: 13 and SEQ ID NO: 15;
(ii) a nucleic acid or fragment thereof that is complementary to any one of the SEQ ID NOs given in (i);
(iii) a nucleic acid encoding an SCE1 polypeptide having, in increasing order of preference, at least 70%, 75%, 80%, 85%, 90%, 95%, 96%, 97%, 98%, 99% or more sequence identity to any one of the amino acid sequences given in SEQ ID NO: 4; SEQ ID NO: 6; SEQ ID NO: 8; SEQ ID NO: 10; SEQ ID NO: 12; SEQ ID NO: 14 and SEQ ID NO: 16;
(iv) a nucleic acid capable of hybridizing under stringent conditions to any one of the nucleic acids given in (i), (ii) or (iii) above.

According to a further embodiment of the present invention, there is therefore provided an isolated polypeptide comprising:
(i) an amino acid sequence having, in increasing order of preference, at least 80%, 85%, 90%, 95%, 96%, 97%, 98%, 99% or 100% sequence identity to any one of the amino acid sequences given in SEQ ID NO: 4; SEQ ID NO: 6; SEQ ID NO: 8; SEQ ID NO: 10; SEQ ID NO: 12; SEQ ID NO: 14 and SEQ ID NO: 16;
(ii) derivatives of any of the amino acid sequences given in (i).

Nucleic acids encoding YEF1 polypeptides may be derived from any natural or artificial source. The nucleic acid may be modified from its native form in composition and/or genomic environment through deliberate human manipulation. Preferably the YEF1 polypeptide-encoding nucleic acid is from a plant, further preferably from a dicotyledonous plant, more preferably from the family Solanum, most preferably the nucleic acid is from Lycorpersicum esculenturn.

Nucleic acids encoding subgroup III Grx polypeptides may be derived from any natural or artificial source. The nucleic acid may be modified from its native form in composition and/or genomic environment through deliberate human manipulation. Preferably the subgroup III Grx polypeptide-encoding nucleic acid is from a plant, further preferably from a dicotyledonous plant, more preferably from the family Brassicaceae, preferably from the genus Arabidopsis and most preferably from Arabidopsis thaliana.

Nucleic acids encoding Sister of FT proteins or homologues thereof may be derived from any natural or artificial source. The nucleic acid may be modified from its native form in composition and/or genomic environment through deliberate human manipulation. Preferably the Sister of FT-encoding nucleic acid is from a plant, further preferably from a dicotyledonous plant, more preferably from the family Brassicaceae, more preferably from the genus Arabidopsis, most preferably from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants. Furthermore the term “yield-related trait” as defined herein may encompass an alteration of the ratio of roots to shoots (root:shoot ratio). In the case of PRE-like sequences, the result in increased yield does not encompass increased oil content of seeds.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression in a plant of a nucleic acid encoding a PRE-like polypeptide, or SCE1, or YEF1, or subgroup III Grx polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per square meter (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression in a plant of a nucleic acid encoding a PRE-like polypeptide, or SCE1, or YEF1, or subgroup III Grx polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi, nematodes and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Concerning Sister of FT sequences, an altered root:shoot ratio occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having an altered root:shoot ratio relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises modulating expression in a plant of a nucleic acid encoding a PRE-like polypeptide, or SCE1, or YEF1, or subgroup III Grx polypeptide.

Concerning Sister of FT sequences, performance of the methods of the invention gives plants grown under non-stress conditions or under mild drought conditions altered root:shoot ratio relative to control plants grown under comparable conditions. Therefore, according to the present invention, there is provided a method for altering the root:shoot ratio in plants grown under non-stress conditions or under mild drought conditions, which method comprises modulating expression in a plant of a nucleic acid encoding a Sister of FT protein or a homologue thereof.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises modulating expression in a plant of a nucleic acid encoding a PRE-like, an SCE1, a YEF1, a subgroup III Grx polypeptide. Nutrient deficiency may result from a lack of nutrients such as nitrogen, phosphates and other phosphorous-containing compounds, potassium, calcium, cadmium, magnesium, manganese, iron and boron, amongst others.

Concerning Sister of FT sequences, performance of the methods of the invention gives plants grown under conditions of nutrient deficiency, particularly under conditions of nitrogen deficiency, an altered root:shoot ratio relative to control plants grown under comparable conditions. Therefore, according to the present invention, there is provided a method for altering the root:shoot ratio in plants grown under conditions of nutrient deficiency, which method comprises modulating expression in a plant of a nucleic acid encoding a Sister of FT protein or a homologue thereof. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a PRE-like polypeptide, or SCE1, or YEF1, or subgroup III Grx polypeptide, or a Sister of FT protein or a homologue thereof as defined above.

The invention also provides genetic constructs and vectors to facilitate introduction and/or expression in plants of nucleic acids encoding PRE-like polypeptides, or SCE1, or YEF1, or subgroup III Grx polypeptides, or Sister of FT proteins or homologues thereof. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a PRE-like polypeptide, or SCE1, or YEF1, or subgroup III Grx polypeptide, or a Sister of FT protein or a homologue thereof as defined above;
(b) one or more control sequences capable of driving expression of the nucleic acid sequence of (a); and optionally
(c) a transcription termination sequence.

Preferably, the nucleic acid encoding a PRE-like polypeptide is as defined above. The term “control sequence” and “termination sequence” are as defined herein. Preferably, the construct comprises an expression cassette essentially similar or identical to SEQ ID NO 6, comprising the GOS2 promoter and the nucleic acid encoding the PRE-like polypeptide.

Preferably, the nucleic acid encoding an SCE1 polypeptide is as defined above. The term “control sequence” and “termination sequence” are as defined herein.

Preferably, the nucleic acid encoding a YEF1 polypeptide is as defined above. The term “control sequence” and “termination sequence” are as defined herein.

Preferably, the nucleic acid encoding a subgroup III Grx polypeptide is as defined above. The term “control sequence” and “termination sequence” are as defined herein.

Preferably, the nucleic acid encoding a Sister of FT protein or a homologue thereof is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. Preferably the constitutive promoter is also a ubiquitous promoter. See the “Definitions” section herein for definitions of the various promoter types.

Concerning subgroup III Grx sequences, advantageously, any type of promoter, whether natural or synthetic, may be used to drive expression of the nucleic acid sequence. A green tissue-specific promoter is particularly useful in the methods. See the “Definitions” section herein for definitions of the various promoter types.

It should be clear that the applicability of the present invention is not restricted to the PRE-like polypeptide-encoding nucleic acid represented by SEQ ID NO: 1, nor is the applicability of the invention restricted to expression of a PRE-like polypeptide-encoding nucleic acid when driven by a constitutive promoter.

It should also be clear that the applicability of the present invention is not restricted to the SCE1 polypeptide-encoding nucleic acid represented by SEQ ID NO: 199, nor is the applicability of the invention restricted to expression of an SCE1 polypeptide-encoding nucleic acid when driven by a constitutive promoter.

Furthermore, it should be clear that the applicability of the present invention is not restricted to the YEF1 polypeptide-encoding nucleic acid represented by SEQ ID NO: 248, nor is the applicability of the invention restricted to expression of a YEF1 polypeptide-encoding nucleic acid when driven by a constitutive promoter.

It should be clear that the applicability of the present invention is not restricted to the subgroup III Grx polypeptide-encoding nucleic acid represented by SEQ ID NO: 282, nor is the applicability of the invention restricted to expression of a subgroup III Grx polypeptide-encoding nucleic acid when driven by a green tissue-specific promoter.

It should be clear that the applicability of the present invention is not restricted to the Sister of FT-encoding nucleic acid represented by SEQ ID NO: 439, nor is the applicability of the invention restricted to expression of a Sister of FT-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 5, SEQ ID NO: 247, SEQ ID NO: 281, or SEQ ID NO: 441 most preferably the constitutive promoter is as represented by SEQ ID NO: 5, SEQ ID NO: 247, SEQ ID NO: 281, or SEQ ID NO: 441. See Table 2a in the “Definitions” section herein for further examples of constitutive promoters.

Concerning the subgroup III Grx sequences, the green tissue-specific promoter is preferably a protochlorophyllid reductase promoter, preferably represented by a nucleic acid sequence substantially similar to SEQ ID NO: 436, most preferably the constitutive promoter is as represented by SEQ ID NO: 436. See Table 2g in the “Definitions” section herein for further examples of green tissue-specific promoters.

Optionally, one or more terminator sequences may be used in the construct introduced into a plant. Concerning the subgroup III Grx sequences preferably, the construct comprises an expression cassette essentially similar or identical to SEQ ID NO 282, together with the protochlorophyllid reductase promoter essentially similar or identical to SEQ ID NO: 436, and the T-zein+T-rubisco transcription terminator sequence. Concerning Sister of FT sequences, preferably, the construct comprises an expression cassette essentially similar or identical to SEQ ID NO 439, comprising the GOS2 promoter, and the T-zein+T-rubisco transcription terminator sequence.

Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and expression in a plant of any nucleic acid encoding a PRE-like polypeptide, or SCE1, or YEF1, or subgroup III Grx polypeptide as defined hereinabove. Concerning Sister of FT sequences, the invention also provides a method for the production of transgenic plants having an altered root:shoot ratio relative to control plants, comprising introduction and expression in a plant of any nucleic acid encoding a Sister of FT protein or a homologue thereof as defined hereinabove.

More specifically, the present invention provides a method for the production of transgenic plants having increased enhanced yield-related traits, particularly increased yield or increased seed yield, which method comprises:
(i) introducing and expressing in a plant or plant cell a PRE-like polypeptide, or SCE1, or YEF1, or subgroup III Grx polypeptide-encoding nucleic acid; and
(ii) cultivating the plant cell under conditions promoting plant growth and development.

The nucleic acid of (i) may be any of the nucleic acids capable of encoding a PRE-like polypeptide, or SCE1, or YEF1, or subgroup III Grx polypeptide as defined herein.

Concerning Sister of FT sequences, more specifically, the present invention provides a method for the production of transgenic plants having an altered root:shoot ratio, which method comprises:
(i) introducing and expressing in a plant or plant cell a Sister of FT-encoding nucleic acid; and
(ii) cultivating the plant cell under conditions promoting plant growth and development.

The nucleic acid of (i) may be any of the nucleic acids capable of encoding a Sister of FT protein or a homologue thereof as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Hofgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a PRE-like polypeptide, or SCE1, or YEF1, or subgroup III Grx polypeptide, or a Sister of FT protein or a homologue thereof as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating expression of a PRE-like polypeptide, or SCE1, or YEF1, or subgroup III Grx polypeptide, or a Sister of FT protein or a homologue thereof is by introducing and expressing in a plant a nucleic acid encoding a PRE-like polypeptide, or SCE1, or YEF1, or subgroup III Grx polypeptide, or a Sister of FT protein or a homologue thereof; however the effects of performing the method, i.e. altering the root:shoot ratio in plants and/or enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding PRE-like polypeptides as described herein and use of these PRE-like polypeptides in enhancing any of the aforementioned yield-related traits in plants. The present invention also encompasses use of nucleic acids encoding Sister of FT proteins or homologues thereof as described herein and use of these Sister of FT proteins or homologues thereof in altering plant root:shoot ratio.

Nucleic acids encoding a PRE-like, an SCE1, a YEF1, or a subgroup III Grx polypeptide described herein, or the PRE-like, SCE1, YEF1, or subgroup III Grx polypeptides themselves, may find use in breeding programmes in which a DNA marker is identified which may be genetically linked to a PRE-like, an SCE1, a YEF1, or a subgroup III Grx polypeptide-encoding gene. The nucleic acids/genes, or the PRE-like, the SCE1, the YEF1, or the subgroup III Grx polypeptides themselves may be used to define a molecular marker. This DNA or protein marker may then be used in breeding programmes to select plants having an altered root:shoot ratio and/or having enhanced yield-related traits as defined hereinabove in the methods of the invention. Furthermore, nucleic acids encoding Sister of FT protein or a homologue thereof described herein, or the Sister of FT proteins or homologues thereof themselves, may find use in breeding programmes in which a DNA marker is identified which may be genetically linked to a Sister of FT-encoding gene. The nucleic acids/genes, or the Sister of FT proteins or homologues thereof themselves may be used to define a molecular marker. This DNA or protein marker may then be used in breeding programmes to select plants having an altered root:shoot ratio.

Allelic variants of a PRE-like polypeptide, or SCE1, or YEF1, or subgroup III Grx polypeptide-encoding nucleic acid/gene, or a Sister of FT-encoding may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give an altered root:shoot ratio and/or increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding PRE-like polypeptides, or SCE1, or YEF1, or subgroup III Grx polypeptides or Sister of FT proteins or homologues thereof may also be used as probes for genetically and physically mapping the genes that they are a part of, and as markers for traits linked to those genes. Such information may be useful in plant breeding in order to develop lines with desired phenotypes. Such use of PRE-like polypeptide, or SCE1, or YEF1, or subgroup III Grx polypeptide-encoding nucleic acids, or Sister of FT-encoding nucleic acids requires only a nucleic acid sequence of at least 15 nucleotides in length. The PRE-like polypeptide, or SCE1, or YEF1, or subgroup III Grx polypeptide-encoding nucleic acids, or Sister of FT-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the PRE-like polypeptide, or SCE1, or YEF1, or subgroup III Grx polypeptide-encoding nucleic acids, or Sister of FT-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the PRE-like polypeptide, or SCE1, or YEF1, or subgroup III Grx polypeptide-encoding nucleic acids, or Sister of FT-encoding nucleic acid in the genetic map previously obtained using this population (Botstein et al. (1980) Am. J. Hum. Genet. 32:314-331). The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features. Furthermore, the methods according to the present invention result in plants having an altered root:shoot ratio, as described hereinbefore. These traits may also be combined with other economically advantageous traits, such as further yield-enhancing traits, tolerance to other abiotic and biotic stresses, traits modifying various architectural features and/or biochemical and/or physiological features.

### Items

1. A method for enhancing yield-related traits in plants relative to control plants, comprising modulating expression in a plant of a nucleic acid encoding a PRE-like polypeptide.
2. Method according to item 1, wherein said PRE-like polypeptide comprises one or more of the following motifs: Motif 1 (SEQ ID NO: 7), Motif 2 (SEQ ID NO: 8) and Motif 3 (SEQ ID NO: 9).
3. Method according to item 1 or 2, wherein said modulated expression is effected by introducing and expressing in a plant a nucleic acid encoding a PRE-like polypeptide.
4. Method according to any preceding item, wherein said nucleic acid encoding a PRE-like polypeptide encodes any one of the proteins listed in Table A1 or is a portion of such a nucleic acid, or a nucleic acid capable of hybridising with such a nucleic acid.
5. Method according to any preceding item, wherein said nucleic acid sequence encodes an orthologue or paralogue of any of the proteins given in Table A1.
6. Method according to any preceding item, wherein said enhanced yield-related traits comprise increased yield, preferably increased seed yield relative to control plants, provided that said increased seed yield does not encompass increased seed oil content.
7. Method according to any one of items 1 to 6, wherein said enhanced yield-related traits are obtained under non-stress conditions.
8. Method according to any one of items 1 to 6, wherein said enhanced yield-related traits are obtained under conditions of drought stress, salt stress or nitrogen deficiency.
9. Method according to any one of items 3 to 8, wherein said nucleic acid is operably linked to a constitutive promoter, preferably to a GOS2 promoter, most preferably to a GOS2 promoter from rice.
10. Method according to any preceding item, wherein said nucleic acid encoding a PRE-like polypeptide is of plant origin, preferably from a dicotyledonous plant, further preferably from the family Poaceae, more preferably from the genus Triticum, most preferably from Triticum aestivum.
11. Plant or part thereof, including seeds, obtainable by a method according to any preceding item, wherein said plant or part thereof comprises a recombinant nucleic acid encoding a PRE-like polypeptide.
12. Construct comprising:
   (a) nucleic acid encoding a PRE-like polypeptide as defined in items 1 or 2;
   (b) one or more control sequences capable of driving expression of the nucleic acid sequence of (a); and optionally
   (c) a transcription termination sequence.
13. Construct according to item 12, wherein one of said control sequences is a constitutive promoter, preferably a GOS2 promoter, most preferably a GOS2 promoter from rice.
14. Use of a construct according to item 12 or 13 in a method for making plants having increased yield, particularly increased seed yield relative to control plants.
15. Plant, plant part or plant cell transformed with a construct according to item 12 or 13.
16. Method for the production of a transgenic plant having increased yield, particularly increased biomass and/or increased seed yield relative to control plants, comprising:
   (i) introducing and expressing in a plant a nucleic acid encoding a PRE-like polypeptide as defined in item 1 or 2; and
   (ii) cultivating the plant cell under conditions promoting plant growth and development.
17. Transgenic plant having increased yield, particularly increased seed yield, relative to control plants, resulting from modulated expression of a nucleic acid encoding a PRE-like polypeptide as defined in item 1 or 2, or a transgenic plant cell derived from said transgenic plant.
18. Transgenic plant according to item 11, 15 or 17, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
19. Harvestable parts of a plant according to item 18, wherein said harvestable parts are preferably shoot biomass and/or seeds.
20. Products derived from a plant according to item 18 and/or from harvestable parts of a plant according to item 19.
21. Use of a nucleic acid encoding a PRE-like polypeptide for increasing yield, particularly for increasing seed yield in plants, relative to control plants.
22. A method for enhancing yield-related traits in plants relative to control plants, comprising modulating expression in a plant of a nucleic acid encoding an SCE1, SUMO Conjugating Enzyme 1, polypeptide and optionally selecting for plants having enhanced yield-related traits.
23. Method according to item 22, wherein said SCE1 polypeptide comprises a sequence having at least one of the following:
   (i) 50%, 55%, 60%, 65%, 70%, 75%, 80%, 85%, 90%, 95%, 96%, 97%, 98%, 99% or more sequence identity to the amino acid sequence of any of the polypeptides of Table A2;
   (ii) 50%, 55%, 60%, 65%, 70%, 75%, 80%, 85%, 90%, 95%, 96%, 97%, 98%, 99% or more sequence identity to the amino acid sequence of any of the UBC domains as set forth in Table C2 of Example 4.
24. Method according to item 22 or 23, wherein said modulated expression is effected by introducing and expressing in a plant a nucleic acid encoding an SCE1 polypeptide.
25. Method according to any one of items 22 to 24, wherein said nucleic acid encoding an SCE1 polypeptide encodes any one of the proteins listed in Table A2 or is a portion of such a nucleic acid, or a nucleic acid capable of hybridising with such a nucleic acid.
26. Method according to any one of items 22 to 25, wherein said nucleic acid sequence encodes an orthologue or paralogue of any of the proteins given in Table A2.
27. Method according to any one of items 22 to 26, wherein said enhanced yield-related traits comprise increased biomass, preferably shoot and/or root biomass relative to control plants.
28. Method according to any one of items 22 to 27, wherein said enhanced yield-related traits are obtained under conditions of nitrogen deficiency.
29. Method according to any one of items 24 to 28, wherein said nucleic acid is operably linked to a constitutive promoter, preferably to a GOS2 promoter, most preferably to a GOS2 promoter from rice.
30. Method according to any preceding item, wherein said nucleic acid encoding an SCE1 polypeptide is of plant origin, preferably from a dicotyledonous plant, further preferably from the family Brasicaceae, most preferably from Arabidopsis thaliana.
31. Plant or part thereof, including seeds, obtainable by a method according to any preceeding item, wherein said plant or part thereof comprises a recombinant nucleic acid encoding an SCE1 polypeptide.
32. An isolated nucleic acid molecule comprising any one of the following:
   (i) a nucleic acid represented by SEQ ID NO: 201; SEQ ID NO: 203; SEQ ID NO: 205; SEQ ID NO: 207; SEQ ID NO: 209; SEQ ID NO: 211 and SEQ ID NO: 213;
   (ii) a nucleic acid or fragment thereof that is complementary to any one of the SEQ ID NOs given in (i);
   (iii) a nucleic acid encoding an SCE1 polypeptide having, in increasing order of preference, at least 70%, 75%, 80%, 85%, 90%, 95%, 96%, 97%, 98%, 99% or more sequence identity to any one of the amino acid sequences given in SEQ ID NO: 202; SEQ ID NO: 204; SEQ ID NO: 206; SEQ ID NO: 208; SEQ ID NO: 210; SEQ ID NO: 212 and SEQ ID NO: 214;
   (iv) a nucleic acid capable of hybridizing under stringent conditions to any one of the nucleic acids given in (i), (ii) or (iii) above.
33. An isolated polypeptide comprising:
   a. an amino acid sequence having, in increasing order of preference, at least 80%, 85%, 90%, 95%, 96%, 97%, 98%, 99% or 100% sequence identity to any one of the amino acid sequences given in SEQ ID NO: 202; SEQ ID NO: 204; SEQ ID NO: 206; SEQ ID NO: 208; SEQ ID NO: 210; SEQ ID NO: 212 and SEQ ID NO: 214;
   b. a nucleic acid capable of hybridizing under derivatives of any of the amino acid sequences given in (i).
34. Construct comprising:
   (i) nucleic acid encoding an SCE1 polypeptide as defined in items 22, 23 or 33, or a nucleic acid according to item 32;
   (ii) one or more control sequences capable of driving expression of the nucleic acid sequence of (a); and optionally
   (iii) a transcription termination sequence.
35. Construct according to item 34, wherein one of said control sequences is a constitutive promoter, preferably a GOS2 promoter, most preferably a GOS2 promoter from rice.
36. Use of a construct according to item 34 or 35 in a method for making plants having increased yield, particularly increased biomass relative to control plants.
37. Plant, plant part or plant cell transformed with a construct according to item 34 or 35.
38. Method for the production of a transgenic plant having increased yield, preferably increased seed yield relative to control plants, comprising:
   (i) introducing and expressing in a plant a nucleic acid encoding an SCE1 polypeptide as defined in item 22, 23 or 33, or a nucleic acid according to item 32; and
   (ii) cultivating the plant cell under conditions promoting plant growth and development; and optionally
   (iii) selecting for plants having enhanced yield-related traits
39. Transgenic plant having increased yield, particularly increased biomass, relative to control plants, resulting from modulated expression of a nucleic acid encoding an SCE1 polypeptide as defined in item 22, 23 or 33 or a transgenic plant cell derived from said transgenic plant.
40. Transgenic plant according to item 31, 37 or 39, or a transgenic plant cell derived thereof, wherein said plant is a crop plant or a monocot or a cereal, such as rice, maize, wheat, barley, millet, rye, triticale, sorghum and oats.
41. Harvestable parts of a plant according to item 40, wherein said harvestable parts are preferably shoot biomass and/or seeds.
42. Products derived from a plant according to item 40 and/or from harvestable parts of a plant according to item 41.
43. Use of a nucleic acid encoding an SCE1 polypeptide in increasing yield, particularly in increasing shoot and/or biomass in plants, relative to control plants.
44. A method for enhancing yield-related traits in plants relative to control plants, comprising modulating expression in a plant of a nucleic acid encoding a YEF1 polypeptide comprising an NPD1 domain (Novel Protein Domain 1), an RRM (RNA Recognition Motif) domain and optionally a CCCH (C3H Zinc Finger) domain.
45. Method according to item 44, wherein said YEF1 polypeptide comprises the following domains:
   (i) an NPD1 domain or a domain having in increasing order of preference at least 50%, 55%, 60%, 65%, 70%, 75%, 80%, 85%, 90%, 95% or more sequence identity to any of the NPD1 domains as set forth in Table C3 of Example 4,
   (ii) an RRM domain or a domain having in increasing order of preference at least 50%, 55%, 60%, 65%, 70%, 75%, 80%, 85%, 90%, 95% or more sequence identity to any of the RRM domains as set forth in Table C3 of Example 4; and
      wherein the domains of (i) and/or (ii) occur in increasing order of preference one, two, three, four, up to ten times.
46. Method according to items 44 or 45 wherein said YEF1 polypeptide comprises at least one of the following motifs:
   (i) Motif I or a motif having at least 75%, 80%, 85%, 90% or 95% sequence identity to SEQ ID NO: 277.
   (ii) Motif II or a motif having at least 50%, 55%, 60%, 65%, 70%, 75%, 80%, 85%, 90% or 95% sequence identity to SEQ ID NO: 278.
47. Method according to items 44 to 46 wherein said YEF1 polypeptides comprises a CCCH domain or a domain having in increasing order of preference at least 50%, 55%, 60%, 65%, 70%, 75%, 80%, 85%, 90%, 95% or more sequence identity to any of the CCCH domains as set forth in Table C3 of Example 4.
48. Method according to items 44 to 47, wherein said modulated expression is effected by introducing and expressing in a plant a nucleic acid encoding a YEF1 polypeptide.
49. Method according to any one of items 44 to 48, wherein said nucleic acid encoding a YEF1 polypeptide encodes any one of the proteins listed in Table A3 or is a portion of such a nucleic acid, or a nucleic acid capable of hybridising with such a nucleic acid.
50. Method according to any one of items 44 to 49, wherein said nucleic acid sequence encodes an orthologue or paralogue of any of the proteins given in Table A3.
51. Method according to any one of items 44 to 50, wherein said enhanced yield-related traits comprise increased yield, preferably increased seed yield relative to control plants.
52. Method according to any one of items 44 to 51, wherein said enhanced yield-related traits are obtained under non-stress conditions.
53. Method according to any one of items 44 to 52, wherein said enhanced yield-related traits are obtained under conditions of drought stress.
54. Method according to any one of items 48 to 51, wherein said nucleic acid is operably linked to a constitutive promoter, preferably to a GOS2 promoter, most preferably to a GOS2 promoter from rice.
55. Method according to any one of items 48 to 54, wherein said nucleic acid encoding a YEF1 polypeptide is of plant origin, preferably from a dicotyledonous plant, further preferably from the family Solanaceae, more preferably from the genus Solanum, most preferably from Lycorpersicum esculenturn.
56. Plant or part thereof, including seeds, obtainable by a method according to any preceding item, wherein said plant or part thereof comprises a recombinant nucleic acid encoding YEF1 polypeptide.
57. Construct comprising:
   a. nucleic acid encoding a YEF1 polypeptide as defined in items 44 to 47;
   b. one or more control sequences capable of driving expression of the nucleic acid sequence of (a); and optionally
   c. a transcription termination sequence.
58. Construct according to item 57, wherein one of said control sequences is a constitutive promoter, preferably a GOS2 promoter, most preferably a GOS2 promoter from rice.
59. Use of a construct according to item 57 or 58 in a method for making plants having increased yield, particularly increased biomass and/or increased seed yield relative to control plants.
60. Plant, plant part or plant cell transformed with a construct according to item 57 or 58.
61. Method for the production of a transgenic plant having increased yield, particularly increased biomass and/or increased seed yield relative to control plants, comprising:
   (i) introducing and expressing in a plant a nucleic acid encoding a YEF1 polypeptide as defined in item 44 to 47; and
   (ii) cultivating the plant cell under conditions promoting plant growth and development.
62. Transgenic plant having increased yield, particularly increased biomass and/or increased seed yield, relative to control plants, resulting from modulated expression of a nucleic acid encoding a YEF1 polypeptide as defined in item 44 to 47, or a transgenic plant cell derived from said transgenic plant.
63. Transgenic plant according to item 56, 60 or 62, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
64. Harvestable parts of a plant according to item 63, wherein said harvestable parts are preferably shoot biomass and/or seeds.
65. Products derived from a plant according to item 63 and/or from harvestable parts of a plant according to item 64.
66. Use of a nucleic acid encoding a YEF1 polypeptide in increasing yield, particularly in increasing seed yield and/or shoot biomass in plants, relative to control plants.
67. A method for enhancing yield-related traits in plants relative to control plants, comprising modulating expression in a plant of a nucleic acid encoding a subgroup III Grx polypeptide.
68. Method according to item 67, wherein said subgroup III Grx polypeptide comprises a CCxx active centre, preferably a CCxS active centre, most preferably a CCMS active centre.
69. Method according to item 67 or 68, wherein said modulated expression is effected by introducing and expressing in a plant a nucleic acid encoding a subgroup III Grx polypeptide.
70. Method according to any one of items 67 to 69, wherein said nucleic acid encoding a subgroup III Grx polypeptide encodes any one of the proteins listed in Table A4 or is a portion of such a nucleic acid, or a nucleic acid capable of hybridising with such a nucleic acid.
71. Method according to any one of items 67 to 70, wherein said nucleic acid sequence encodes an orthologue or paralogue of any of the proteins given in Table A4.
72. Method according to any one of items 67 to 71, wherein said enhanced yield-related traits comprise increased yield, preferably increased biomass and/or increased seed yield relative to control plants.
73. Method according to any one of items 67 to 72, wherein said enhanced yield-related traits are obtained under non-stress conditions.
74. Method according to any one of items 69 to 73, wherein said nucleic acid is operably linked to a green tissue-specific promoter, preferably to a protochlorophyllid reductase promoter, most preferably to a protochlorophyllid reductase promoter as represented by SEQ ID NO: 155.
75. Method according to any one of items 67 to 74, wherein said nucleic acid encoding a subgroup III Grx polypeptide is of plant origin, preferably from a dicotyledonous plant, further preferably from the family Brassicaceae, more preferably from the genus Arabidopsis, most preferably from Arabidopsis thaliana.
76. Plant or part thereof, including seeds, obtainable by a method according to any preceding item, wherein said plant or part thereof comprises a recombinant nucleic acid encoding a subgroup III Grx polypeptide.
77. Construct comprising:
   (i) nucleic acid encoding a subgroup III Grx polypeptide as defined in items 67 or 68;
   (ii) one or more control sequences capable of driving expression of the nucleic acid sequence of (a); and optionally (iii) a transcription termination sequence.
78. Construct according to item 77, wherein one of said control sequences is a green tissue-specific promoter, preferably a protochlorophyllid reductase promoter, most preferably a protochlorophyllid reductase promoter as represented by SEQ ID NO: 436.
79. Use of a construct according to item 77 or 78 in a method for making plants having increased yield, particularly increased biomass and/or increased seed yield relative to control plants.
80. Plant, plant part or plant cell transformed with a construct according to item 77 or 78.
81. Method for the production of a transgenic plant having increased yield, particularly increased biomass and/or increased seed yield relative to control plants, comprising:
   (i) introducing and expressing in a plant a nucleic acid encoding a subgroup III Grx polypeptide as defined in item 67 or 68; and
   (ii) cultivating the plant cell under conditions promoting plant growth and development.
82. Transgenic plant having increased yield, particularly increased biomass and/or increased seed yield, relative to control plants, resulting from modulated expression of a nucleic acid encoding a subgroup III Grx polypeptide as defined in item 67 or 68, or a transgenic plant cell derived from said transgenic plant.
83. Transgenic plant according to item 76, 80 or 82, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
84. Harvestable parts of a plant according to item 83, wherein said harvestable parts are preferably shoot biomass and/or seeds.
85. Products derived from a plant according to item 83 and/or from harvestable parts of a plant according to item 84.
86. Use of a nucleic acid encoding a subgroup III Grx polypeptide in increasing yield, particularly in increasing seed yield and/or shoot biomass in plants, relative to control plants.
87. A method for altering the ratio of roots to shoots in plants relative to that of control plants, comprising modulating expression in a plant of a nucleic acid encoding a Sister of FT polypeptide or a homologue thereof having in increasing order of preference at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid sequence represented by SEQ ID NO: 440.
88. Method according to item 87, wherein the nucleic acid encoding a Sister of FT polypeptide or a homologue thereof, when used in the construction of a phylogenetic tree of FT sequences, clusters with the group comprising the amino acid sequence represented by SEQ ID NO: 440 rather than with any other group.
89. Method according to item 87 or 88, wherein said nucleic acid encoding a Sister of FT polypeptide or a homologue thereof is a portion of the nucleic acid represented by SEQ ID NO: 1, or is a portion of a nucleic acid encoding an orthologue or paralogue of the amino acid sequence of SEQ ID NO: 2, wherein the portion is at least 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, consecutive nucleotides in length, the consecutive nucleotides being of SEQ ID NO: 439, or of a nucleic acid encoding an orthologue or paralogue of the amino acid sequence of SEQ ID NO: 440.
90. Method according to any one of items 87 to 89, wherein the nucleic acid encoding a Sister of FT polypeptide or a homologue thereof is capable of hybridising to the nucleic acid represented by SEQ ID NO: 439 or is capable of hybridising to a nucleic acid encoding an orthologue, paralogue or homologue of SEQ ID NO: 440.
91. Method according to any one of items 87 to 90, wherein said nucleic acid encoding a Sister of FT polypeptide or a homologue thereof encodes an orthologue or paralogue of the sequence represented by SEQ ID NO: 440.
92. Method according to any one of items 87 to 92, wherein said modulated expression is effected by introducing and expressing in a plant a nucleic acid encoding a Sister of FT polypeptide or a homologue thereof.
93. Method according to any one of items 87 to 93, wherein said altered root:shoot ratio is obtained under non-stress conditions.
94. Method according to item 92 or 93, wherein said nucleic acid is operably linked to a constitutive promoter, preferably to a GOS2 promoter, most preferably to a GOS2 promoter from rice.
95. Method according to any one of items 87 to 94, wherein said nucleic acid encoding a Sister of FT polypeptide is of plant origin, preferably from a dicotyledonous plant, further preferably from the family Brassicaceae, more preferably from the genus Arabidopsis, most preferably from Arabidopsis thaliana.
96. Plant or part thereof, including seeds, obtainable by a method according to any preceding item, wherein said plant or part thereof comprises a recombinant nucleic acid encoding a Sister of FT polypeptide or a homologue thereof.
97. Construct comprising:
   (i) nucleic acid encoding a Sister of FT polypeptide or a homologue thereof as defined in any of items 87 to 91;
   (ii) one or more control sequences capable of driving expression of the nucleic acid sequence of (a); and optionally
   (iii) a transcription termination sequence.
98. Construct according to item 97, wherein one of said control sequences is a constitutive promoter, preferably a GOS2 promoter, most preferably a GOS2 promoter from rice.
99. Use of a construct according to item 96 or 97 in a method for making plants having an altered root:shoot ratio relative to control plants.
100. Plant, plant part or plant cell transformed with a construct according to item 96 or 97.
101. Method for the production of a transgenic plant having an altered root:shoot ratio relative to control plants, comprising:
   (i) introducing and expressing in a plant a nucleic acid encoding a Sister of FT polypeptide or a homologue thereof as defined in any one of items 87 to 92; and
   (ii) cultivating the plant cell under conditions promoting plant growth and development.
102. Transgenic plant having an altered root:shoot ratio relative to control plants, resulting from modulated expression of a nucleic acid encoding a Sister of FT polypeptide or a homologue thereof as defined in any one of items 87 to 92.
103. Transgenic plant according to item 96, 100 or 102, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.
104. Products derived from a plant according to item 103.
105. Use of a nucleic acid encoding a Sister of FT polypeptide or a homologue thereof in altering the root:shoot ration of plants relative to control plants.

### DESCRIPTION OF FIGURES

The present invention will now be described with reference to the following figures in which: FIG. 1 represents the domain structure of a PRE-like protein (SEQ ID NO: 2) with the conserved HLH domain as identified with HMMPfam indicated in bold. The numbered lines under the sequence refer to the motifs described above.

FIG. 2 represents a multiple alignment of some PRE-like polypeptides. The identifiers are as follows: TaPRE-like: SEQ ID NO: 2, Triticum aestivum; TA36504: SEQ ID NO: 159, Sorghum bicolor, TA57848: SEQ ID NO: 53, Glycine max; CA783850: SEQ ID NO: 59, Glycine sofa; TC110752: SEQ ID NO: 95, Medicago truncatula; X11.633: SEQ ID NO: 123, Populus trichocarpa; 129.2: SEQ ID NO: 125, Populus trichocarpa; TA18273: SEQ ID NO: 37, Camellia sinensis; GSVIVT120001: SEQ ID NO: 173, Vitis vinifera; AT1G74500: SEQ ID NO: 23, Arabidopsis thaliana; TA3862: SEQ ID NO: 165, Triphysaria versicolor, AT3G47710: SEQ ID NO: 25, Arabidopsis thaliana. The asterisks indicate absolute sequence conservation, the colons indicate highly conserved substitutions and the dots indicate conserved substitutions.

FIG. 3 shows a phylogenetic tree of PRE-like proteins. The sequence identifiers are as used in Table A, TaPRE-like corresponds to SEQ ID NO: 2.

FIG. 4 represents the binary vector for increased expression in Oryza sativa of a PRE-like encoding nucleic acid under the control of a rice GOS2 promoter (pGOS2).

FIG. 5 details examples of PRE-like sequences useful in performing the methods according to the present invention.

FIG. 6 represents the sequence of Arath_SCE1-1, SEQ ID NO: 200, with conserved UBC domain indicated in bold and the active-site Cysteinee amino acid residue boxed. Amino acid residues proposed to interact with the E3 ligase are underlined.

FIG. 7 represents a multiple alignment of the SCE1 polypeptides given in Table A. A consensus sequence is also given. Highly conserved residues are indicated in the consensus sequence.

FIG. 8 represents the binary vector for increased expression in Oryza sativa of an SCE1-encoding nucleic acid under the control of a rice GOS2 promoter (pGOS2).

FIG. 9 details examples of SCE1 sequences useful in performing the methods according to the present invention.

FIG. 10 represents the amino acid of SEQ ID NO: 249 wherein the conserved domains and motifs are highlighted. BOX I: NPD1 domain; BOX II: C3H domain; BOX III: RRM domain. Motif I is indicated in lowercase bold letters; Motif II is underlined. The three Cysteine and Histidine residues responsible for Zinc coordination in the C3H motif are indicated in bold.

FIG. 11 represents a protein sequence multiple alignment of YEF1 polypeptides. A consensus sequence is given.

FIG. 12 shows a phylogenetic tree containing YEF1 polypeptides. The phylogenetic tree was made using a multiple alignment of the polypeptides given in Table A. Additionally two Arabidopsis thaliana protein which comprise a C3H and an RRM domain but lack the NPD1 domain are included in the tree, At1g07360.1 and At3g27700.1, which have the Genebank accession numbers NP_{—}563788 and NP_{—}851008 respectively.

FIG. 13 represents the binary vector for increased expression in Oryza sativa of Le_YEF1_{—}1 nucleic acid under the control of a rice GOS2 promoter (pGOS2).

FIG. 14 details examples of YEF1 sequences useful in performing the methods according to the present invention.

FIG. 15 represents confirmed or proposed roles for plant Grxs.

FIG. 16 represents the phylogenetic tree of Grxs from Arabidopsis thaliana, Populus trichocarpa, and Oryza sativa sequences. The phylogenetictree was constructed using ClustalW.

FIG. 17 represents the phylogenetic tree of plant glutaredoxins.

FIG. 18 represents the phylogenetic tree of selected glutaredoxin proteins. The alignment was generated using “CLUSTALW”, and a neighbour-joining tree was calculated. The circular tree was drawn using “Dendroscope”.

FIG. 19 represents the binary vector for increased expression in Oryza sativa of a subgroup III Grx-encoding nucleic acid under the control of a green tissue-specific protochlorophyllid reductase promoter.

FIG. 20 details examples of Group III Grx sequences useful in performing the methods according to the present invention.

FIG. 21 shows the binary vector for increased expression in Oryza sativa of a Sister of FT-encoding nucleic acid under the control of a rice GOS2 promoter (pGOS2)

FIG. 22 details examples of Sister of FT sequences useful in performing the methods according to the present invention.

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Sequences Related to the Nucleic Acid Sequences and the Polypeptide Sequences Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table A provides a list of nucleic acid sequences related to the nucleic acid sequence used in the methods of the present invention. The term “table A” used in this specification is to be taken to specify the content of table A1, table A2, table A3, and/or table A4.

The term “table A1” used in this specification is to be taken to specify the content of table A1. The term “table A2” used in this specification is to be taken to specify the content of table A2. The term “table A3” used in this specification is to be taken to specify the content of table A3. The term “table A4” used in this specification is to be taken to specify the content of table A4. In one preferred embodiment, the term “table A” means table A1. In another preferred embodiment, the term “table A” means table A2. In another preferred embodiment, the term “table A” means table A3. In another preferred embodiment, the term “table A” means table A4.

TABLE A1
Examples of PRE-like polypeptides:

| | | | Protein |
|---|---|---|---|
| | | Nucleic acid | SEQ |
| identifier | Plant source | SEQ ID NO: | ID NO: |
| TaPRE-like | Triticum aestivum | 1 | 2 |
| XVII.359 | Populus trichocarpa | 16 | 15 |
| BE205620 | Allium cepa | 18 | 17 |
| TA8292 | Antirrhinum majus | 20 | 19 |
| AT1G26945 | Arabidopsis thaliana | 22 | 21 |
| AT1G74500 | Arabidopsis thaliana | 24 | 23 |
| AT3G47710 | Arabidopsis thaliana | 26 | 25 |
| AT3G28857 | Arabidopsis thaliana | 28 | 27 |
| AT5G39860 | Arabidopsis thaliana | 30 | 29 |
| AT5G15160 | Arabidopsis thaliana | 32 | 31 |
| DV481273 | Brachypodium | 34 | 33 |
| | distachyon | | |
| EL408974 | Cathamus tinctorius | 36 | 35 |
| TA18273 | Camellia sinensis | 38 | 37 |
| TA16547 | Camellia sinensis | 40 | 39 |
| TA6224 | Coffea canephora | 42 | 41 |
| DY672743 | Fragaria vesca | 44 | 43 |
| AJ752013 | Gerbera hybrid | 46 | 45 |
| AJ758453 | Gerbera hybrid | 48 | 47 |
| TA56389 | Glycine max | 50 | 49 |
| TA62505 | Glycine max | 52 | 51 |
| TA57848 | Glycine max | 54 | 53 |
| CD416537 | Glycine max | 56 | 55 |
| TA53762 | Glycine max | 58 | 57 |
| CA783850 | Glycine soja | 60 | 59 |
| BE052528 | Gossypium arboretum | 62 | 61 |
| DW498223 | Gossypium hirsutum | 64 | 63 |
| DT527245 | Gossypium hirsutum | 66 | 65 |
| DW505403 | Gossypium hirsutum | 68 | 67 |
| DW501889 | Gossypium hirsutum | 70 | 69 |
| TA766 | Hedyotis terminalis | 72 | 71 |
| EL487276 | Helianthus paradoxus | 74 | 73 |
| EL488459 | Helianthus paradoxus | 76 | 75 |
| EL465600 | Helianthus tuberosus | 78 | 77 |
| TA42071 | Hordeum vulgare | 80 | 79 |
| TA44490 | Hordeum vulgare | 82 | 81 |
| DY976394 | Lactuca sativa | 84 | 83 |
| TA3169 | Lactuca virosa | 86 | 85 |
| CO541258 | Malus x domestica | 88 | 87 |
| TA43070 | Malus x domestica | 90 | 89 |
| TA36763 | Malus x domestica | 92 | 91 |
| TA34851 | Malus x domestica | 94 | 93 |
| TC110752 | Medicago truncatula | 96 | 95 |
| BI268948 | Medicago truncatula | 98 | 97 |
| TC110807 | Medicago truncatula | 100 | 99 |
| EH367818 | Nicotiana benthamiana | 102 | 101 |
| TA21468 | Nicotiana tabacum | 104 | 103 |
| Os04g54900 | Oryza sativa | 106 | 105 |
| Os03g07540 | Oryza sativa | 108 | 107 |
| Os02g51320 | Oryza sativa | 110 | 109 |
| Os06g12210 | Oryza sativa | 112 | 111 |
| DN151440 | Panicum virgatum | 114 | 113 |
| CV297566 | Petunia x hybrida | 116 | 115 |
| CV297594 | Petunia x hybrida | 118 | 117 |
| TA4110 | Petunia x hybrida | 120 | 119 |
| CV532618 | Phaseolus vulgaris | 122 | 121 |
| XII.633 | Populus trichocarpa | 124 | 123 |
| 129.2 | Populus trichocarpa | 126 | 125 |
| AJ823214 | Prunus persica | 128 | 127 |
| BU045110 | Prunus persica | 130 | 129 |
| BU048569 | Prunus persica | 132 | 131 |
| AJ823124 | Prunus persica | 134 | 133 |
| BU043331 | Prunus persica | 136 | 135 |
| TA5285 | Ricinus communis | 138 | 137 |
| CA090192 | Saccharum officinarum | 140 | 139 |
| CV167880 | Salvia miltiorrhiza | 142 | 141 |
| CV166470 | Salvia miltiorrhiza | 144 | 143 |
| BE705205 | Secale cereale | 146 | 145 |
| CO553461 | Senecio squalidus | 148 | 147 |
| DY660883 | Senecio vulgaris | 150 | 149 |
| AW647879 | Solanum lycopersicum | 152 | 151 |
| CV503041 | Solanum tuberosum | 154 | 153 |
| TA43072 | Solanum tuberosum | 156 | 155 |
| TA44221 | Solanum tuberosum | 158 | 157 |
| TA36504 | Sorghum bicolor | 160 | 159 |
| TA33922 | Sorghum bicolor | 162 | 161 |
| EH277818 | Spartina alterniflora | 164 | 163 |
| TA3862 | Triphysaria versicolor | 166 | 165 |
| TA89858 | Triticum aestivum | 168 | 167 |
| TA103938 | Triticum aestivum | 170 | 169 |
| TA98487 | Triticum aestivum | 172 | 171 |
| GSVIVT00000120001 | Vitis vinifera | 174 | 173 |
| GSVIVT00037009001 | Vitis vinifera | 176 | 175 |
| GSVIVT00000123001 | Vitis vinifera | 178 | 177 |
| GSVIVT00020927001 | Vitis vinifera | 180 | 179 |
| DT602195 | Welwitschia mirabilis | 182 | 181 |
| TA215077 | Zea mays | 184 | 183 |
| TA170348 | Zea mays | 186 | 185 |
| DY238348 | Zea mays | 188 | 187 |
| TA207044 | Zea mays | 190 | 189 |
| CK367883 | Zea mays | 192 | 191 |
| TA2164 | Zingiber officinale | 194 | 193 |
| TA5496 | Zingiber officinale | 196 | 195 |

TABLE A2
Examples of SCE1 nucleic acids and polypeptides:

| | | Nucleic acid | Protein |
|---|---|---|---|
| Plant Source | Origin species | SEQ ID NO: | SEQ ID NO: |
| Arath_SCE1_1 | Arabidopsis thaliana | 199 | 200 |
| Helan_SCE1_1 | Helianus annuus | 201 | 202 |
| Triae_SCE1_1 | Triticum aestivum | 203 | 204 |
| Horvu_SCE1_1 | Hordeum vulgare | 205 | 206 |
| Glyma_SCE_1 | Glycine max | 207 | 208 |
| Zeama_SCE1_1 | Zea mays | 209 | 210 |
| Zeama_SCE1_2 | Zea mays | 211 | 212 |
| Zeama_SCE1_3 | Zea mays | 213 | 214 |
| Orysa_SCE1_1 | Oryza sativa | 215 | 216 |
| Orysa_SCE1_2 | Oryza sativa | 217 | 218 |
| Orysa_SCE1_3 | Oryza sativa | 219 | 220 |
| Vitvi_SCE1_1 | Vitis vinifera | 221 | 222 |
| Nicbe_SCE1_1 | Nicotiana benthamiana | 223 | 224 |
| Popul_SCE1_1 | Populus x canadensis | 225 | 226 |
| Tritu_SCE1_1 | Triticum turgidum | 227 | 228 |
| PopTr_SCE1_1 | Populus trichocarpa | 229 | 230 |
| PopTr_SCE1_2 | Populus trichocarpa | 231 | 232 |
| Phypa_SCE1_1 | Physcomitrlla patens | 233 | 234 |
| Phypa_SCE1_2 | Vitis vinifera | 235 | 236 |
| Chlre_SCE1_1 | Chlamydomonas | 237 | 238 |
| | reinhardtii | | |
| Pruar_SCE1_1 | Prunus armeniaca | 239 | 240 |
| Ostta_SCE1_1 | Ostreococus tauri | 241 | 242 |
| Picsi_SCE1_1 | Picea sitchensis | 243 | 244 |

TABLE A3
Examples of YEF1 polypeptides:

| | | Nucleic | |
|---|---|---|---|
| | | acid | |
| | | SEQ | Polypeptide |
| Sequence name | Origin species | ID NO: | SEQ ID NO: |
| Le_YEF1_1 | Lycopersicum | 248 | 249 |
| | esculentum | | |
| Pinus\r\ADW16853 | Pinus radiata | 250 | 251 |
| Euc\grandis\ADW16464 | Eucalyptus grandis | 252 | 253 |
| Pinus\r\ADW16852 | Pinus radiata | 254 | 255 |
| Pt\scaff_220.7\[2234] | Populus trichocarpa | 256 | 257 |
| Pt\scaff_III.1611\[2309] | Populus trichocarpa | 258 | 259 |
| At3g51950.1 | Arabidopsis thaliana | 260 | 261 |
| At2g05160.1 | Arabidopsis thaliana | 262 | 263 |
| Os\LOC_Os03g21160.1 | Oryza sativa | 264 | 265 |
| Os\LOC_Os07g48410.1 | Oryza sativa | 266 | 267 |
| Os\LOC_Os03g21140.1 | Oryza sativa | 268 | 269 |
| Zm TA1731224577 | Zea mays | 270 | 271 |
| Vv\CAN64426 | Vitis vinifera | 272 | 273 |
| Vv\CAN62156 | Vitis vinifera | 274 | 275 |

TABLE A4
Examples of nucleic acid sequences related to SEQ ID NO: 282
and polypeptide sequences related to SEQ ID NO: 283:

| | | Nucleic | |
|---|---|---|---|
| | | acid | Protein |
| | | SEQ | SEQ |
| Name | Plant source | ID NO: | ID NO: |
| At1g03020 | Arabidopsis thaliana | 284 | 285 |
| At1g03850 | Arabidopsis thaliana | 286 | 287 |
| At1g06830 | Arabidopsis thaliana | 288 | 289 |
| At1g28480 | Arabidopsis thaliana | 290 | 291 |
| At2g30540 | Arabidopsis thaliana | 292 | 293 |
| At2g47870 | Arabidopsis thaliana | 294 | 295 |
| At2g47880 | Arabidopsis thaliana | 296 | 297 |
| At3g02000 | Arabidopsis thaliana | 298 | 299 |
| At3g21450 | Arabidopsis thaliana | 300 | 301 |
| At3g21460 | Arabidopsis thaliana | 302 | 303 |
| At3g62930 | Arabidopsis thaliana | 304 | 305 |
| At3g62950 | Arabidopsis thaliana | 306 | 307 |
| At3g62960 | Arabidopsis thaliana | 308 | 309 |
| At4g15660 | Arabidopsis thaliana | 310 | 311 |
| At4g15670 | Arabidopsis thaliana | 312 | 313 |
| At4g15680 | Arabidopsis thaliana | 314 | 315 |
| At4g15690 | Arabidopsis thaliana | 316 | 317 |
| At4g15700 | Arabidopsis thaliana | 318 | 319 |
| At4g33040 | Arabidopsis thaliana | 320 | 321 |
| At5g11930 | Arabidopsis thaliana | 322 | 323 |
| At5g14070 | Arabidopsis thaliana | 324 | 325 |
| CD820020 | Brassica napus | 326 | 327 |
| DY020133 | Brassica napus | 328 | 329 |
| DY022103 | Brassica napus | 330 | 331 |
| ES268095 | Brassica napus | 332 | 333 |
| TA30664_3708 | Brassica napus | 334 | 335 |
| TA32617_3708 | Brassica napus | 336 | 337 |
| CDS7086 | Medicago truncatula | 338 | 339 |
| Os01g09830 | Oryza sativa | 340 | 341 |
| Os01g13950 | Oryza sativa | 342 | 343 |
| Os01g26912 | Oryza sativa | 344 | 345 |
| Os01g47760 | Oryza sativa | 346 | 347 |
| Os01g70990 | Oryza sativa | 348 | 349 |
| Os02g30850 | Oryza sativa | 350 | 351 |
| Os04g32300 | Oryza sativa | 352 | 353 |
| Os05g05730 | Oryza sativa | 354 | 355 |
| Os05g10930 | Oryza sativa | 356 | 357 |
| Os05g48930 | Oryza sativa | 358 | 359 |
| Os07g05630 | Oryza sativa | 360 | 361 |
| Os11g43520 | Oryza sativa | 362 | 363 |
| Os11g43530 | Oryza sativa | 364 | 365 |
| Os11g43550 | Oryza sativa | 366 | 367 |
| Os11g43580 | Oryza sativa | 368 | 369 |
| Os12g35330 | Oryza sativa | 370 | 371 |
| Os12g35340 | Oryza sativa | 372 | 373 |
| TC13595 | Picea abies | 374 | 375 |
| TC18426 | Picea abies | 376 | 377 |
| TC18846 | Picea abies | 378 | 379 |
| TC25571 | Picea abies | 380 | 381 |
| 136027_e_gw1.125.81.1 | Physcomitrella patens | 382 | 383 |
| CO170466 | Pinus taeda | 384 | 385 |
| TA14421_3352 | Pinus taeda | 386 | 387 |
| TA27091_3352 | Pinus taeda | 388 | 389 |
| CDS5551 | Populus trichocarpa | 390 | 391 |
| scaff_77.14 | Populus trichocarpa | 392 | 393 |
| scaff_III.1368 | Populus trichocarpa | 394 | 395 |
| scaff_XIV.1520 | Populus trichocarpa | 396 | 397 |
| scaff_XIV.1522 | Populus trichocarpa | 398 | 399 |
| scaff_XIV.784 | Populus trichocarpa | 400 | 401 |
| scaff_XIV.786 | Populus trichocarpa | 402 | 403 |
| CD871873 | Triticum aestivum | 404 | 405 |
| CN011047 | Triticum aestivum | 406 | 407 |
| TA102057_4565 | Triticum aestivum | 408 | 409 |
| TA99595_4565 | Triticum aestivum | 410 | 411 |
| GSVIVT00006974001 | Vitis vinifera | 412 | 413 |
| GSVIVT00019806001 | Vitis vinifera | 414 | 415 |
| GSVIVT00019807001 | Vitis vinifera | 416 | 417 |
| GSVIVT00023580001 | Vitis vinifera | 418 | 419 |
| GSVIVT00023582001 | Vitis vinifera | 420 | 421 |
| GSVIVT00023583001 | Vitis vinifera | 422 | 423 |
| GSVIVT00037903001 | Vitis vinifera | 424 | 425 |
| AI977949 | Zea mays | 426 | 427 |
| DN209858 | Zea mays | 428 | 429 |
| DN222454 | Zea mays | 430 | 431 |
| EC883167 | Zea mays | 432 | 433 |
| TA19029_4577999 | Zea mays | 434 | 435 |

In some instances, related sequences are tentatively assembled and publicly disclosed by research institutions, such as The Institute for Genomic Research (TIGR). The Eukaryotic Gene Orthologs (EGO) database is used to identify such related sequences, either by keyword search or by using the BLAST algorithm with the nucleic acid or polypeptide sequence of interest.

### Example 2

### Alignment of Polypeptide Sequences

### Example 2.1

### Alignment of PRE-Like Polypeptide Sequences

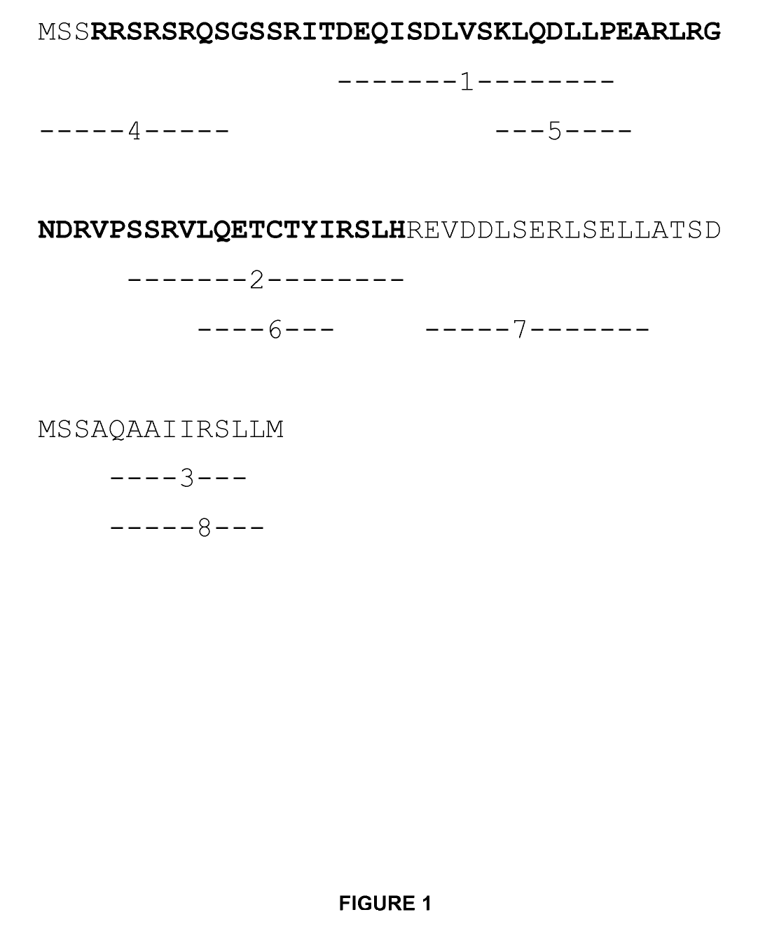

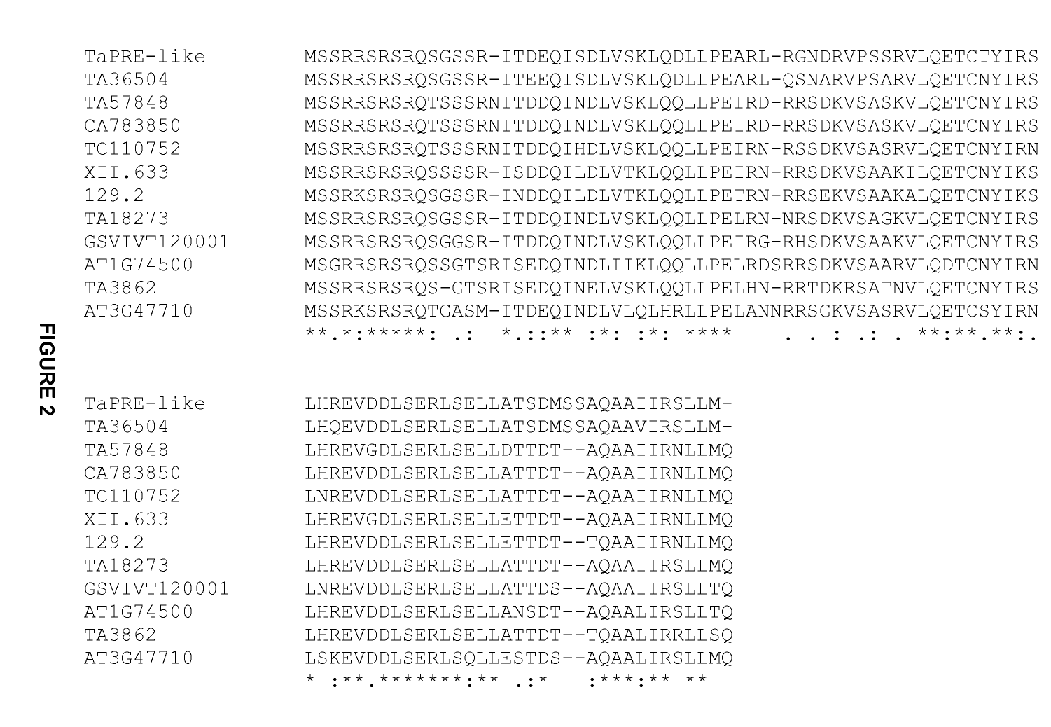

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Minor manual editing may be done to further optimise the alignment. Sequence conservation among PRE-like polypeptides is essentially throughout the whole sequence. A number of PRE-like polypeptides are aligned in FIG. 2.

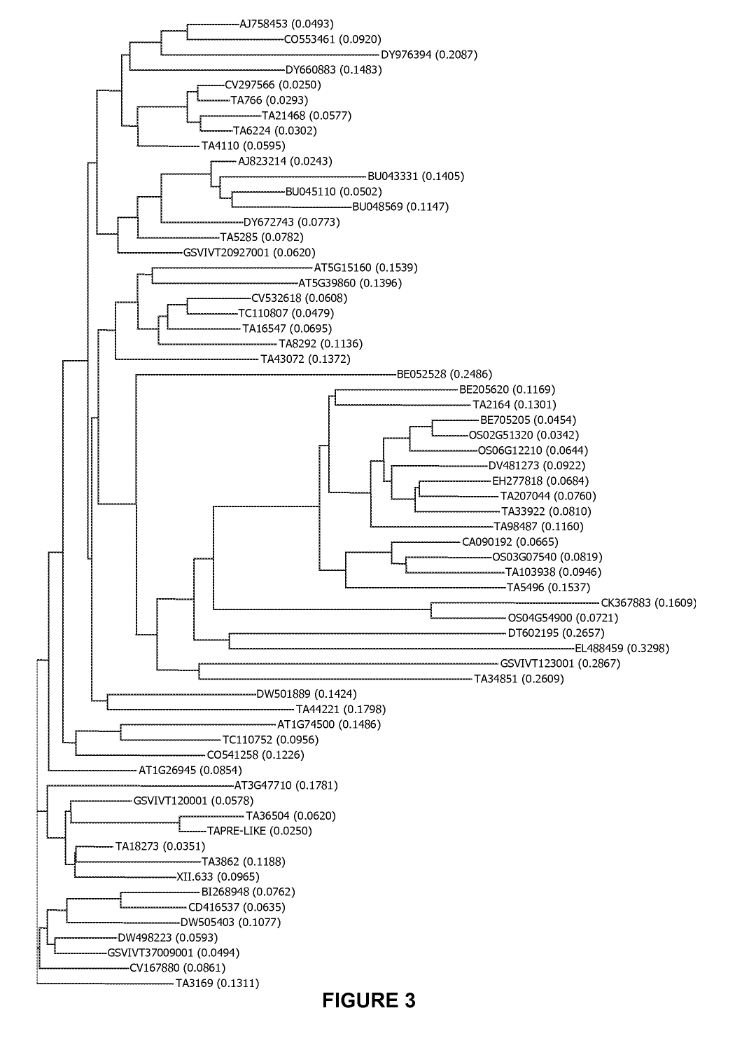

A phylogenetic tree of PRE-like polypeptides (FIG. 3) was constructed using a neighbour-joining clustering algorithm as provided in the AlignX programme from the Vector NTI (Invitrogen). As input, an msf file prepared with EMMA (EMBOSS, gap opening penalty 11, gap extension penalty 1) was used.

### Example 2.2

### Alignment of SCE1 Polypeptide Sequences

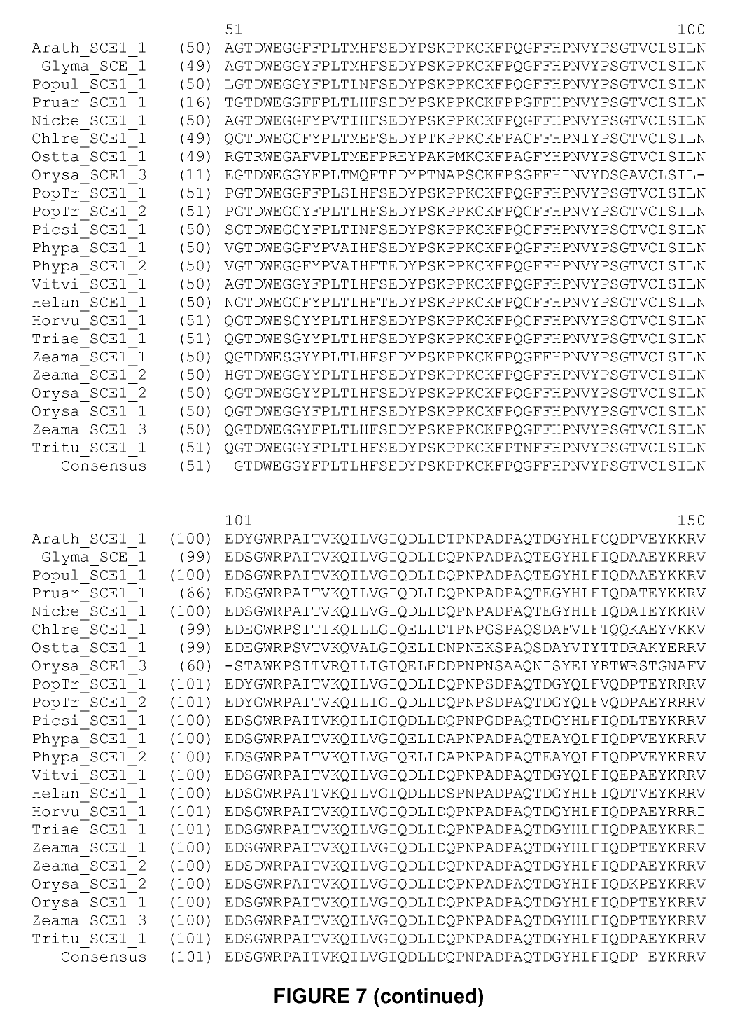

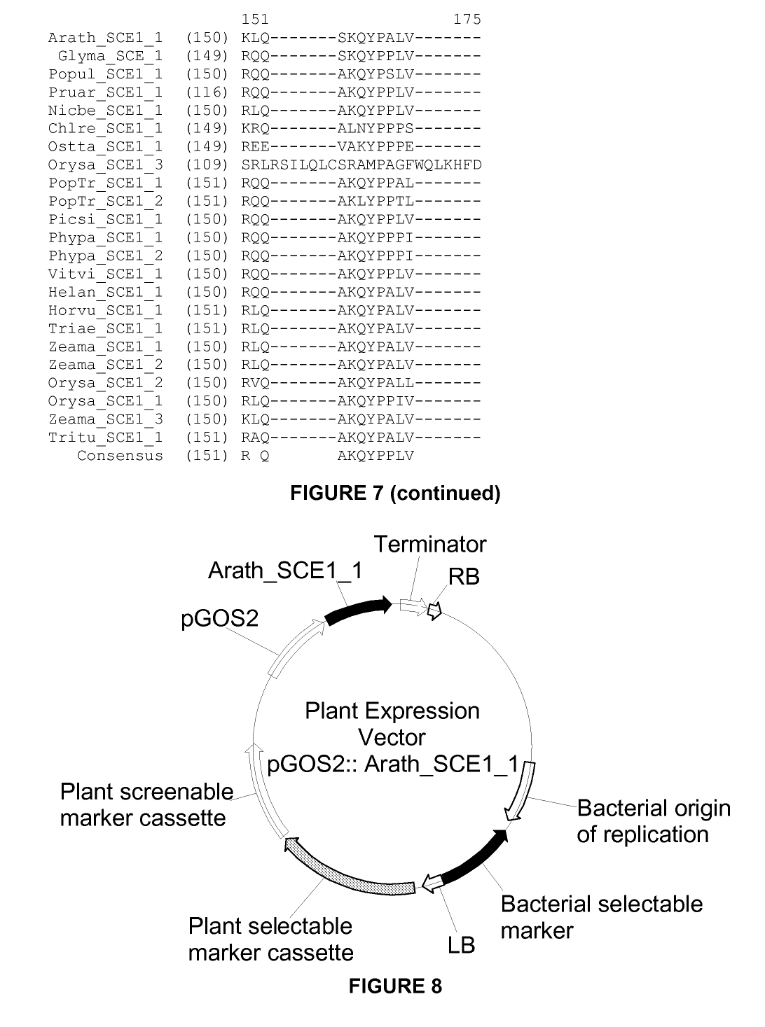

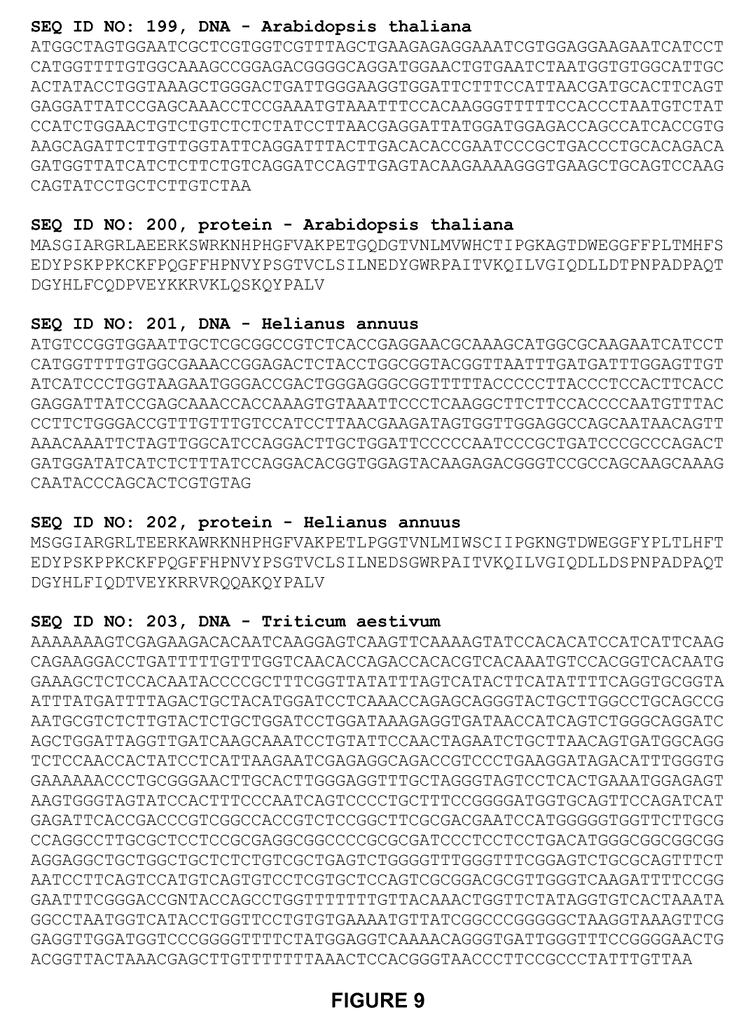

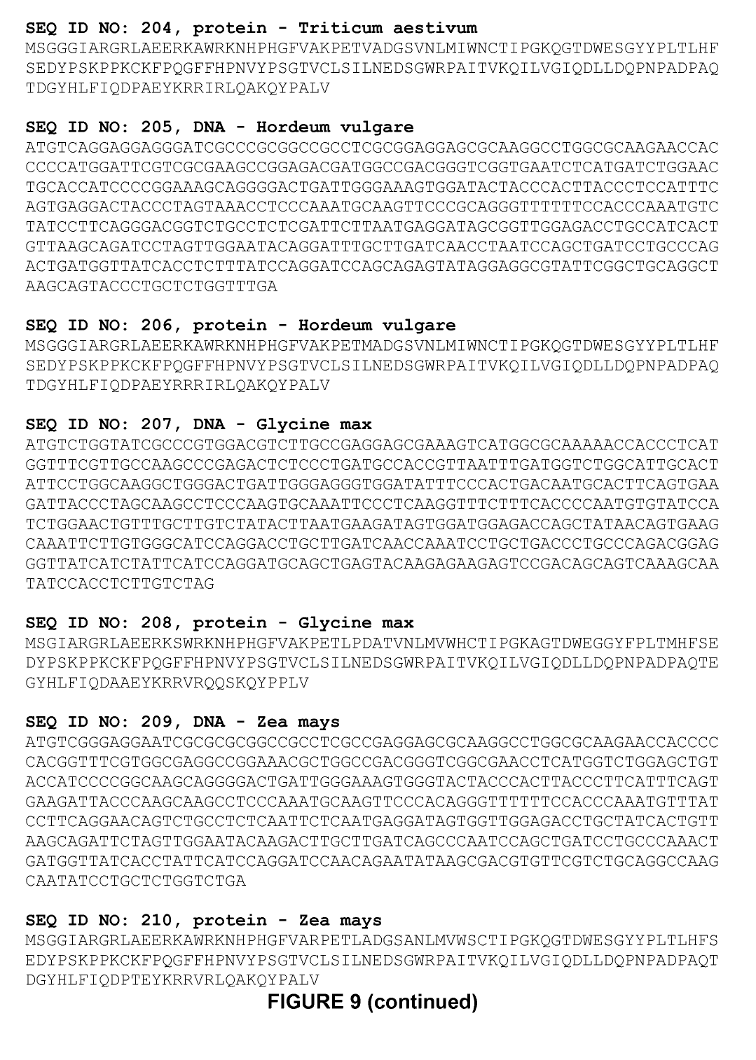

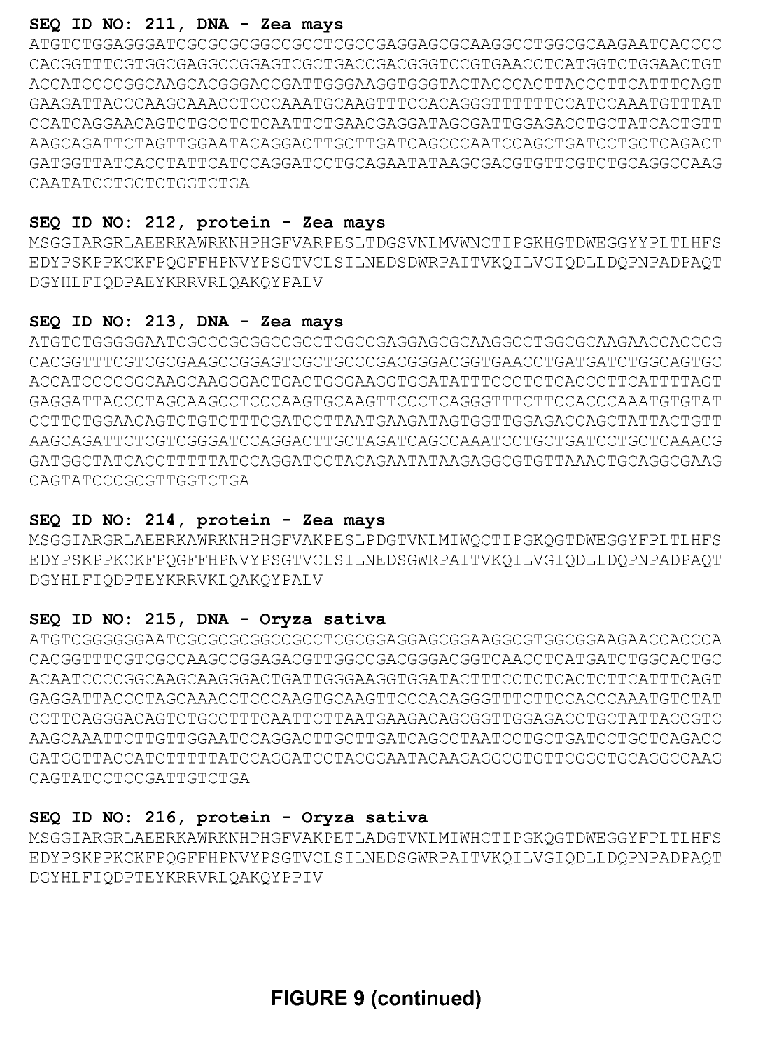

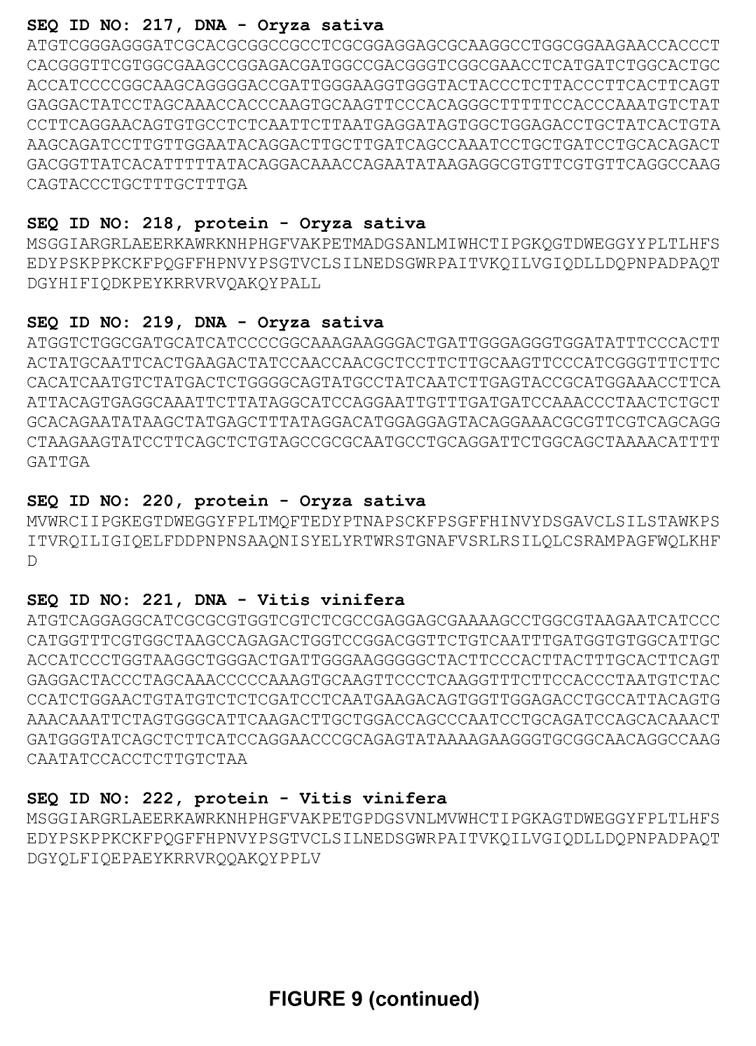

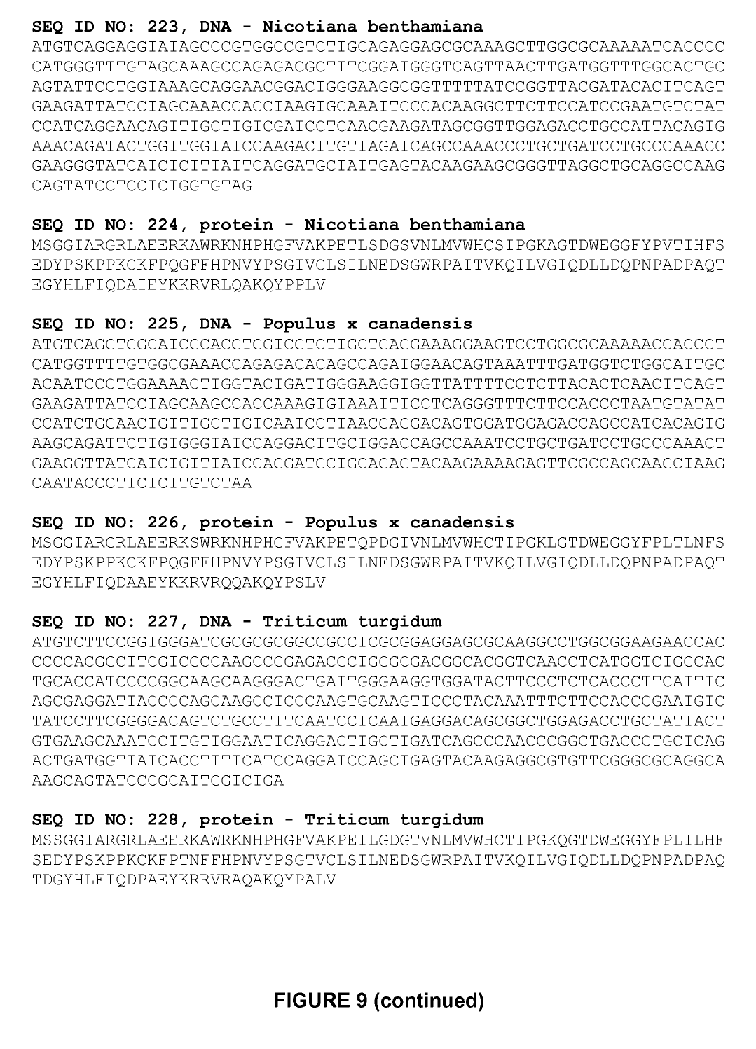

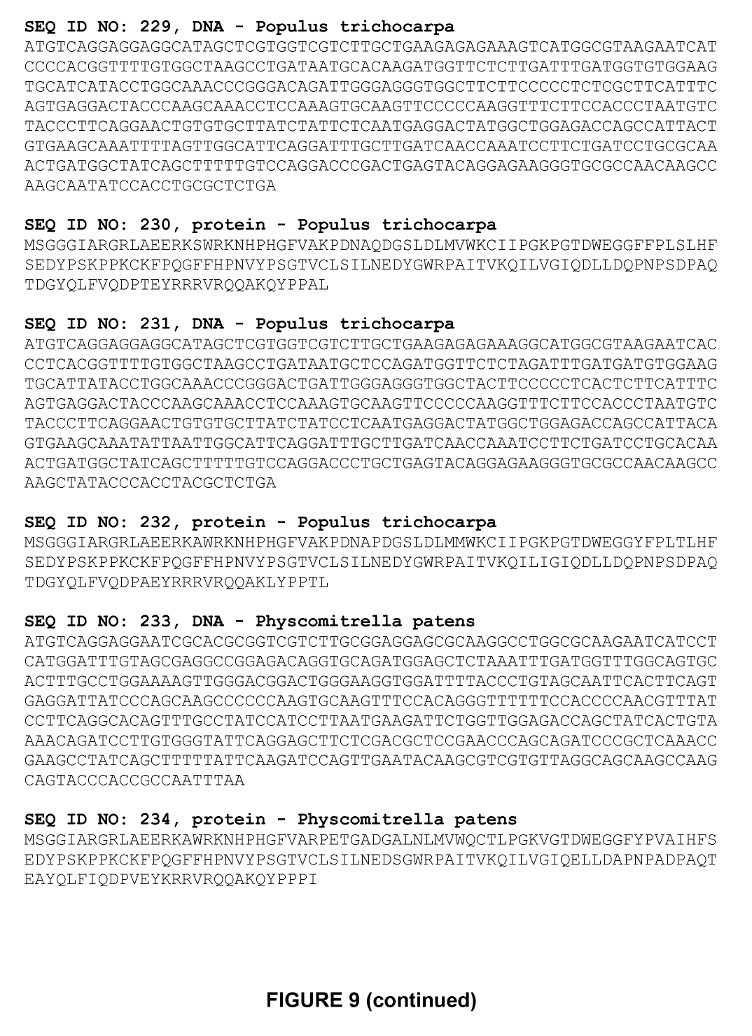

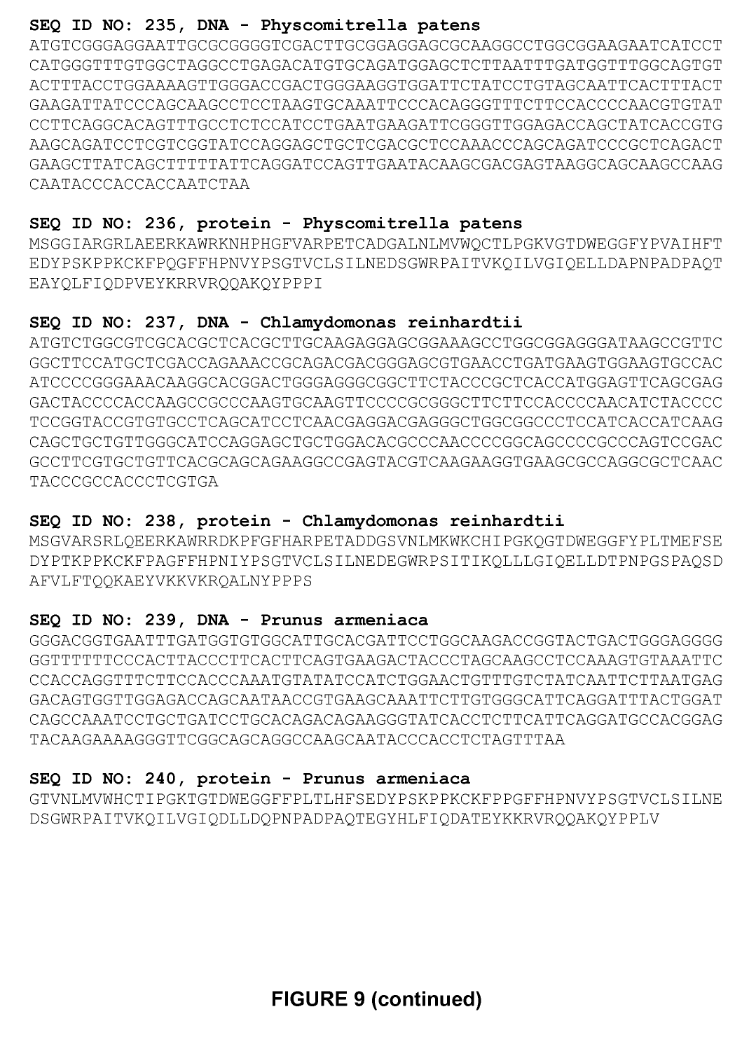

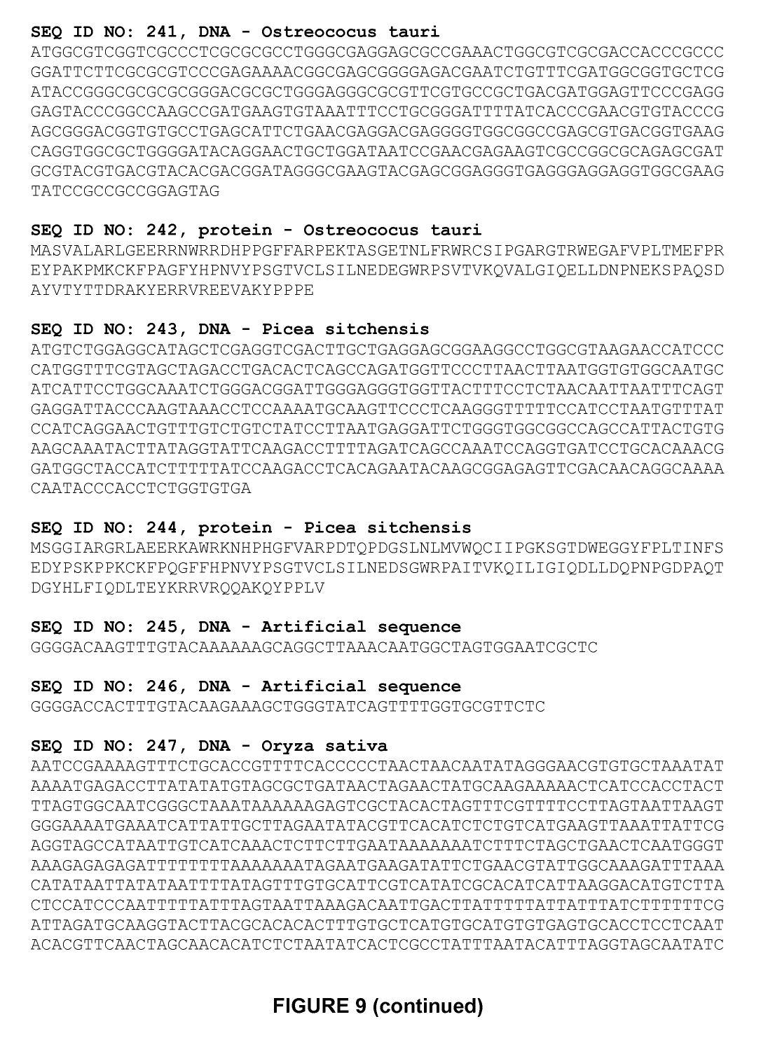

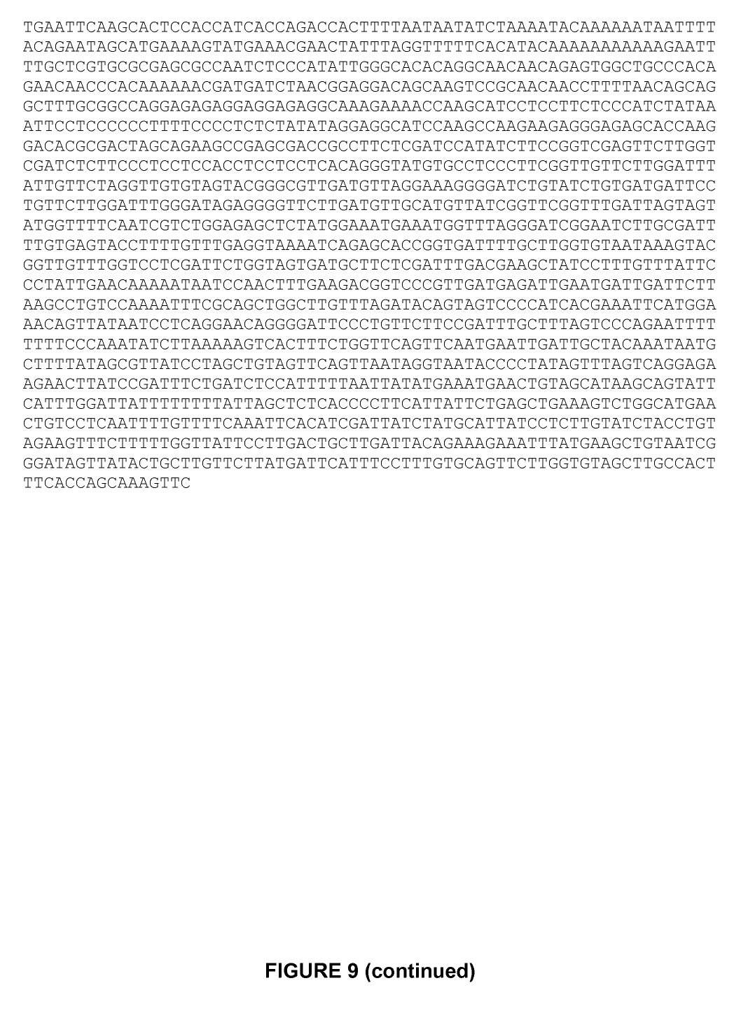

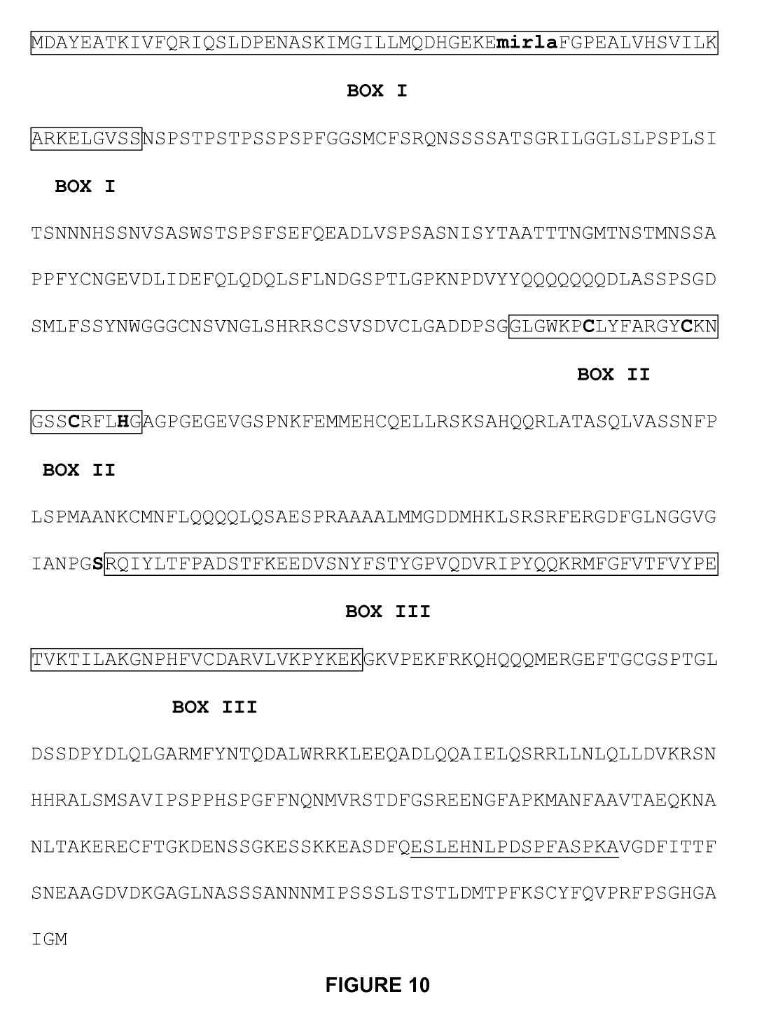

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Sequence conservation among SCE1 polypeptides shown is highest in the region comprising the UBC domain of the polypeptides. The SCE1 polypeptides are aligned in FIG. 7.

### Example 2.3

### Alignment of YEF1 Polypeptide Sequences

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Sequence conservation among YEF1 polypeptides is essentially in the N-terminal and central part of the protein along the NPD1, the C3H and the RRM domains of the polypeptides, the C-terminal domain usually being more variable in sequence length and composition. The YEF1 polypeptides are aligned in FIG. 12.

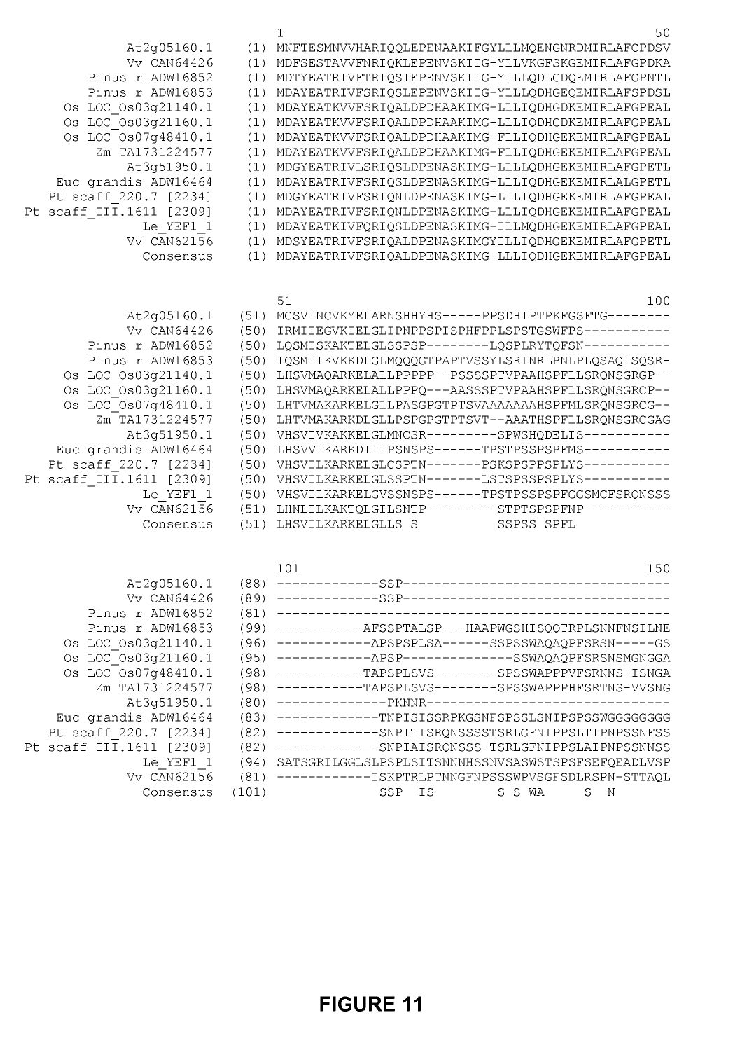

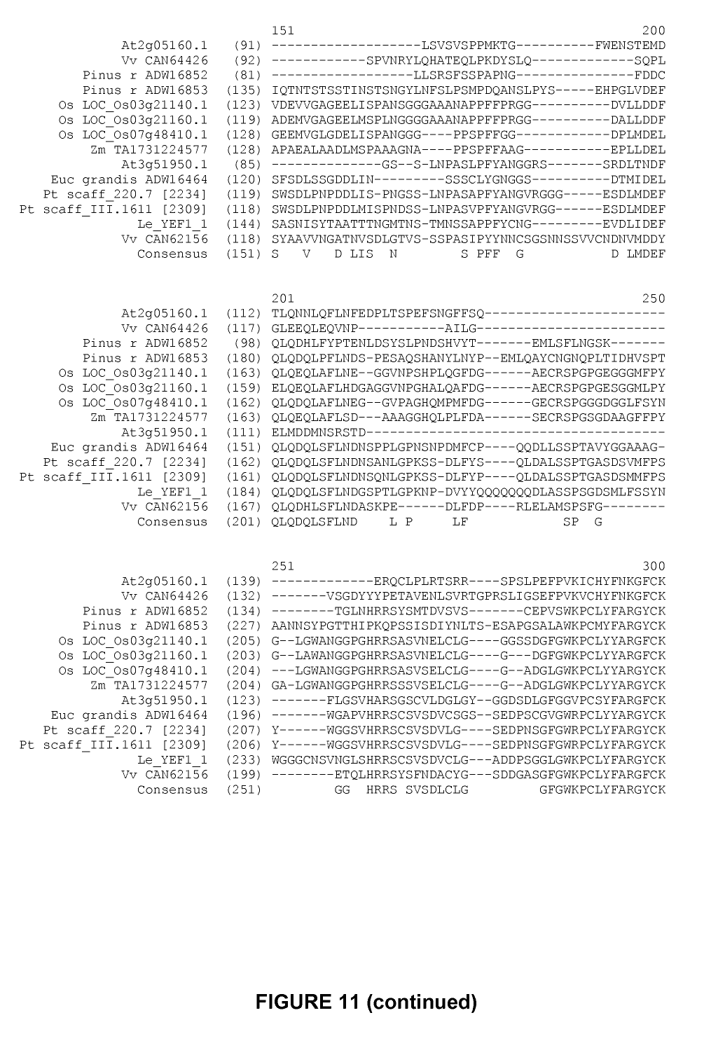

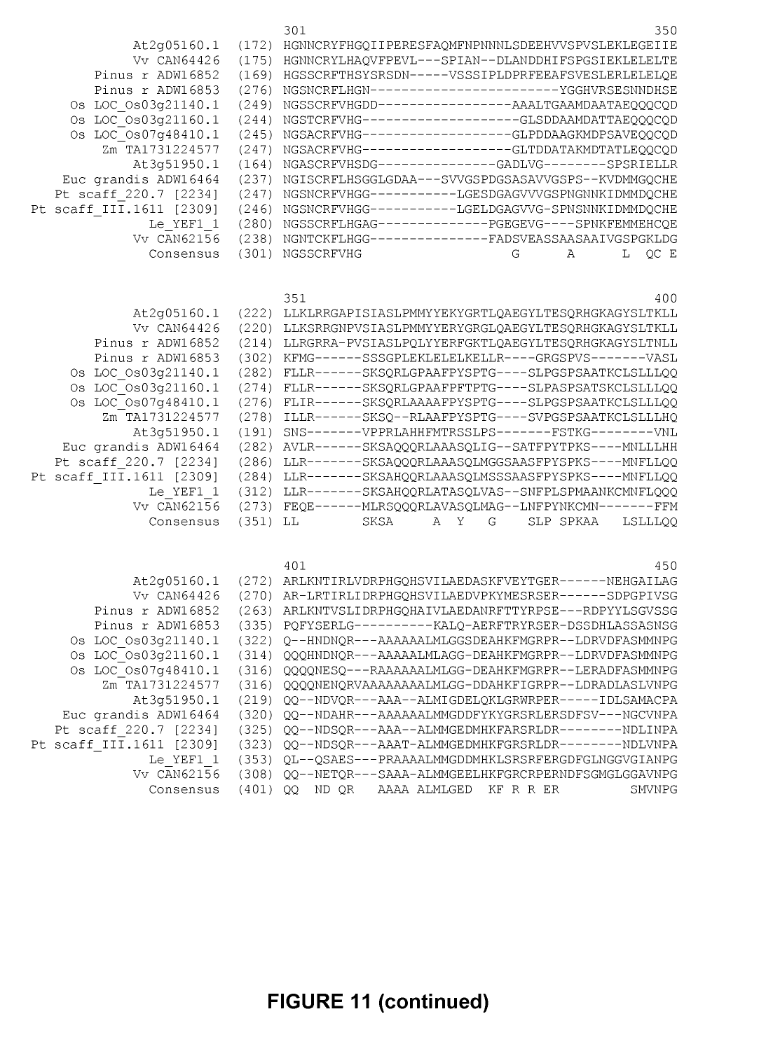

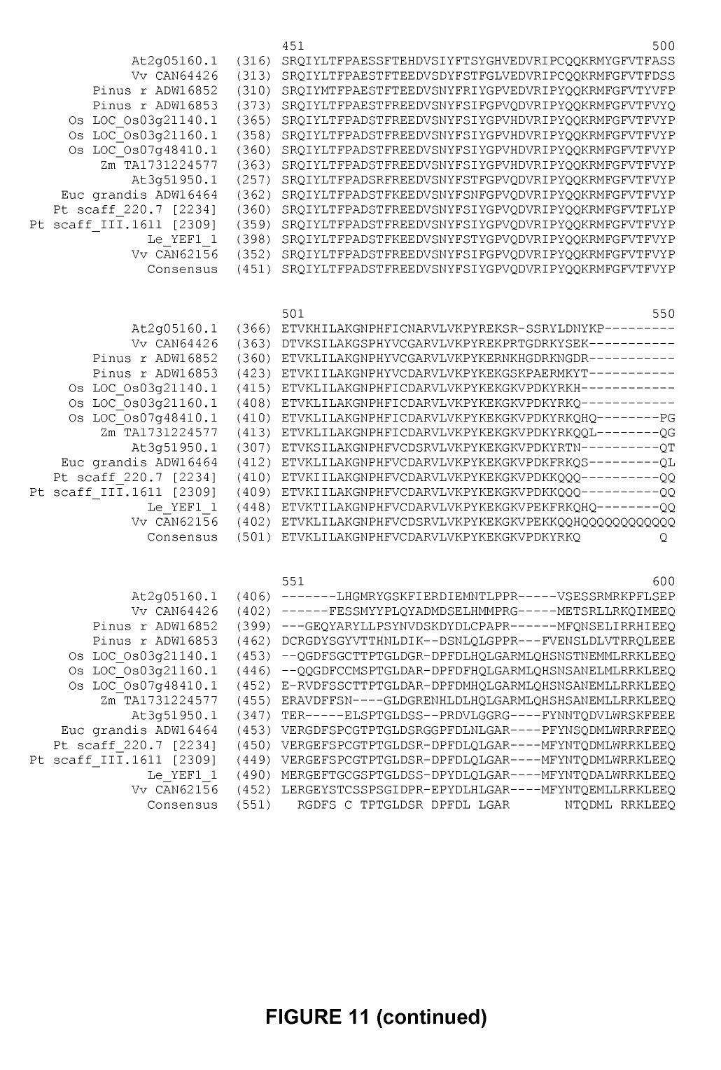

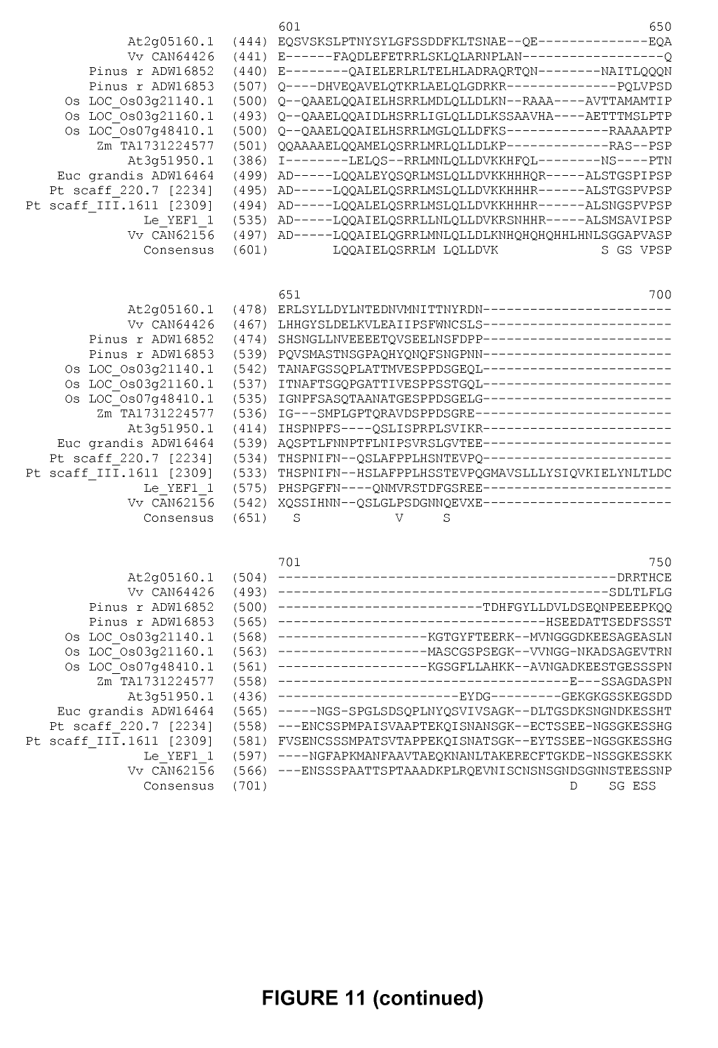

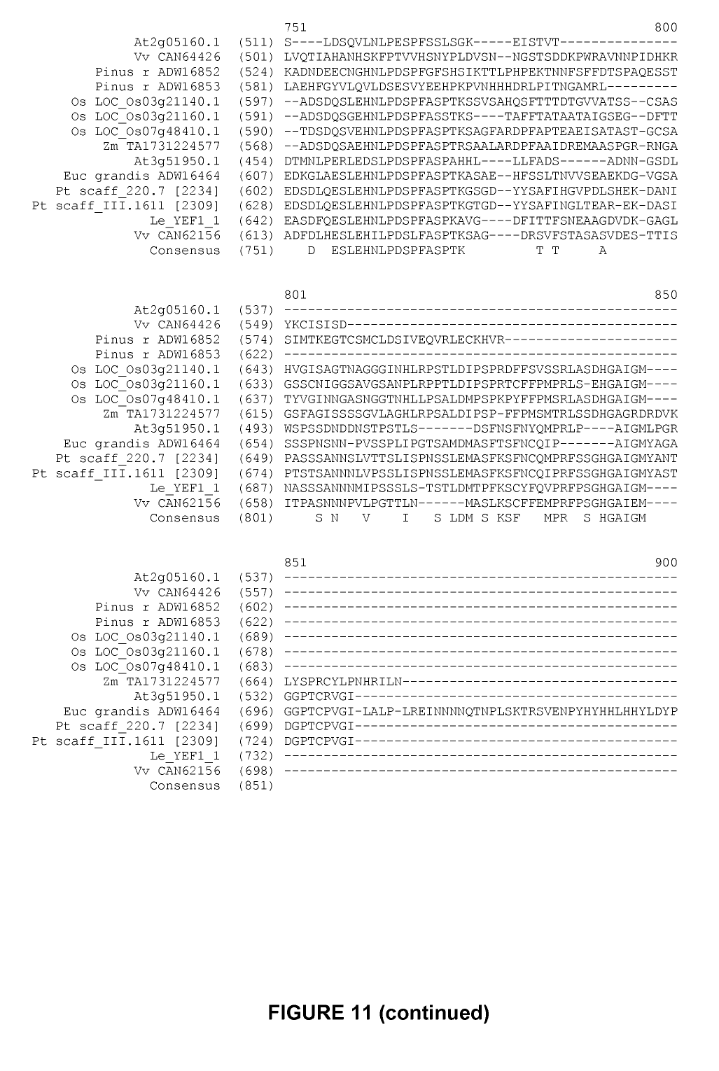

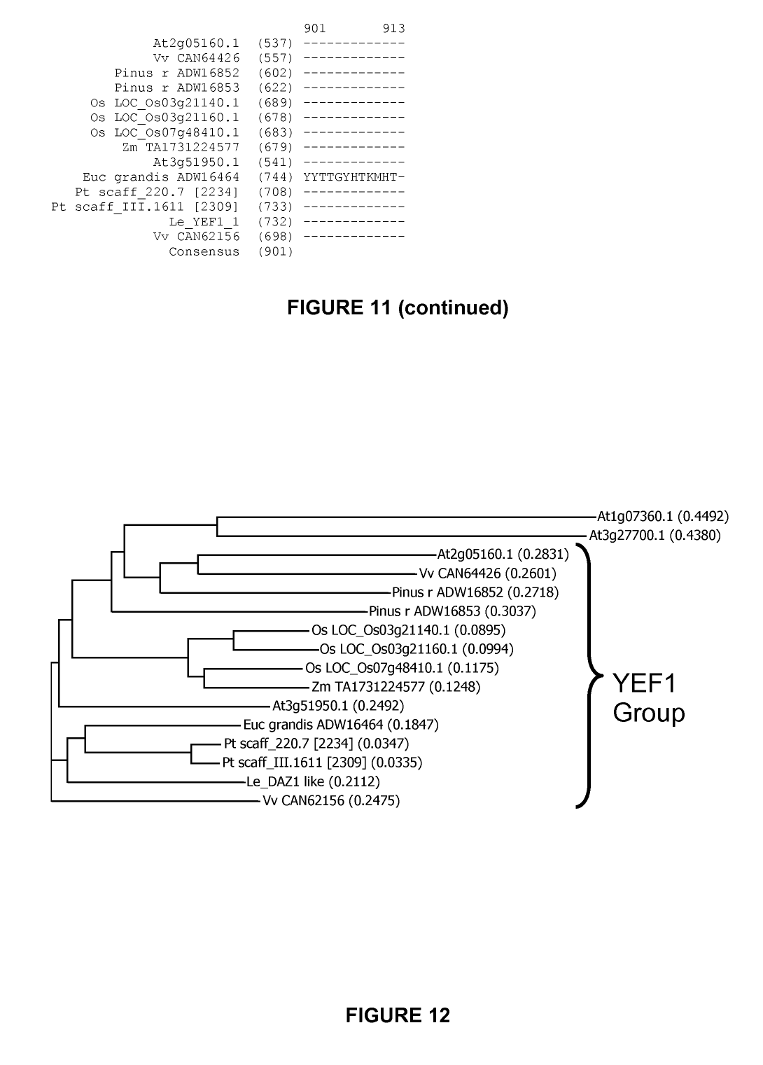

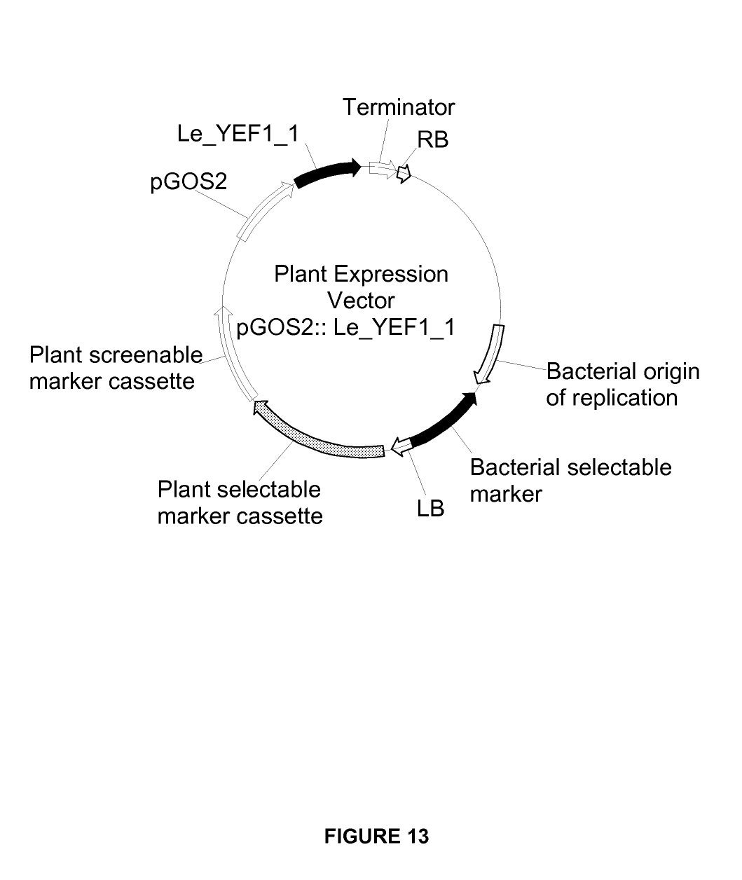

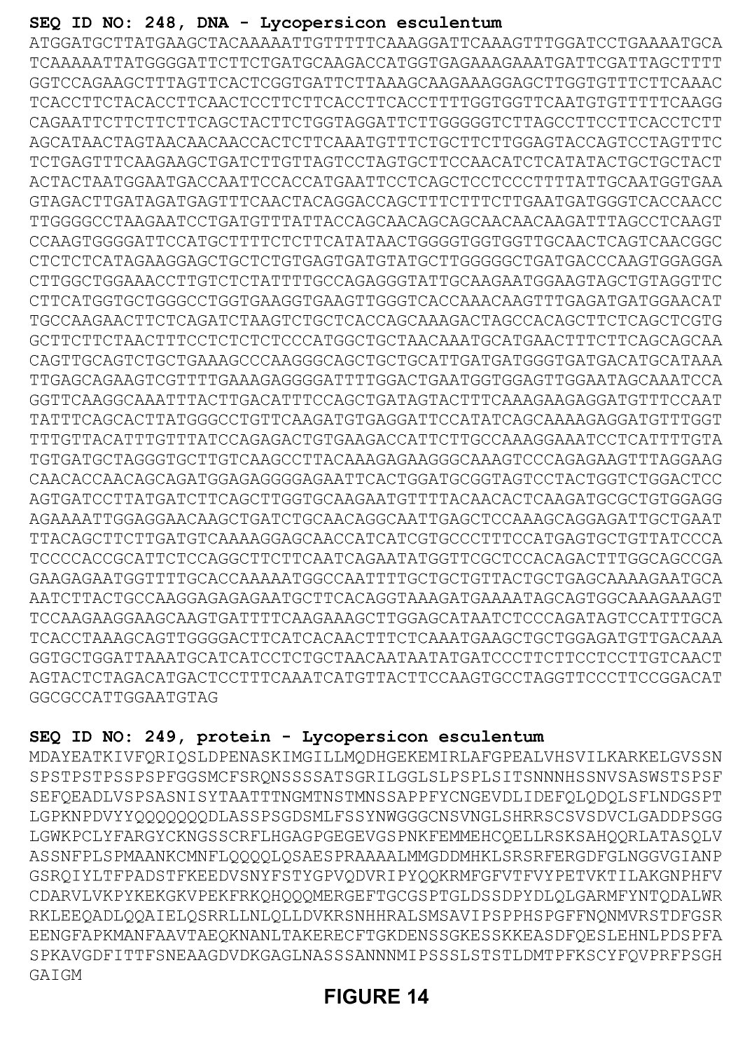

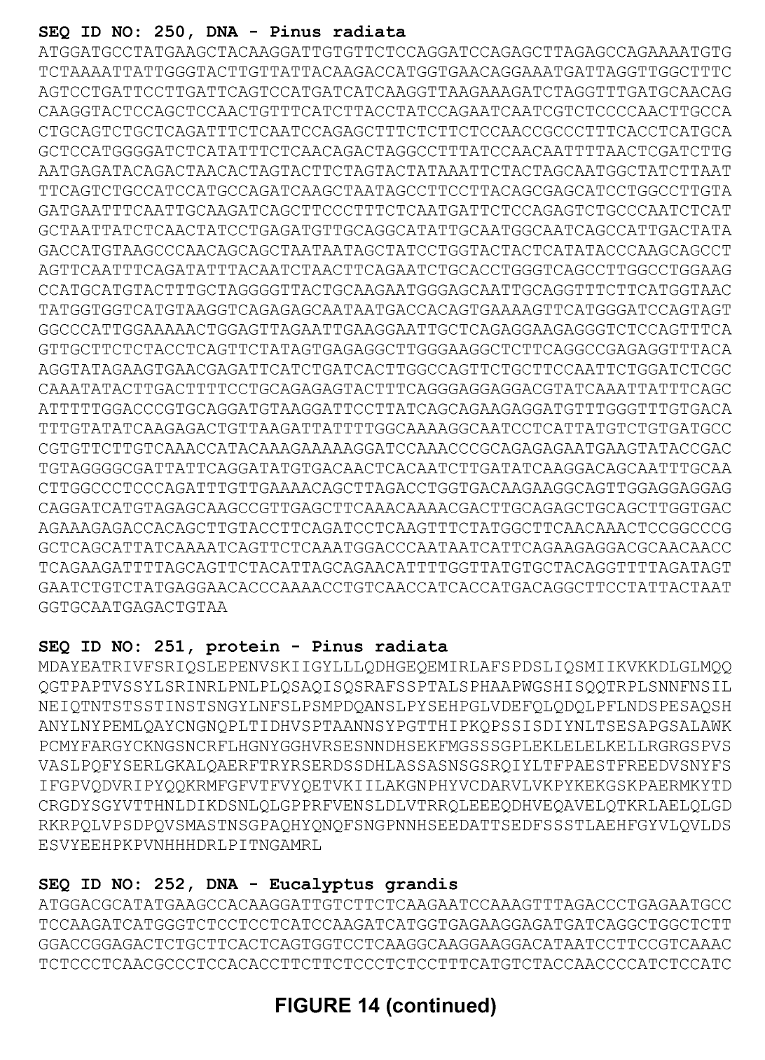

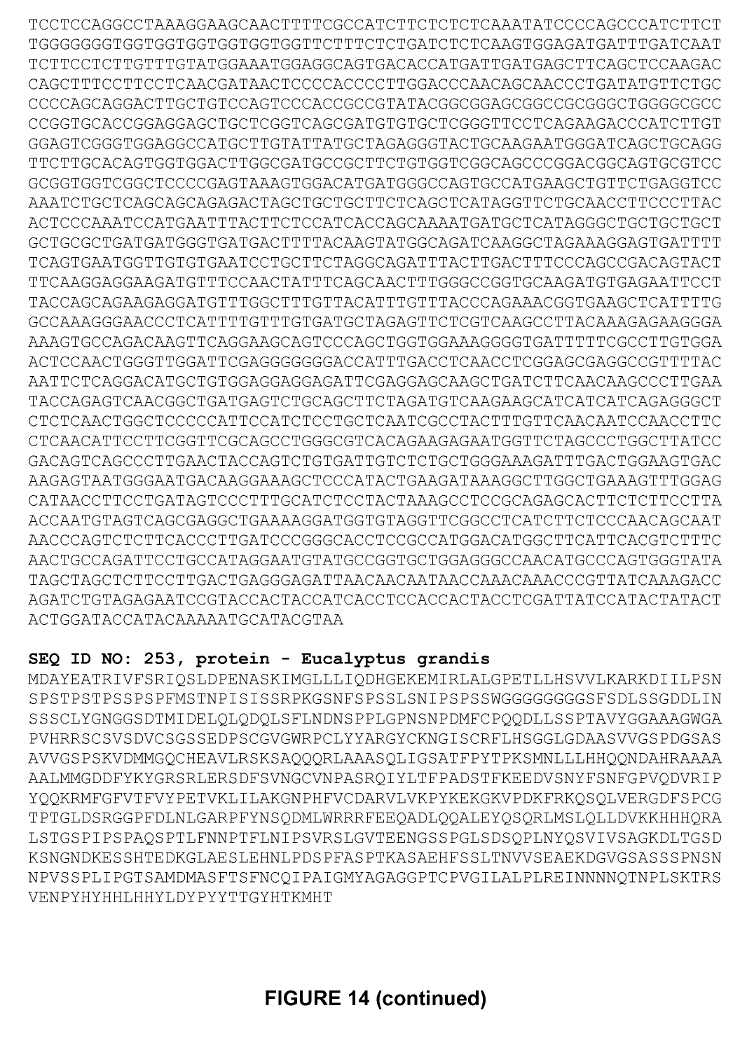

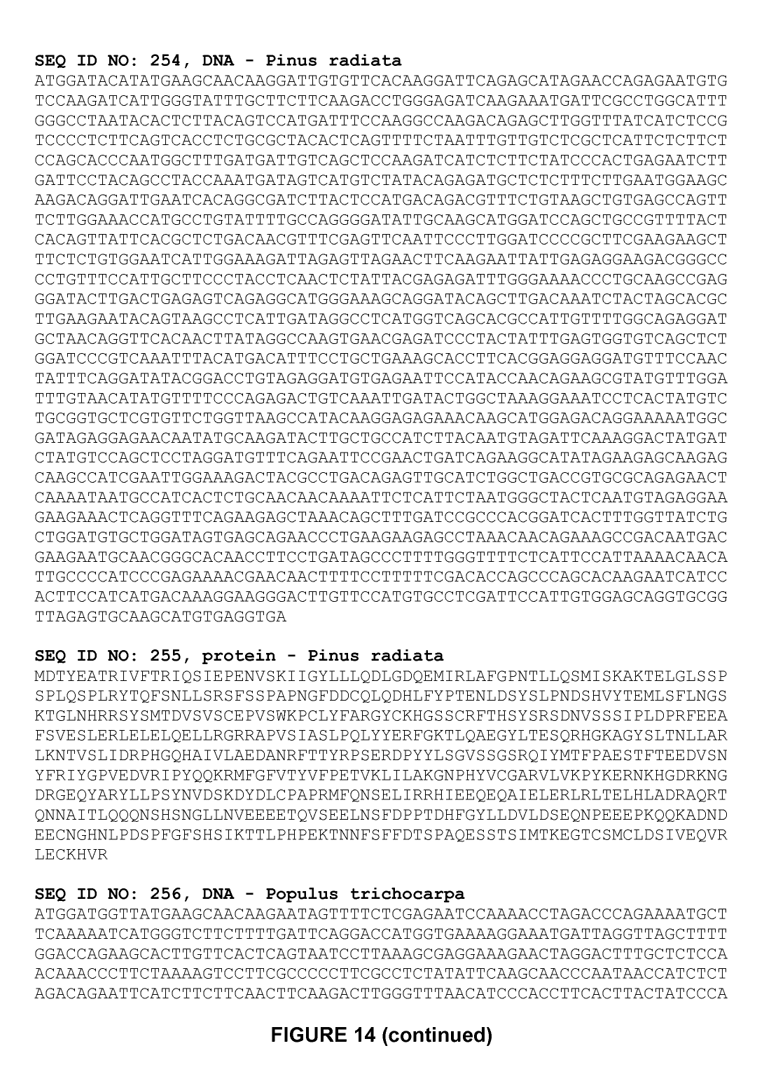

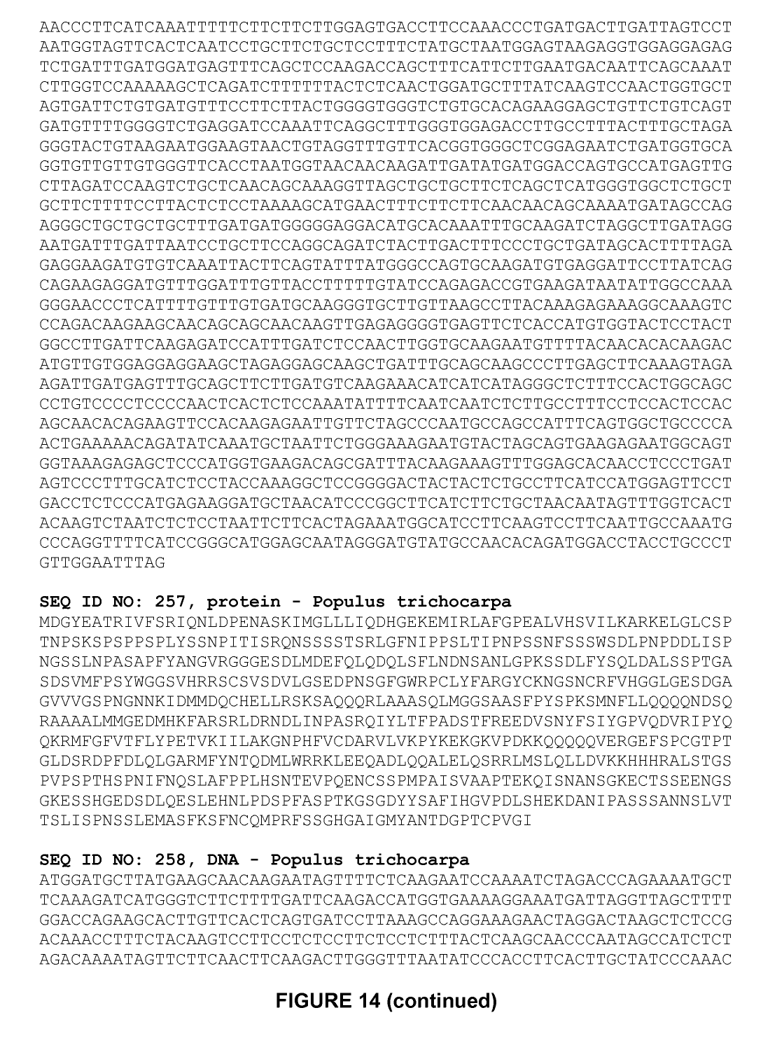

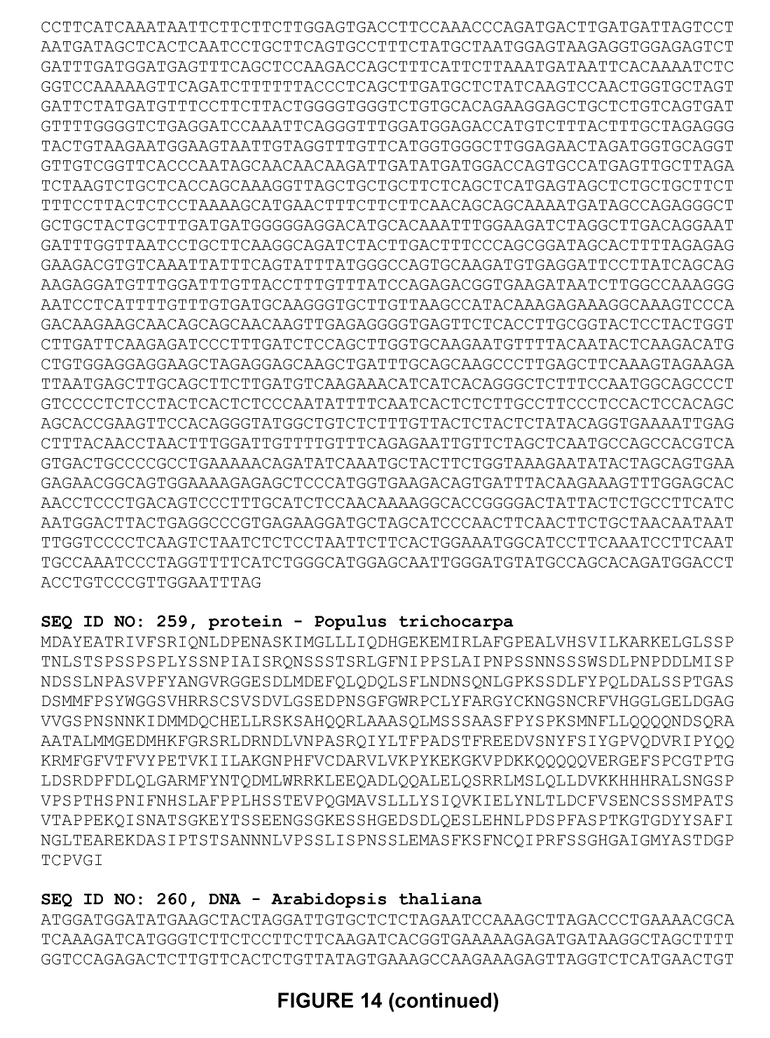

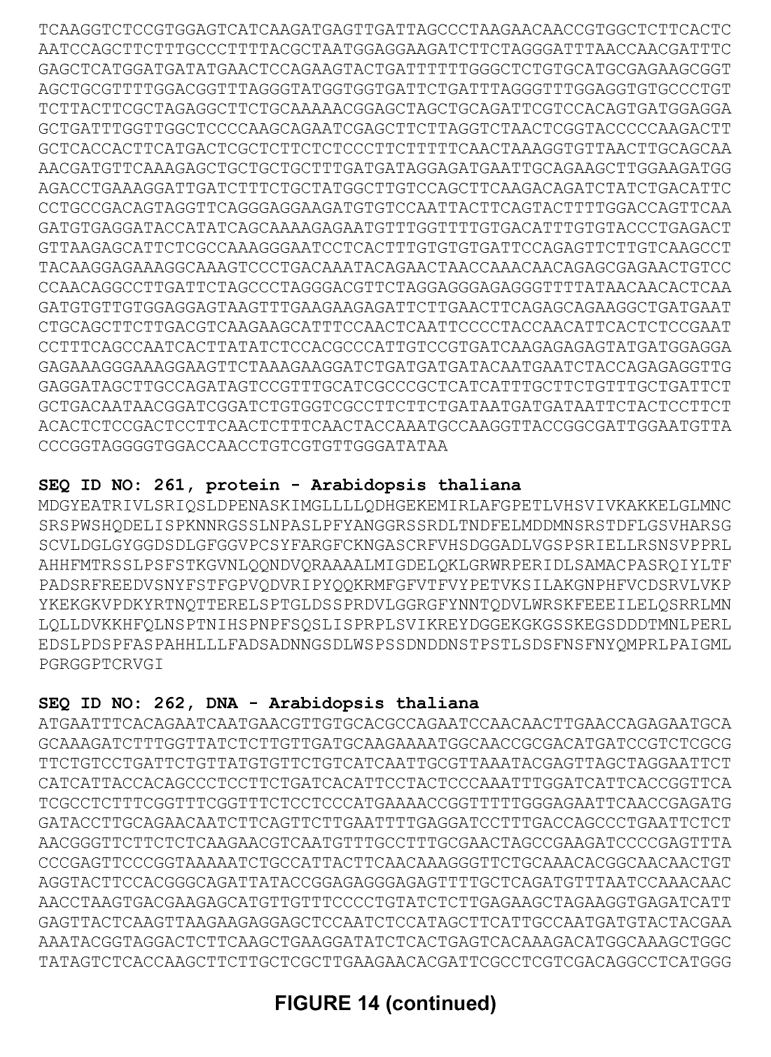

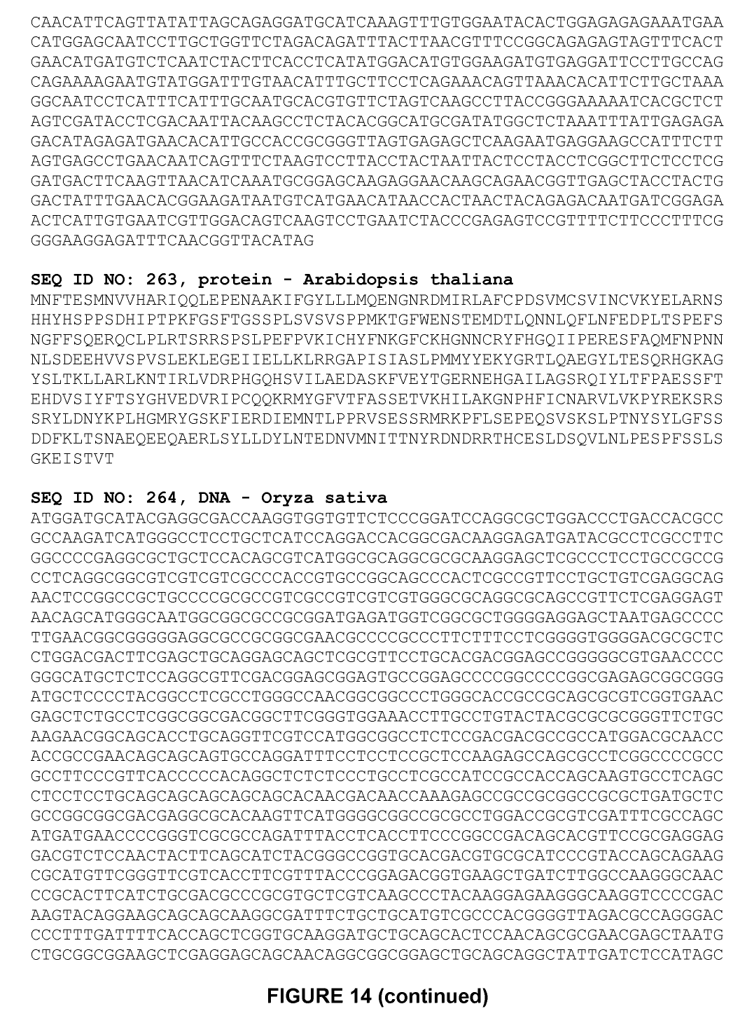

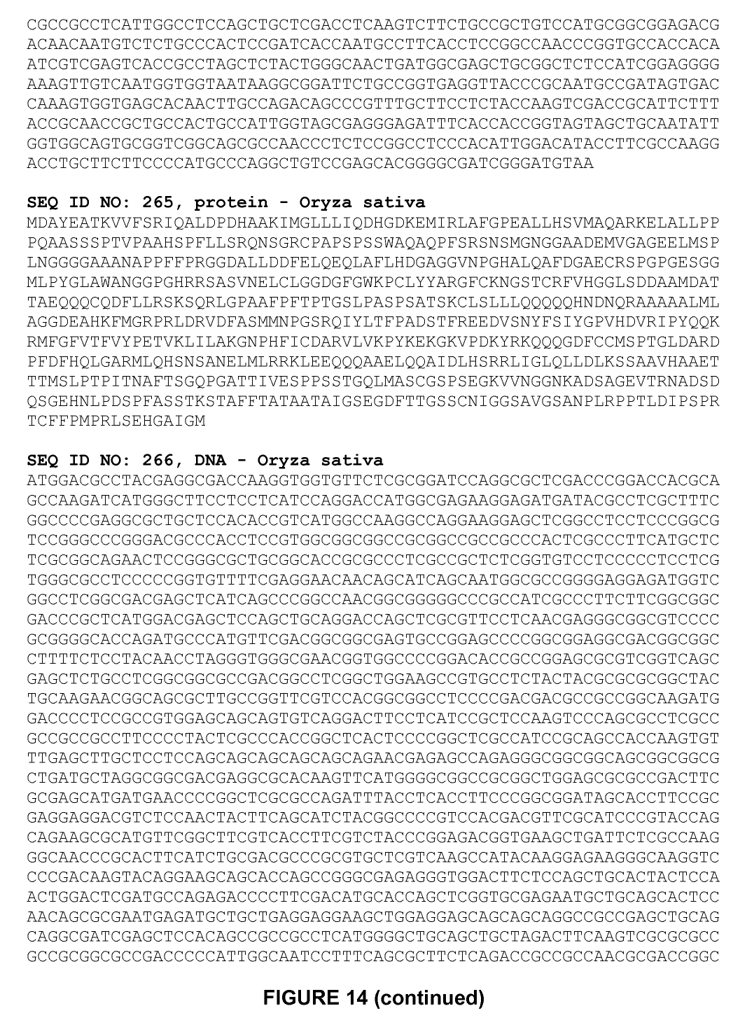

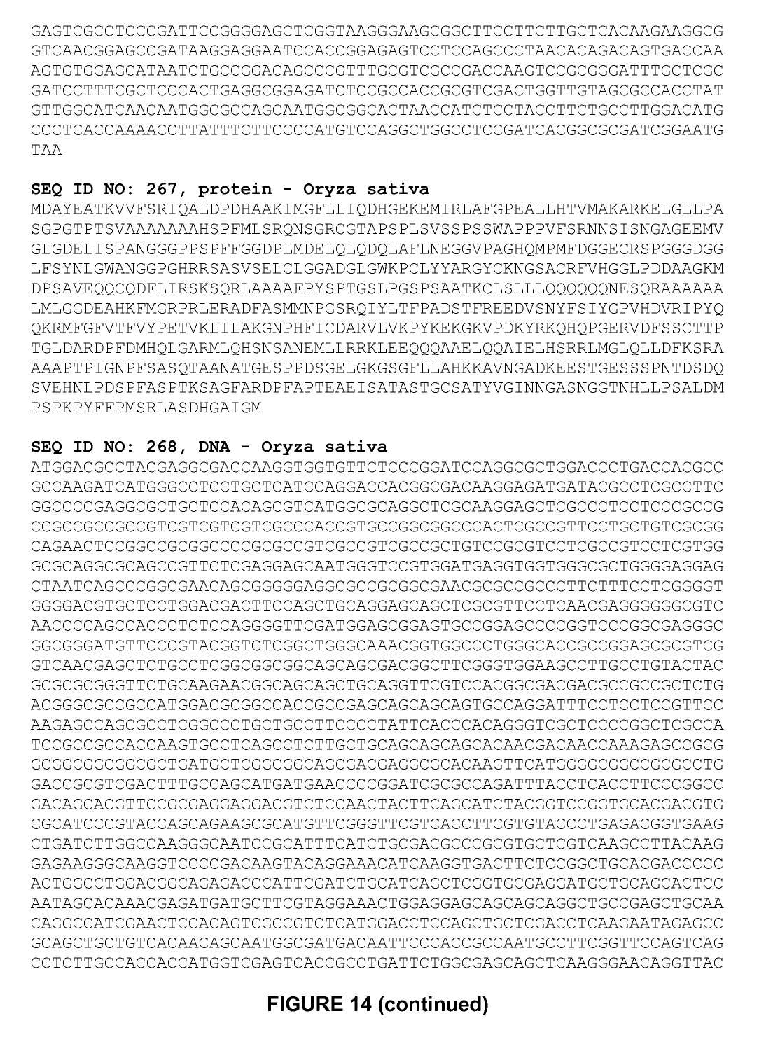

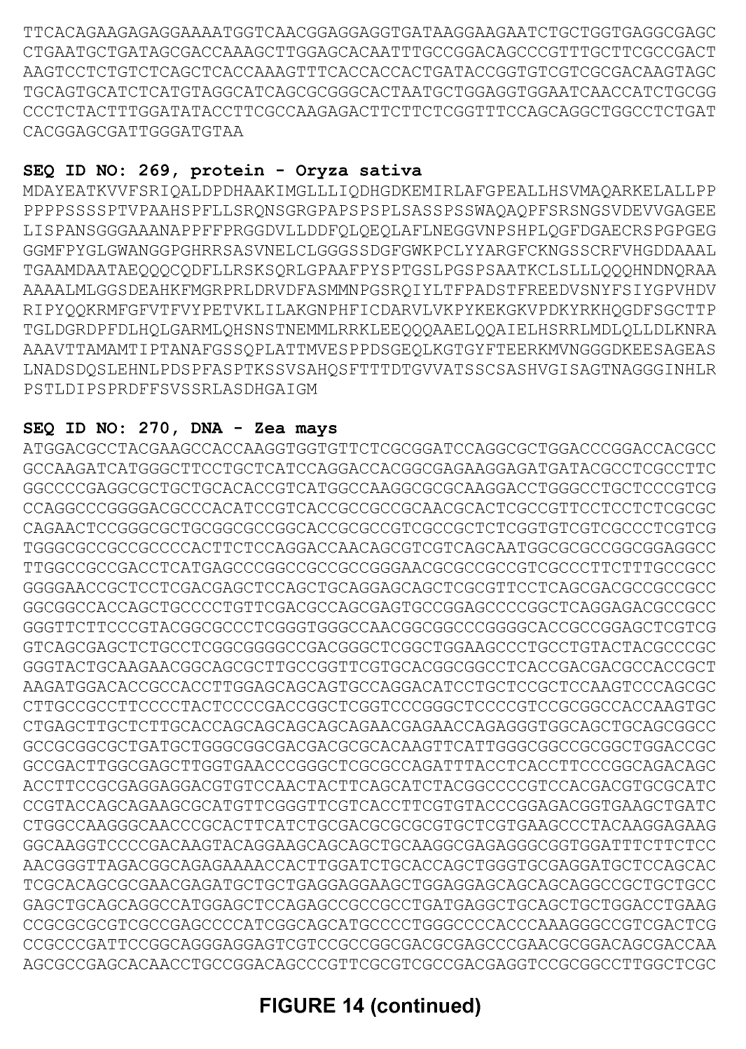

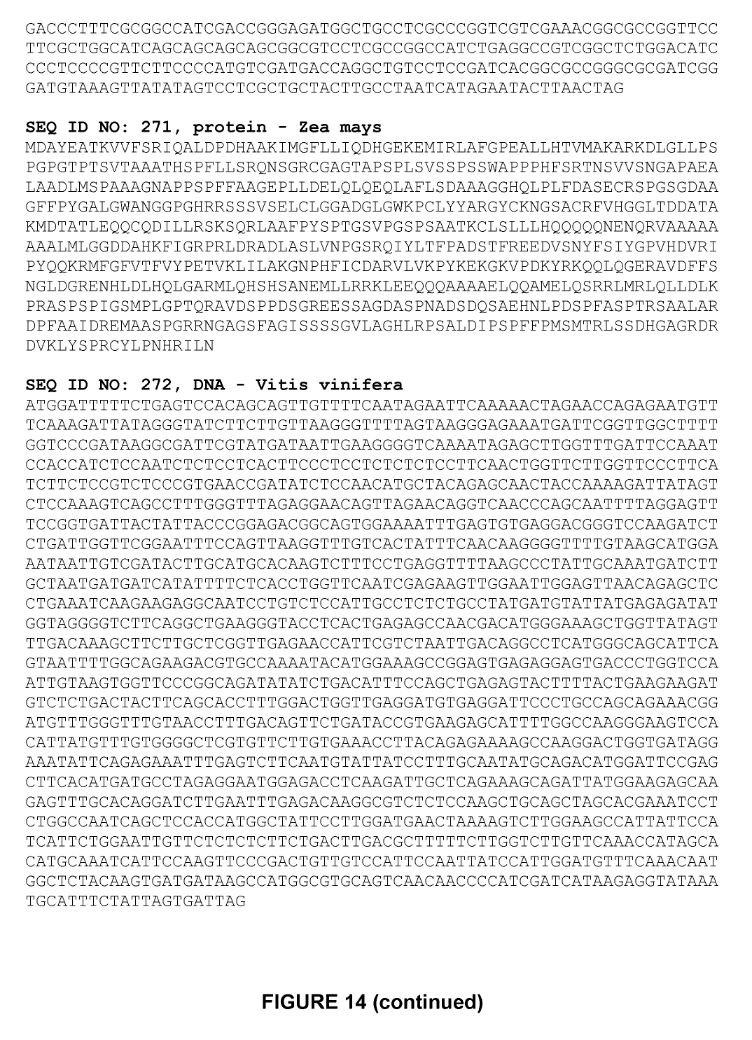

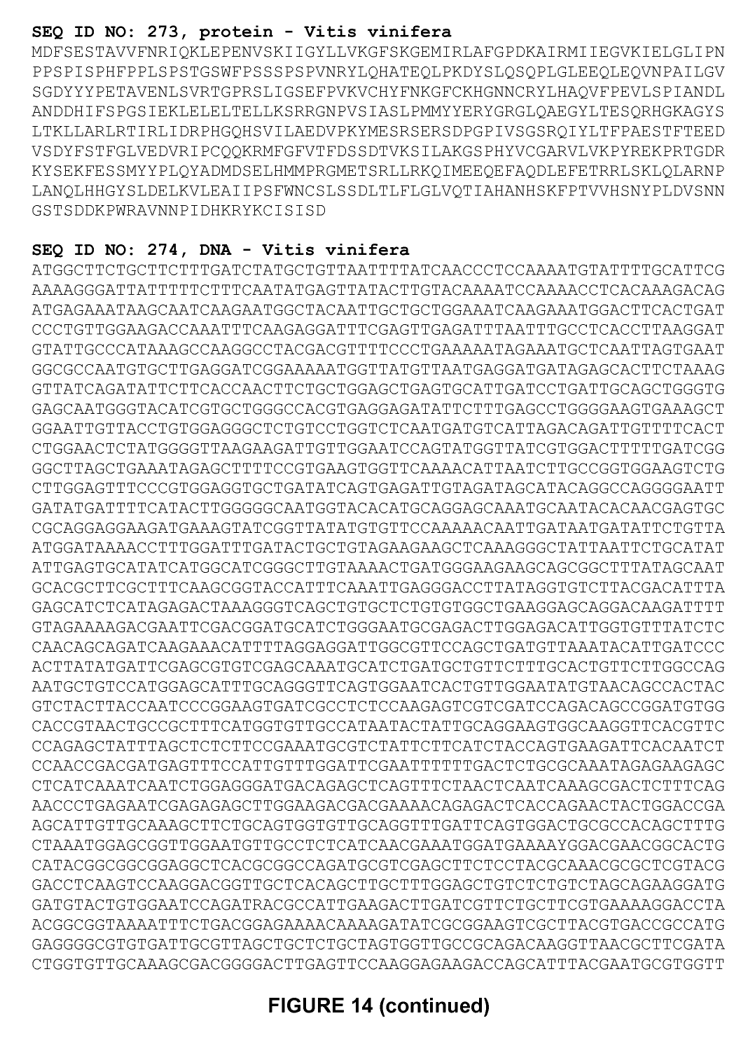

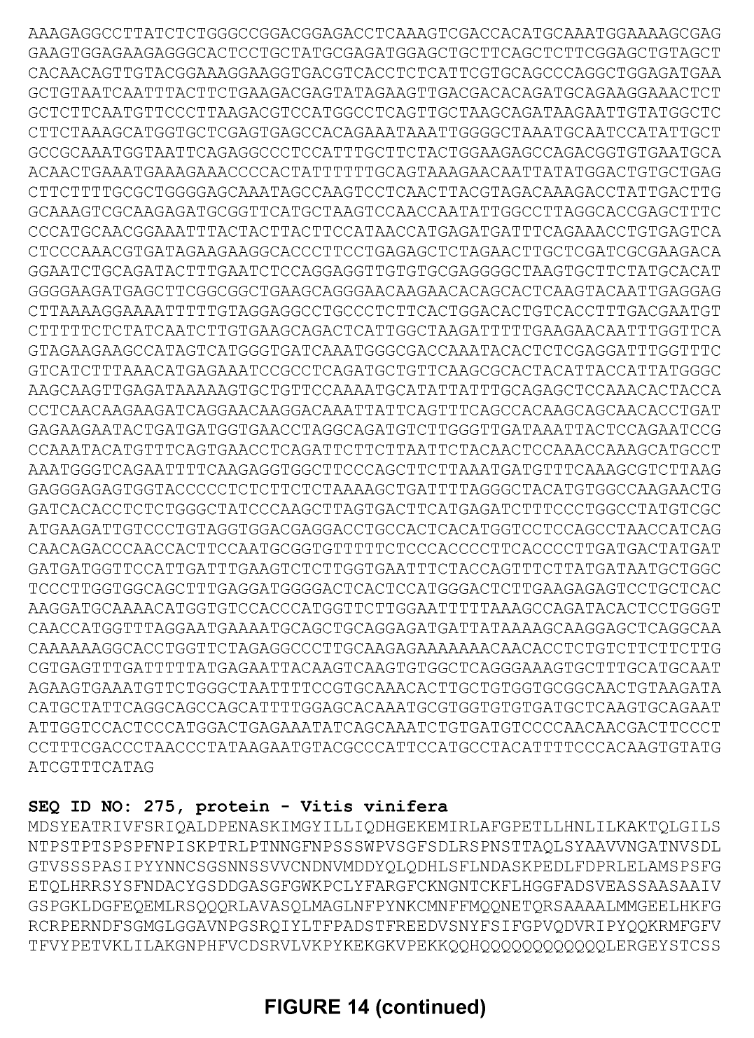

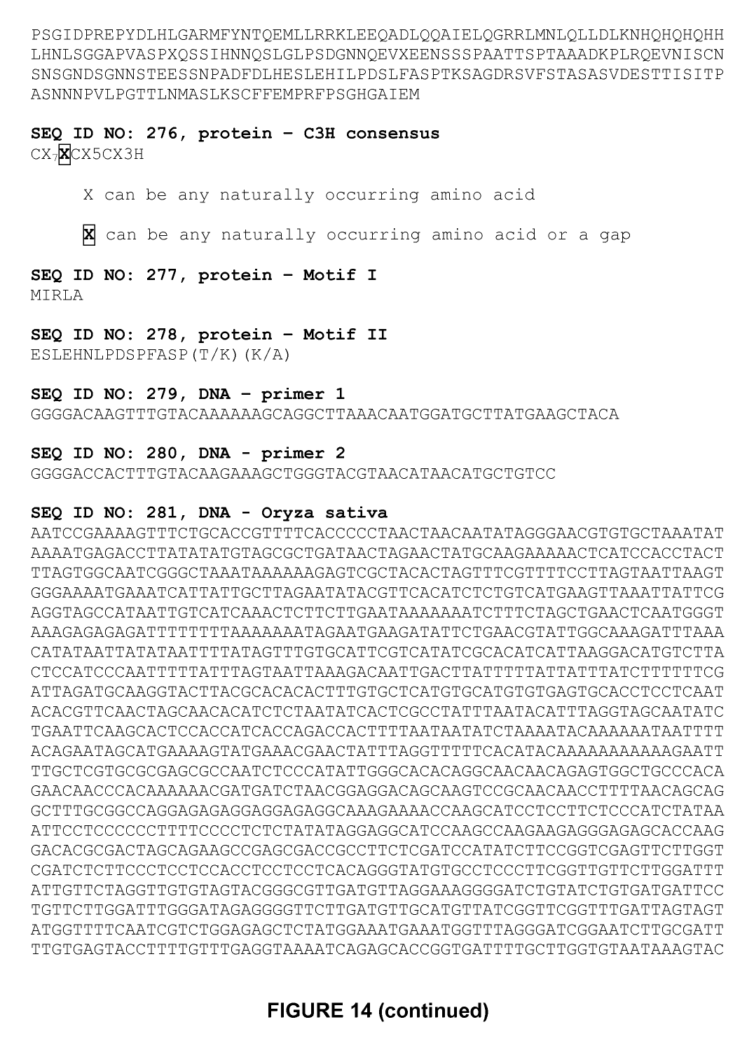

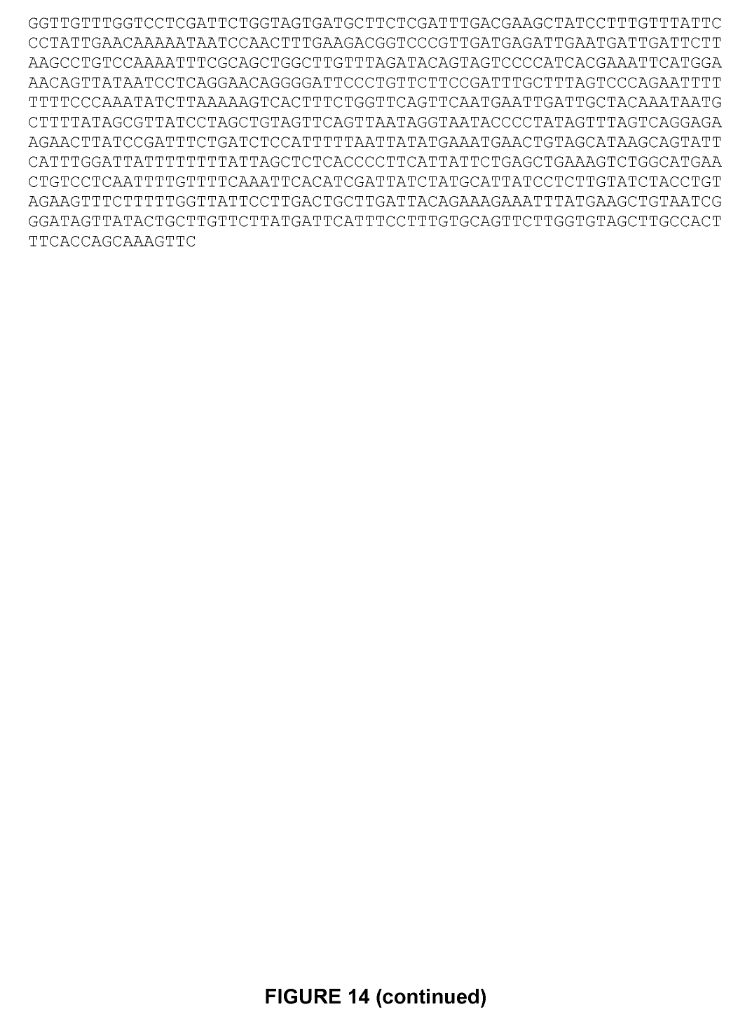

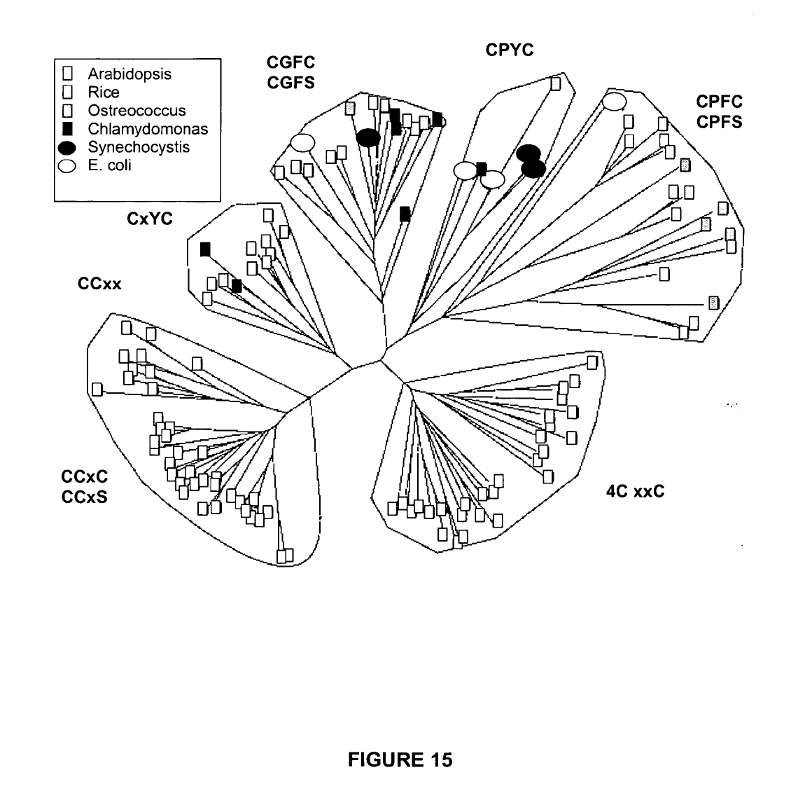

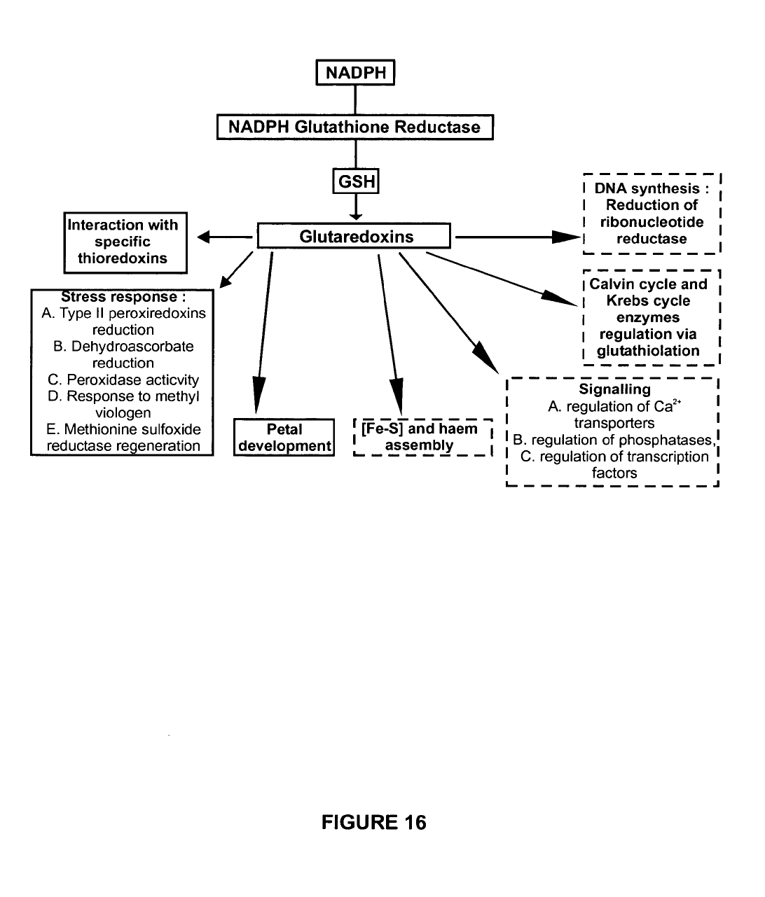

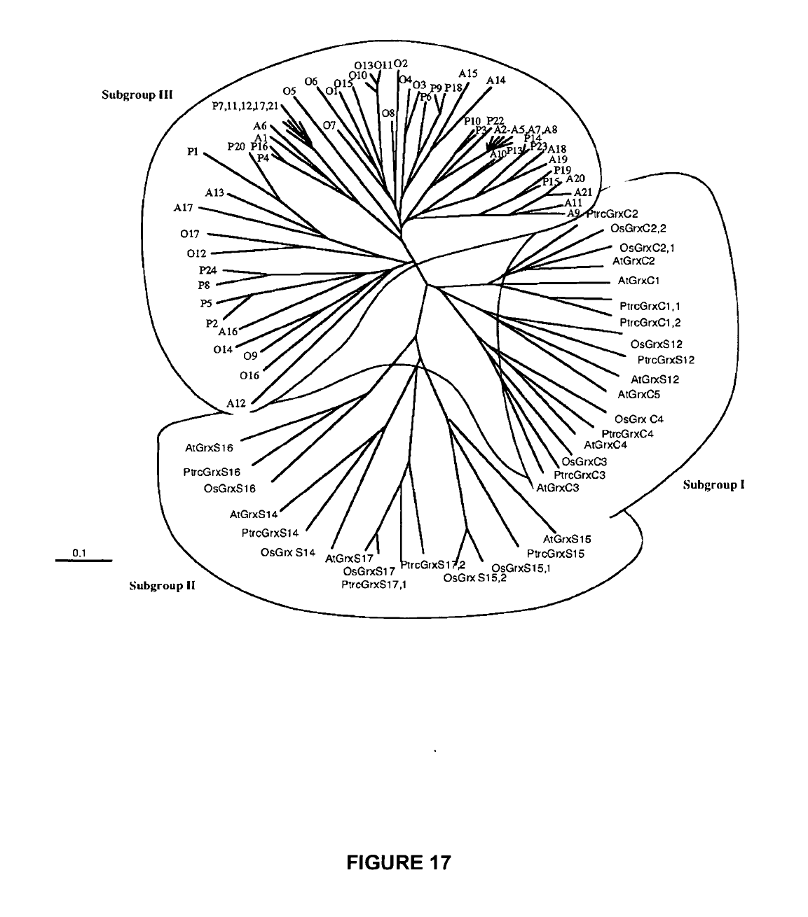

A phylogenetic tree of YEF1 polypeptides (FIG. 11) was constructed using a neighbour-joining clustering algorithm as provided in the AlignX programme from the Vector NTI (Invitrogen).

### Example 2.4

### Alignment of Subgroup III Grx Polypeptide Sequences

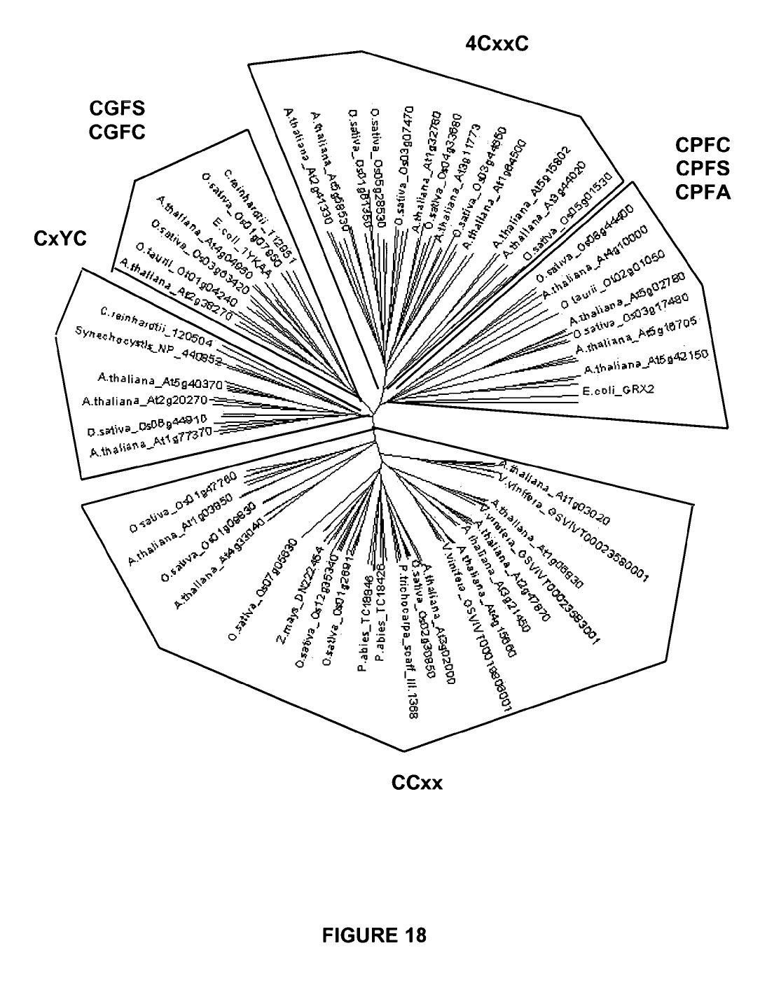

Alignment of polypeptide sequences was performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Minor manual editing was done to further optimise the alignment. A phylogenetic tree of Grx polypeptides (FIG. 18) was constructed using a neighbour-joining clustering algorithm as provided in the AlignX programme from the Vector NTI (Invitrogen).

### Example 2.5

### Alignment of Sister of FT Proteins or Homologues Thereof

Alignment of polypeptide sequences is performed using the AlignX programme from the Vector NTI (Invitrogen) which is based on the popular Clustal W algorithm of progressive alignment (Thompson et al. (1997) Nucleic Acids Res 25:4876-4882; Chenna et al. (2003). Nucleic Acids Res 31:3497-3500). Default values are for the gap open penalty of 10, for the gap extension penalty of 0.1 and the selected weight matrix is Blosum 62 (if polypeptides are aligned). Minor manual editing is done to further optimise the alignment. A phylogenetic tree is constructed using a neighbour-joining clustering algorithm provided in the AlignX programme from the Vector NTI (Invitrogen).

### Example 3

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix:Blosum62
First Gap: 12
Extending gap:2

Results of the software analysis are shown in Table B for the global similarity and identity over the full length of the polypeptide sequences.

The term “table B” used in this specification is to be taken to specify the content of table B1, table B2, table B3, and/or table B4.

The term “table B1” used in this specification is to be taken to specify the content of table B1. The term “table B2” used in this specification is to be taken to specify the content of table B2. The term “table B3” used in this specification is to be taken to specify the content of table B3. The term “table B4” used in this specification is to be taken to specify the content of table B4. In one preferred embodiment, the term “table B” means table B1. In another preferred embodiment, the term “table B” means table B2. In another preferred embodiment, the term “table B” means table B3. In another preferred embodiment, the term “table B” means table B4.

### Example 3.1

### PRE-Like Polypeptides

The percentage identity between the PRE-like polypeptide sequences useful in performing the methods of the invention can be as low as 47.4% amino acid identity compared to SEQ ID NO: 2.

TABLE B1
MatGAT results for global similarity and identity between SEQ ID NO: 2 (TaPRE-like) and
other PRE-like sequences (identifiers as in Table A), calculated over the full length of the
polypeptide sequences. % ID and % SIM are percentage of respectively sequence identity
and similarity.

| | % ID | % SIM | | % ID | % SIM |
|---|---|---|---|---|---|
| TaPRE-like vs. GSVIV4 | 73.9 | 87 | TaPRE-like vs. TA36763 | 63.4 | 80.4 |
| TaPRE-like vs. DY672743 | 67 | 83 | TaPRE-like vs. DT527245 | 71.7 | 84.8 |
| TaPRE-like vs. AT1G26945 | 75.5 | 89.4 | TaPRE-like vs. DT602195 | 55.8 | 71.8 |
| TaPRE-like vs. TA4110 | 62 | 76.1 | TaPRE-like vs. TC110752 | 76.6 | 89.1 |
| TaPRE-like vs. TA8292 | 60.2 | 80.4 | TaPRE-like vs. TA2164 | 57 | 78.3 |
| TaPRE-like vs. TA6224 | 67 | 82.8 | TaPRE-like vs. TA3862 | 69.9 | 83.7 |
| TaPRE-like vs. TA36504 | 91.3 | 96.7 | TaPRE-like vs. AT3G47710 | 68.1 | 85.9 |
| TaPRE-like vs. CO541258 | 69.8 | 83.3 | TaPRE-like vs. TA89858 | 54.8 | 81.5 |
| TaPRE-like vs. TA207044 | 52.1 | 77.2 | TaPRE-like vs. EL465600 | 58.5 | 76.1 |
| TaPRE-like vs. XII.633 | 72 | 87 | TaPRE-like vs. TA44490 | 52.1 | 78.3 |
| TaPRE-like vs. TA5496 | 55.9 | 70.7 | TaPRE-like vs. TA42071 | 57 | 81.5 |
| TaPRE-like vs. TA44221 | 68.4 | 86.3 | TaPRE-like vs. EL487276 | 63 | 82.6 |
| TaPRE-like vs. TA215077 | 55.4 | 79.3 | TaPRE-like vs. AJ752013 | 69.9 | 83.9 |
| TaPRE-like vs. DY660883 | 63 | 77.2 | TaPRE-like vs. CK367883 | 48.6 | 68.6 |
| TaPRE-like vs. BE705205 | 59.1 | 81.5 | TaPRE-like vs. CA090192 | 57 | 72.8 |
| TaPRE-like vs. BU045110 | 61.3 | 79.3 | TaPRE-like vs. DW498223 | 76.1 | 87 |
| TaPRE-like vs. TA170348 | 53.8 | 78.3 | TaPRE-like vs. BI268948 | 68.8 | 87.1 |
| TaPRE-like vs. CD416537 | 69.9 | 86 | TaPRE-like vs. TA53762 | 70.7 | 84.8 |
| TaPRE-like vs. TA62505 | 74.2 | 88.2 | TaPRE-like vs. BU048569 | 53.7 | 76.3 |
| TaPRE-like vs. AJ758453 | 65.2 | 80.4 | TaPRE-like vs. DW501889 | 69.1 | 81.9 |
| TaPRE-like vs. 129.2 | 71 | 85.9 | TaPRE-like vs. DN151440 | 52.7 | 67.4 |
| TaPRE-like vs. TA4303 | 65.6 | 83.7 | TaPRE-like vs. EL408974 | 64.1 | 80.4 |
| TaPRE-like vs. TA43072 | 69.6 | 82.6 | TaPRE-like vs. TA3169 | 69.9 | 83.9 |
| TaPRE-like vs. AT3G28857 | 61.3 | 80.4 | TaPRE-like vs. TA5285 | 69.6 | 79.3 |
| TaPRE-like vs. CV503041 | 66.3 | 82.6 | TaPRE-like vs. GSVIV0 | 47.4 | 71.7 |
| TaPRE-like vs. CV2972 | 64.1 | 81.5 | TaPRE-like vs. CO553461 | 64.1 | 83.7 |
| TaPRE-like vs. Os02g51320 | 57 | 79.3 | TaPRE-like vs. TA21468 | 64.5 | 80.6 |
| TaPRE-like vs. TC110807 | 66.7 | 82.6 | TaPRE-like vs. XVII.359 | 69.6 | 80.4 |
| TaPRE-like vs. CV532618 | 67.7 | 82.6 | TaPRE-like vs. Os04g54900 | 58.7 | 78.8 |
| TaPRE-like vs. TA33922 | 52.7 | 79.3 | TaPRE-like vs. CV167880 | 73.9 | 85.9 |
| TaPRE-like vs. TA98487 | 54.3 | 79.3 | TaPRE-like vs. BE205620 | 59.6 | 78.3 |
| TaPRE-like vs. GSVIV1 | 75 | 84.8 | TaPRE-like vs. TA56389 | 75.3 | 89.2 |
| TaPRE-like vs. AT1G74500 | 69.5 | 86 | TaPRE-like vs. TA18273 | 79.6 | 89.1 |
| TaPRE-like vs. TA103938 | 55.9 | 75 | TaPRE-like vs. EH367818 | 68.5 | 83.7 |
| TaPRE-like vs. AT5G15160 | 54.3 | 76.6 | | | |

### Example 3.2

### SCE1 Polypeptides

Results of the MatGAT software analysis are shown in Table B2 for the global similarity and identity over the full length of the polypeptide sequences. Percentage identity is given below the diagonal and percentage similarity is given above the diagonal (normal face).

The percentage identity between the SCE1 polypeptide sequences useful in performing the methods of the invention can be as low as 57.5% amino acid identity compared to SEQ ID NO: 200.

TABLE B2
MatGAT results for global similarity and identity over the full length of the polypeptide sequences.

| | | 1. | 2. | 3. | 4. | 5. | 6. | 7. | 8. | 9. | 10. | 11. | 12. |
|---|---|---|---|---|---|---|---|---|---|---|---|---|---|
| 1. | Glyma | | 95.6 | 96.9 | 76.7 | 73.6 | 97.5 | 82.4 | 93.8 | 95.6 | 94.4 | 54.1 | 96.9 |
| | SCE1_1 | | | | | | | | | | | | |
| 2. | Picsi | 88.1 | | 96.2 | 76.2 | 72.5 | 96.9 | 83.1 | 92.5 | 95.6 | 93.8 | 53.8 | 95.6 |
| | SCE1_1 | | | | | | | | | | | | |
| 4. | Popul | 94.4 | 90 | | 76.2 | 73.1 | 97.5 | 81.9 | 95 | 95.6 | 95.6 | 53.8 | 96.2 |
| | SCE1_1 | | | | | | | | | | | | |
| 5. | Pruar | 74.2 | 70 | 75 | | 59.1 | 76.2 | 66 | 73.9 | 76.2 | 74.4 | 63.9 | 76.2 |
| | SCE1_1 | | | | | | | | | | | | |
| 6. | Ostta | 57.9 | 57.5 | 56.2 | 47.2 | | 73.1 | 77.4 | 72 | 73.1 | 73.1 | 51.6 | 72.5 |
| | SCE1_1 | | | | | | | | | | | | |
| 5. | Vitvi | 93.1 | 90.6 | 93.1 | 72.5 | 57.5 | | 83.1 | 95 | 96.9 | 95 | 54.4 | 96.9 |
| | SCE1_1 | | | | | | | | | | | | |
| 7. | Chlre | 67.9 | 69.4 | 68.1 | 56 | 64.8 | 70 | | 80.7 | 81.9 | 81.2 | 58.5 | 81.2 |
| | SCE1_1 | | | | | | | | | | | | |
| 8. | Tritu | 91.3 | 87.6 | 91.9 | 72 | 57.1 | 93.2 | 68.3 | | 95.7 | 95 | 55.3 | 94.4 |
| | SCE1_1 | | | | | | | | | | | | |
| 9. | Orysa | 91.9 | 89.4 | 91.9 | 73.1 | 58.1 | 93.8 | 69.4 | 94.4 | | 97.5 | 55 | 98.1 |
| | SCE1_1 | | | | | | | | | | | | |
| 10. | Orysa | 88.1 | 87.5 | 89.4 | 69.4 | 57.5 | 91.2 | 69.4 | 91.3 | 93.1 | | 56.9 | 96.9 |
| | SCE1_2 | | | | | | | | | | | | |
| 11. | Orysa | 40.5 | 40.2 | 39.1 | 47.9 | 35.3 | 39.7 | 38.2 | 38.9 | 39.7 | 38.5 | | 55 |
| | SCE1_3 | | | | | | | | | | | | |
| 12. | Nicbe | 91.2 | 88.1 | 91.2 | 73.1 | 56.9 | 91.2 | 70 | 89.4 | 91.2 | 89.4 | 38.5 | |
| | SCE1_1 | | | | | | | | | | | | |
| 13. | Triae | 88.2 | 87 | 89.4 | 68.9 | 56.5 | 91.9 | 68.9 | 92.5 | 93.8 | 93.2 | 38.9 | 89.4 |
| | SCE1_1 | | | | | | | | | | | | |
| 14. | Zeama | 88.8 | 90 | 89.4 | 70.6 | 57.5 | 91.9 | 69.4 | 92.5 | 94.4 | 93.8 | 39.7 | 90.6 |
| | SCE1_1 | | | | | | | | | | | | |
| 15. | Zeama | 89.4 | 88.8 | 90 | 70.6 | 57.5 | 92.5 | 69.4 | 92.5 | 92.5 | 91.2 | 40.2 | 90.6 |
| | SCE1_1 | | | | | | | | | | | | |
| 16. | Zeama | 90.6 | 89.4 | 91.2 | 71.9 | 56.2 | 92.5 | 68.8 | 93.8 | 96.2 | 91.9 | 39.7 | 89.4 |
| | SCE1_1 | | | | | | | | | | | | |
| 17. | Horvu | 87.6 | 86.3 | 88.8 | 68.3 | 56.5 | 91.3 | 68.9 | 91.9 | 93.2 | 93.2 | 38.9 | 88.8 |
| | SCE1_1 | | | | | | | | | | | | |
| 18. | Helan | 88.1 | 86.9 | 88.8 | 70.6 | 57.5 | 89.4 | 68.1 | 89.4 | 90.6 | 89.4 | 40.2 | 88.8 |
| | SCE1_1 | | | | | | | | | | | | |
| 19. | Arath | 90.6 | 83.1 | 88.8 | 70.6 | 58.1 | 88.8 | 70 | 88.8 | 88.8 | 85.6 | 40.8 | 88.1 |
| | SCE1_1 | | | | | | | | | | | | |
| 20. | PopTr | 83.2 | 87.6 | 83.9 | 64.6 | 56.5 | 88.2 | 68.3 | 84.5 | 85.7 | 84.5 | 40 | 82 |
| | SCE1_1 | | | | | | | | | | | | |
| 21. | PopTr | 83.2 | 86.3 | 83.9 | 67.1 | 55.9 | 87 | 67.1 | 83.9 | 85.7 | 83.9 | 39.4 | 83.2 |
| | SCE1_2 | | | | | | | | | | | | |
| 22. | Phypa | 84.4 | 85.6 | 84.4 | 66.9 | 58.8 | 87.5 | 69.4 | 83.9 | 86.2 | 85 | 37.4 | 87.5 |
| | SCE1_1 | | | | | | | | | | | | |
| 23. | Phypa | 83.8 | 85 | 83.8 | 66.2 | 58.8 | 86.2 | 68.8 | 83.2 | 85.6 | 84.4 | 37.9 | 86.9 |
| | SCE1_2 | | | | | | | | | | | | |

| | | 13. | 14. | 15. | 16. | 17. | 18. | 19. | 20. | 21. | 22. | 23. |
|---|---|---|---|---|---|---|---|---|---|---|---|---|
| 1. | Glyma | 94.4 | 93.1 | 95 | 95.6 | 94.4 | 93.1 | 93.1 | 92.5 | 91.9 | 92.5 | 92.5 |
| | SCE1_1 | 92.5 | 93.1 | 93.1 | 95.6 | 92.5 | 94.4 | 92.5 | 93.8 | 94.4 | 93.1 | 93.1 |
| 2. | Picsi | | | | | | | | | | | |
| | SCE1_1 | 95.7 | 94.4 | 96.2 | 96.9 | 95.7 | 94.4 | 93.8 | 93.2 | 92.5 | 93.8 | 93.8 |
| 4. | Popul | | | | | | | | | | | |
| | SCE1_1 | 73.9 | 73.8 | 74.4 | 75.6 | 73.9 | 73.8 | 73.1 | 72.7 | 73.9 | 73.1 | 73.1 |
| 5. | Pruar | | | | | | | | | | | |
| | SCE1_1 | 72 | 71.9 | 71.2 | 72.5 | 71.4 | 71.9 | 72.5 | 71.4 | 72 | 74.4 | 74.4 |
| 6. | Ostta | | | | | | | | | | | |
| | SCE1_1 | 95.7 | 94.4 | 96.2 | 96.9 | 95.7 | 93.8 | 95 | 94.4 | 93.8 | 95.6 | 95 |
| 5. | Vitvi | | | | | | | | | | | |
| | SCE1_1 | 80.7 | 80 | 81.2 | 81.9 | 80.7 | 81.2 | 81.2 | 82.6 | 82 | 83.1 | 83.1 |
| 7. | Chlre | | | | | | | | | | | |
| | SCE1_1 | 96.3 | 94.4 | 96.3 | 96.3 | 96.3 | 91.9 | 93.2 | 91.3 | 91.3 | 91.3 | 91.3 |
| 8. | Tritu | | | | | | | | | | | |
| | SCE1_1 | 97.5 | 97.5 | 97.5 | 98.8 | 97.5 | 93.1 | 94.4 | 92.5 | 93.8 | 94.4 | 94.4 |
| 9. | Orysa | | | | | | | | | | | |
| | SCE1_1 | 96.9 | 97.5 | 96.9 | 97.5 | 96.9 | 93.1 | 93.8 | 90.7 | 91.3 | 93.1 | 93.1 |
| 10. | Orysa | | | | | | | | | | | |
| | SCE1_2 | 55.3 | 55.6 | 55.6 | 56.9 | 55.3 | 56.2 | 54.4 | 55.3 | 55.3 | 53.8 | 53.8 |
| 11. | Orysa | | | | | | | | | | | |
| | SCE1_3 | 96.3 | 95.6 | 96.9 | 96.9 | 96.3 | 93.8 | 95 | 91.9 | 92.5 | 95 | 95 |
| 12. | Nicbe | | | | | | | | | | | |
| | SCE1_1 | | 98.1 | 97.5 | 96.9 | 100 | 93.2 | 93.8 | 92.5 | 92.5 | 92.5 | 92.5 |
| 13. | Triae | | | | | | | | | | | |
| | SCE1_1 | 95 | | 96.9 | 97.5 | 98.1 | 93.1 | 93.1 | 90.7 | 91.9 | 92.5 | 92.5 |
| 14. | Zeama | | | | | | | | | | | |
| | SCE1_1 | 93.8 | 95 | | 97.5 | 97.5 | 94.4 | 94.4 | 92.5 | 92.5 | 92.5 | 92.5 |
| 15. | Zeama | | | | | | | | | | | |
| | SCE1_1 | 93.2 | 93.8 | 93.8 | | 96.9 | 94.4 | 95 | 93.8 | 94.4 | 93.1 | 93.1 |
| 16. | Zeama | | | | | | | | | | | |
| | SCE1_1 | 98.8 | 94.4 | 93.2 | 92.5 | | 93.2 | 93.8 | 92.5 | 92.5 | 92.5 | 92.5 |
| 17. | Horvu | | | | | | | | | | | |
| | SCE1_1 | 89.4 | 90 | 89.4 | 91.2 | 88.8 | | 92.5 | 91.3 | 91.3 | 91.9 | 91.9 |
| 18. | Helan | | | | | | | | | | | |
| | SCE1_1 | 86.3 | 86.9 | 86.9 | 89.4 | 85.7 | 86.2 | | 90.7 | 91.9 | 92.5 | 91.9 |
| 19. | Arath | | | | | | | | | | | |
| | SCE1_1 | 85.1 | 83.9 | 84.5 | 85.1 | 85.7 | 82.6 | 80.1 | | 97.5 | 91.9 | 91.9 |
| 20. | PopTr | | | | | | | | | | | |
| | SCE1_1 | 83.9 | 84.5 | 83.9 | 84.5 | 84.5 | 82.6 | 83.2 | 94.4 | | 93.2 | 93.2 |
| 21. | PopTr | | | | | | | | | | | |
| | SCE1_2 | 84.5 | 86.9 | 85.6 | 84.4 | 83.9 | 84.4 | 82.5 | 80.7 | 82.6 | | 99.4 |
| 22. | Phypa | | | | | | | | | | | |
| | SCE1_1 | 83.9 | 86.2 | 85 | 83.8 | 83.2 | 85 | 81.2 | 80.1 | 82 | 98.8 | |
| 23. | Phypa | | | | | | | | | | | |
| | SCE1_2 | | | | | | | | | | | |

### Example 3.3

### YEF1 Polypeptides

Results of the software analysis are shown in Table B for the global similarity and identity over the full length of the polypeptide sequences. Percentage identity is given above the diagonal in bold and percentage similarity is given below the diagonal (normal face).

The percentage identity between the YEF1 polypeptide sequences of Table B3 and useful in performing the methods of the invention can be as low as 25.5% amino acid identity compared to SEQ ID NO: 249 (named 5. Le_YEF1_{—}1 in Table B3).

TABLE B3
MatGAT results for global similarity and identity over the full length of YEF1 polypeptide sequences.
The name and sequence of the

| Name or YEF1 polypeptide | | 1 | 2 | 3 | 4 | 5 | 6 | 7 | 8 | 9 | 10 | 11 | 12 | 13 | 14 |
|---|---|---|---|---|---|---|---|---|---|---|---|---|---|---|---|
| 1. | Zm\TA1731224577 | | 32.8 | 36.3 | 45.2 | 45.8 | 47.9 | 46.4 | 39.7 | 27.9 | 63.8 | 72.0 | 65.5 | 28.5 | 43.0 |
| 2. | Pinus\r\ADW16852 | 51.2 | | 38.8 | 34.9 | 34.6 | 35.3 | 34.1 | 35.7 | 38.7 | 33.1 | 33.7 | 33.9 | 42.6 | 35.7 |
| 3. | Pinus\r\ADW16853 | 53.2 | 59.4 | | 35.4 | 36.7 | 37.0 | 36.6 | 34.2 | 30.3 | 34.3 | 36.1 | 34.8 | 35.3 | 35.8 |
| 4. | Euc\grandis\ADW16464 | 64.9 | 51.1 | 53.3 | | 54.8 | 63.7 | 62.7 | 45.7 | 25.7 | 44.3 | 47.8 | 46.7 | 28.3 | 52.1 |
| 5. | Le_YEF1_1 | 63.1 | 50.5 | 51.4 | 70.6 | | 60.8 | 59.3 | 42.6 | 25.5 | 46.6 | 49.4 | 48.2 | 27.6 | 51.1 |
| 6. | Pf\scaff_220.7\[2234] | 66.1 | 51.8 | 54.6 | 79.3 | 76.6 | | 89.4 | 49.4 | 27.8 | 47.2 | 49.9 | 49.9 | 27.2 | 53.6 |
| 7. | Pf\scaff_III.1611\[2309] | 64.3 | 49.6 | 53.6 | 77.0 | 76.5 | 92.2 | | 47.7 | 26.6 | 46.2 | 49.6 | 48.0 | 27.5 | 51.5 |
| 8. | At3g51950.1 | 52.5 | 52.6 | 50.1 | 57.8 | 55.4 | 59.8 | 57.7 | | 29.7 | 41.1 | 41.3 | 40.1 | 29.9 | 41.7 |
| 9. | At2g05160.1 | 43.4 | 56.2 | 46.4 | 43.6 | 41.5 | 42.4 | 41.9 | 50.2 | | 26.6 | 27.3 | 27.5 | 47.4 | 26.7 |
| 10. | Os\LOC_Os03g21160.1 | 77.4 | 51.7 | 52.9 | 63.9 | 63.2 | 65.2 | 63.5 | 54.8 | 42.5 | | 70.6 | 78.2 | 28.7 | 44.5 |
| 11. | Os\LOC_Os07g48410.1 | 84.3 | 50.6 | 53.7 | 67.5 | 64.8 | 66.9 | 66.8 | 54.1 | 44.0 | 81.1 | | 74.2 | 30.3 | 46.4 |
| 12. | Os\LOC_Os03g21140.1 | 77.9 | 50.9 | 53.2 | 65.6 | 64.0 | 64.8 | 63.7 | 54.1 | 43.8 | 84.9 | 83.9 | | 30.0 | 45.2 |
| 13. | Vv\CAN64426 | 46.0 | 58.1 | 51.2 | 43.0 | 42.5 | 44.6 | 44.5 | 50.0 | 64.2 | 44.3 | 45.9 | 44.9 | | 29.8 |
| 14. | Vv\CAN62156 | 62.6 | 51.8 | 54.8 | 72.2 | 68.7 | 70.9 | 68.7 | 55.7 | 41.0 | 64.3 | 65.1 | 64.8 | 45.1 | |

### Example 3.4

### Subgroup III Grx Polypeptides

Results of the software analysis are shown in Table B for the global similarity and identity over the full length of the polypeptide sequences. Percentage identity is given above the diagonal in bold and percentage similarity is given below the diagonal (normal face).

### Example 4

### Identification of Domains Comprised in Polypeptide Sequences Useful in Performing the Methods of the Invention

The Integrated Resource of Protein Families, Domains and Sites (InterPro) database is an integrated interface for the commonly used signature databases for text- and sequence-based searches. The InterPro database combines these databases, which use different methodologies and varying degrees of biological information about well-characterized proteins to derive protein signatures. Collaborating databases include SWISS-PROT, PROSITE, TrEMBL, PRINTS, ProDom and Pfam, Smart and TIGRFAMs. Pfam is a large collection of multiple sequence alignments and hidden Markov models covering many common protein domains and families. Pfam is hosted at the Sanger Institute server in the United Kingdom. Interpro is hosted at the European Bioinformatics Institute in the United Kingdom.

### Example 4.1

### PRE Polypeptides

The results of the InterPro scan of the polypeptide sequence as represented by SEQ ID NO: 2 are presented in Table C1.

TABLE C1
InterPro and SMART scan results (major accession numbers) of
the polypeptide sequence as represented by SEQ ID NO: 2.

| | | | Amino acid |
|---|---|---|---|
| | Accession | | coordinates |
| Database | number | Accession name | on SEQ ID NO 2 |
| ProfileScan | PS50888 | HLH | 4-60 |
| superfamily | SSF47459 | Helix-loop-helix | 1-90 |
| | | DNA-binding domain | |
| SMART | SM00353 | HLH | 16-65 |

### Example 4.2

### SCE1 Polypeptides

The results of the InterPro scan of the SCE1 polypeptides sequence as represented by SEQ ID NO: 200 by SEQ ID NO: 216 are presented in Table C2.

TABLE C2
InterPro scan results (major accession numbers) of the polypeptide sequence
represented by SEQ ID NO: 200.

| query | Intepro | | | Description | | | | | |
|---|---|---|---|---|---|---|---|---|---|
| sequence | accession | Accession | Description | Alias | Short name | e (E) value | Start | End | Method |
| Arath | IPR000608 | PD000461 | Ubiquitin- | UBC | UBQ_conjugat | 7.00E−92 | 5 | 156 | BlastProDom |
| SCE1_1 | | | conjugating | | | | | | |
| | | | enzyme, E2 | | | | | | |
| Arath | IPR000608 | PF00179 | Ubiquitin- | UBC | UQ_con | 3.3E−70 | 9 | 153 | HMMPfam |
| SCE1_1 | | | conjugating | | | | | | |
| | | | enzyme, E2 | | | | | | |
| Arath | IPR000608 | SM00212 | Ubiquitin- | UBC | UBCc | 1.00E−67 | 8 | 158 | HMMSmart |
| SCE1_1 | | | conjugating | | | | | | |
| | | | enzyme, E2 | | | | | | |
| Arath | IPR000608 | PS00183 | Ubiquitin- | UBC | UBIQUITIN_CONJUGAT_1 | 0 | 83 | 97 | ProfileScan |
| SCE1_1 | | | conjugating | | | | | | |
| | | | enzyme, E2 | | | | | | |
| Arath | IPR000608 | PS50127 | Ubiquitin- | UBC | UBIQUITIN_CONJUGAT_2 | 35.839 | 8 | 147 | ProfileScan |
| SCE1_1 | | | conjugating | | | | | | |
| | | | enzyme, E2 | | | | | | |
| Orysa | IPR000608 | PD000461 | Ubiquitin- | UBC | UBQ_conjugat | 8.00E−91 | 5 | 156 | BlastProDom |
| SCE1_1 | | | conjugating | | | | | | |
| | | | enzyme, E2 | | | | | | |
| Orysa | IPR000608 | PF00179 | Ubiquitin- | UBC | UQ_con | 9.3E−68 | 9 | 153 | HMMPfam |
| SCE1_1 | | | conjugating | | | | | | |
| | | | enzyme, E2 | | | | | | |
| Orysa | IPR000608 | SM00212 | Ubiquitin- | UBC | UBCc | 4.7E−66 | 8 | 158 | HMMSmart |
| SCE1_1 | | | conjugating | | | | | | |
| | | | enzyme, E2 | | | | | | |
| Orysa | IPR000608 | PS00183 | Ubiquitin- | UBC | UBIQUITIN_CONJUGAT_1 | 0 | 83 | 97 | ProfileScan |
| SCE1_1 | | | conjugating | | | | | | |
| | | | enzyme, E2 | | | | | | |
| Orysa | IPR000608 | PS50127 | Ubiquitin- | UBC | UBIQUITIN_CONJUGAT_2 | 35.707 | 8 | 147 | ProfileScan |
| SCE1_1 | | | conjugating | | | | | | |
| | | | enzyme, E2 | | | | | | |
| Orysa | IPR000608 | PD000461 | Ubiquitin- | UBC | UBQ_conjugat | 6.00E−91 | 5 | 156 | BlastProDom |
| SCE1_2 | | | conjugating | | | | | | |
| | | | enzyme, E2 | | | | | | |
| Orysa | IPR000608 | PF00179 | Ubiquitin- | UBC | UQ_con | 1.1E−65 | 9 | 151 | HMMPfam |
| SCE1_2 | | | conjugating | | | | | | |
| | | | enzyme, E2 | | | | | | |
| Orysa | IPR000608 | SM00212 | Ubiquitin- | UBC | UBCc | 2.7E−64 | 8 | 158 | HMMSmart |
| SCE1_2 | | | conjugating | | | | | | |
| | | | enzyme, E2 | | | | | | |
| Orysa | IPR000608 | PS00183 | Ubiquitin- | UBC | UBIQUITIN_CONJUGAT_1 | 0 | 83 | 97 | ProfileScan |
| SCE1_2 | | | conjugating | | | | | | |
| | | | enzyme, E2 | | | | | | |
| Orysa | IPR000608 | PS50127 | Ubiquitin- | UBC | UBIQUITIN_CONJUGAT_2 | 35.76 | 8 | 147 | ProfileScan |
| SCE1_2 | | | conjugating | | | | | | |
| | | | enzyme, E2 | | | | | | |
| Orysa | IPR000608 | PD000461 | Ubiquitin- | UBC | Q8H8G9_EEEEE_Q8H8G9; | 2.00E−36 | 1 | 97 | BlastProDom |
| SCE1_3 | | | conjugating | | | | | | |
| | | | enzyme, E2 | | | | | | |
| Orysa | IPR000608 | PF00179.15 | Ubiquitin- | UBC | Ubiquitin- | 2.00E−29 | 1 | 115 | HMMPfam |
| SCE1_3 | | | conjugating | | conjugating | | | | |
| | | | enzyme, E2 | | enzyme | | | | |
| Orysa | IPR000608 | SM00212 | Ubiquitin- | UBC | no description | 2.8E−24 | 1 | 120 | HMMSmart |
| SCE1_3 | | | conjugating | | | | | | |
| | | | enzyme, E2 | | | | | | |
| Orysa | IPR000608 | PS50127 | Ubiquitin- | UBC | UBIQUITIN_CONJUGAT_2 | 26.416 | 1 | 106 | ProfileScan |
| SCE1_3 | | | conjugating | | | | | | |
| | | | enzyme, E2 | | | | | | |

### Example 4.3

### YEF1 Polypeptides

The conserved protein domains present in YEF1 polypeptide polypeptide sequences as defined in Table A are shown in Table C3.

TABLE C3
Conserved protein domains present in YEF1 polypeptide sequences as
defined in Table A3 are shown. The amino acid coordinates defining the
location of the conserved domains are indicated The conserved C3H and
RRM domains were identified by analysing The results of the InterPro
scan as described above. Amino acid coordinates according to the pfam
scan are shown. The NPD1 domain was identified by analysing the
multiple protein alignment of FIG. 12.

| | Amino acid coordinates | | |
|---|---|---|---|
| | New protein | C3H | RRM |
| | domain 1 (NPD1) | (PF00642)* | (PF00076)** |
| Pinus\r\ADW16852 | 1-65 | 156-181 | 316-393 |
| Pinus\r\ADW16853 | 1-64 | 159-184 | 313-390 |
| Euc\grandis\ADW16464 | 1-64 | 153-178 | 310-387 |
| Le_YEF1_1 | 1-64 | 260-285 | 373-450 |
| Pt\scaff_220.7\[2234] | 1-64 | 233-258 | 365-442 |
| Pt\scaff_III.1611\[2309] | 1-64 | 228-253 | 358-435 |
| At3g51950.1 | 1-64 | 229-254 | 360-437 |
| At2g05160.1 | 1-64 | 148-173 | 257-334 |
| Os\LOC_Os03g21160.1 | 1-64 | 221-246 | 362-439 |
| Os\LOC_Os07g48410.1 | 1-64 | 231-256 | 360-437 |
| Os\LOC_Os03g21140.1 | 1-64 | 230-255 | 359-436 |
| Zm TA1731224577 | 1-64 | 231-256 | 363-440 |
| Vv\CAN64426 | 1-64 | 264-289 | 398-475 |
| Vv\CAN62156 | 1-65 | 222-247 | 352-429 |

*PF00642 is the accession number of the C3H (CCCH) domain in the pfam database (Bateman et al. 2002).
**PF00076 is the accession number of the RRM domain (RRM recognition motif) in the pfam database (Bateman et al. 2002).

### Example 4.4

### Subgroup III Grx Polypeptides

The results of the InterPro scan of the polypeptide sequence as represented by SEQ ID NO: 283 are presented in Table C4.

TABLE C4
InterPro scan results (major accession numbers) of the polypeptide sequence
represented by SEQ ID NO: 283.

| IPR code | database | entry | domain | start | end | e-value | annotation |
|---|---|---|---|---|---|---|---|
| IPR002109 | HMMPfam | PF00462 | Glutaredoxin | 13 | 75 | 1.10E−15 | Glutaredoxin |
| IPR011905 | HMMTigr | TIGR02189 | GlrX-like_plant | 4 | 102 | 3.21E−65 | Glutaredoxin-like, plant II |
| IPR012335 | Gene3D | G3DSA:3.40.30.10 | Thioredoxin_fold | 2 | 101 | 1.80E−24 | Thioredoxin fold |
| IPR012336 | superfamily | SSF52833 | Thiordxn-like_fd | 1 | 101 | 2.10E−20 | Thioredoxin-like fold |
| IPR014025 | FPrintScan | PR00160 | GLUTAREDOXIN | 13 | 31 | 2.70E−07 | Glutaredoxin subgroup |
| IPR014025 | FPrintScan | PR00160 | GLUTAREDOXIN | 58 | 71 | 2.70E−07 | Glutaredoxin subgroup |
| IPR014025 | FPrintScan | PR00160 | GLUTAREDOXIN | 72 | 85 | 2.70E−07 | Glutaredoxin subgroup |
| NULL | HMMPanther | PTHR10168 | PTHR10168 | 1 | 102 | 1.20E−69 | NULL |
| NULL | HMMPanther | PTHR10168:SF18 | PTHR10168:SF18 | 1 | 102 | 1.20E−69 | NULL |

### Example 5

### Topology Prediction of the Polypeptide Sequences Useful in Performing the Methods of the Invention

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no). The “plant” organism group is selected, no cutoffs defined, and the predicted length of the transit peptide requested.

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;
TMHMM, hosted on the server of the Technical University of Denmark

### Example 5.1

### PRE-Like Polypeptides

The results of TargetP 1.1 analysis of the polypeptide sequence as represented by SEQ ID NO: 2 are presented Table D1. The “plant” organism group has been selected, no cutoffs defined, and the predicted length of the transit peptide requested. The subcellular localization of the polypeptide sequence as represented by SEQ ID NO: 2 may be the chloroplast, however this prediction may not be significant, given the reliability class of 4. When analysed by PLOC (Park and Kanehisa, Bioinformatics, 19 1656-1663 2003) the sequence is predicted to have a nuclear localisation, which is in agreement with the findings for the Arabidopsis orthologue (Lee et al., 2006).

TABLE D1
TargetP 1.1 analysis of the polypeptide sequence as
represented by SEQ ID NO: 2

| | |
|---|---|
| Length (AA) | 92 |
| Chloroplastic transit peptide | 0.657 |
| Mitochondrial transit peptide | 0.419 |
| Secretory pathway signal peptide | 0.006 |
| Other subcellular targeting | 0.114 |
| Predicted Location | C |
| Reliability class | 4 |
| Predicted transit peptide length | 17 |

### Example 5.2

### Subgroup III Grx Polypeptides

The results of TargetP 1.1 analysis of the polypeptide sequence as represented by SEQ ID NO: 2 are presented Table D2. The “plant” organism group has been selected, no cutoffs defined, and the predicted length of the transit peptide requested. The subcellular localization of the polypeptide sequence as represented by SEQ ID NO: 283 is likely cytoplasmic.

TABLE D2
TargetP 1.1 analysis of the polypeptide sequence as represented
by SEQ ID NO: 283

Aminoacids: 102
Molecular weight: 11039
Theoretical pI: 6.49
Psort: cytoplasm 0.450 or mitochondral 0.441
PA-SUB: no prediction.
SignalP: no signal peptide predicted.
TargetP: other 0.59, quality 4 (unsure)
SubLoc: cytoplasmic (accuracy 74%)
MitoProt: probability of mitochondrial taregting 0.27
PTS1: not targeted to peroxisomes

### Example 6

### Functional Assays for the Relevant Sequences

### Example 6.1

### PRE-Like Polypeptides

A bioassay for testing PRE-like activity in transgenic plants is provided in Lee et al. (2006): seeds of plants overexpressing PRE1 had a significant higher germination rate in the presence of paclobutrazol (an inhibitor of gibberellin synthesis), compared to wild type plants.

### Example 6.2

### Functional Assay for the SCE1 Polynucleotide and Polypeptide

Activity of SCE1 nucleic acids and SCE1 polypeptide is assayed by methods well known in the art (Castillo et al. 2004; Bernier-Villamor et al. (2002); Lois et al 2003).

In vivo functional activity of a Arath_SCE1_{—}1 nucleic acid is analysed by complementation of the S. cerevisiae ubc9-2 mutant (YW098) essentially as described by Castillo et al. 2004. Briefly transformants of the temperature sensitive mutant (YWO98) harboring the SCE1 nucleic acid are streaked on selective plates and are incubated at 25 and 37° C. in the absence or presence of doxycycline (10_g/ml). Proliferation of yeast in the plates is recorded after at 3-10 days incubation.

in vitro the activity of Arath_SCE1_{—}1 polypeptide is assayed essentially as described by Lois et al. 2003. SUMO conjugation is assayed with RanGAP1 peptide (amino acids 420 to 589) as described by Bernier-Villamor et al. (2002). Briefly, reactions mixtures are prepare to contain 2 μM glutathione S-transferase (GST)-RanGAP1, 0.3 μM human E1, 0.3 μM HsUBC9 or 3 μM AtSCE1a, and 8 μM HsSUMO1 in the reaction buffer (1 mM ATP, 50 mM NaCI, 20 mM Hepes, pH 7.5, 0.1% Tween 20, 5 mM MgCl2, and 0.1 mM DTT). After incubation at 37° C. for 4 h, reactions are stopped by the addition of protein-loading buffer and the mixture is boiled for 5 min. Three microliters of each reaction mixture is resolved by SDS-PAGE and transferred to polyvinylidene difluoride membranes (Immobilon-P; Millipore, Bedford, Mass.), and SUMO conjugation to GST-RanGAP is examined by protein gel blot analysis using anti-HsSUMO1 polyclonal antibody (diluted 1:1000; Alexis, San Diego, Calif.).

### Example 6.3

### Functional Assay for the Polypeptide of SEQ ID NO: 283

Subgroup III Grx polypeptides catalyse the reduction of disulfide bonds in proteins converting glutathione (GSH) to glutathione disulfide (GSSG). GSSG is in turn recycled to GSH by the enzyme glutathione reductase at the expense of NADPH.

### Example 7

### Cloning of the Nucleic Acid Sequence Used in the Methods of the Invention

### Example 7.1

### PRE-Like Polypeptides

The nucleic acid sequence used in the methods of the invention was amplified by PCR using as template a custom-made Triticum aestivum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm09663 (SEQ ID NO: 3; sense, start codon in bold): 5′-ggggacaagtttgtacaaaaaagcaggctt a aacaatgtcgagccgtaggtcaa-3′ and prm09664 (SEQ ID NO: 4; reverse, complementary): 5′-ggggaccactttgtacaagaaagctgggtccggctctacatcagcaag-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pPRE-like. Plasmid pDONR201 was purchased from Invitrogen, as part of the Gateway® technology.

The entry clone comprising SEQ ID NO: 1 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 5) for root specific expression was located upstream of this Gateway cassette.

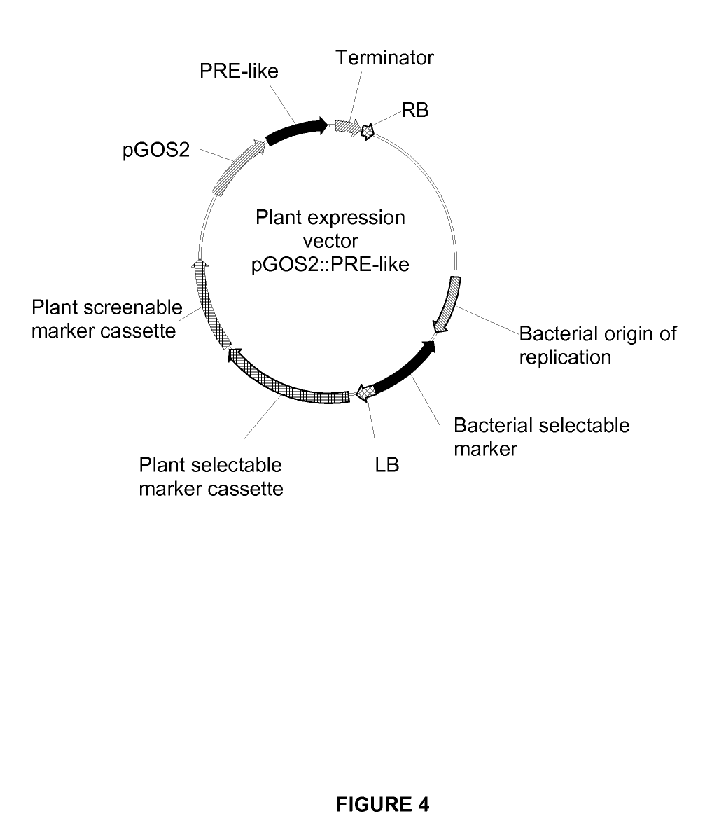

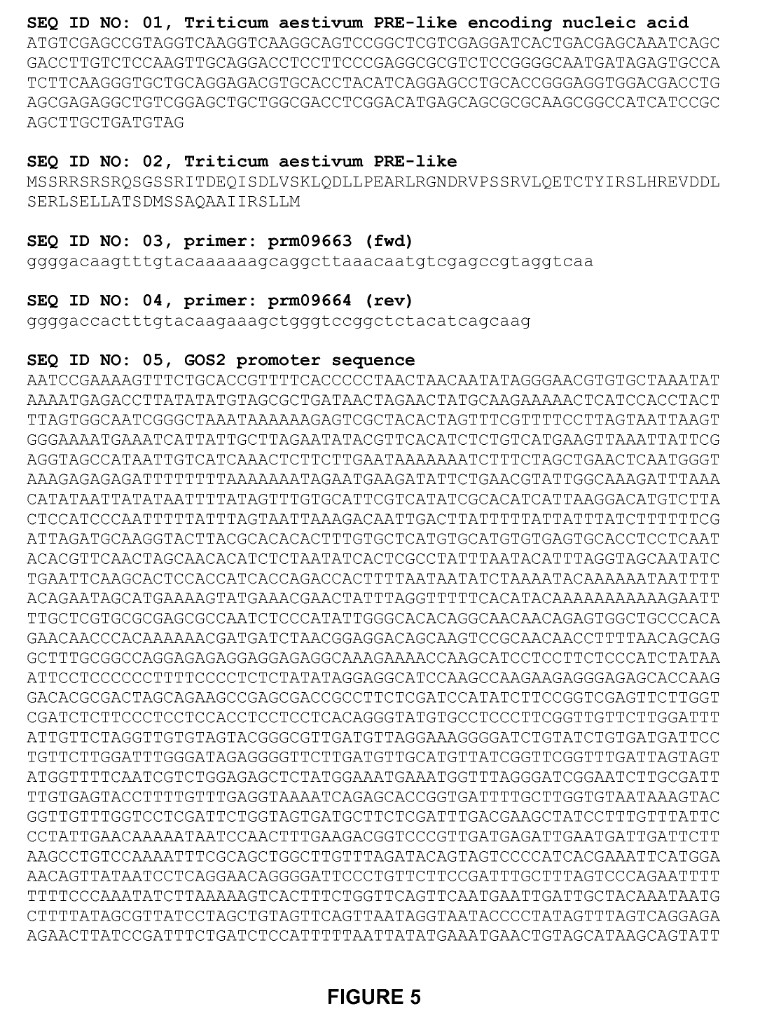

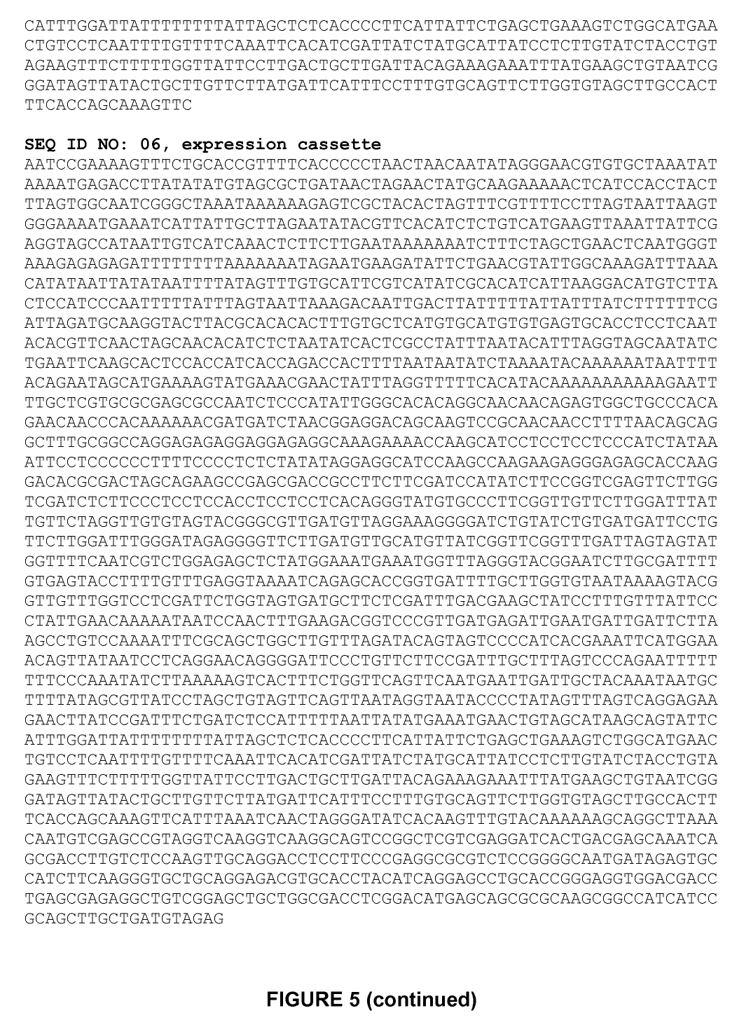

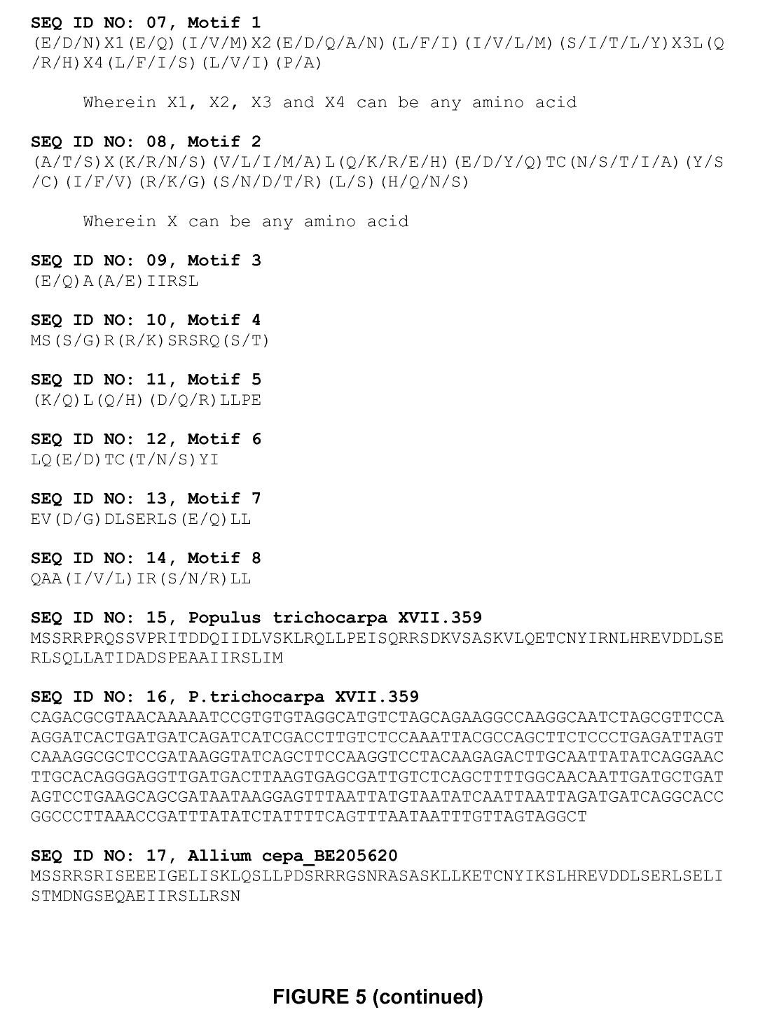

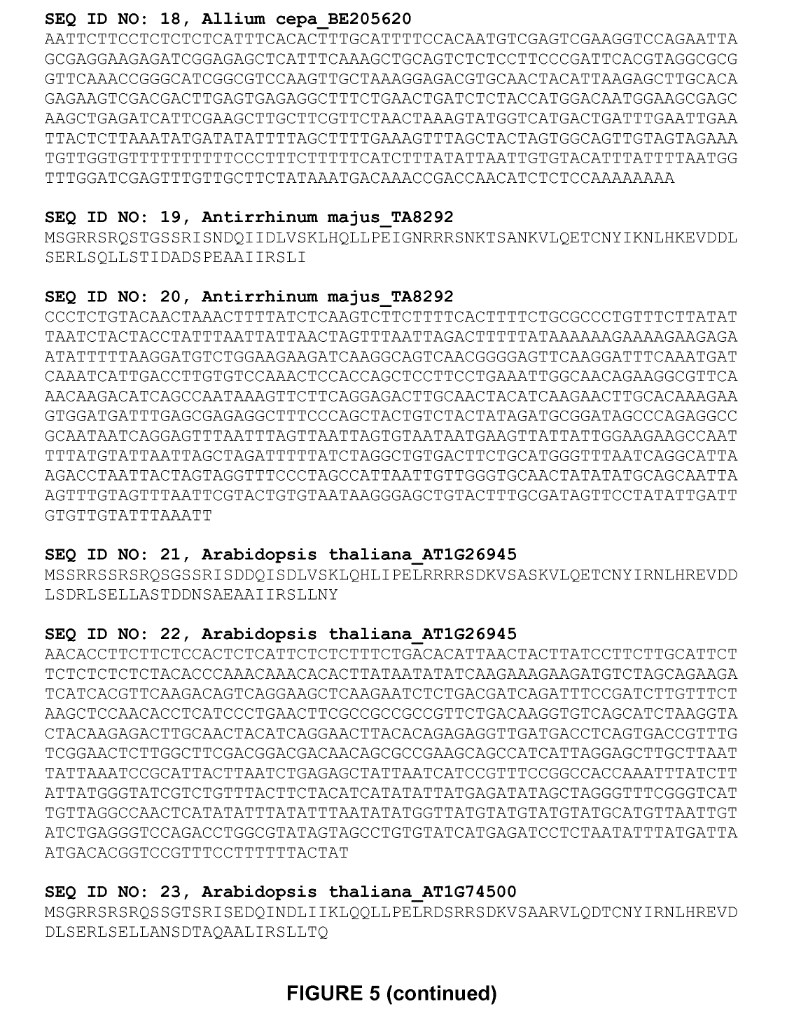

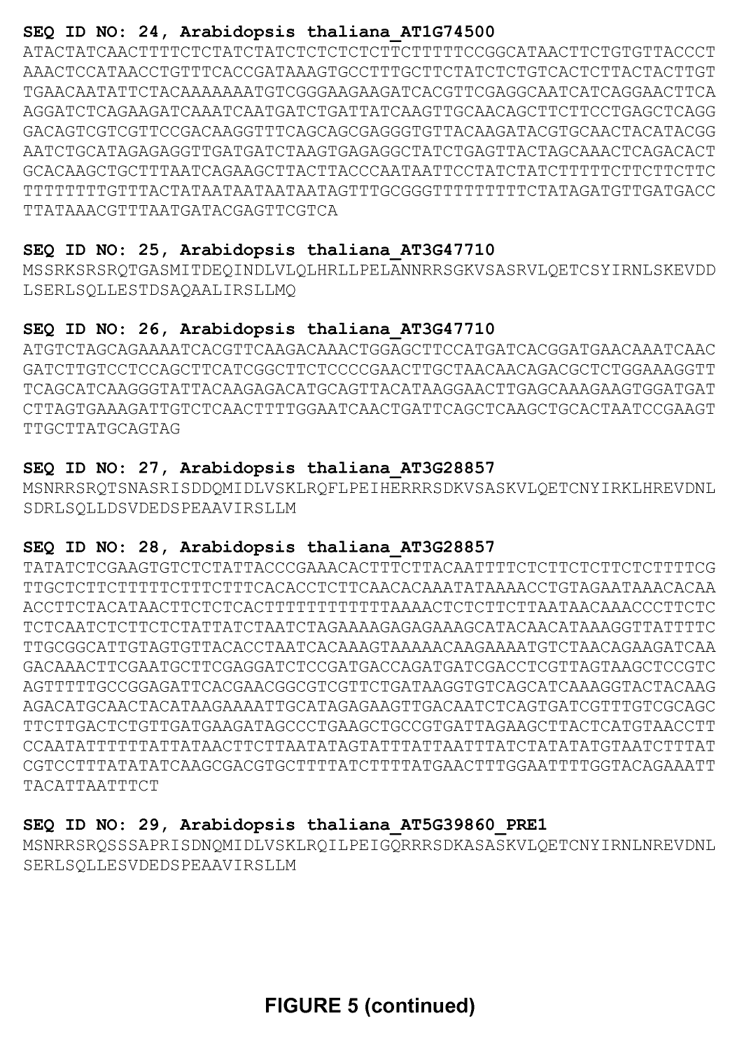

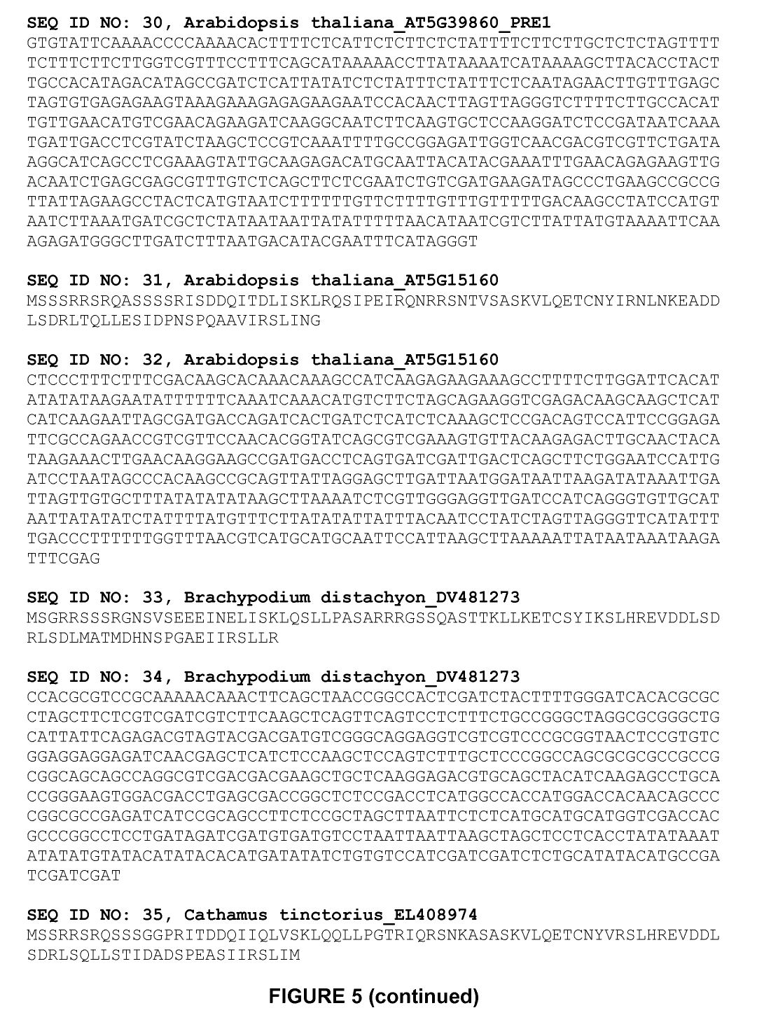

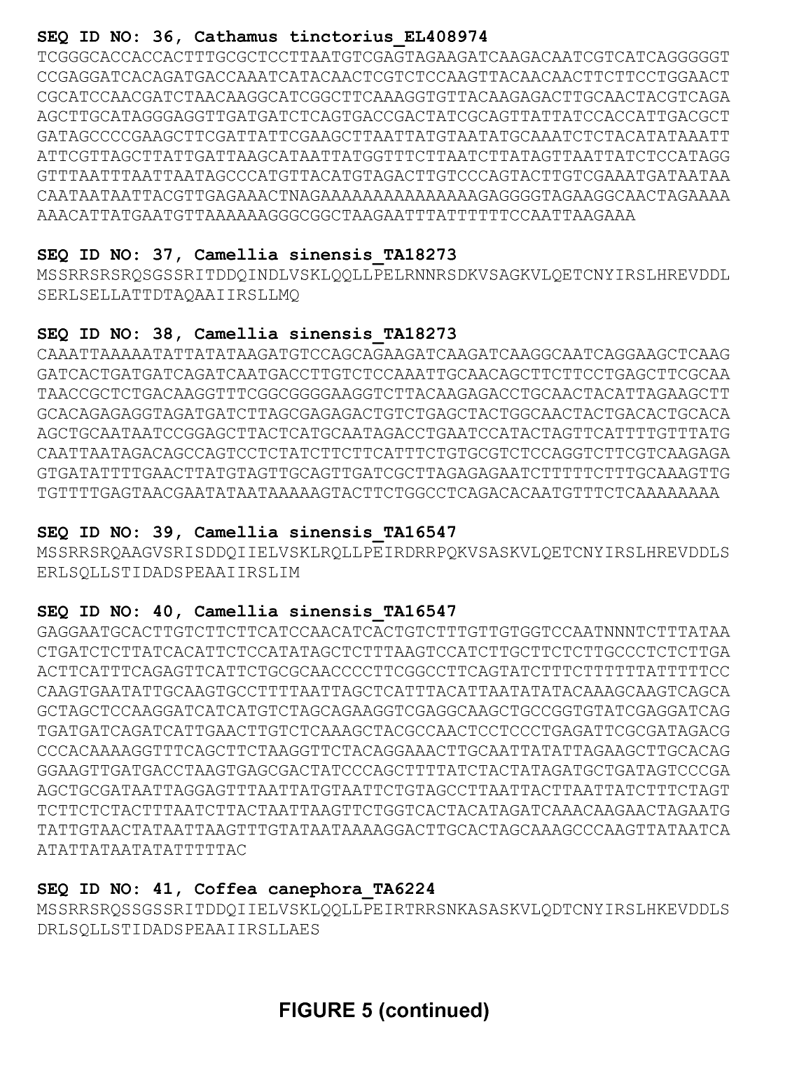

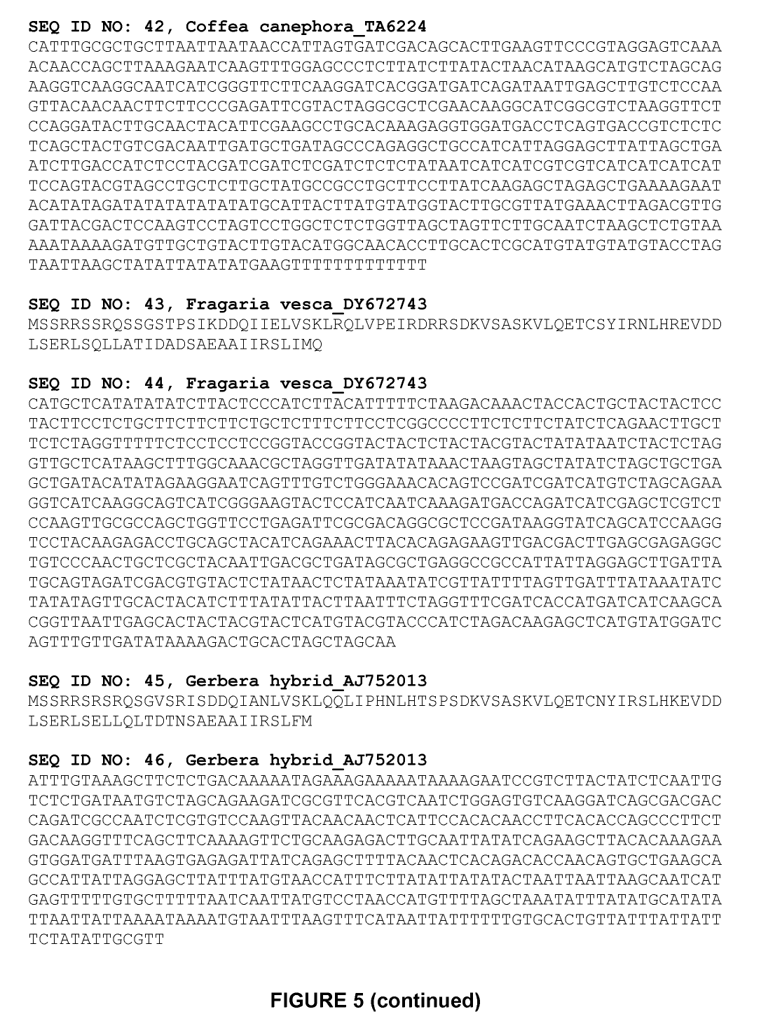

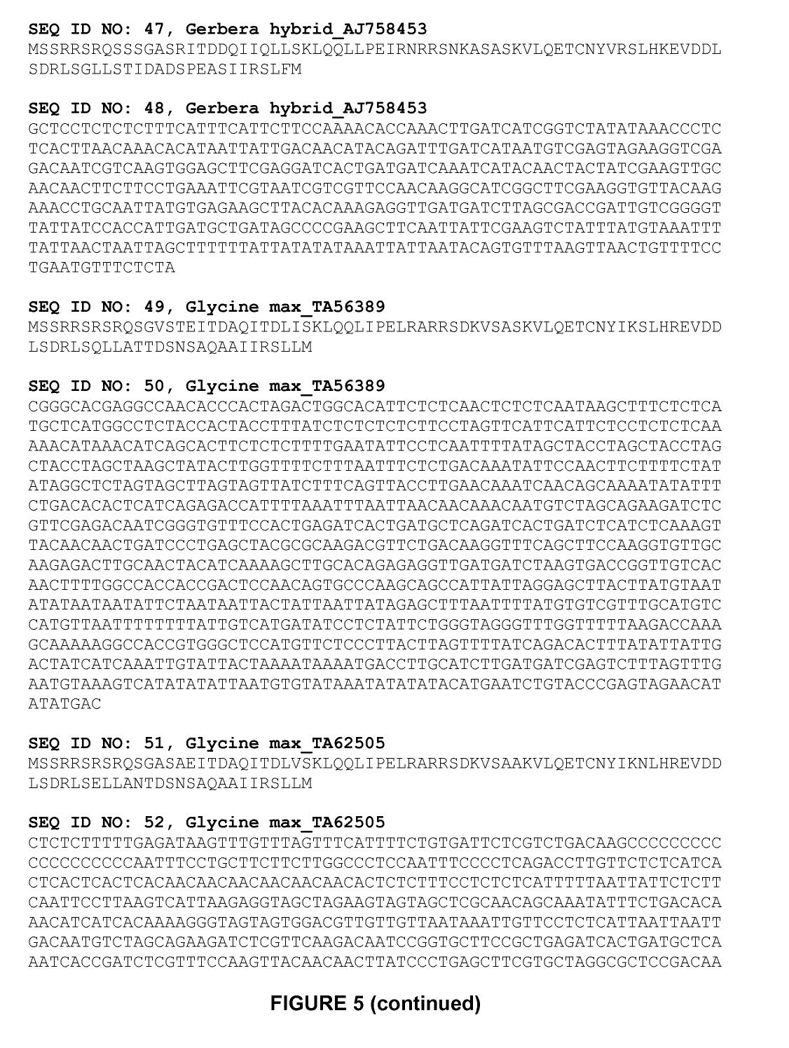

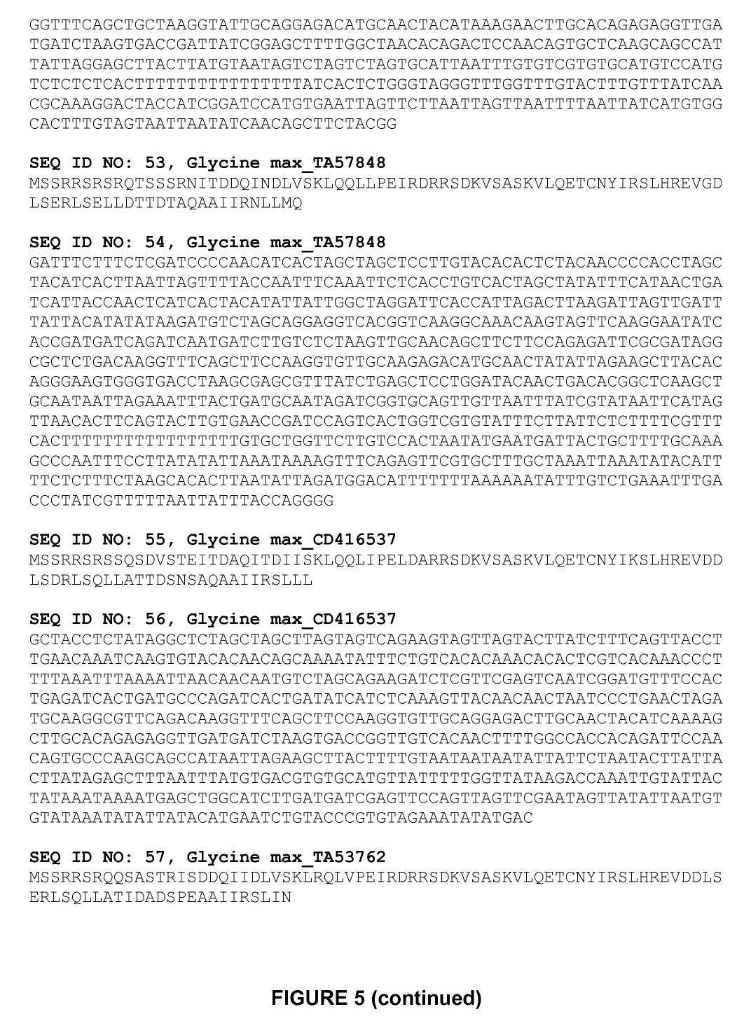

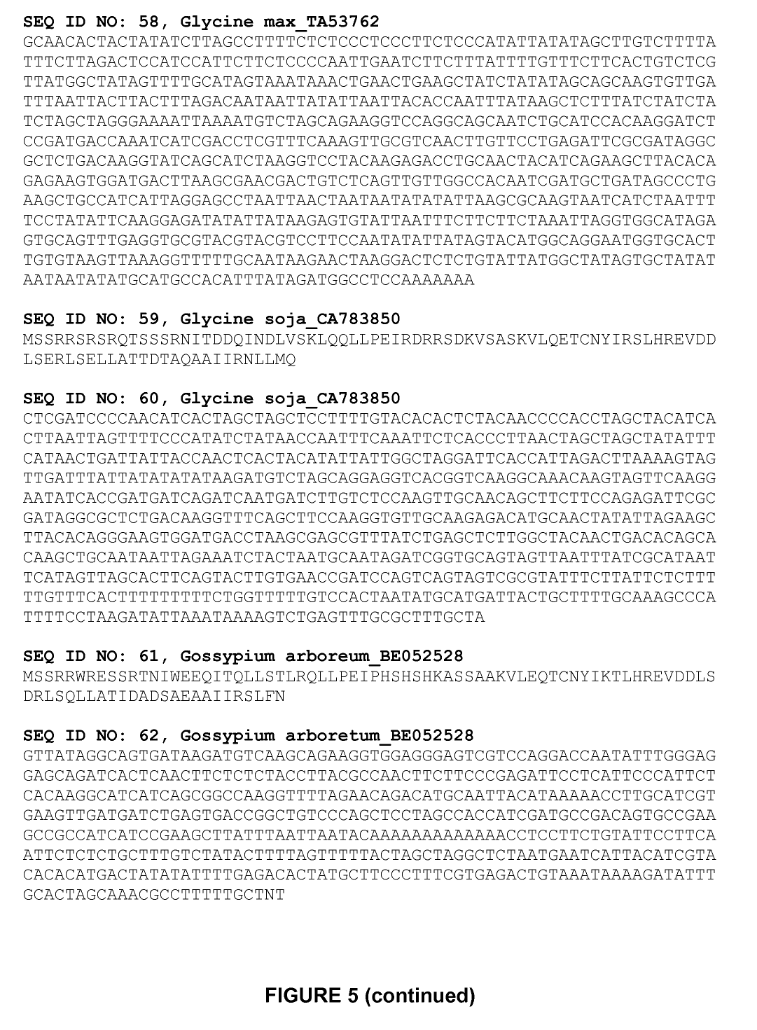

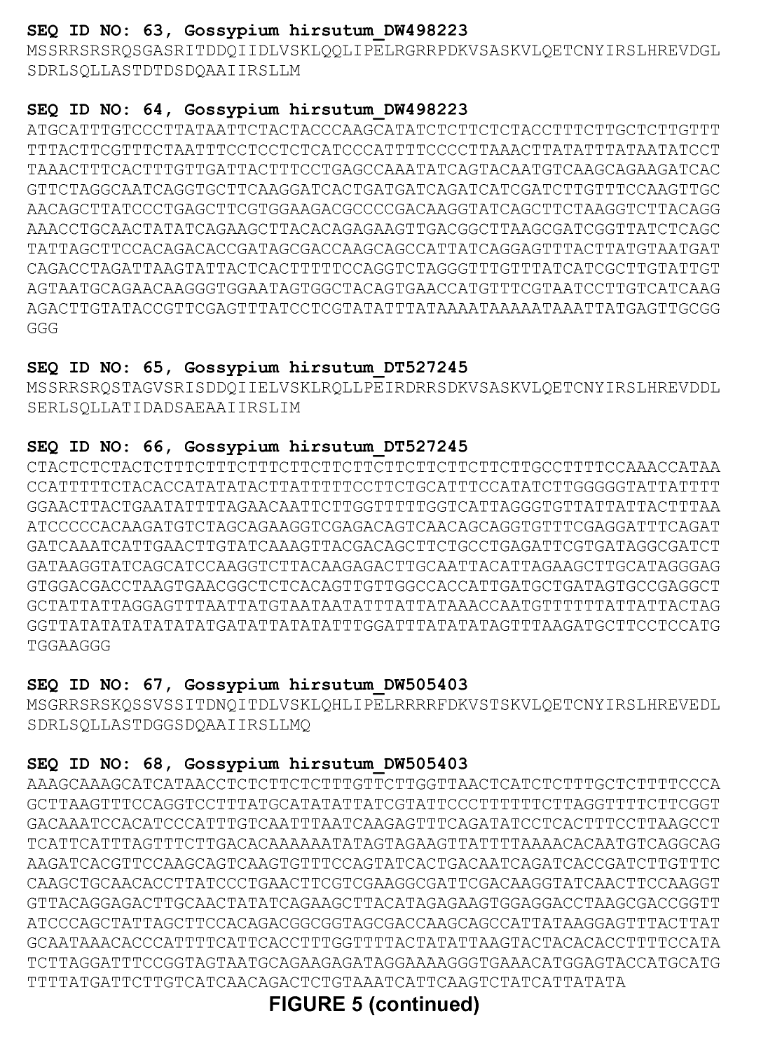

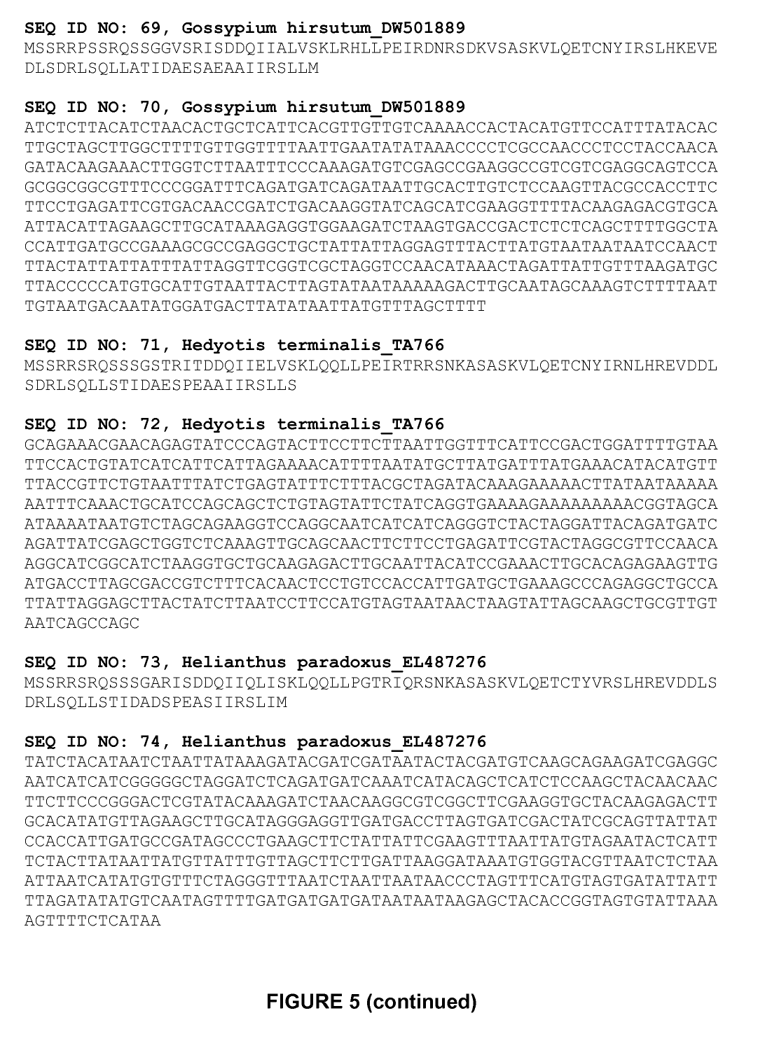

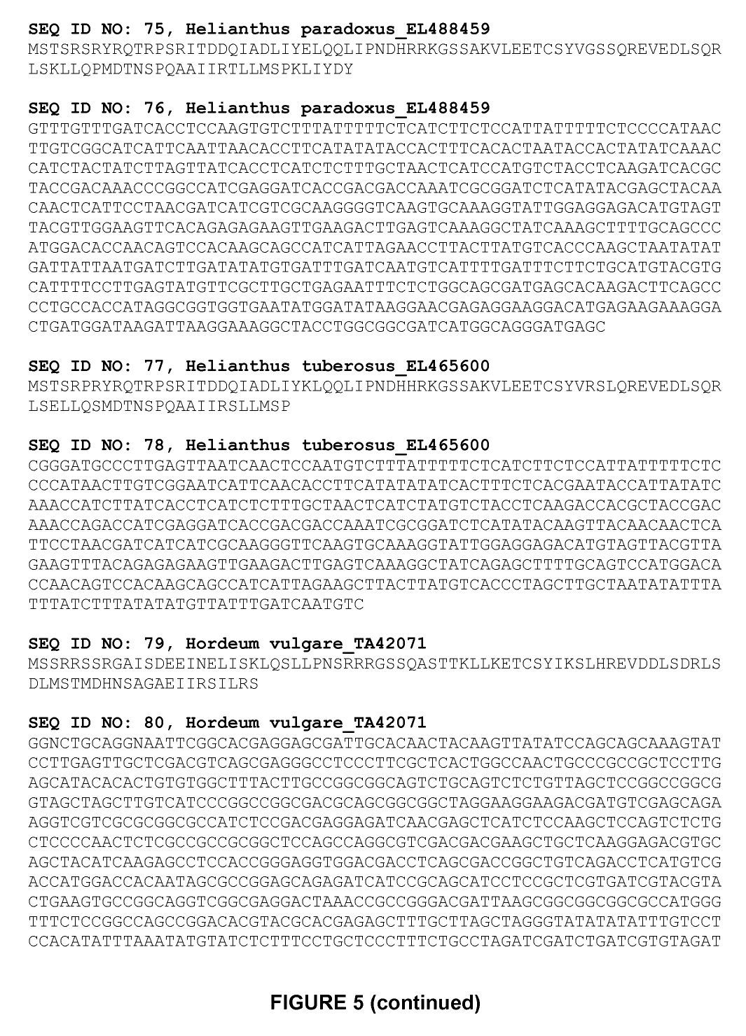

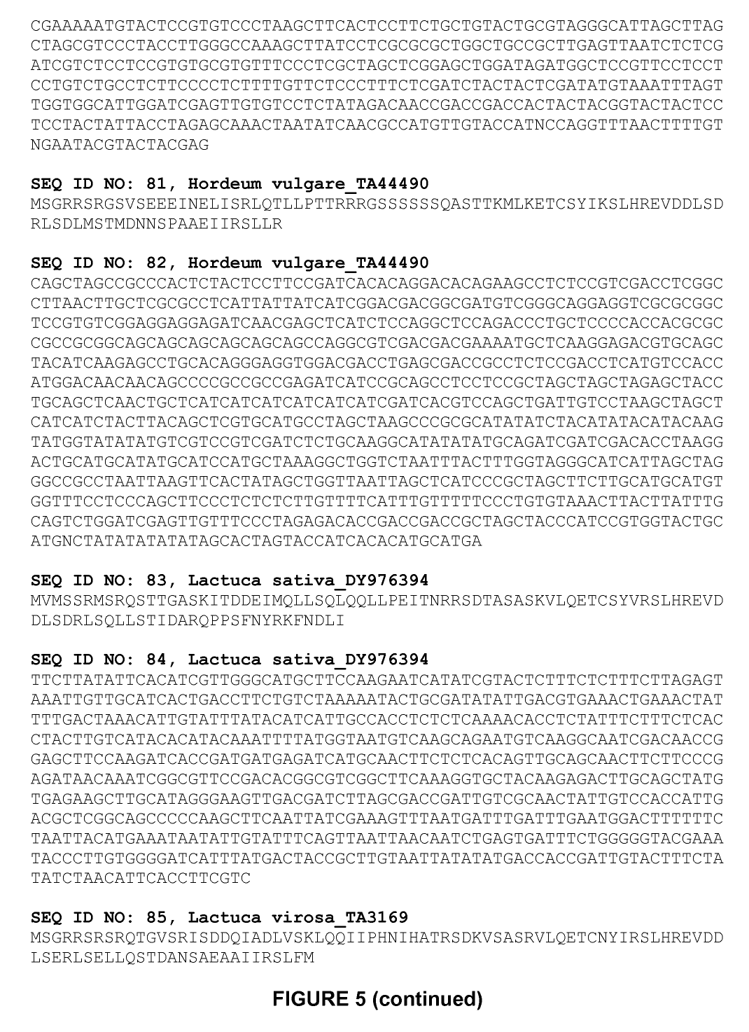

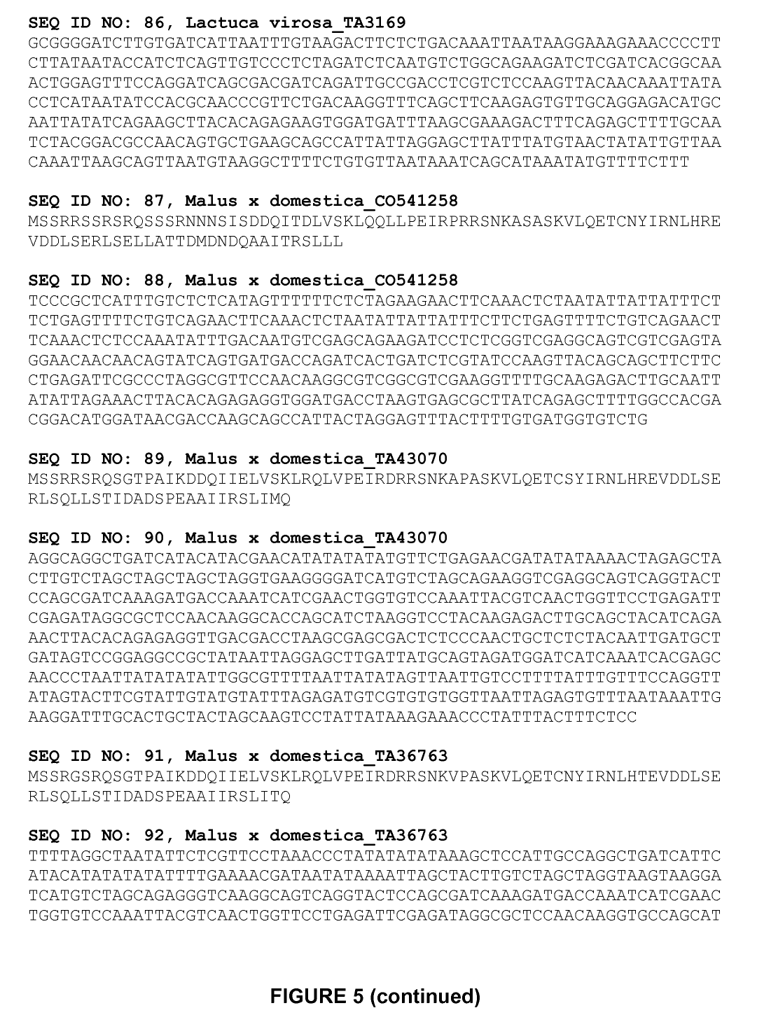

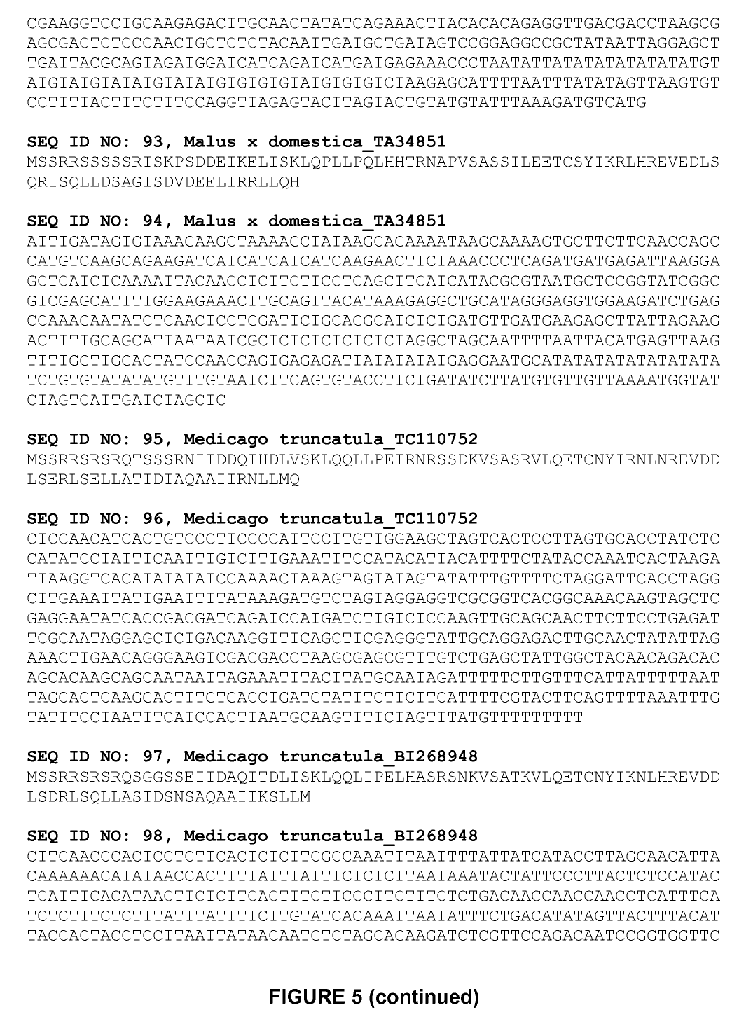

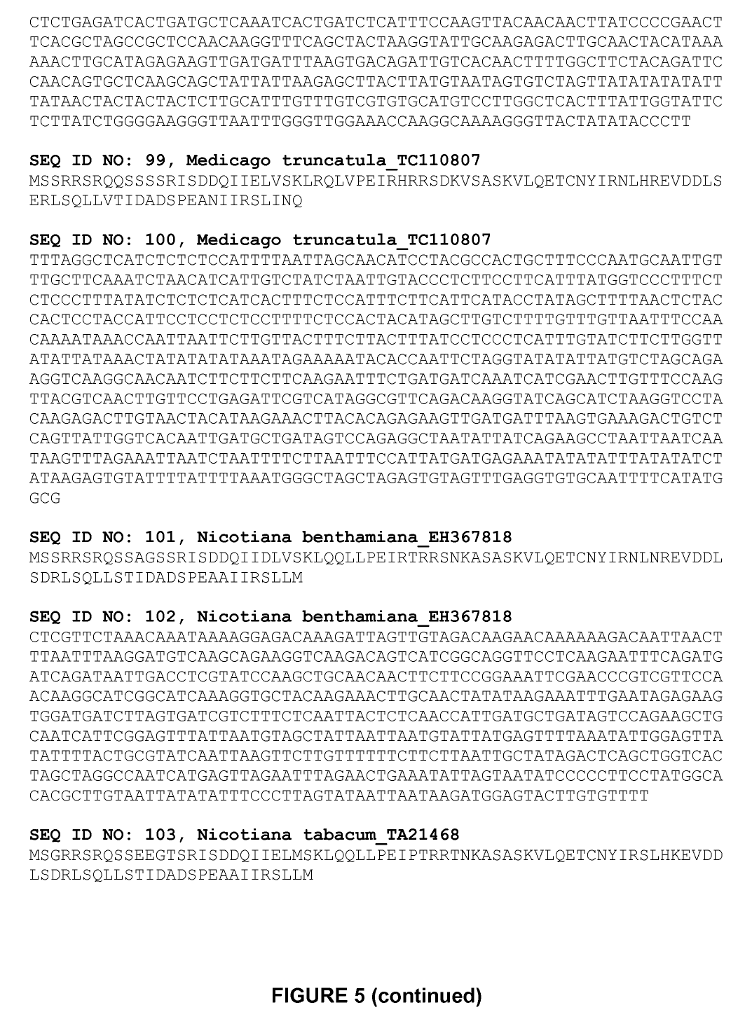

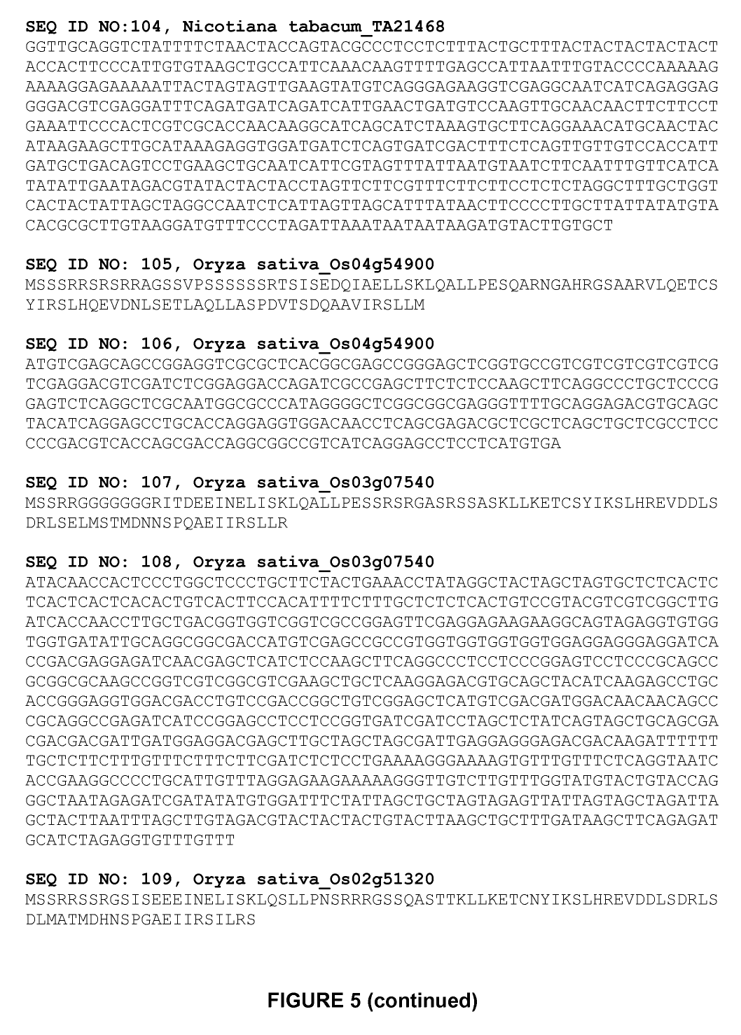

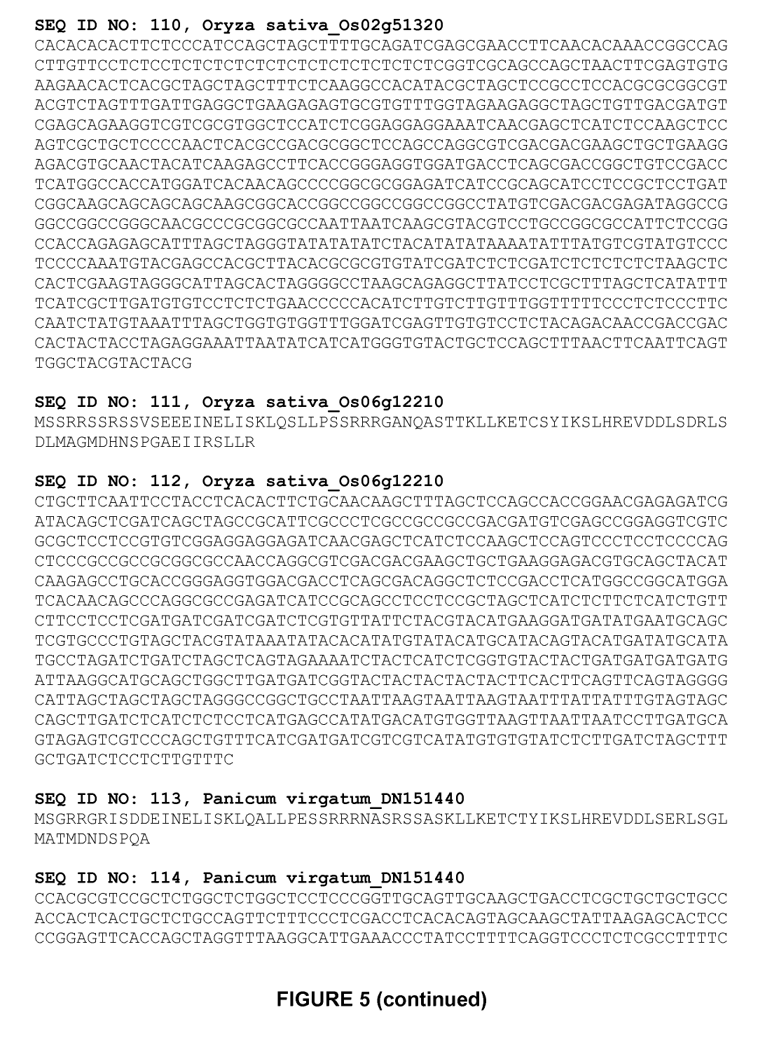

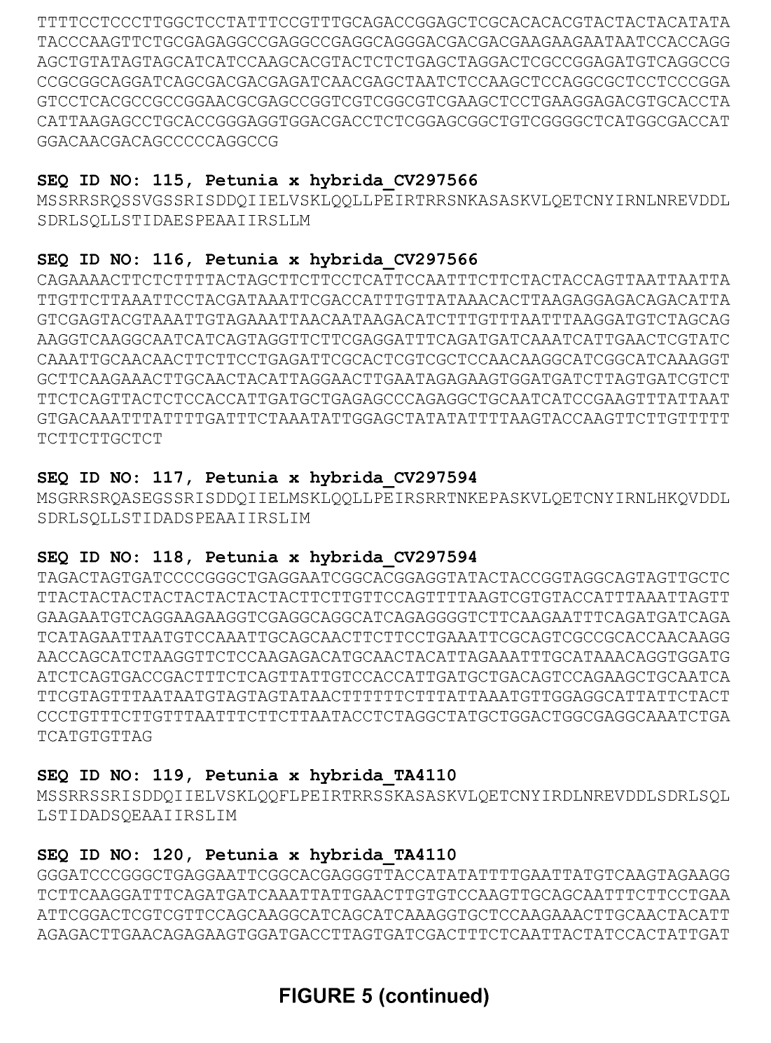

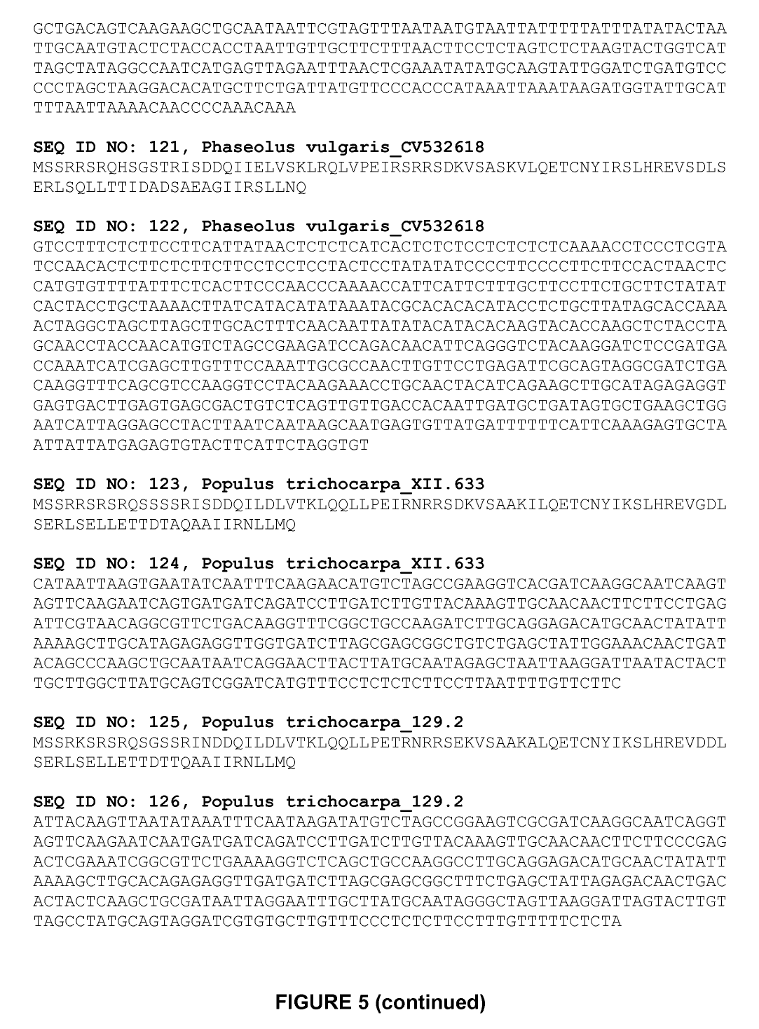

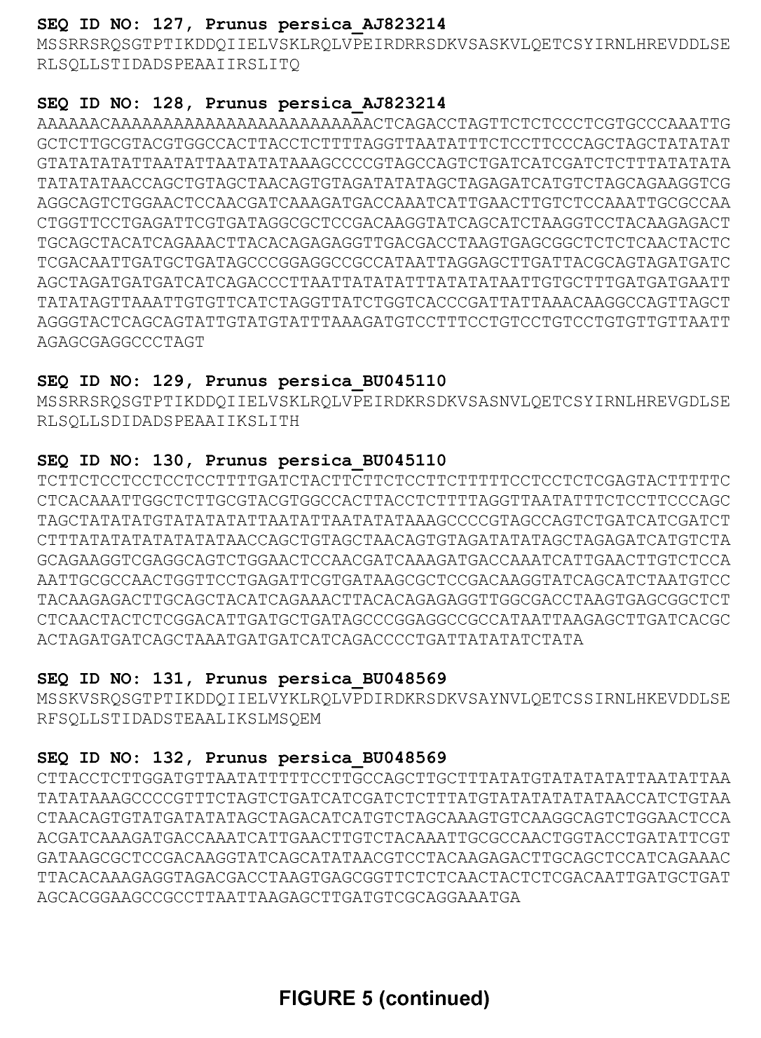

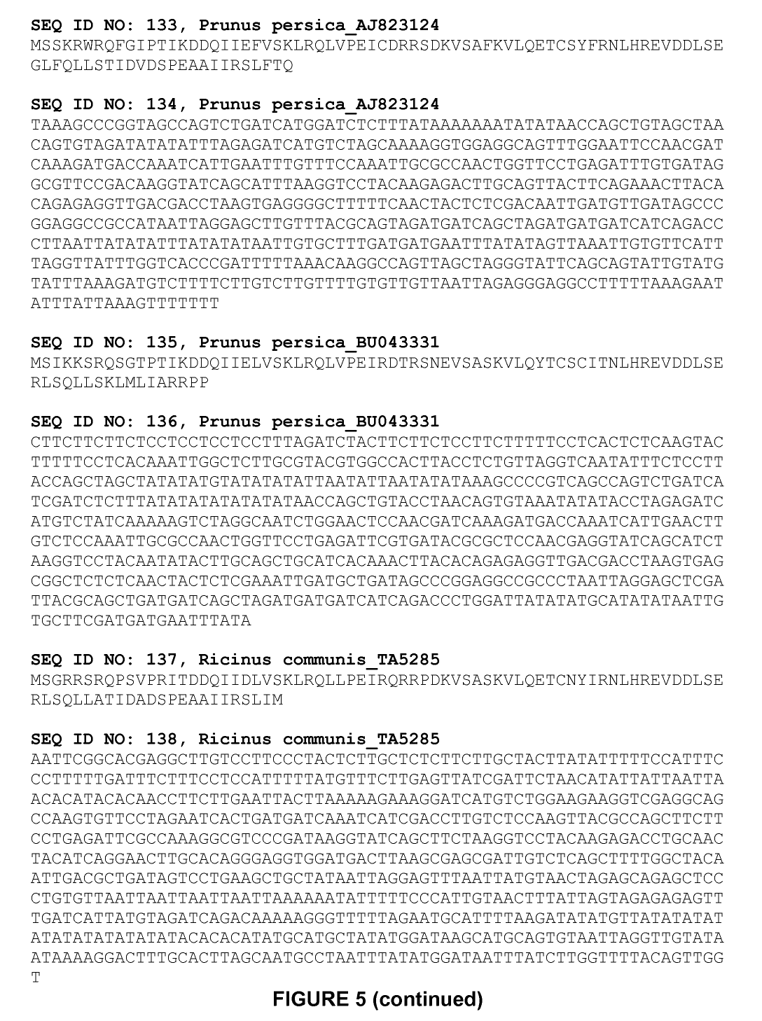

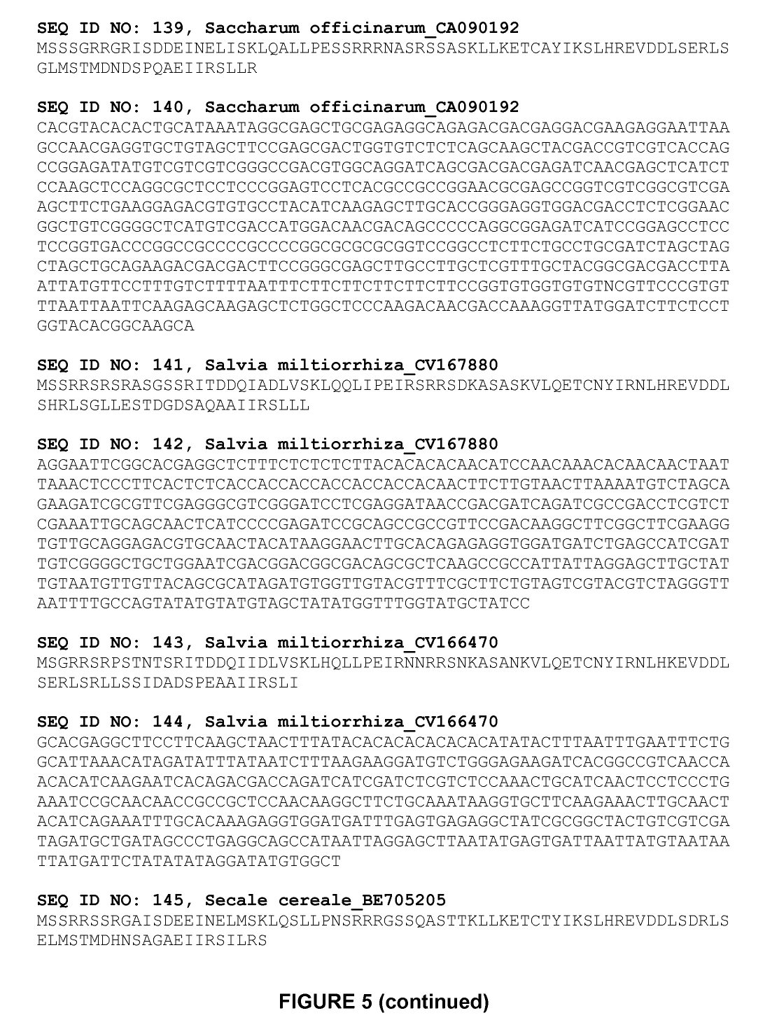

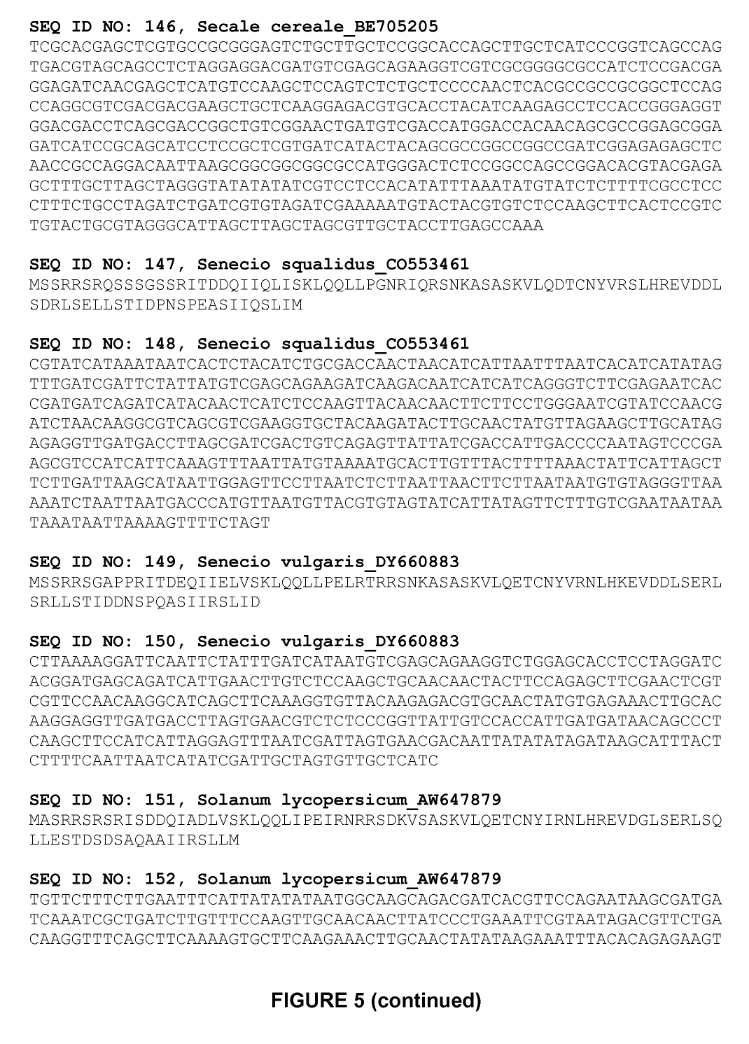

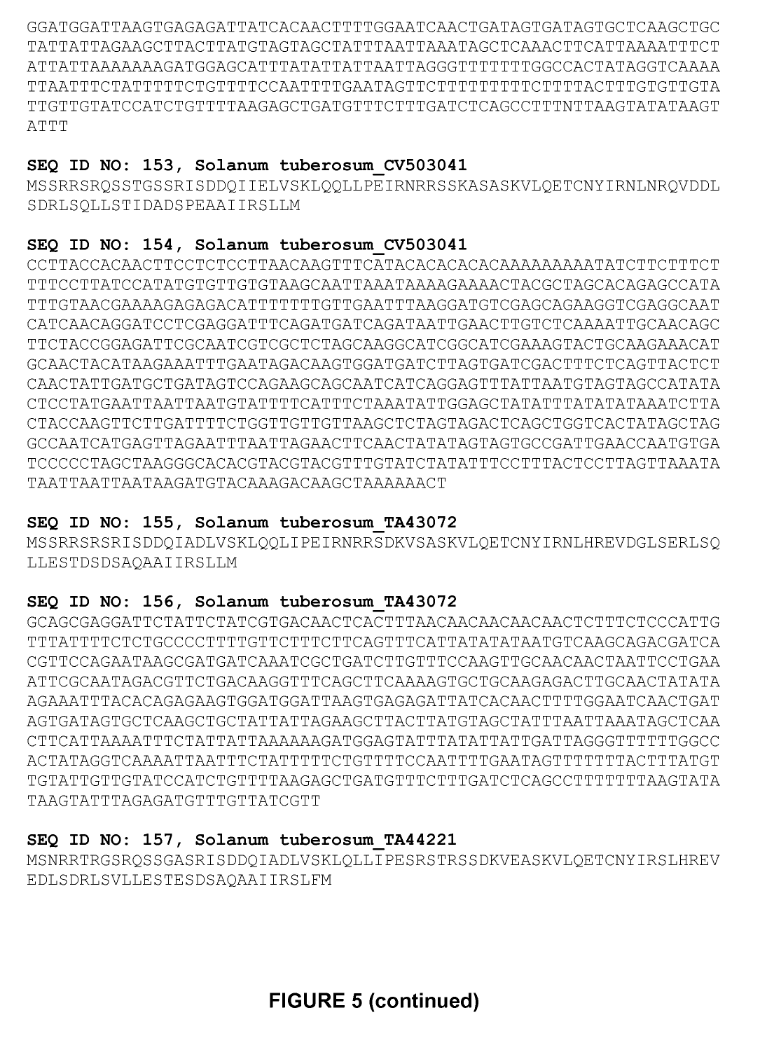

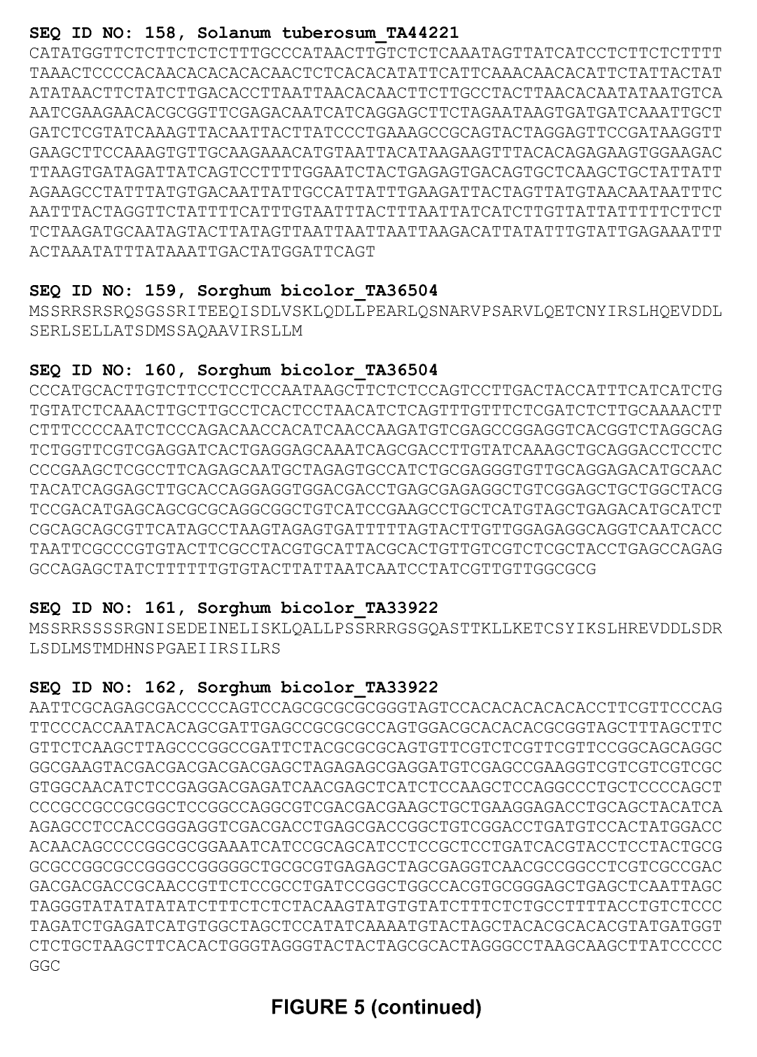

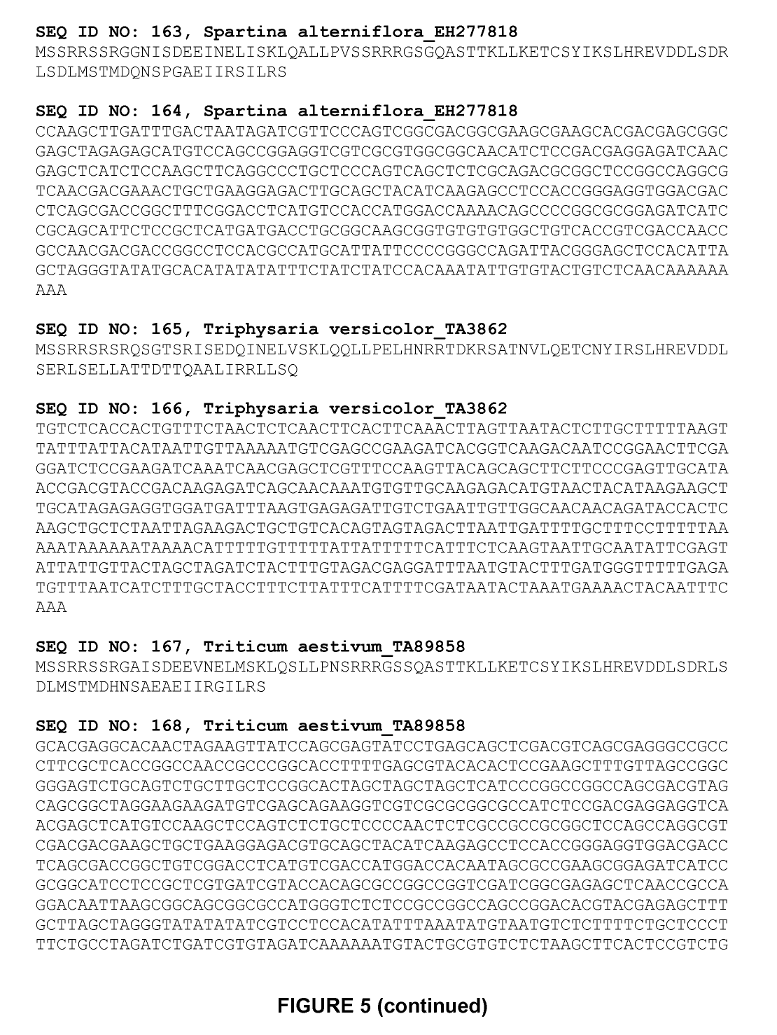

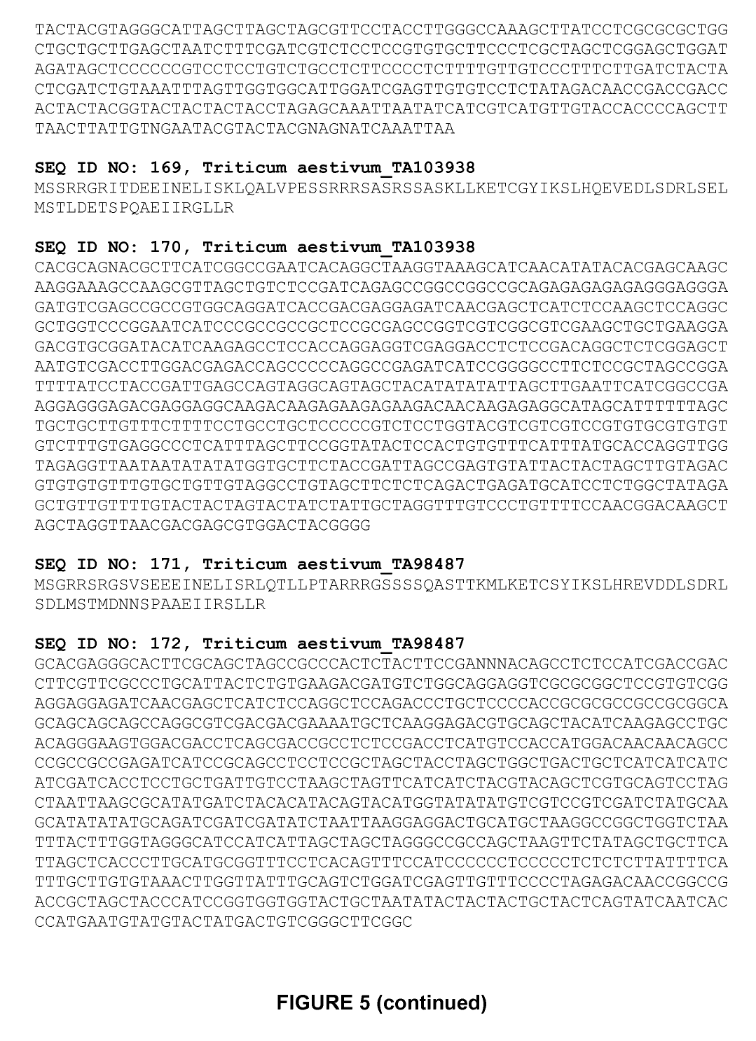

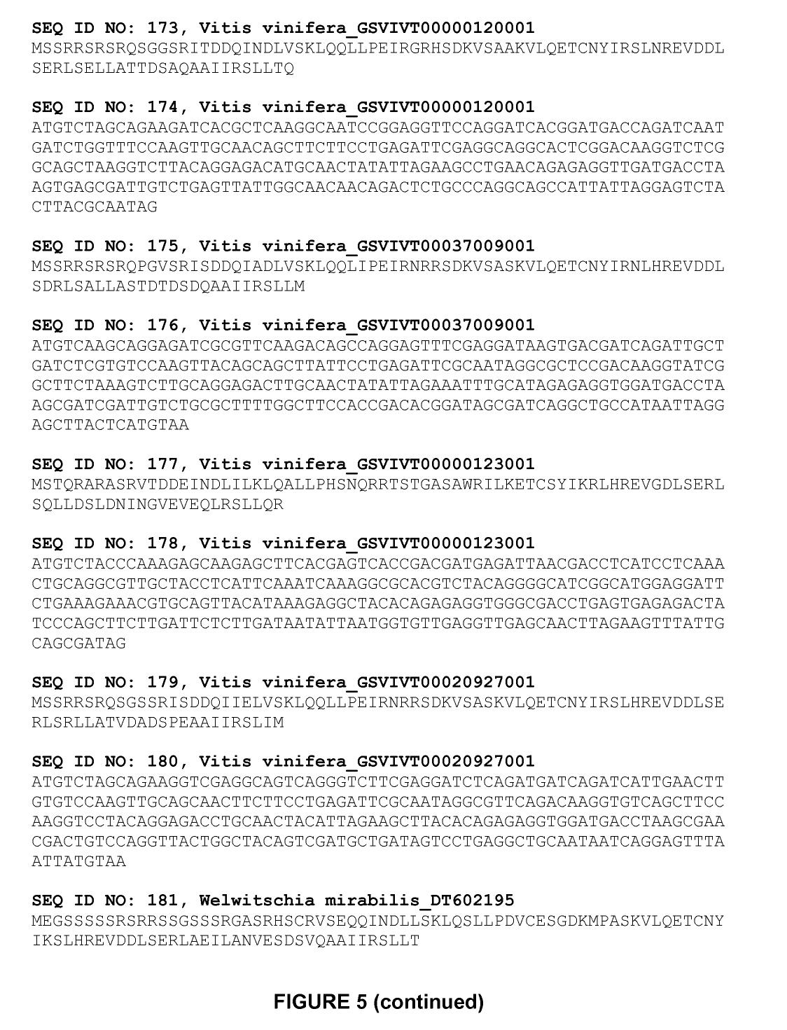

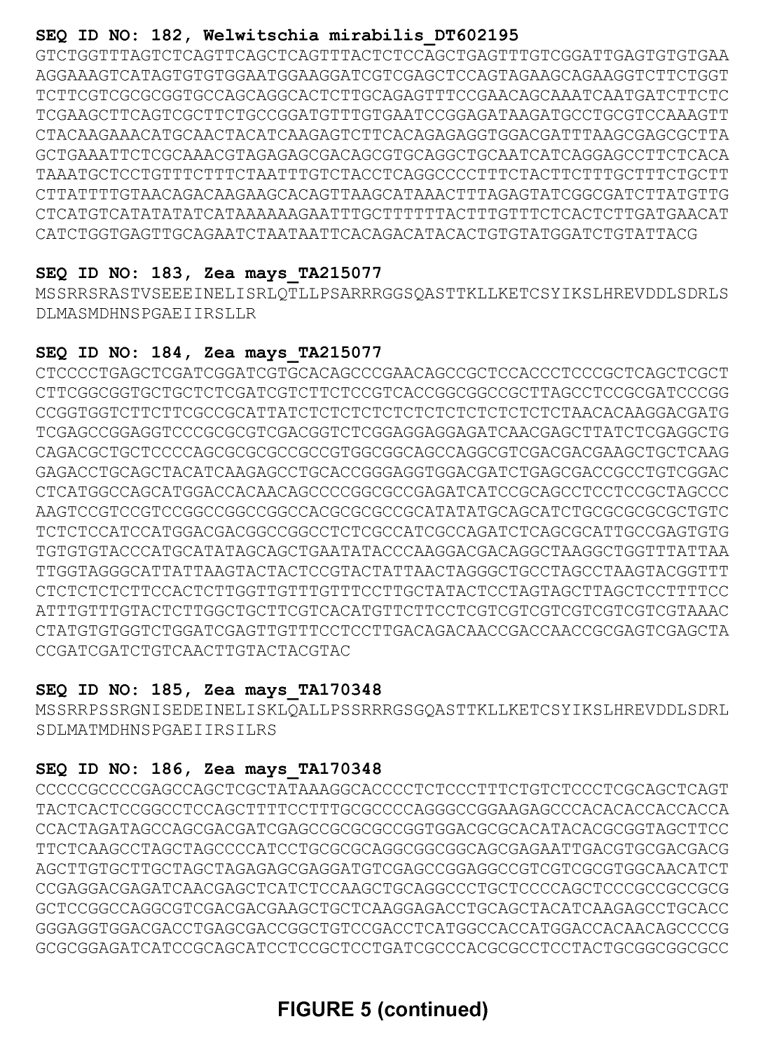

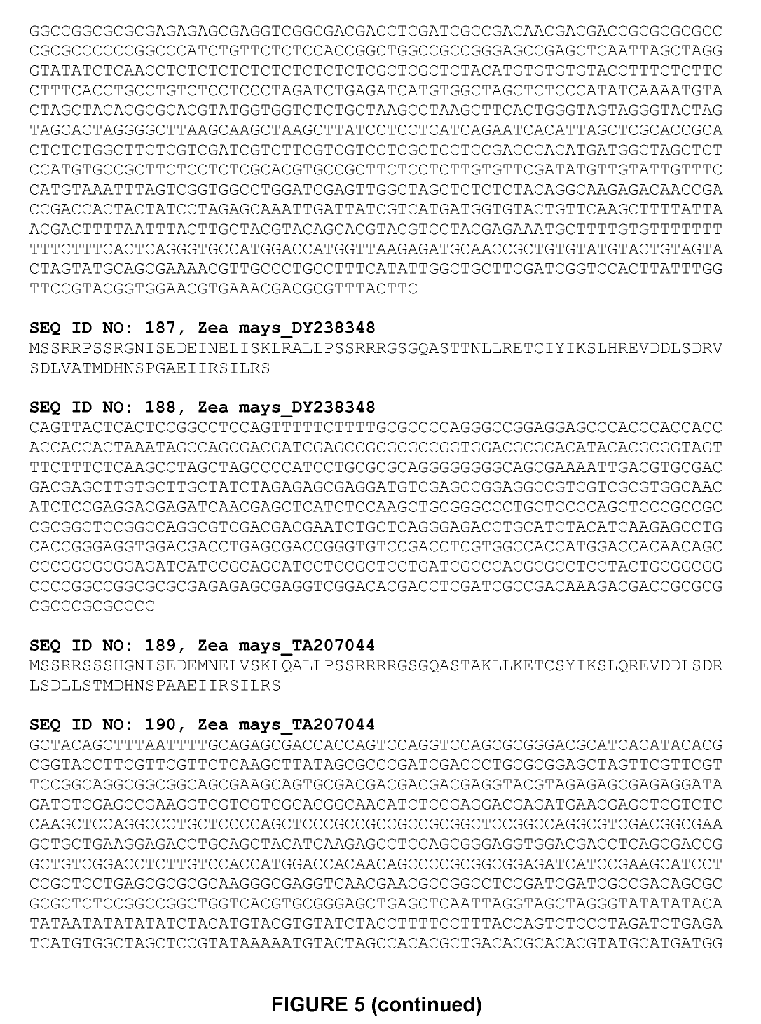

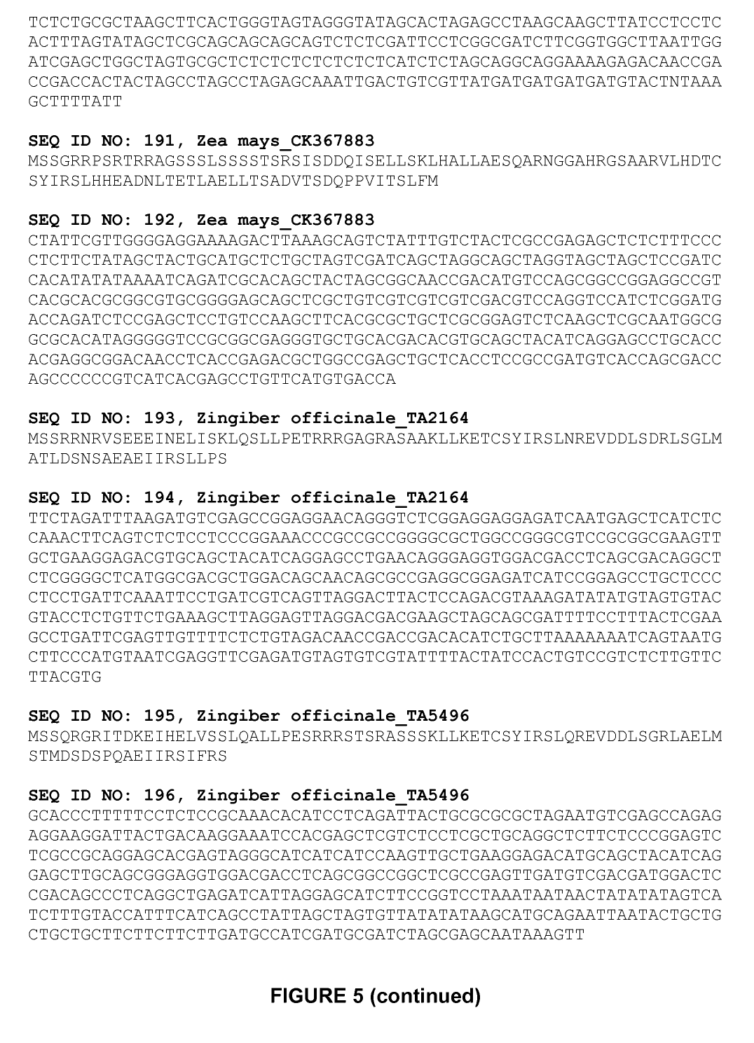

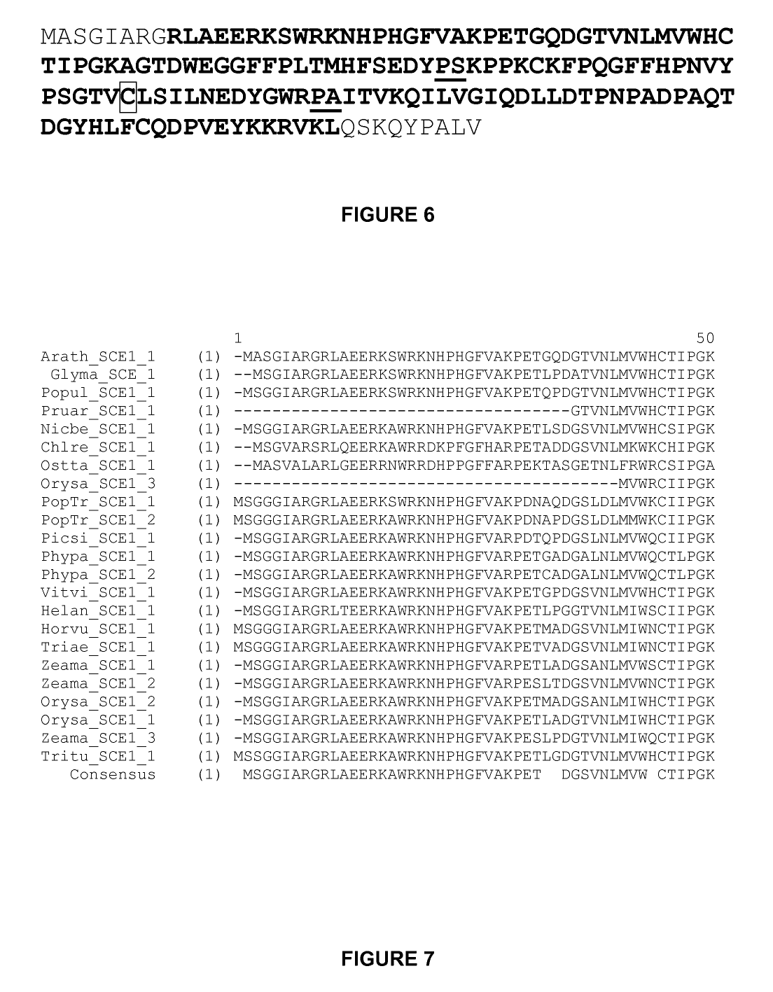

After the LR recombination step, the resulting expression vector pGOS2::PRE-like (FIG. 4) was transformed into Agrobacterium strain LBA4044 according to methods well known in the art.

### Example 7.2

### SCE1 Polypeptides

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were: 5′-ggggacaagtttgtacaaaaaagcaggcttaaacaatggctagtggaatcgctc-3′ (SEQ ID NO: 245); and 5′-ggggaccactttgtacaagaaagctgggtatcagttttggtgcgttctc-3′ (SEQ ID NO: 246) which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pArath_SCE1_{—}1. Plasmid pDONR201 was purchased from Invitrogen, as part of the Gateway® technology.

The entry clone comprising SEQ ID NO: 199 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 247) for constitutive specific expression was located upstream of this Gateway cassette.

After the LR recombination step, the resulting expression vector pGOS2::Arath_SCE1_{—}1 (FIG. 8) was transformed into Agrobacterium strain LBA4044 according to methods well known in the art.

### Example 7.3

### YEF1 Polypeptides

The nucleic acid sequence used in the methods of the invention was amplified by PCR using as template a custom-made Lycopersicum esculentum seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were: 5′-GGGGACAAGTTTGTACAAAAAAGCAGGCTTAAACAATGGATGCTTATGAAGCTACA-3′ (SEQ ID NO: 279) and 5′-GGGGACCACTTTGTACAAGAAAGCTGGGTACGTAACATAACATGCTG TCC-3′ (SEQ ID NO: 280), which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pYEF1_{—}1. Plasmid pDONR201 was purchased from Invitrogen, as part of the Gateway® technology.

The entry clone comprising SEQ ID NO: 248 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 281) for root specific expression was located upstream of this Gateway cassette.

After the LR recombination step, the resulting expression vector pGOS2::Le_YEF1_{—}1 (FIG. 12) was transformed into Agrobacterium strain LBA4044 according to methods well known in the art.

### Example 7.4

### Subgroup III Grx

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm09053 (SEQ ID NO: 437; sense, start codon in bold): 5′-ggggacaagtttgtacaaaaaagcagg cttaaacaatggatatgataacgaagatg-3′ and prm09054 (SEQ ID NO: 438; reverse, complementary): 5′-ggggaccactttgtacaagaaagctgggtaaaaacatgataagtcaaa cc-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

The entry clone comprising SEQ ID NO: 282 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A protochlorophyllid reductase promoter (SEQ ID NO: 436) for green tissue-specific expression was located upstream of this Gateway cassette.

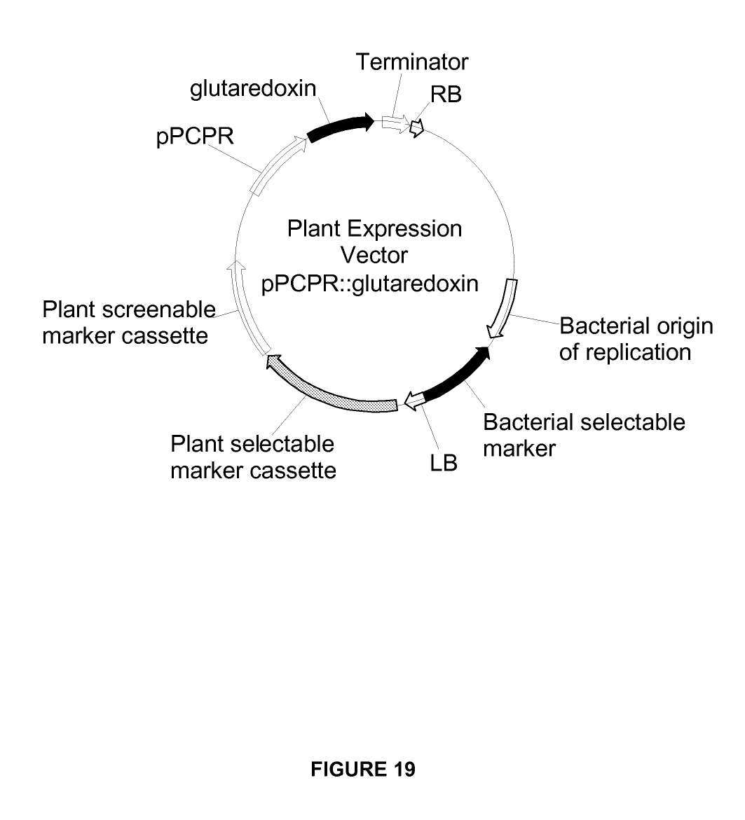

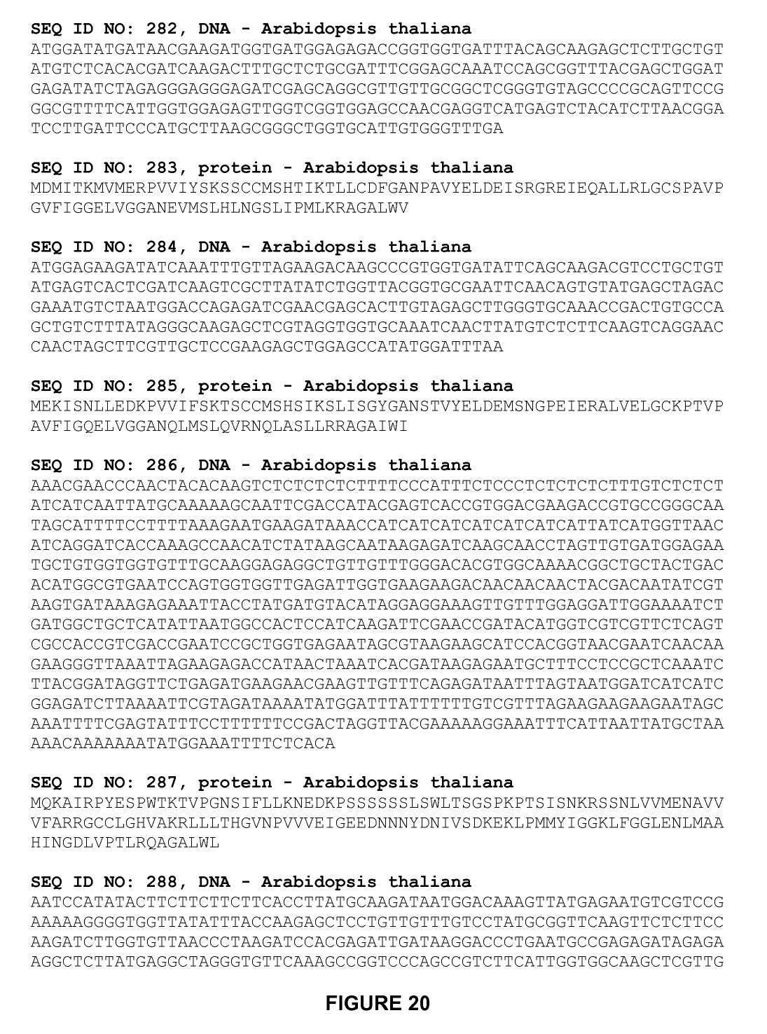

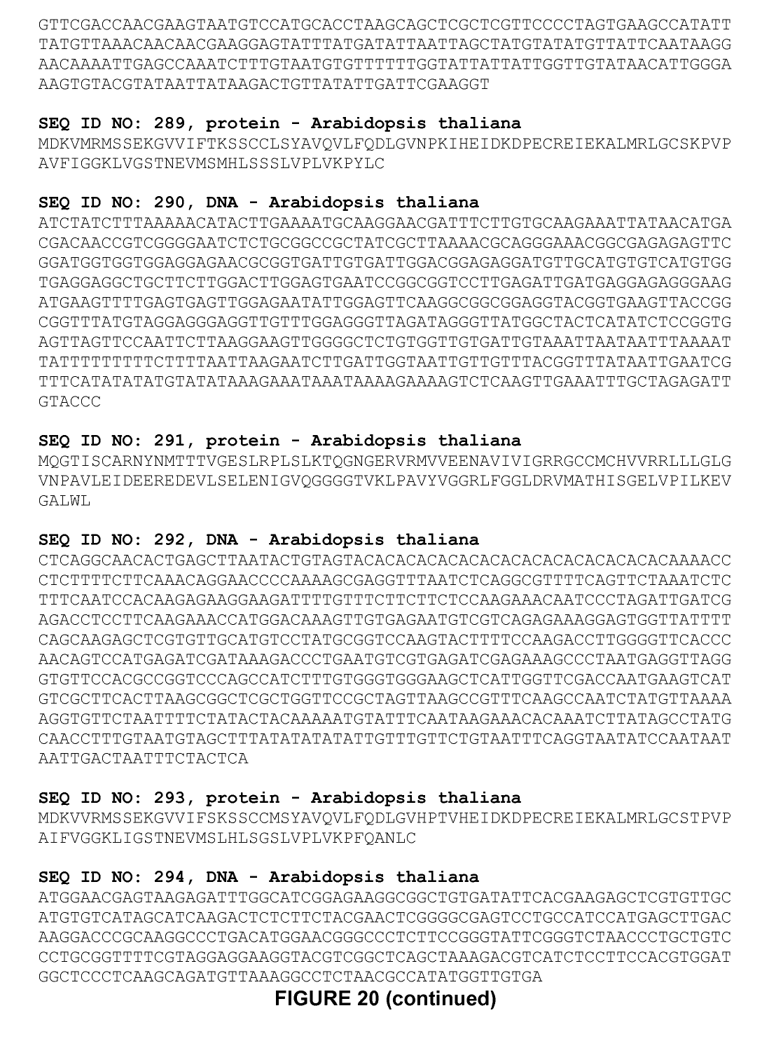

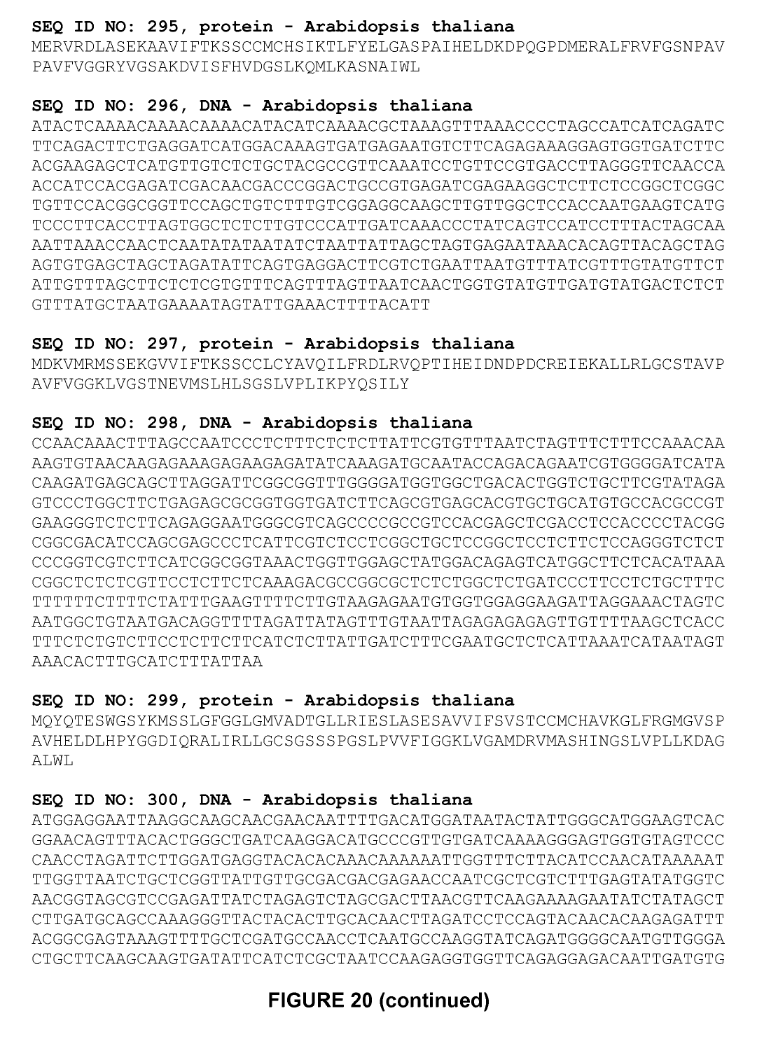

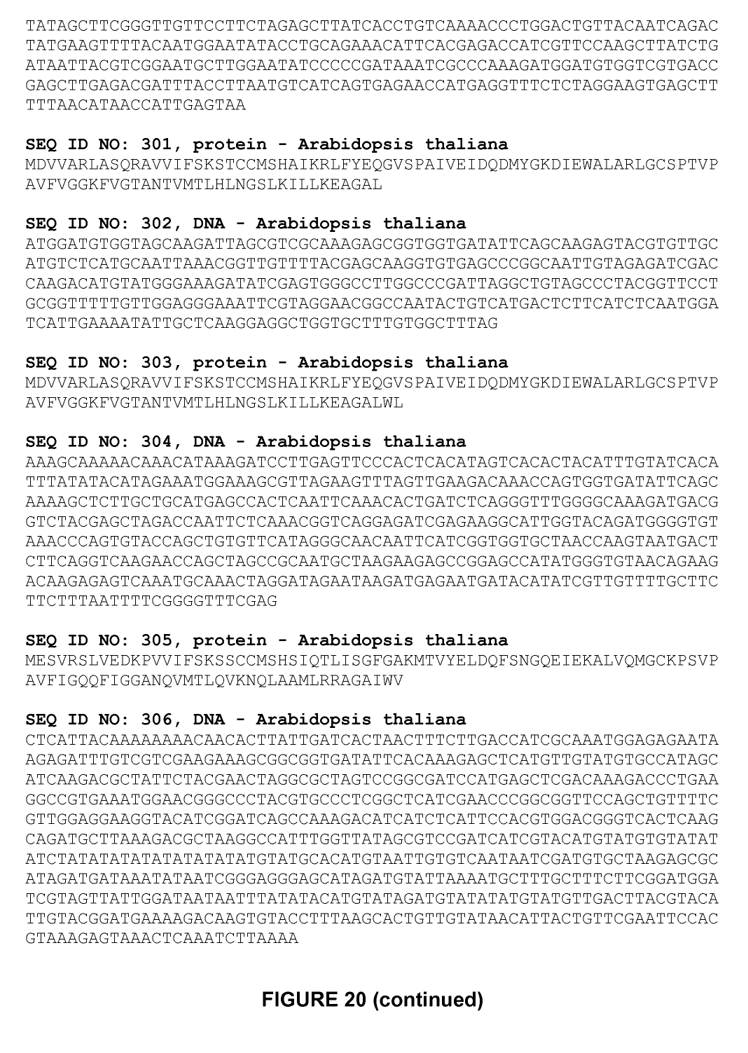

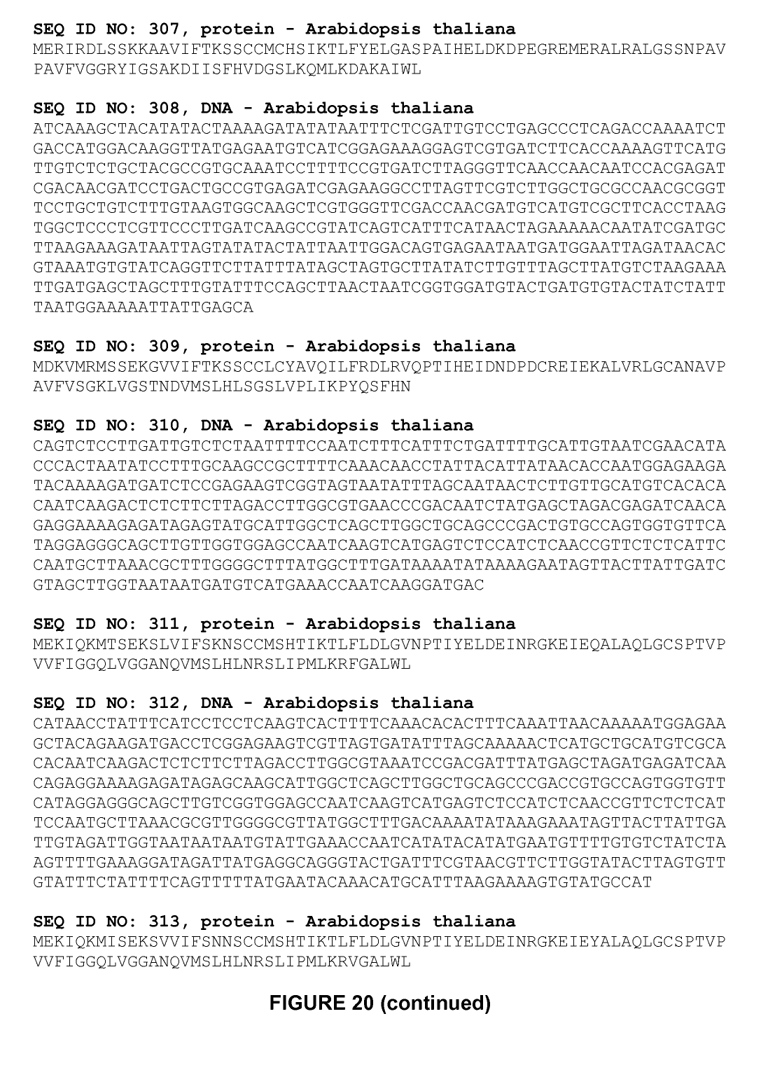

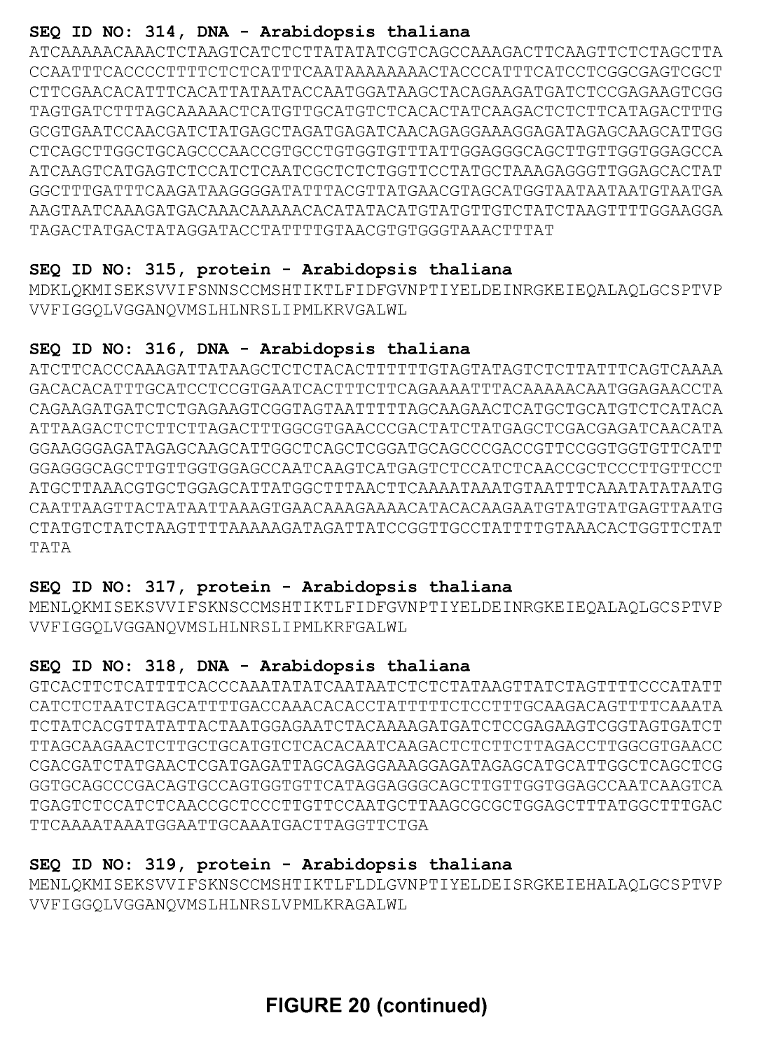

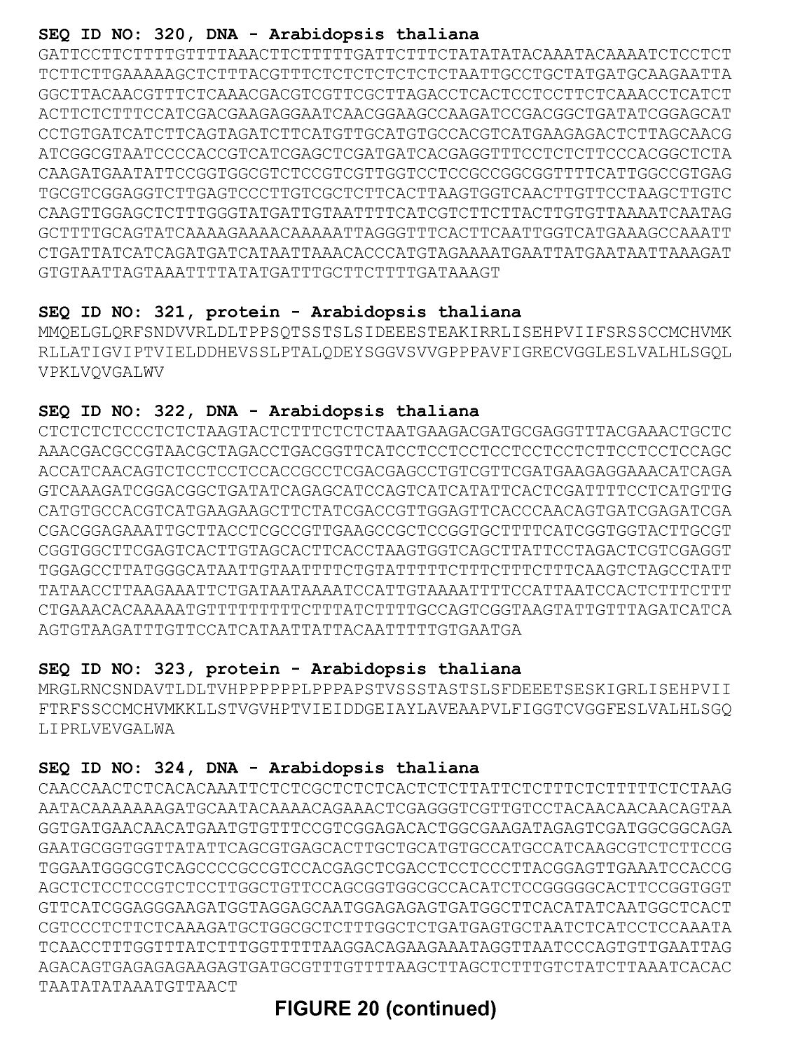

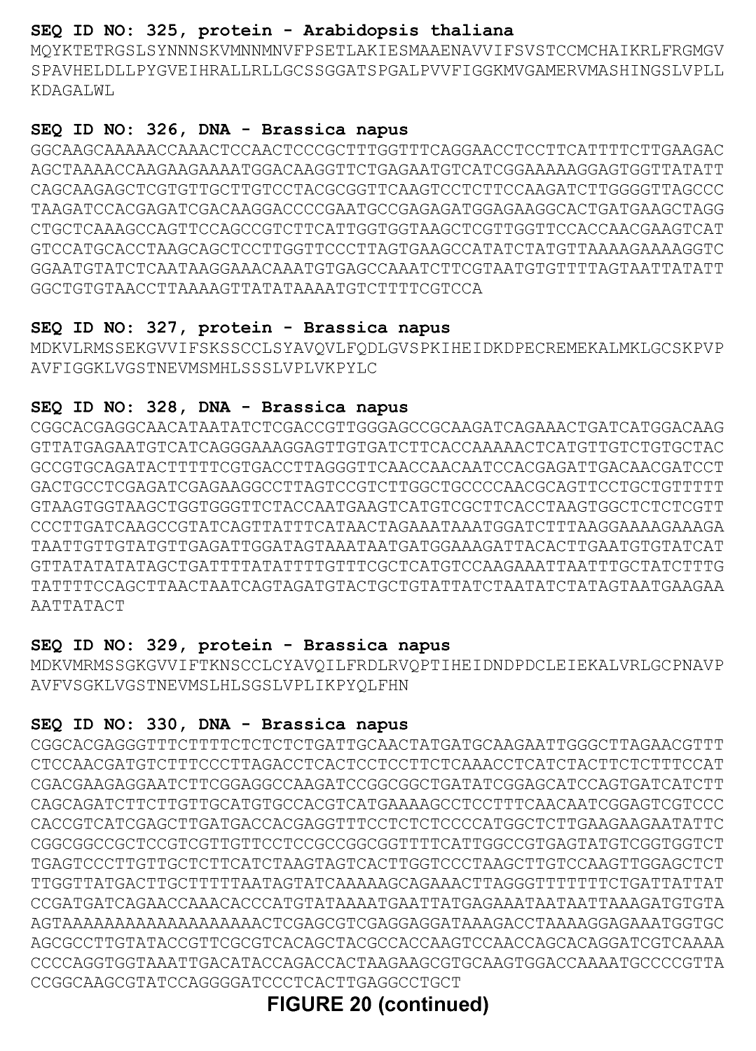

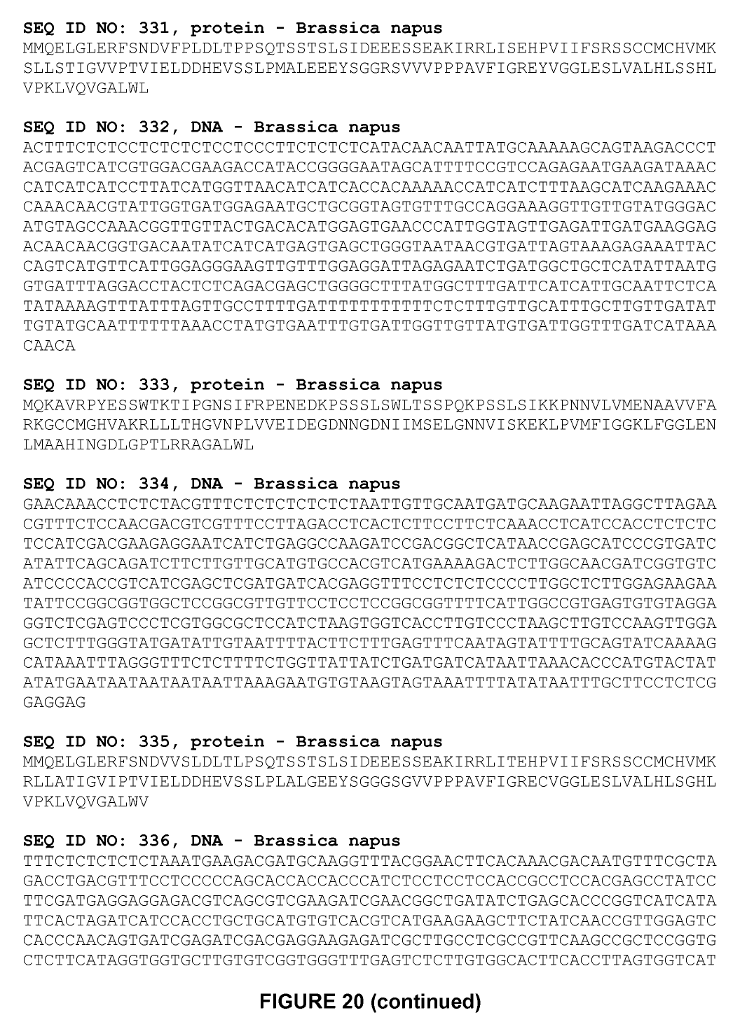

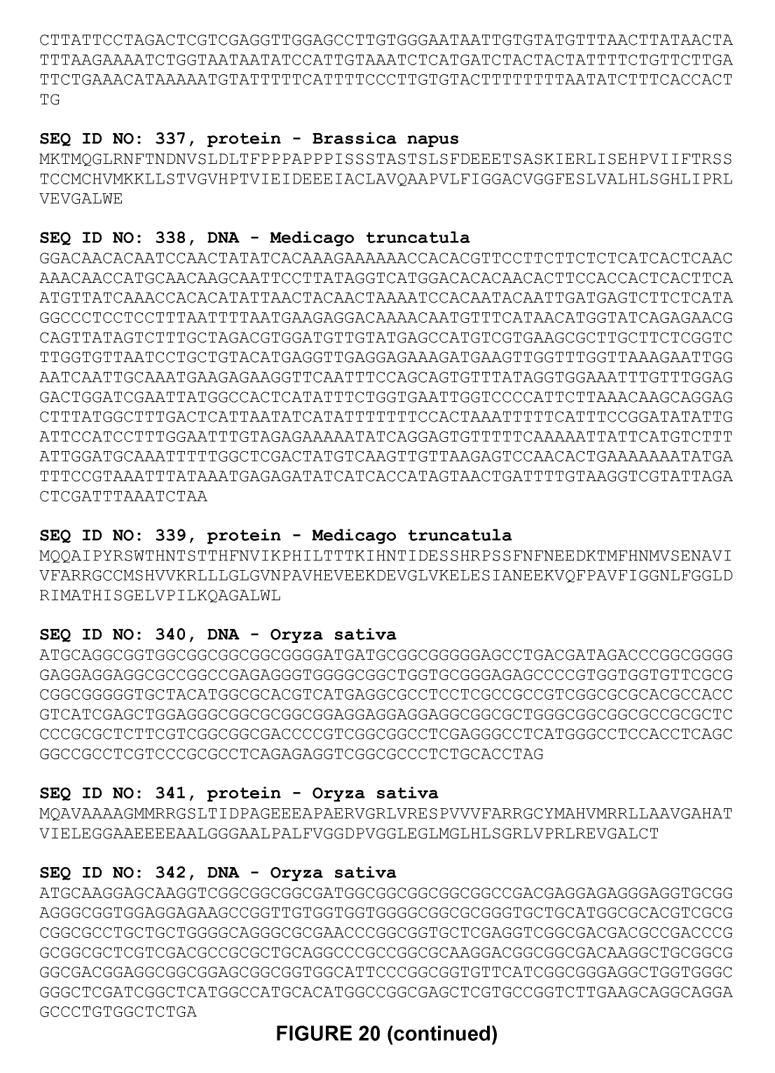

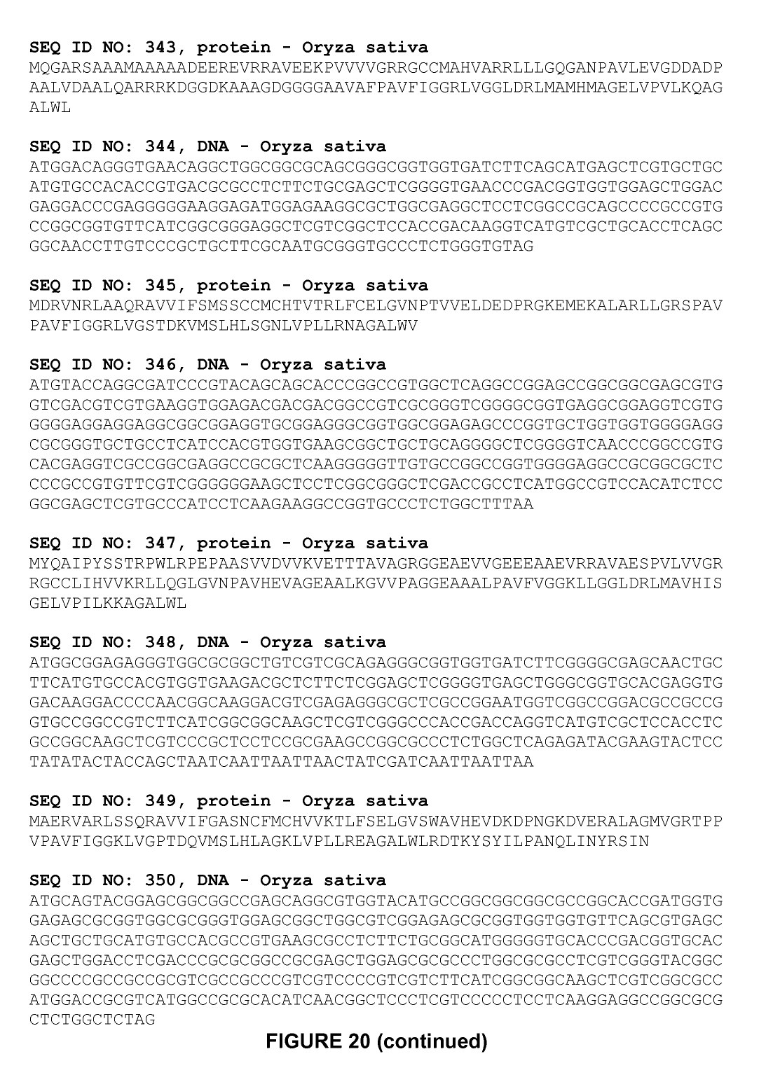

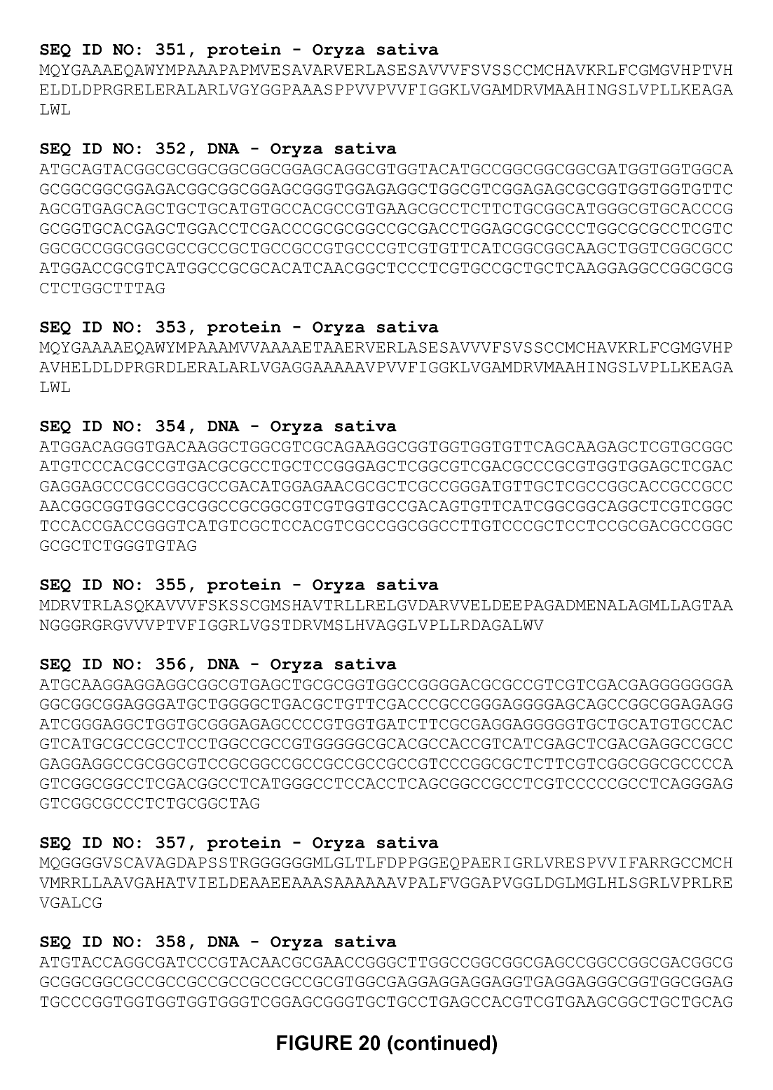

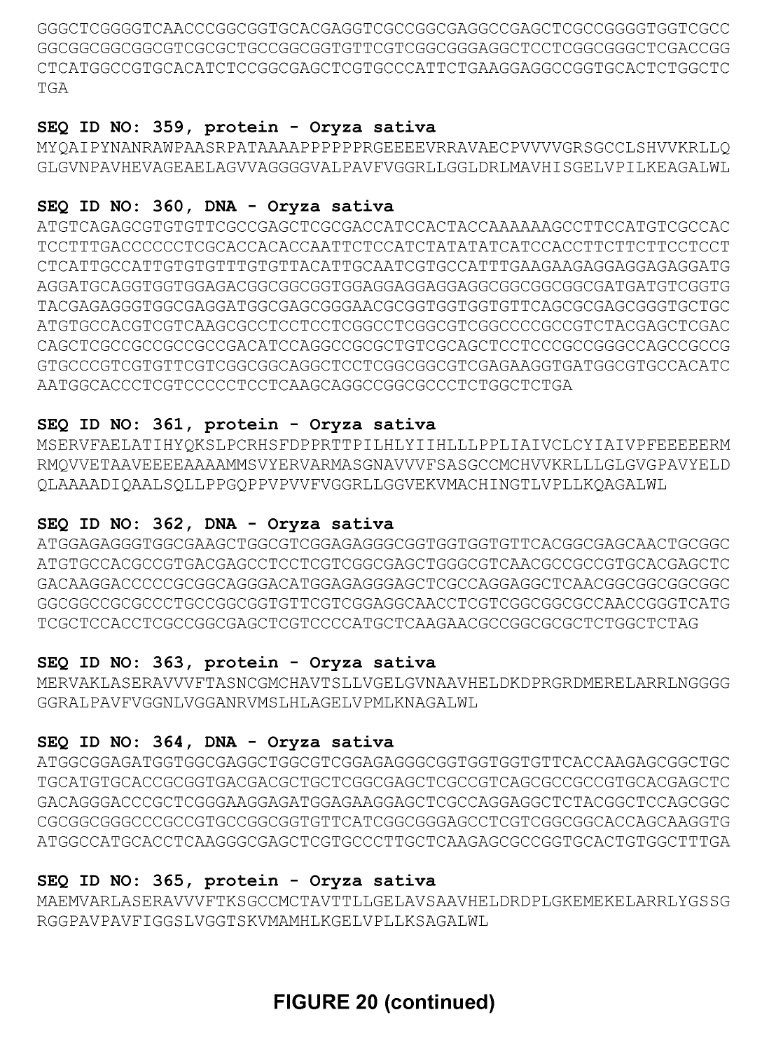

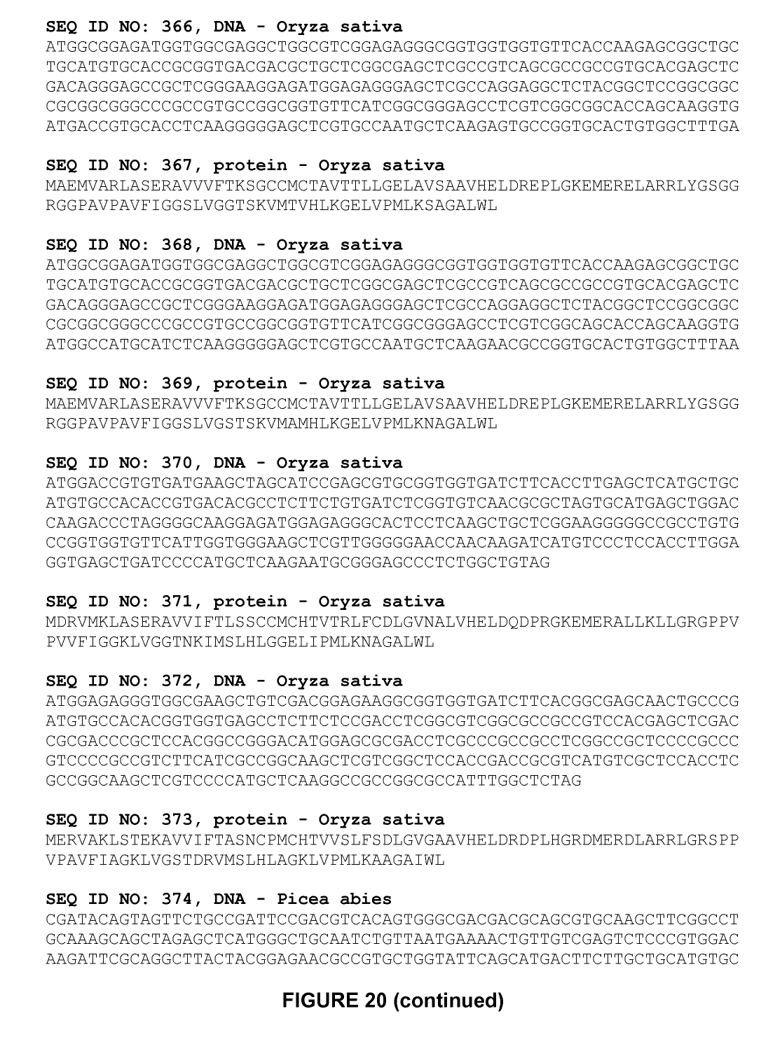

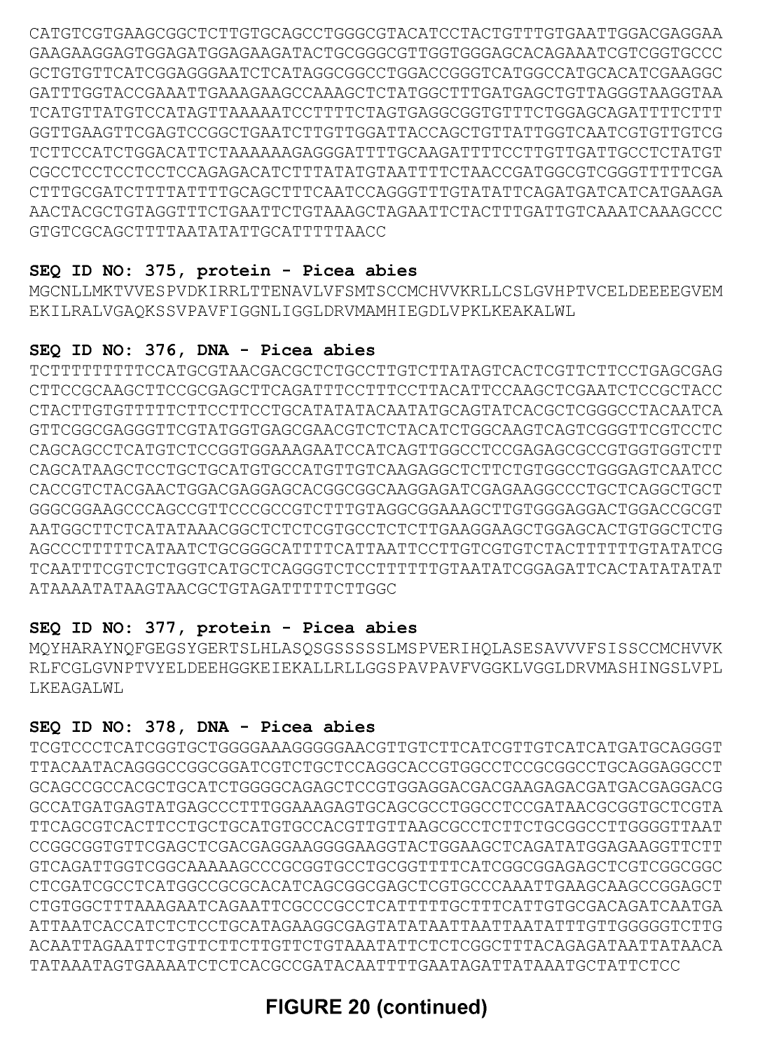

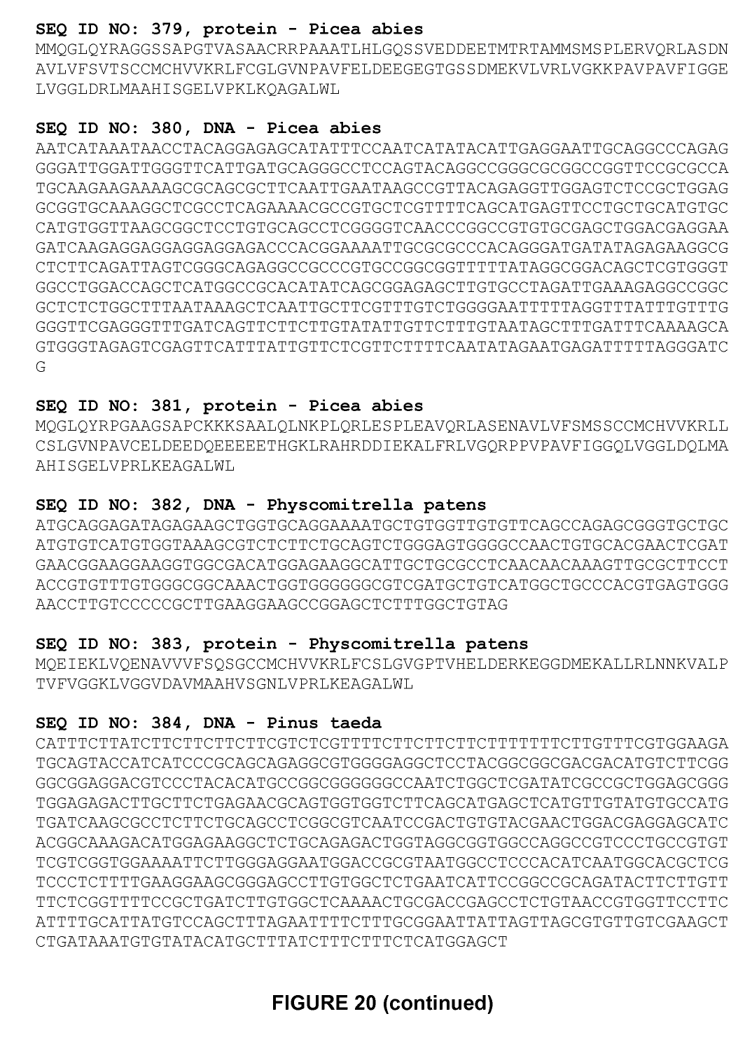

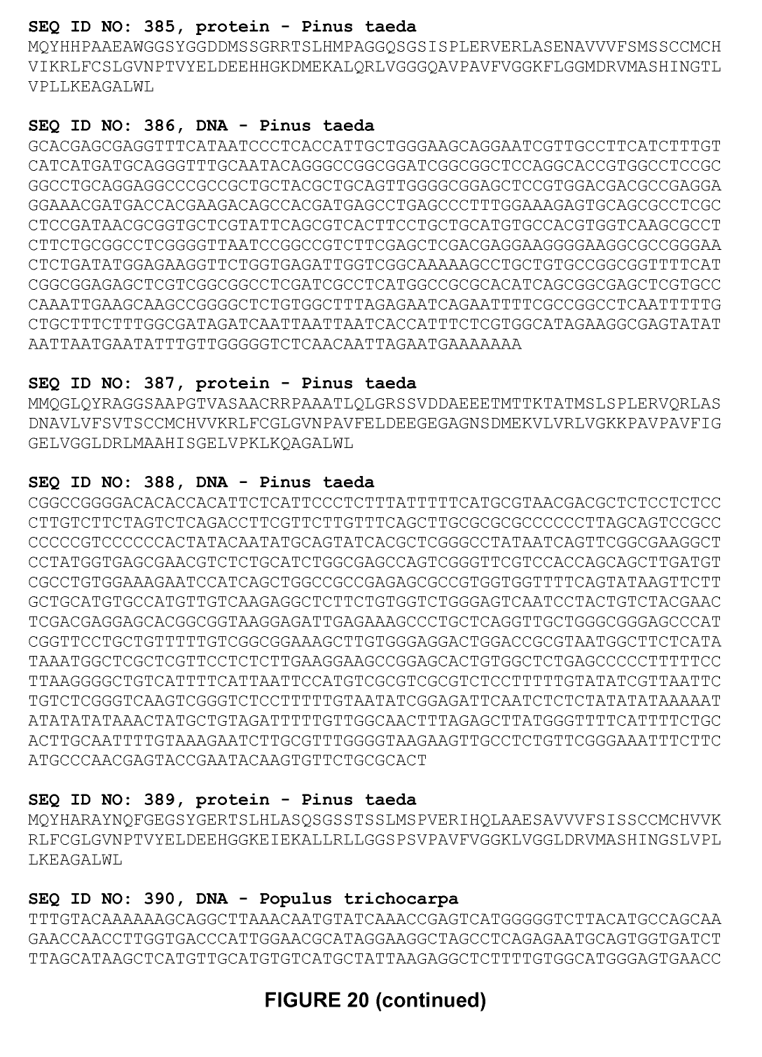

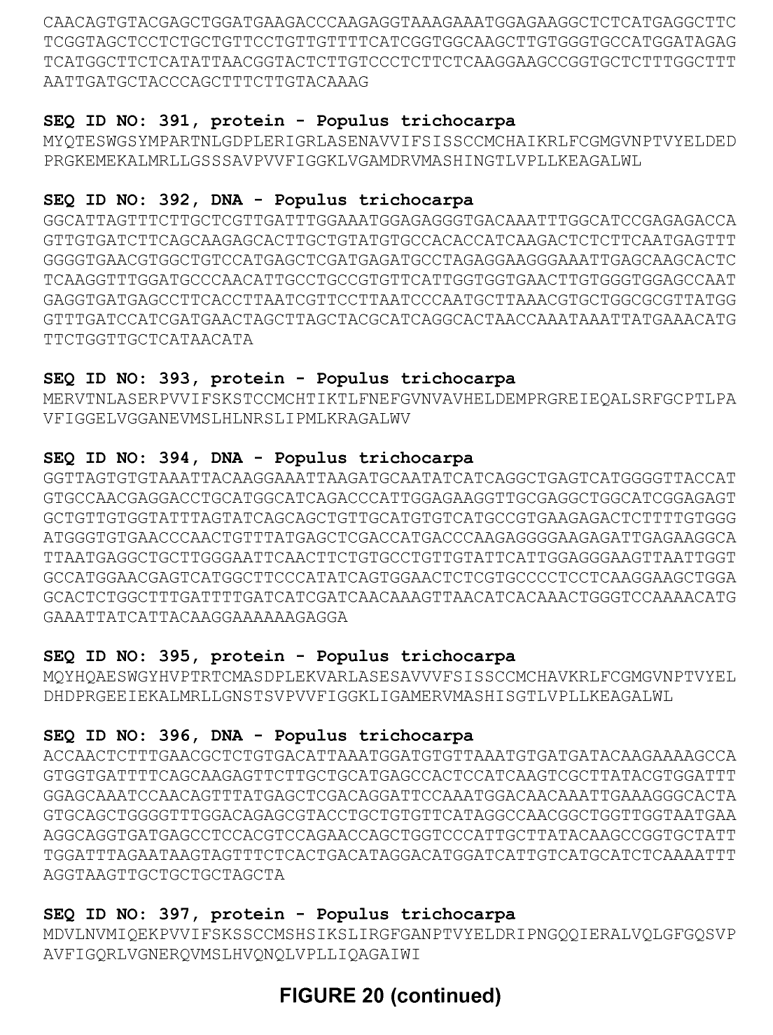

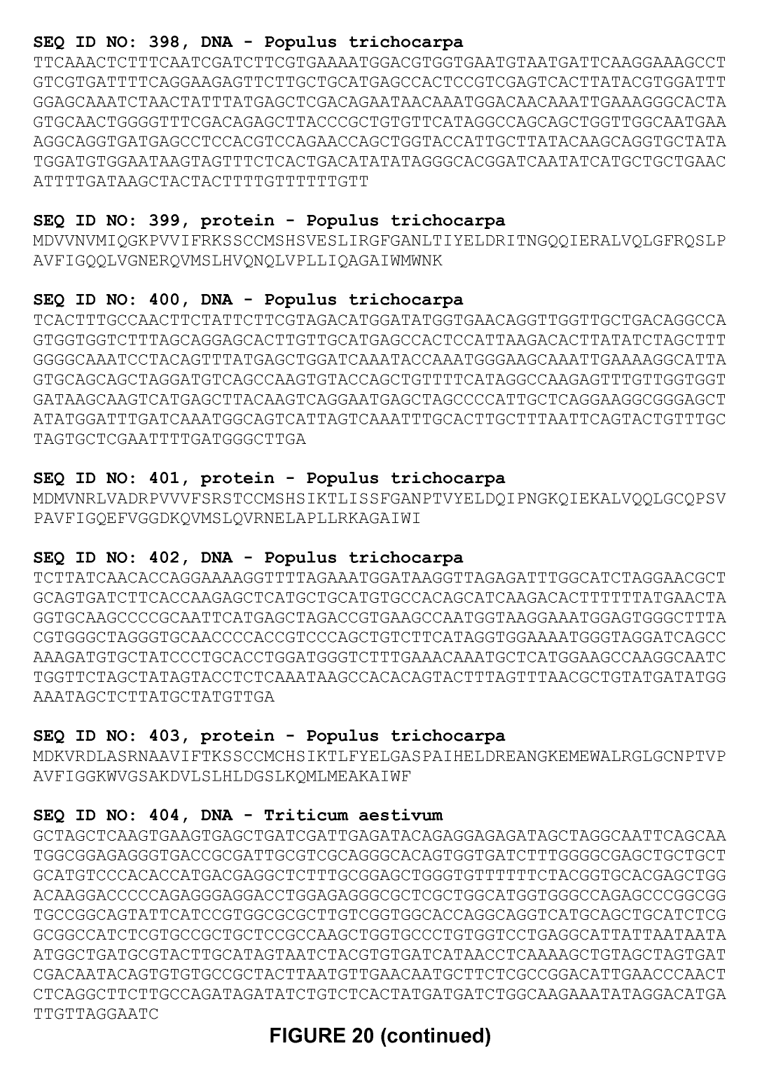

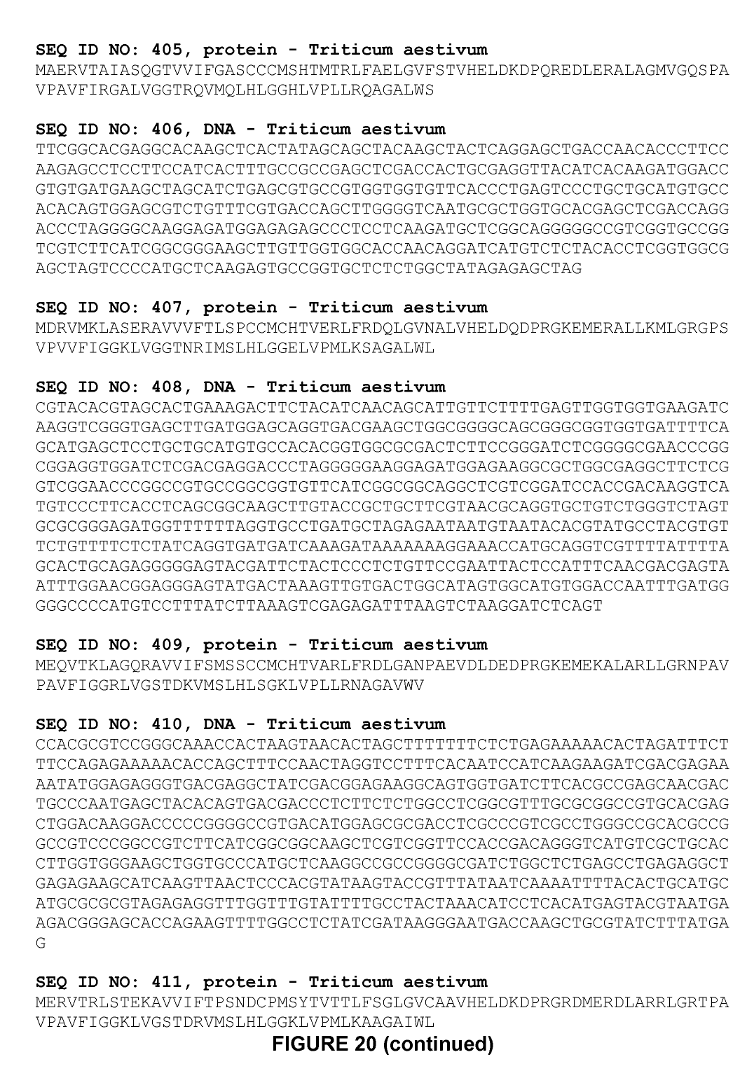

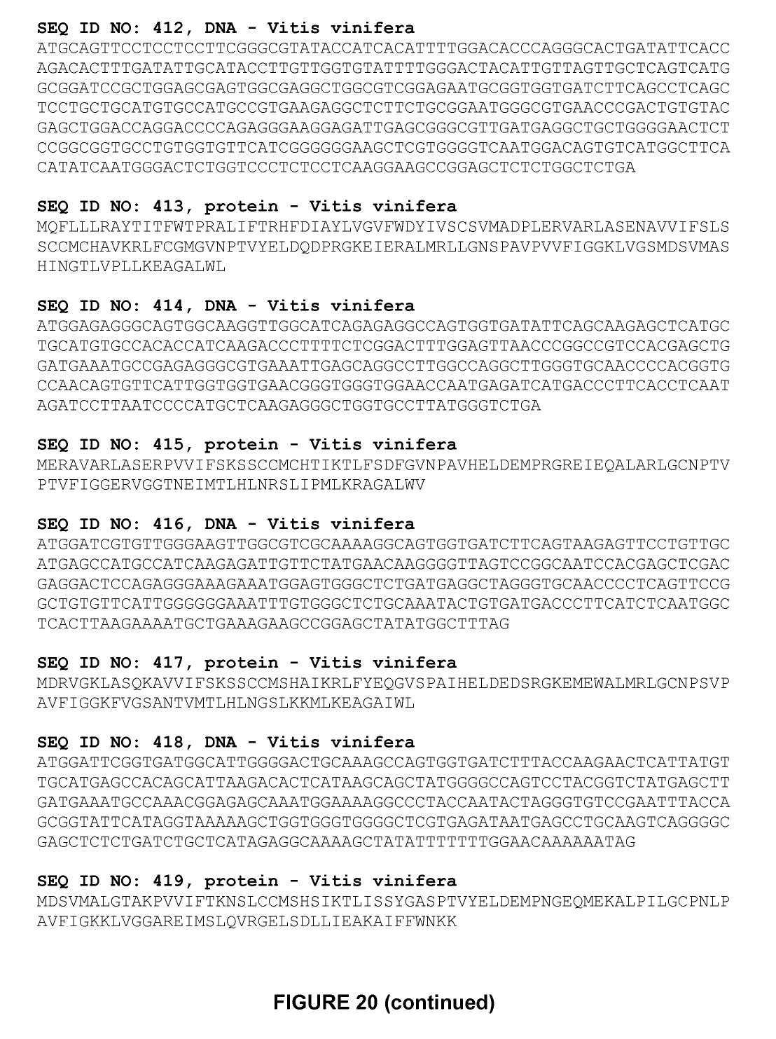

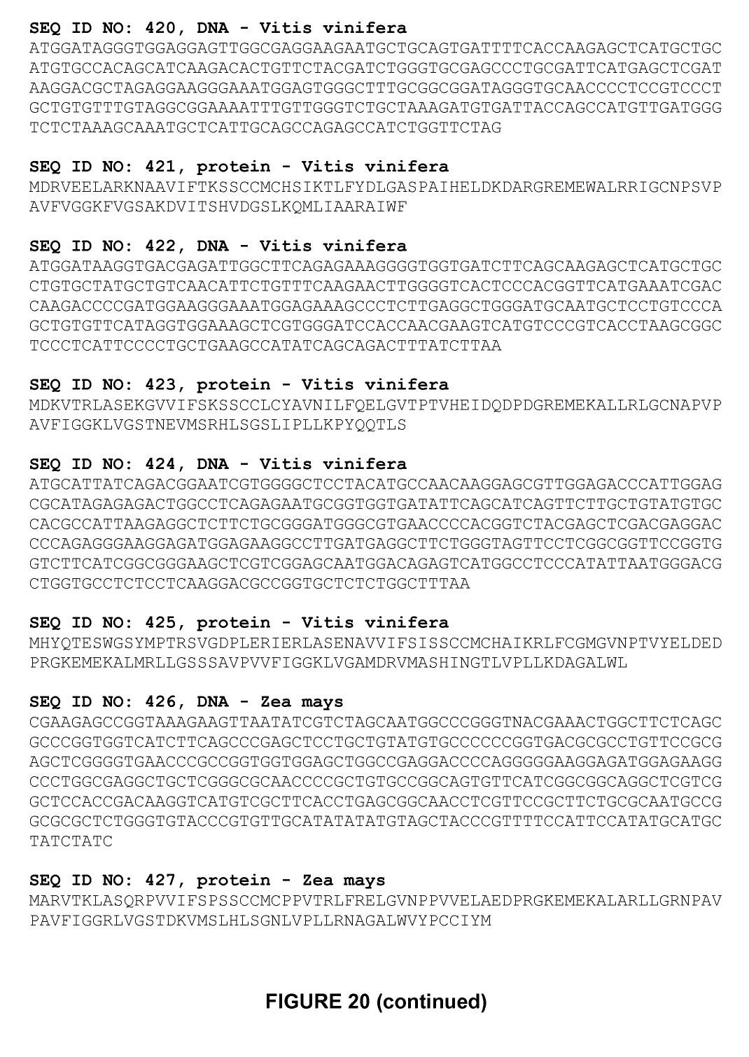

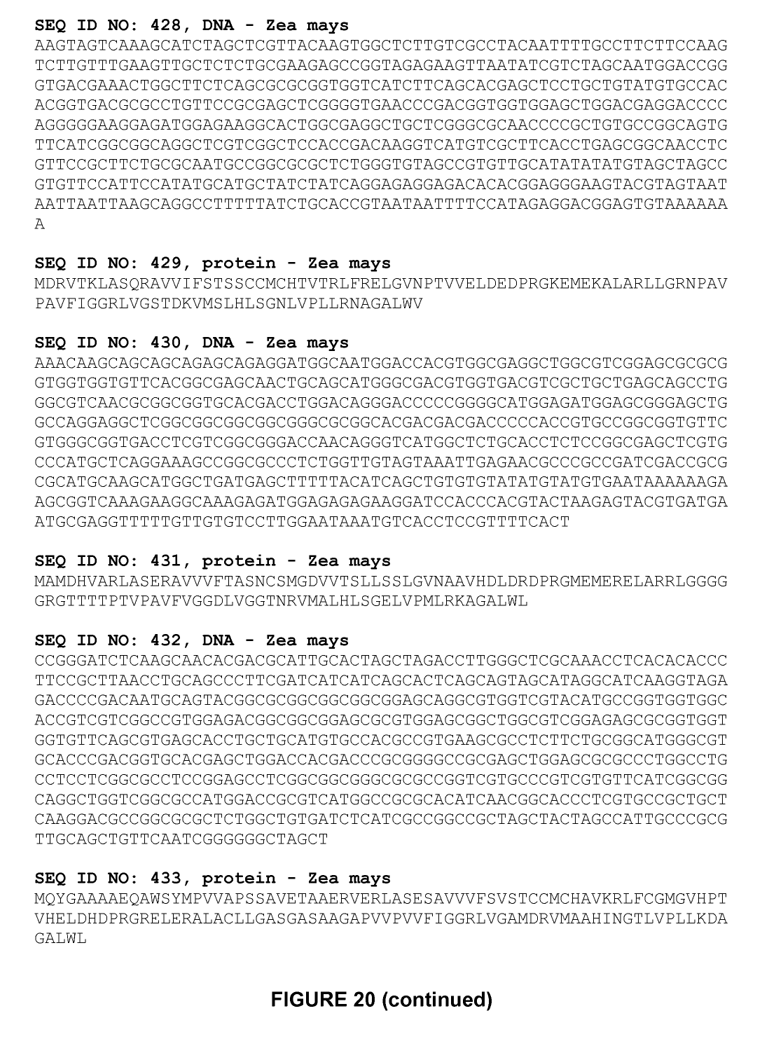

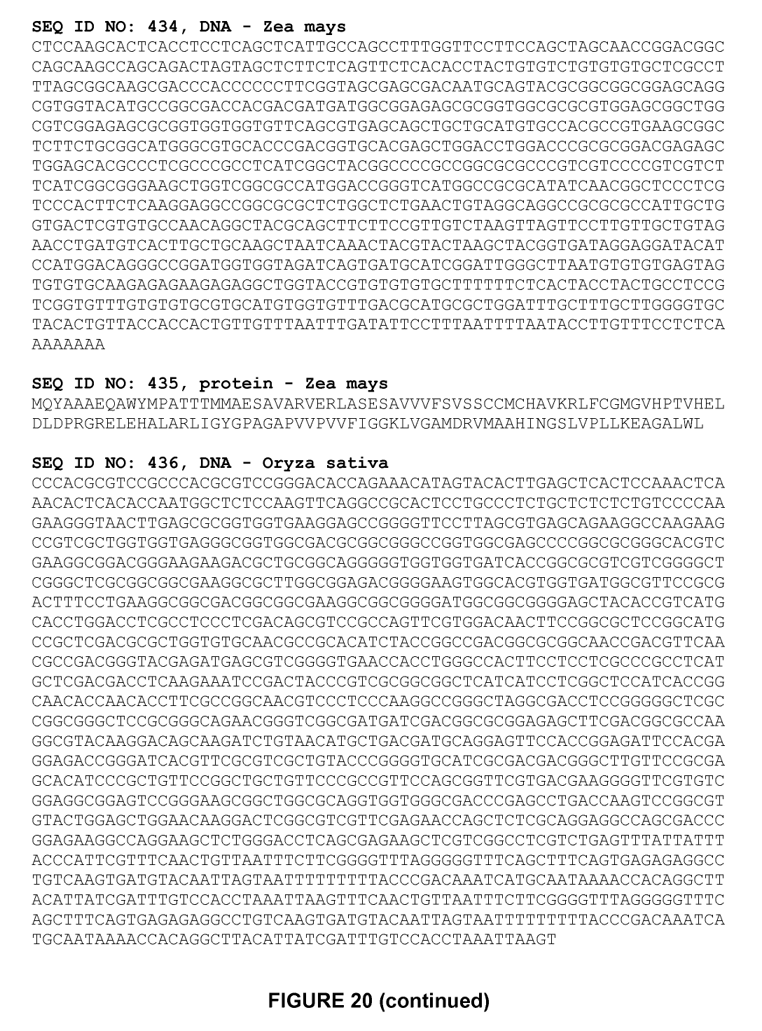

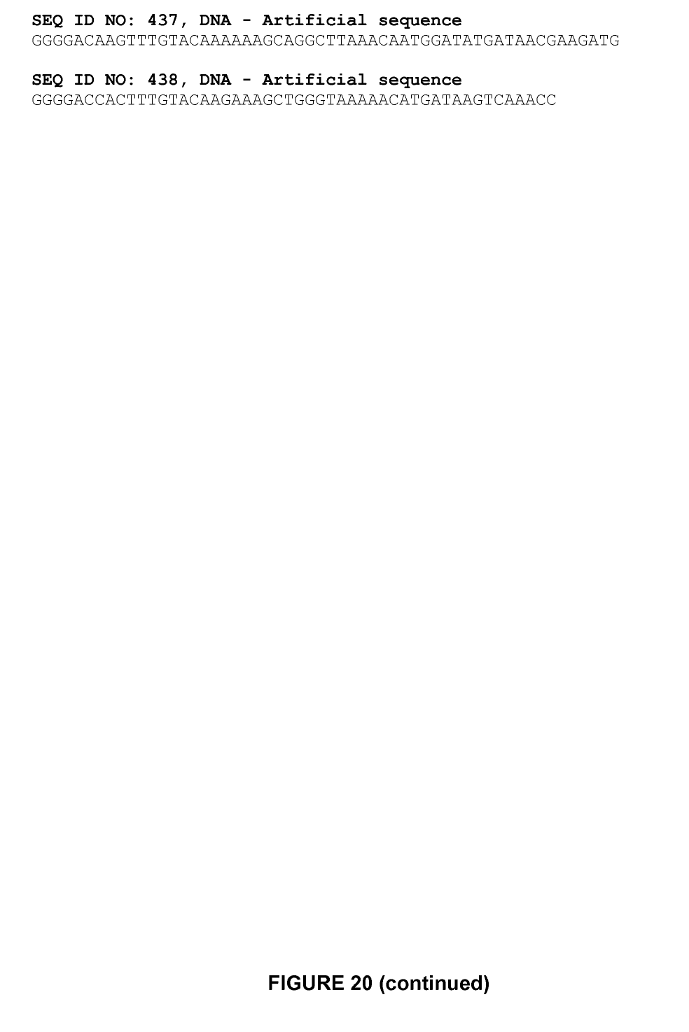

After the LR recombination step, the resulting expression vector pPCPR::Grx (FIG. 19) was transformed into Agrobacterium strain LBA4044 according to methods well known in the art.

### Example 7.5

### Sister of FT

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm4759 (SEQ ID NO: 442; sense, start codon in bold): 5′-ggggacaagtttgtacaaaaaagcaggctt aaacaatgtctttaagtcgtagagatcc-3′ and prm4760 (SEQ ID NO: 443; reverse, complementary): 5′-ggggaccactttgtacaagaaagctgggtgtacgcatctacgttcttc tt-3′, which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, pGOS2::Sister of FT. Plasmid pDONR201 was purchased from Invitrogen, as part of the Gateway° technology.

The entry clone comprising SEQ ID NO: 439 was then used in an LR reaction with a destination vector used for Oryza sativa transformation. This vector contained as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 441) for constitutive expression was located upstream of this Gateway cassette.

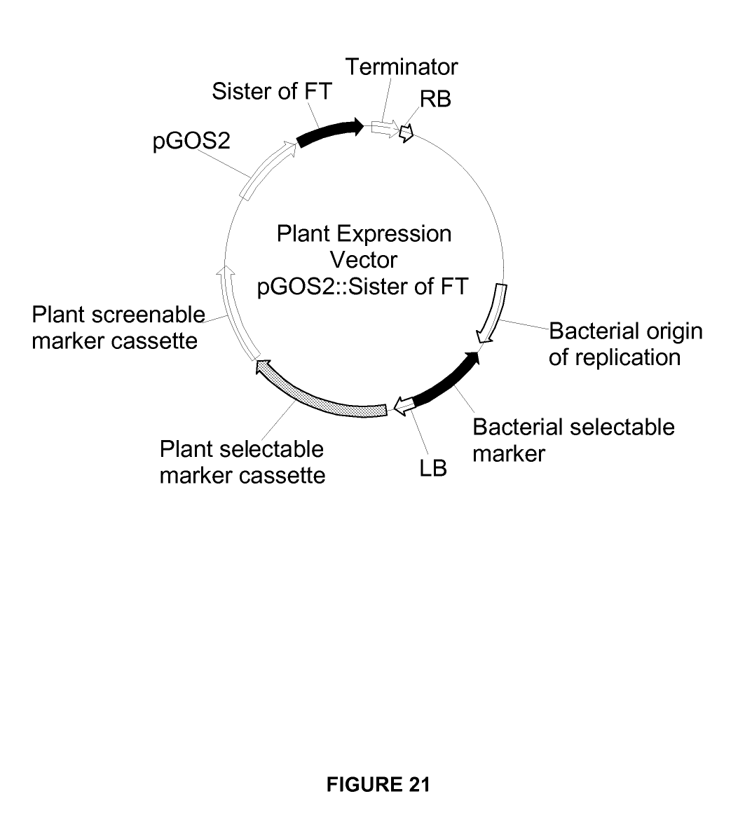

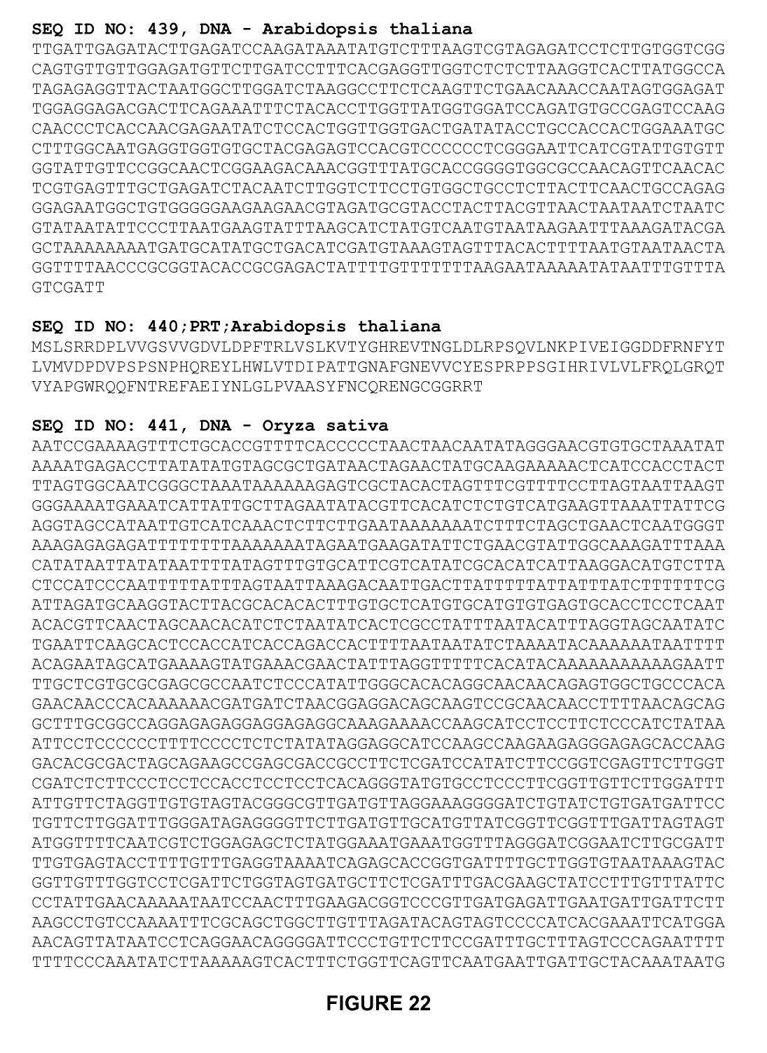

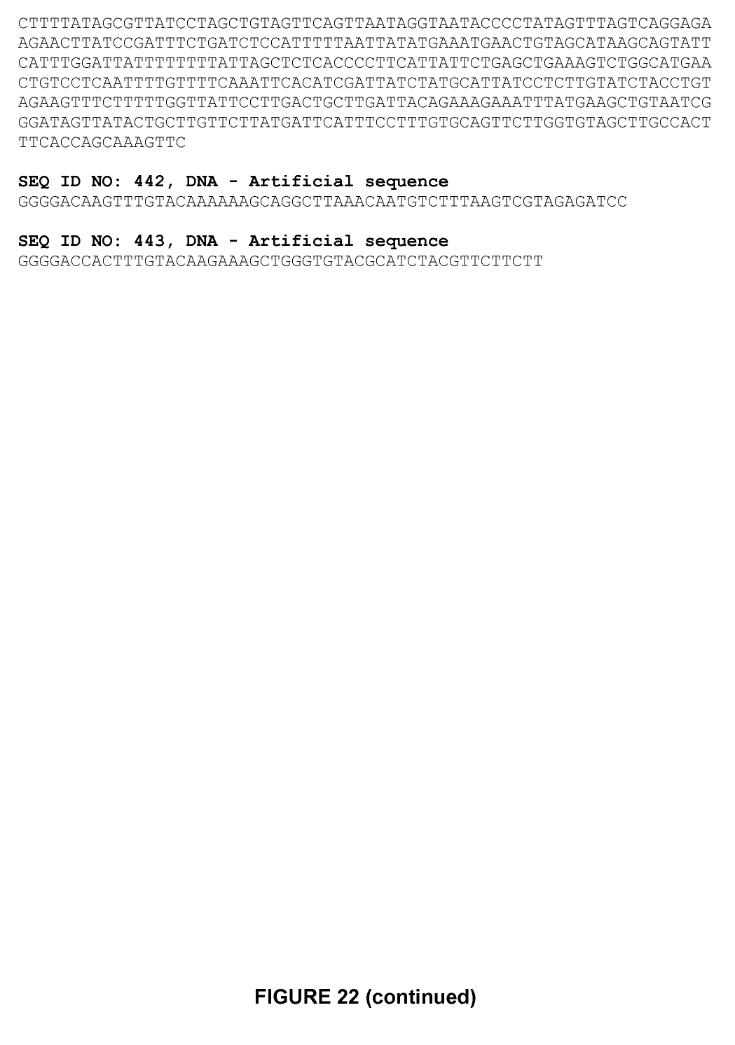

After the LR recombination step, the resulting expression vector pGOS2::Sister of FT (FIG. 21) was transformed into Agrobacterium strain LBA4044 according to methods well known in the art.

### Example 8

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and washed in distilled water with 500 μg/ml cefotaxime. The seeds are then transferred to SH-medium with 5 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation.

### Example 9

### Phenotypic Evaluation Procedure

### Example 9.1

### PRE-Like Sequences

### 9.1.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approach the heading stage. They are then transferred to a “dry” section where irrigation is withheld. Humidity probes are inserted in randomly chosen pots to monitor the soil water content (SWC). When SWC goes below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was

the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters are then measured.

### 9.1.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

### 9.1.3 Parameters Measured

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight.

### Example 9.2

### SCE1 Sequences

### 9.2.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are watered at regular intervals to ensure that water and nutrients are not limiting to satisfy plant needs to complete growth and development.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC is below certain thresholds, the plants are automatically re-watered continuously until a normal level is reached again. The plants are then re-transferred again to normal conditions. The rest of the cultivation (plant maturation, seed harvest) is the same as for plants not grown under abiotic stress conditions. Growth and yield parameters are recorded as detailed for growth under normal conditions.

### Nitrogen Use Efficiency Screen

Rice plants from T2 seeds were grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters are then measured.

### 9.2.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 9.2.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 9.3

### YEF1 Sequences

### 9.3.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Plants grown under non-stress conditions are watered at regular intervals to ensure that availability of water and nutrients are not limiting to satisfy plant needs to complete growth and development.

Four T1 events are further evaluated in the T2 generation following the same evaluation procedure as for the T1 generation but with more individuals per event. From the stage of sowing until the stage of maturity the plants are passed several times through a digital imaging cabinet. At each time point digital images (2048×1536 pixels, 16 million colours) were taken of each plant from at least 6 different angles.

### Drought Screen

Plants from T2 seeds were grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution was used during the first two weeks after transplanting the plantlets in the greenhouse. After the first two weeks, 25 mM of salt (NaCl) was added to the nutrient solution, until the plants were harvested. Seed-related parameters were then measured.

### 9.3.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

9.3.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 9.4

### Subgroup III Grx

### 9.4.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They were then transferred to a “dry” section where irrigation was withheld. Humidity probes were inserted in randomly chosen pots to monitor the soil water content (SWC). When SWC went below certain thresholds, the plants were automatically re-watered continuously until a normal level was reached again. The plants were then re-transferred again to normal conditions. The rest of the cultivation (plant maturation, seed harvest) was the same as for plants not grown under abiotic stress conditions. Growth and yield parameters are recorded as detailed for growth under normal conditions.

### Nitrogen Use Efficiency Screen

Rice plants from T2 seeds are grown in potting soil under normal conditions except for the nutrient solution. The pots were watered from transplantation to maturation with a specific nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### Salt Stress Screen

Plants are grown on a substrate made of coco fibers and argex (3 to 1 ratio). A normal nutrient solution was used during the first two weeks after transplanting the plantlets in the greenhouse. After the first two weeks, 25 mM of salt (NaCl) was added to the nutrient solution, until the plants were harvested. Seed-related parameters were then measured.

### 9.4.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 9.4.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour was determined by counting the total number of pixels from aboveground plant parts discriminated from the background. This value was averaged for the pictures taken on the same time point from different angles and was converted to a physical surface value expressed in square mm by calibration. The results described below are for plants three weeks post-germination.

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### Example 9.5

### Sister of FT Sequences

### 9.5.1 Evaluation Setup

Approximately 35 independent T0 rice transformants were generated. The primary transformants were transferred from a tissue culture chamber to a greenhouse for growing and harvest of T1 seed. Six events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### Drought Screen

Plants from T2 seeds are grown in potting soil under normal conditions until they approached the heading stage. They are then transferred to a “dry” section where irrigation is withheld. Humidity probes are inserted in randomly chosen pots to monitor the soil water content (SWC). When SWC falls below certain thresholds, the plants are automatically watered continuously until a normal level is reached. The plants are then re-transferred to normal conditions. The rest of the cultivation (plant maturation, seed harvest) is the same as for plants not grown under abiotic stress conditions. Parameters are recorded as detailed for growth under normal conditions.

### Nitrogen Use Efficiency Screen

Rice plants from T2 seeds are grown in potting soil under normal conditions except for the nutrient solution. The pots are watered from transplantation to maturation with a specific nutrient solution containing reduced N nitrogen (N) content, usually between 7 to 8 times less. The rest of the cultivation (plant maturation, seed harvest) is the same as for plants not grown under abiotic stress. Parameters are recorded as detailed for growth under normal conditions.

### Salt Stress Screen

Plants are grown on a substrate made of coco fibers and argex (3 to 1 ratio). A normal nutrient solution is used during the first two weeks after transplanting the plantlets in the greenhouse. After the first two weeks, 25 mM of salt (NaCl) is added to the nutrient solution, until the plants are harvested. Seed-related parameters were then measured.

### 9.5.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 9.5.3 Parameters Measured

### Biomass-Related Parameter Measurement

From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

### Example 10

### Results of the Phenotypic Evaluation of the Transgenic Plants

### Example 10.1

### PRE-Like Sequences

All 6 tested lines showed an increase of thousand kernel weight (TKW). The overall increase for thousand kernel weight was more than 5%, with a p-value <0.0000. An increase in TKW was also observed in plants grown under nitrogen deficiency. All 6 lines showed an increase in TKW.

### Example 10.2

### SCE1 Sequences

The results of the evaluation of transgenic rice plants expressing an Arath_SCE1_{—}1 nucleic acid under the non-stress conditions screen (YS: yield screen) and under nitrogen use deficiency screen (NUE) are presented below. In the YS screen, an increase of at least 5% was observed for aboveground biomass (AreaMax), and root biomass (RootMax) in the transgenic plants with respect of their corresponding nullyzygous control plants (Table E1). In the NUE screen an increase of at least 5% was observed for aboveground biomass (AreaMax), early vigour (EmerVigor), number of first panicles (firstpan) and total number of seeds per plant (nrtotalseed), in the transgenic plants with respect of their corresponding nullyzygous control plants (Table E2).

TABLE E1
Results evaluation in YS: yield screen.

| | % increase in transgenic plants |
|---|---|
| Parameter | versus the nullizygous |
| AreaMax | 13.3 |
| RootMax | 8 |

TABLE E2
Results evaluation in NUE screen.

| | % increase in transgenic plants |
|---|---|
| Parameter | versus the nullizygous |
| AreaMax | 17.8 |
| EmerVigor | 22.8 |
| firstpan | 7.5 |
| nrtotalseed | 16 |

### Example 10.3

### YEF1 Sequences

The results of the evaluation of transgenic rice plants expressing a Le_YEF1_{—}1 nucleic acid (SEQ ID NO: is given in Table A3) under non-stress conditions and drought stress conditions are presented below. An increase of at least 5% for the total weight of the seeds, the number of filled seeds, the seed filling rate, the harvest index and of at least 3% for the thousand kernel weight was observed in the transgenic plants compared to their respective nullyzygous controls when grown under the drought conditions (Table E3). Plant evaluation under the yield screen revealed an increase of at least 5% for the total weight of the seeds and/or at least 3% for the thousand kernel weight (Table E4).

TABLE E3
Plant evaluation results under drought conditions.

| | % increase in transgenic plant |
|---|---|
| Yield-related parameter | versus control nullizygous plant |
| total weight of the seeds | 53 |
| number of filled seeds | 40 |
| seed filling rate | 33 |
| harvest index | 54 |
| thousand kernel weight | 13 |

TABLE E4
Plant evaluation results under non-stress conditions.

| | % increase in transgenic plant |
|---|---|
| Yield-related parameter | versus control nullizygous plant |
| total weight of the seeds | 8 |
| thousand kernel weight | 8 |

### Example 10.4

### Subgroup III Grx Sequences

The results of the evaluation of transgenic rice plants expressing a subgroup III Grx nucleic acid represented by SEQ ID NO: 282 under non-stress conditions are presented below. The overall percentage difference of all events compared to corresponding nullizygotes is given.

| Parameter | % Difference |
|---|---|
| Aboveground area | 5.7% |
| Emergence vigour | 25.1% |
| Total seed weight | 17.7% |
| Total No. seeds | 9.3% |
| No. filled seeds | 15.0% |
| Fill rate | 5.8% |
| Flowers per panicle | 5.5% |
| Harvest index | 11.5% |
| TKW | 2.9% |

### Example 10.5

### Sister of FT Sequences

The results of the evaluation of transgenic rice plants expressing an Sister of FT nucleic acid according to SEQ ID NO: 439 under non-stress conditions give a greater than two-fold increase in the root:shoot index of transgenic plants compared to nullizygotes.

1. A method for enhancing yield-related traits in plants relative to control plants, comprising modulating expression in a plant of a nucleic acid encoding a PRE-like polypeptide, or an SCE1 polypeptide, or a YEF1 polypeptide, or a subgroup III Grx polypeptide, or a Sister of FT protein, and wherein said nucleic acid encodes a Sister of FT protein the yield-related trait is an alteration of the ratio of roots to shoots comprising modulating expression in a plant of a nucleic acid encoding a Sister of FT polypeptide or a homologue thereof having at least overall sequence identity to the amino acid sequence represented by SEQ ID NO: 440.

2. The method of claim 1, wherein said PRE-like polypeptide comprises one or more of the following motifs: Motif 1 (SEQ ID NO: 7), Motif 2 (SEQ ID NO: 8) and Motif 3 (SEQ ID NO: 9).

3. The method of claim 1, wherein said modulated expression is effected by introducing and expressing in a plant a nucleic acid encoding a PRE-like polypeptide.

4. The method of claim 1, wherein said nucleic acid encoding a PRE-like polypeptide encodes any one of the proteins listed in Table A or is a portion of such a nucleic acid, or a nucleic acid capable of hybridizing with such a nucleic acid.

5. The method of claim 1, wherein said nucleic acid sequence encodes an orthologue or paralogue of any of the proteins given in Table A.

6. The method of claim 1, wherein said enhanced yield-related traits comprise increased yield, or increased seed yield relative to control plants, provided that said increased seed yield does not encompass increased seed oil content.

7. The method of claim 1, wherein said enhanced yield-related traits are obtained under non-stress conditions.

8. The method of claim 1, wherein said enhanced yield-related traits are obtained under conditions of drought stress, salt stress or nitrogen deficiency.

9. The method of claim 3, wherein said nucleic acid is operably linked to a constitutive promoter, preferably to a GOS2 promoter, or a GOS2 promoter from rice.

10. The method of claim 1, wherein said nucleic acid encoding a PRE-like polypeptide is of plant origin, from a dicotyledonous plant, from the family Poaceae, from the genus Triticum, or from Triticum aestivum.

11. A plant or part thereof, including seeds, obtainable by a the method of claim 1, wherein said plant or part thereof comprises a recombinant nucleic acid encoding a PRE-like polypeptide.

12. A construct comprising:
(i) the nucleic acid encoding a PRE-like polypeptide as defined in claim 2;
(ii) one or more control sequences capable of driving expression of the nucleic acid sequence of (a); and optionally
(iii) a transcription termination sequence.

13. The construct of claim 12, wherein one of said control sequences is a constitutive promoter, a GOS2 promoter, or a GOS2 promoter from rice.

14. A method for making plants having increased yield or increased seed yield relative to control plants, comprising utilizing the construct of claim 12.

15. A plant, plant part or plant cell transformed with the construct of claim 12.

16. A method for the production of a transgenic plant having increased yield, particularly increased biomass and/or increased seed yield relative to control plants, comprising:
(i) introducing and expressing in a plant a nucleic acid encoding a PRE-like polypeptide as defined in claim 2; and
(ii) cultivating the plant cell under conditions promoting plant growth and development.

17. A transgenic plant having increased yield or increased seed yield, relative to control plants, resulting from modulated expression of a nucleic acid encoding a PRE-like polypeptide as defined in claim 2, or a transgenic plant cell derived from said transgenic plant.

18. The transgenic plant to of claim 11, or a transgenic plant cell derived thereof, wherein said plant is a crop plant or a monocot or a cereal, such as rice, maize, wheat, barley, millet, rye, triticale, sorghum emmer, spelt, secale, einkorn, teff, milo and oats.

19. Harvestable parts of the plant of claim 18, wherein said harvestable parts are shoot biomass and/or seeds.

20. Products derived from the plant of claim 18 and/or from harvestable parts of said plant.

21. A method for increasing yield or increasing seed yield in plants, relative to control plants, comprising utilizing a nucleic acid encoding a PRE-like polypeptide.
